SEQUENCE LISTING (24 sequences)

SEQ ID NO: 1 - protein, 245 aa, zea mays
GPPKVPPGPNITTNYNGKWLTARATWYGQPNGAGAPDNGGACGIKNVNLPPYSGMTACGNVPIFKDGKGCGSCYEVRCKEKPECSGNPVTVYITDMNYEPIAPYHFDLSGKAFGSLAKPGLNDKIRHCGIMDVEFRRVRCKYPAGQKIVFHIEKGCNPNYLAVLVKYVADDGDIVLMEIQDKLSAEWKPMKLSWGAIWRMDTAKALKGPFSIRLTSESGKKVIAKDVIPANWRPDAVYTSNVQFY

SEQ ID NO: 2 - protein, 250 aa, Artificial
  Description of artificial sequence  conserved domains of expansin proteins.
MAFSYSPFSSLFLLPFFFVFTFADYGGWQSGHATFYGGGDASGTMGGACGYGNLYSQGYGTNTVALSTALFNNGLSCGACFEMTCTNDPKWCLPGTIRVTATNFCPPNFALPNNNGGWCNPPLQHFDMAEPAFLQIAQYRAGIVPVSFRRVPCMKKGGVRFTINGHSYFNLVLITNVGGAGDVHSVSIKGSRTGWQSMSRNWGQNWQSNNYLNGQGLSFQVTLSDGRTLTAYNLVPSNWQFGQTYEGPQF

SEQ ID NO: 3 - protein, 4 aa, artificial
  Description of artificial sequence   conserved motif of expansin proteins.
TWYG

SEQ ID NO: 4 - protein, 5 aa, artificial
  Description of artificial sequence  conserved motif of expansin proteins.
GGACG

SEQ ID NO: 5 - protein, 9 aa, artificial
  Description of artificial sequence motif in the N-terminal extension in EXPB1.
VPPGPNITT

SEQ ID NO: 6 - protein, 13 aa, artificial
  Description of artificial sequence amino acid residues of EXPB1 that were studied.
GPPKVPPGXNITT

SEQ ID NO: 7 - protein, 13 aa, artificial
  Description of artificial sequence amino acid residues of EXPB1 that were studied.
NYNGKWLTARATW

SEQ ID NO: 8 - protein, 13 aa, artificial
  Description of artificial sequence amino acid residues of EXPB1 that were studied.
YGQPNGAGAPDNG

SEQ ID NO: 9 - protein, 13 aa, artificial
  Description of artificial sequence amino acid residues of EXPB1 that were studied.
GACGIKNVNLPPY

SEQ ID NO: 10 - protein, 13 aa, artificial
  Description of artificial sequence amino acid residues of EXPB1 that were studied.
SGMTACGNVPIFK

SEQ ID NO: 11 - protein, 13 aa, artificial
  Description of artificial sequence amino acid residues of EXPB1 that were studied.
DGKGCGSCYEVRC

SEQ ID NO: 12 - protein, 13 aa, artificial
  Description of artificial sequence amino acid residues of EXPB1 that were studied.
KEKPECSGNPVTV

SEQ ID NO: 13 - protein, 13 aa, artificial
  Description of artificial sequence amino acid residues of EXPB1 that were studied.
YITDMNYEPIAPY

SEQ ID NO: 14 - protein, 13 aa, artificial
  Description of artificial sequence amino acid residues of EXPB1 that were studied.
HFDLSGKAFGSLA

SEQ ID NO: 15 - protein, 13 aa, artificial
  Description of artificial sequence amino acid residues of EXPB1 that were studied.
KPGLNDKIRHCGI

SEQ ID NO: 16 - protein, 13 aa, artificial
  Description of artificial sequence amino acid residues of EXPB1 that were studied.
MDVEFRRVRCKYP

SEQ ID NO: 17 - protein, 13 aa, artificial
  Description of artificial sequence amino acid residues of EXPB1 that were studied.
AGQKIVFHIEKGC

SEQ ID NO: 18 - protein, 13 aa, artificial
  Description of artificial sequence amino acid residues of EXPB1 that were studied.
NPNYLAVLVKYVA

SEQ ID NO: 19 - protein, 13 aa, artificial
  Description of artificial sequence amino acid residues of EXPB1 that were studied.
DDGDIVLMEIQDK

SEQ ID NO: 20 - protein, 13 aa, artificial
  Description of artificial sequence amino acid residues of EXPB1 that were studied.
LSAEWKPMKLSWG

SEQ ID NO: 21 - protein, 13 aa, artificial
  Description of artificial sequence amino acid residues of EXPB1 that were studied.
AIWRMDTAKALKG

SEQ ID NO: 22 - protein, 13 aa, artificial
  Description of artificial sequence amino acid residues of EXPB1 that were studied.
PFSIRLTSESGKK

SEQ ID NO: 23 - protein, 13 aa, artificial
  Description of artificial sequence amino acid residues of EXPB1 that were studied.
VIAKDVIPANWRP

SEQ ID NO: 24 - protein, 11 aa, artificial
  Description of artificial sequence amino acid residues of EXPB1 that were studied.
DAVYTSNVQFY

(19) United States
(12) Patent Application Publication
(10) Pub. No.: US 2008/0201123 A1
(43) Pub. Date: Aug. 21, 2008

(54) Increased activity and efficiency of expansin-like proteins

(75) Inventor: Daniel J. Cosgrove, Port Matilda, PA (US)

Correspondence Address: MCKEE, VOORHEES & SEASE, P.L.C.;ATTN: PENNSYLVANIA STATE UNIVERSITY, 801 GRAND AVENUE, SUITE 3200, DES MOINES, IA, 50309-2721, US

(73) Assignee: THE PENN STATE RESEARCH FOUNDATION, University Park, PA (US)

(21) Appl. No.: 11/893,509

(22) Filed: Aug. 16, 2007

Related U.S. Application Data
(60) Provisional application No. 60/822,716, filed on Aug. 17, 2006.

(51) Int. Cl. G06G 7/48; C07K 14/42; C12Q 1/25

(52) U.S. Cl. 703/11; 530/370; 435/4

(57) ABSTRACT

The invention relates to crystal structure and activities of Beta-expansins and grass pollen allergens and identification of key regions essential to maximize activity and to identify sequence motifs which correlate with activity.

### CROSS-REFERENCE TO RELATED APPLICATIONS

This application claims priority under 35 U.S.C. § 119 of a provisional application Ser. No. 60/822,716 filed Aug. 17, 2006, which application is hereby incorporated by reference in its entirety.

### BACKGROUND OF THE INVENTION

Cell wall proteins play important roles in regulating cell wall extensibility which in turn controls cell enlargement. Among cell wall proteins studied to date, expansins are unique in their ability to induce immediate cell wall extension in vitro and cell expansion in vivo. Expansins are extracellular proteins that promote plant cell wall enlargement, evidently by disrupting noncovalent bonding between cellulose microfibrils and matrix polymers (McQueen-Mason, S., et al., (1994) Proc. Natl. Acad. Sci. USA 91:6574-6578; McQueen-Mason, S., et al., (1992) Plant Cell 4:1425-1433).

Since their first isolation from cucumber hypocotyls, expansin proteins have been identified in many plant species and organs on the basis of activity assays and immunoblotting. Examples include tomato leaves, oat coleoptiles, maize roots, rice internodes, tobacco cell cultures, and various fruits. The original sequencing of cucumber expansin cDNAs has impacted our understanding of expansins in several respects. First, expansin genes have now been identified in many other plant species, and they appear to be restricted largely to the plant kingdom. Second, expansins comprise a large multigene family in the plant species. For example, in Arabidopsis, 31 expansin genes have been identified. Third, studies of expression and localization of expansin mRNA are providing new insights and hypothesis concerning the developmental roles of specific expansin genes. And fourth, sequence comparisons have led to the discovery that another group of proteins known previously as group-1 grass pollen allergens, have expansin activity. These pollen-specific proteins are closely related to a group of sequences known primarily from expressed sequence tag (EST) databases. These EST sequences, together with the group-1 pollen allergens, have now been classified as beta-expansins, whereas the original group of expansins are now classified as alpha-expansins. The α-expansins are described in U.S. Pat. Nos. 5,959,082 and 5,990,283 to Cosgrove et al., which are herein incorporated by reference. β-expansins, in general, are the subject of a previously filed U.S. patent application Ser. No. 09/071,252 filed May 1, 1998. Although these two expansin families have only about 20% amino acid identity, they are similar in size, they share a number of conserved motifs, and they have similar wall-loosening activities.

To date, most studies have focused on α-expansins, and limited work has been done on β-expansins. A soybean cytokinin-induced gene known as CIM1 is now classified as a β-expansin, but the biological function of the CIM1 protein is uncertain. The maize group-1 pollen allergen, Zea m1, has wall-loosening activity with high specificity for grass cell walls. This β-expansin is hypothesized to aid fertilization by loosening the cell walls of the stigma and style, thereby facilitating penetration of the pollen tube. Many other β-expansin sequences are found in the rice EST databases, and most of these sequences come from cDNA libraries made from young seedlings and other plant materials that do not contain pollen. Thus, their biological functions clearly differ from those of the group-1 pollen allergens. These so-called vegetative β-expansins are hypothesized to function in cell enlargement and other processes where wall loosening is required. It is notable that the rice EST collection contains at least 75 entries representing at least 10 distinct β-expansin genes. In contrast, only a single Arabidopsis EST is classified as a β-expansin (although a total of five β-expansin genes are found in the Arabidopsis genome). The disparity in the number of β-expansin entries in the rice and Arabidopsis EST collection, together with the specificity of Zea m1 activity for grass walls, leads to the proposal that β-expansins have evolved specialized function in conjunction with the evolution of the grass cell wall, which has a distinctive set of matrix polysaccharide and structural proteins compared with other land plants. If this is true, one would expect to find an abundance of β-expansin homology in other grasses, with expression in many tissues beside pollen.

Recently, Group 2 and Group 3 allergens (designated group 2/3 allergens or also termed HED2 proteins) have also been shown to have expansin activity. Although these allergens from grass pollen have been studied for many years by immunologists concerned with how they elicit hay fever and related allergic responses in humans, the native activity and biological roles of these proteins have not been examined. Group 2/3 grass pollen allergens are distinguished by pI and immuno-cross reactivity, but accumulating sequence information indicates that they belong to the same protein family, genes for group 2/3 allergens encode a protein with a signal peptide and a mature protein with statistically significant sequence similarity (up to 42% identity) with domain 2 of expansins, with the greatest similarly to group-1 allergen sub-class of β-expansins.

Of the two families of expansins, α- and β-group 2/3 allergens are closest in sequence to the subset β-expansins known to immunologists as the grass pollen group 1 allergens.

Once identified, however, proteins with expansin activity including β-expansins, α-expansins, and group 2/3 allergens, or HED proteins all of which are proteins capable of inducing cell wall extension, have utility not only in the engineered extension of cell walls in living plants but foreseeably in commercial applications where their chemical reactivity. Expansins can disrupt noncovalent associations of cellulose, and as such have particular utility in the paper recycling industry. Paper recycling is a growing concern and will prove more important as the nation's landfill sites become scarcer and more expensive. Paper derives its mechanical strength from hydrogen bonding between paper fibers, which are composed primarily of cellulose. During paper recycling, the hydrogen bonding between paper fibers is disrupted by chemical and mechanical means prior to re-forming new paper products. Proteins which cause cell expansion are thus intrinsically well suited to paper recycling, especially when the proteins are nontoxic and otherwise innocuous, and when the proteins can break down paper products which are resistant to other chemical and enzymatic means of degradation. Use of proteins of this type could thus expand the range of recyclable papers.

Other modes of application of expansins, include production of virgin paper. Pulp for virgin paper is made by disrupting the bonding between plant fibers. For the reasons identified above, expansins are useful in the production of paper pulp from plant tissues. Use of expansins can substitute for harsher chemicals now in use and thereby reduce the financial and environmental costs associated with disposing of these harsh chemicals. The use of expansins can also result in higher quality plant fibers because they would be less degraded than fibers currently obtained by harsher treatments.

Still other modes of applications include the production of ethanol. One of the major limitations and costs associated with ethanol production from cellulose is conversion of cellulose to simple fermentable sugars. Because of the crystalline structure of cellulose, its enzymatic conversion to sugars takes a considerable amount of time and requires large quantities of cellulase enzymes, which are expensive. Likewise for the production of chemically-modified cellulose derivatives, cellulose must be made accessible to reactive chemical agents, this usually requiring high temperature, pressures and harsh chemical conditions. Furthermore, the efficient digestion of straws, hay, and other plant materials by ruminants and other animals is limited by the accessibility of cellulose to the digestive enzymes in the animals' gut. Expansin proteins, particularly, group 2/3/allergans have been shown to made cellulose more easily degraded by cellulase enaymes.

Thus, a continuing need remains for the identification, characterization, and optimization of expansins—proteins which can be characterized as catalysts of the extension of plant cell walls and the weakening of the hydrogen bonds in the pure cellulose.

### SUMMARY OF THE INVENTION

The invention relates to crystal structure and activities of Beta-expansins and grass pollen allergens and identification of key regions essential to maximize activity and to identify sequence motifs which correlate with activity.

According to the invention, Beta-expansin structure has been delineated to identify critical regions for activity. For example the β-expansin molecule consists of two domains closely packed and aligned to form a long shallow groove with potential to bind a glycan backbone. The domain has first residues 19-140 which form a protein fold, the second domain includes 147-245 composed of eight β-strands assembled into two anti-parallel sheets. Essential residues include surface aromatic residues W194 and Y160 which are in line with W25 and Y27. From this data one can extrapolate to identify essential regions of conservation to develop modified expansins with improved properties, efficiencies and the like.

### DETAILED DESCRIPTION OF THE FIGURES

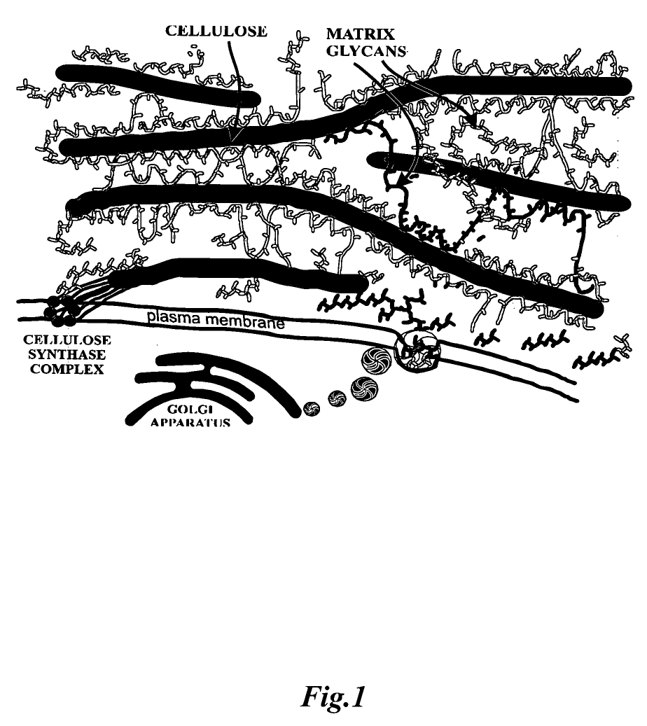

FIG. 1 is a schematic diagram of the plant cell wall. Cellulose microfibrils are synthesized by large complexes in the plasma membrane and are glued together by branched matrix polysaccharides synthesized in the Golgi and deposited by vesicles along the inner surface of the cell wall. The ˜4 nm wide cellulose microfibril in cross-section consists of ˜36 β-(1→4)-D-glucans organized into a crystalline array. Polysaccharides such as arabinoxylan and xyloglucan spontaneously bind to the surface of cellulose and may also be entrapped during coalescence of the β-(1→4)-D-glucans to form the microfibril. Hydrophilic pectins and structural proteins (not shown) also make up the matrix between cellulose microfibrils and influence the wall's physical properties.

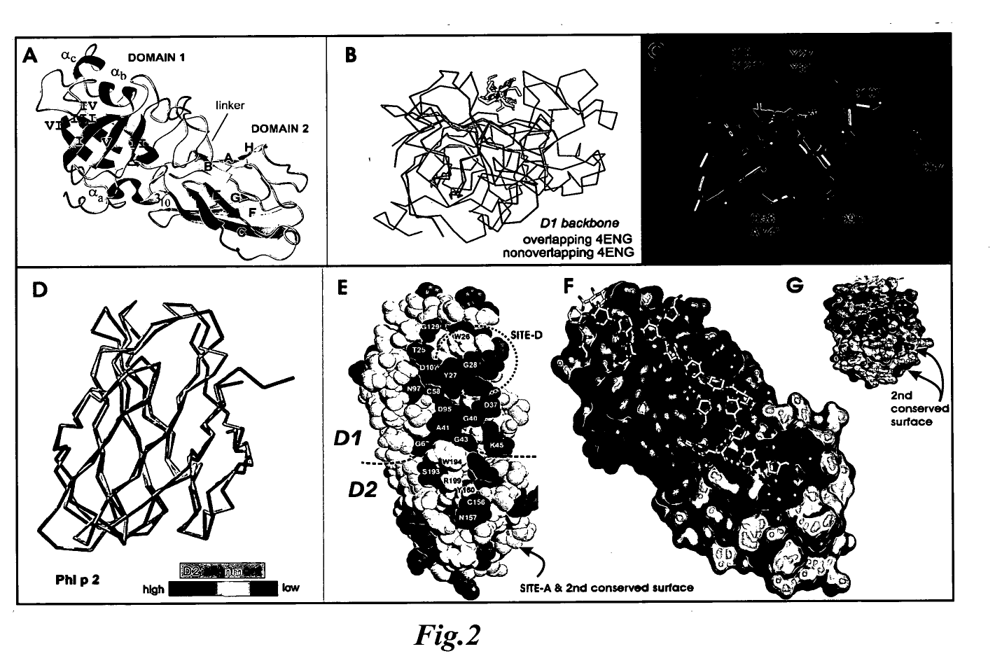

FIG. 2 is a diagram showing the structure of EXPB1 (PDB 2HCZ). A: Ribbon model of EXPB1, showing the overall configuration of the two domains. B: Superposition of the peptide backbone of EXPB1 D1 (shown entirely in red) with the peptide backbone of Humicola Cel45 (PDB code 4ENG), colored green for regions of good alignment with EXPB1, grey otherwise. The yellow residues indicate cellohexaose from the 4ENG model. C: Superposition of residues making up the catalytic site of Humicola Cel45 (blue) and corresponding residues of EXPB1 (red). Other conserved acidic residues this region of EXPB1 are shown in purple. D: Superposition of EXPB1 D2 (colored) and Phl p 2 (grey), a group-2/3 grass pollen allergen (PDB code 1WHO). Coloring scale from best to poorest alignment of peptide backbones: blue-green-yellow-red. E: Top view of the conserved surface of EXPB1, color coded to indicate conservation (red=most conserved; blue=least conserved; white=intermediate). Conserved residues are labelled, and the locations of two antigenic epitopes are indicated (site-D, site-A). F: A model of glucurono-arabinoxylan (yellow and red) was manually fitted to the long open groove of EXPB1 using the program O (66) and subsequently energy minimized using the program CNS (67). Green residues are from D1, cyan residues are from D2 and red residues are the conserved residues identified in panel E. G: End view of same model as in F. Image in E was generated from the program CONSURF (68) using the alignment of 80 EXPB proteins in GenBank and 2HCZ after removal of the N-terminal extension. Images in G and F were generated with PYMOL (DeLano Scientific) after removal of the N-terminal extension.

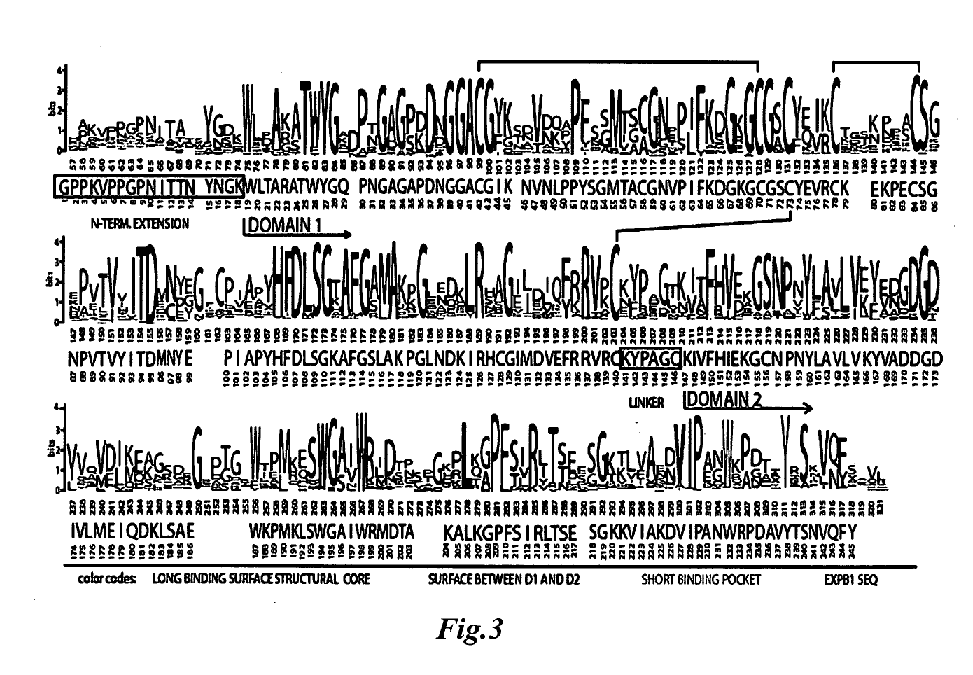

FIG. 3 is a graph showing the EXPB sequence logo based on 80 EXPB proteins from Genbank, aligned with the sequence of maize EXPB1 (green) and color coded to indicate the structural role of the conserved residues. Residues with unspecified role are indicated in grey. The size of the one-letter amino acid code in the sequence logo indicates the degree of conservation on a logarithmic scale. The logo was generated with the web server at world wide web, weblogo.berkeley.edu. Black lines between Cys residues indicate disulfide bonds.

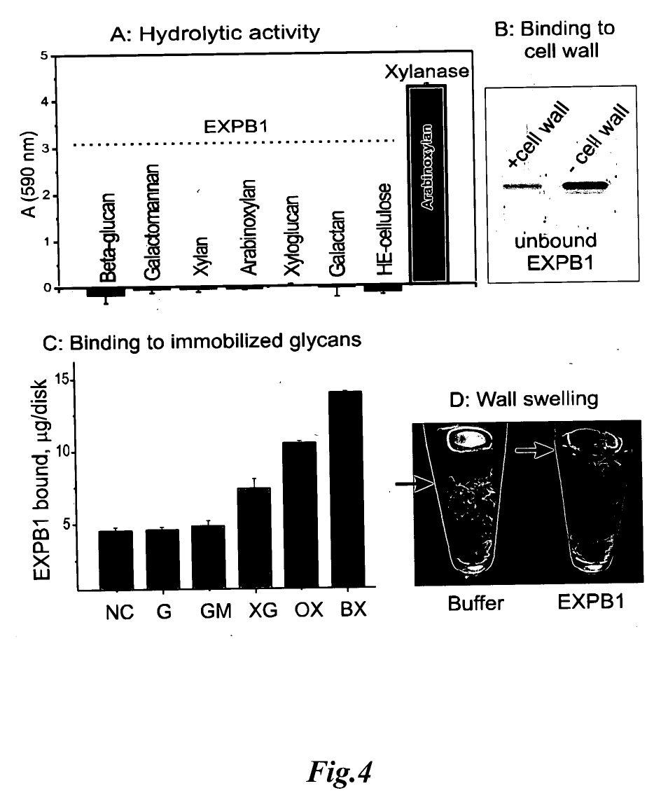

FIG. 4 shows the hydrolytic activity of expansin B1 against various wall polyaccharides and glycans. A: Hydrolytic activity of EXPB1 against various wall polysaccharides. Data are means ±SEM (n=3). The positive control with arabinoxylan is a crude extract of maize pollen containing endoxylanase activity (69). B: Maize cell walls bind EXPB1. After incubation of EXPB1+/−cell wall, protein remaining in the supernatant was analyzed by SDS-PAGE and stained with SYPRO Ruby. C: EXPB1 binding to isolated polysaccharides immobilized onto nitrocellulose membrane; NC=nitrocellulose membrane along; G=β-(1→3), (1→4)-D-glucan, GM=glucomannan; XG=xyloglucan; OX=oat xylan; BX=birch xylan. Data are means ±SEM (n=3). D: Swelling of maize cell walls after 48-h incubation +/−EXPB1. Methods as described in the binding studies.

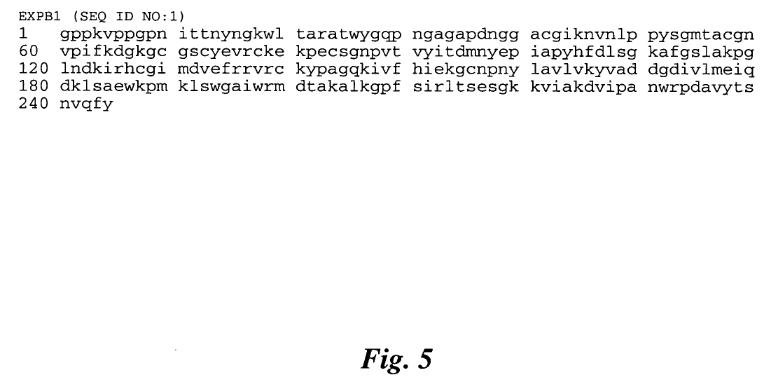

FIG. 5 is the amino acid sequence for Zea m 1 isoform d. (Genbank accession number AAO45608).

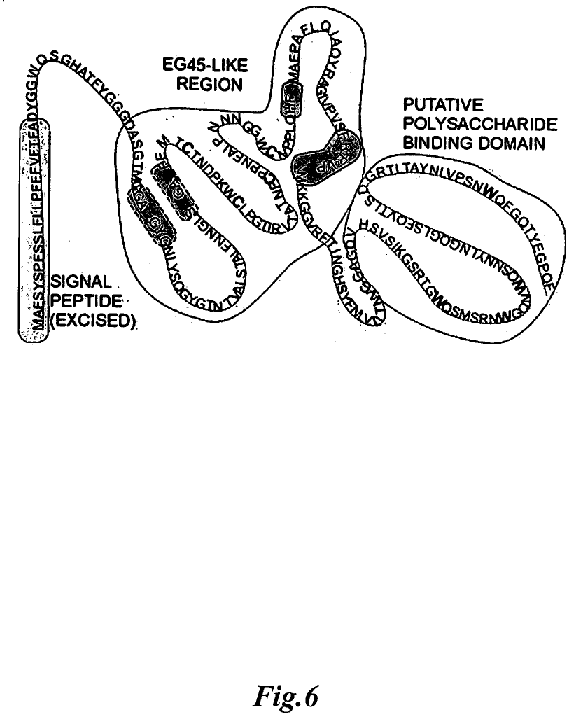

FIG. 6 is a schematic of the conserved domains of expansin proteins.

### DETAILED DESCRIPTION OF THE INVENTION

### Background to Crystallization

It is well-known in the art of protein chemistry, that crystallizing a protein is a difficult process. In fact it is now evident that protein crystallization is the main hurdle in protein structure determination. There are many references which describe the difficulties associated with growing protein crystals. For example, Kierzek, A. M. and Zielenkiewicz, P., (2001), Biophysical Chemistry, 91:1-20, Models of protein crystal growth, and Wiencek, J. M. (1999) Annu. Rev. Biomed. Eng., 1:505-534, New Strategies for crystal growth. It is commonly held that crystallization of protein molecules from solution is the major obstacle in the process of determining protein structures. The reasons for this are many; proteins are complex molecules, and the delicate balance involving specific and non-specific interactions with other protein molecules and small molecules in solution is difficult to predict.

Each protein crystallizes under a unique set of conditions which cannot be predicted in advance. Simply supersaturating the protein to bring it out of solution may not work, the result would, in most cases, be an amorphous precipitate. Many precipitating agents are used, common ones are different salts, and polyethylene glycols, but others are known. In addition, additives such as metals and detergents can be added to modulate the behavior of the protein in solution. Many kits are available (e.g. from Hampton Research), which attempt to cover as many parameters in crystallization space as possible, but in many cases these are just a starting point to optimize crystalline precipitates and crystals which are unsuitable for diffraction analysis. Successful crystallization is aided by a knowledge of the proteins behavior in terms of solubility, dependence on metal ions for correct folding or activity, interactions with other molecules and any other information that is available. Even so, crystallization of proteins is often regarded as a time-consuming process, whereby subsequent experiments build on observations of past trials.

In cases where protein crystals are obtained, these are not necessarily always suitable for diffraction analysis; they may be limited in resolution, and it may subsequently be difficult to improve them to the point at which they will diffract to the resolution required for analysis. Limited resolution in a crystal can be due to several things. It may be due to intrinsic mobility of the protein within the crystal, which can be difficult to overcome, even with other crystal forms. It may be due to high solvent content within the crystal, which consequently results in weak scattering. Alternatively, it could be due to defects within the crystal lattice which mean that the diffracted x-rays will not be completely in phase from unit to unit within the lattice. Any one of these or a combination of these could mean that the crystals are not suitable for structure determination.

Some proteins never crystallize, and after a reasonable attempt it is necessary to examine the protein itself and consider whether it is possible to make individual domains, different N or C-terminal truncations, or point mutations. It is often hard to predict how a protein could be re-engineered in such a manner as to improve crystallizability. Our understanding of crystallization mechanisms are still incomplete and the factors of protein structure which are involved in crystallization are poorly understood.

### Determination of Protein Structure.

A mathematical operation termed a Fourier transform relates the diffraction pattern observed from a crystal and the molecular structure of the protein comprising the crystal. A Fourier transform may be considered to be a summation of sine and cosine waves each with a defined amplitude and phase. Thus, in theory, it is possible to calculate the electron density associated with a protein structure by carrying out an inverse Fourier transform on the diffraction data. This, however, requires amplitude and phase information to be extracted from the diffraction data. Amplitude information may be obtained by analyzing the intensities of the spots within a diffraction pattern. Current technologies for generating x-rays and recording diffraction data lead to loss of all phase information. This “phase information” must be in some way recovered and the loss of this information represents the “crystallographic phase problem”. The phase information necessary for carrying out the inverse Fourier transform can be obtained via a variety of methods. If a protein structure exists a set of theoretical amplitudes and phases may be calculated using the protein model and then the theoretical phases combined with the experimentally derived amplitudes. An electron density map may then be calculated and the protein structure observed.

If there is no known structure of the protein then alternative methods for obtaining phases must be explored. One method is multiple isomorphous replacement (MIR). This relies on soaking “heavy atom” (i.e. platinum, uranium, mercury, etc) compounds into the crystals and observing how their incorporation into the crystals modifies the spot intensities observed in the diffraction pattern. This method relies on the heavy atoms being incorporated into the protein at a finite number of defined sites. It is a pre-requisite of an isomorphous replacement experiment that the heavy atom soaked crystals remain isomorphous. That is, there should be no appreciable alterations in the physical characteristics of the protein crystal (i.e. perturbations to crystallographic cell dimensions, or significant loss of resolution). Perturbations to the physical properties of the crystal are termed non-isomorphisms and prevent this type of experiment being successfully completed. Successful isomorphous incorporation of heavy atoms into a protein crystal results in the intensities of the spots within the diffraction pattern obtained from the crystal being modified, as compared to the data collected from an identical, unsoaked, (native) crystal. The diffraction data obtained from a successful isomorphous replacement experiment are termed a “derivative” dataset. By mathematically analyzing the “native” and “derivative” datasets it is possible to extract preliminary phase information from the datasets. This phase information, when combined with the experimentally obtained amplitudes from the native dataset, enables an electron density map of the unknown protein molecule to be calculated using the Fourier transform method.

An alternative method for obtaining phase information for a protein of unknown structure is to perform a multi-wavelength anomalous dispersion (MAD) experiment. This relies on the absorption of X-rays by electrons at certain characteristic X-ray wavelengths. Different elements have different characteristic absorption edges. Anomalous scattering by atoms within a protein will modify the diffraction pattern obtained from the protein crystal. Thus if a protein contains atoms which are capable of anomalous scattering a diffraction dataset (anomalous dataset) may be collected at an X-ray wavelength at which this anomalous scattering is maximal. By altering the X-ray wavelength to a value at which there is no anomalous scattering a native dataset may then be collected. Similarly to the MIR case, by mathematically processing the anomalous and native datasets the phase information necessary for the calculation of an electron density map may be determined. The most usual way to introduce anomalous scatterers into a protein is to replace the sulphur containing methionine amino acid residues with selenium containing seleno-methionine residues. This is done by generating recombinant protein that is isolated from cells grown on growth media that contain seleno-methionine. Selenium is capable of anomalously scattering X-rays and may thus be used for a MAD experiment. Further methods for phase determination such as single isomorphous replacement (SIR), single isomorphous replacement anomalous scattering (SIRAS) and direct methods exist, but the principles behind them are similar to MIR and MAD.

The final method generally available for the calculation of the phases necessary for the determination of an unknown protein structure is molecular replacement. This method relies upon the assumption that proteins with similar amino acid sequences (primary sequences) will have a similar fold and three-dimensional structure (tertiary structure). Proteins related by amino acid sequence are termed homologous proteins. If an X-ray diffraction dataset has been collected from a crystal whose protein structure is not known, but a structure has been determined for a homologous protein, then molecular replacement can be attempted. Molecular replacement is a mathematical process that attempts to correlate the dataset obtained from a new protein crystal with the theoretical diffraction pattern calculated for a protein of known structure. If the correlation is sufficiently high some phase information can be extracted from the known protein structure and combined with the amplitudes obtained from the new protein dataset. This enables calculation of a preliminary electron density map for the protein of unknown structure.

If an electron density map has been calculated for a protein of unknown structure then the amino acids comprising the protein must be fitted into the electron density for the protein. This is normally done manually, although high resolution data may enable automatic model building. The process of model building and fitting the amino acids to the electron density can be both a time consuming and laborious process. Once the amino acids have been fitted to the electron density it is necessary to refine the structure. Refinement attempts to maximize the correlation between the experimentally calculated electron density and the electron density calculated from the protein model built. Refinement also attempts to optimize the geometry and disposition of the atoms and amino acids within the user-constructed model of the protein structure. Sometimes manual re-building of the structure will be required to release the structure from local energetic minima. There are now several software packages available that enable an experimentalist to carry out refinement of a protein structure. There are certain geometry and correlation diagnostics that are used to monitor the progress of a refinement. These diagnostic parameters are monitored and rebuilding/refinement continued until the experimenter is satisfied that the structure has been adequately refined.

The present invention relates to the crystal structure of EXPB1 (Genbank accession AA045608; PDB accession 2HCZ), which allows the binding location of the polysaccharides to the compound and its activities to be investigated and determined.

Thus in one aspect, the invention provides a three dimensional structure of EXPB1 set out in FIGS. 2 and 3, and uses, described further herein below of the three dimensional structure.

According to the invention, EXPB1 contains two domains (residues 19-140 [D1] and 147-245 [D2]) connected by a short linker (residues 141-146) and aligned end to end so as to make a closely-packed irregular cylinder ˜66 Å long and 26 Å in diameter (FIG. 2A).

The two EXPB1 domains pack close to one another, making contact via H-bonds and salt bridges between basic residues (K65 and R137) in D1 and acidic residues (E217 and D171) in D2. These residues are highly conserved in the EXPB family (see annotated sequence logo in FIG. 3). Additional hydrogen bonding is found between S72 and D173, as well as between the peptide backbone for C42 and A196. The two domains also make contact via a hydrophobic patch consisting of I44, P51, Y52 and Y92 in D1 and L164, Y167 and the hydrocarbon chain of K166 in D2, residues that are mostly well conserved or have conservative substitutions in the EXPB family. Moreover, six highly conserved glycine residues (G43, 67, 69, 71, 172, 195) are found at the surfaces where the two domains make contact. The lack of side chains in the glycine residues permits close packing of the two domains.

The two EXPB1 domains align so as to form a long, shallow groove with highly conserved polar and aromatic residues suitably positioned to bind a twisted polysaccharide chain of 10 xylose residues (FIGS. 2E-G). The groove extends from the conserved G129 at one end of D1, spans across a stretch of conserved residues in D1 and D2 (see numbered residues in FIG. 2E as well as annotated sequence logo in FIG. 3) and ends at N157, a distance of some 47 Å. Many of the conserved residues common to EXPA and EXPB make up this potential binding surface, including residues in the classic expansin motifs TWYG, GGACG, and HFD (see FIG. 3).

Residues that could bind a polysaccharide by van der Waals interactions with the sugar rings include W26, Y27, G40, and G44 from D1 as well as Y160 and W194 from D2. Conserved residues that might stabilize polysaccharide binding by H-bonding include T25, D37, D95 and D107 in D1 and N157, S193 and R199 in D2.

In general aspects, the present invention is concerned with the provision of an EXPB1 structure and its use in modeling the interaction of molecular structures, e.g. potential and existing substrates, inhibitors, analogs, or fragments of such compounds, with this EXPB1 structure.

These and other aspects and embodiments of the present invention are discussed below. The above aspects of the invention, both singly and in combination, all contribute to features of the invention, which are advantageous.

The invention comprises in one paragraph a computer-based method for the analysis of the interaction of a molecular structure with an EXPB1 structure, which comprises: providing a structure comprising a three-dimensional representation of EXPB1 or a portion thereof, which representation comprises all or a portion of the coordinates of any one of figures represented in FIGS. 2 and 3 providing a molecular structure to be fitted to said EXPB1 structure or selected coordinates thereof; and fitting the molecular structure to said EXPB1 structure.

The method of the invention further comprises the steps of obtaining or synthesizing a compound which has said molecular structure; and contacting said compound with EXPB1 protein to determine the ability of said compound to interact with the EXPB1.

The method also include obtaining or synthesizing a compound which has said molecular structure; forming a complex of an EXPB1 substrate protein and said compound; and analyzing said complex by X-ray crystallography to determine the ability of said compound to interact with the EXPB1 substrate.

The method further comprises the steps of: obtaining or synthesizing a compound which has said molecular structure; and determining or predicting how said compound interacts with an EXPB1 substrate; and modifying the compound structure so as to alter the interaction between it and the substrate. The invention also includes a compound having the modified structure identified using the method and which has expansin activity.

A method of obtaining a structure of a target EXPB1 protein of unknown structure, the method comprises the steps of: providing a crystal of said target EXPB1 protein, obtaining an X-ray diffraction pattern of said crystal, calculating a three-dimensional atomic coordinate structure of said target, by modeling the structure of said target EXPB1 protein of unknown structure on the active site structure of any one of FIGS. 2-3.

The invention also includes methods where the molecular structure to be fitted is in the form of a model of a pharmacophore including but not limited to: (a) a wire-frame model; (b) a chicken-wire model; (c) a ball-and-stick model; (d) a space-filling model; (e) a stick-model; (f) a ribbon model; (g) a snake model; (h) an arrow and cylinder model; (i) an electron density map; (j) a molecular surface model.

The invention also includes a computer-based method for the analysis of molecular structures which comprises: (a) providing the coordinates of at least two atoms of an EXPB1 structure as defined in FIGS. 2 and/or 3 (b) providing the structure of a molecular structure to be fitted to the selected coordinates; and (c) fitting the structure to the selected coordinates of the EXPB1 structure. The method further contemplates that the coordinates will be a at least a portion of a binding pocket.

A computer-based method of protein design comprising: (a) providing the coordinates of at least two atoms of an EXPB1 structure as defined in any one of FIGS. 2 and 3 with a square deviation of less than 1.5 Å (“selected coordinates”); (b) providing the structures of a plurality of EXPB1 substrates or potential substrates; (c) fitting the structure of each of the EXPB1 substrates or potential substrates to the selected coordinates; and (d) determining the activity of said EXPB1 structure on said substrate or potential substrate.

A method for identifying a candidate modulator of EXPB1 comprising the steps of: (a) employing a three-dimensional structure of EXPB1, at least one sub-domain thereof, or a plurality of atoms thereof, to characterize at least one EXPB1 binding cavity, the three-dimensional structure being defined by FIGS. 2-3; and (b) identifying the candidate modulator by designing or selecting a compound for interaction with the binding cavity. The method further comprising the step of: (a) obtaining or synthesizing the candidate modulator; and (b) contacting the candidate modulator with EXPB1 to determine the ability of the candidate modulator to interact with EXPB1.

The invention also contemplates a method for determining the structure of a protein, which method comprises: providing the co-ordinates per FIGS. 2-3 or selected coordinates thereof, and either (a) positioning said co-ordinates in the crystal unit cell of said protein so as to provide a structure for said protein, or (b) assigning NMR spectra peaks of said protein by manipulating said co-ordinates.

A method for determining the structure of a compound bound to EXPB1 protein, said method comprising: providing a crystal of EXPB1 protein; soaking the crystal with the compound to form a complex; and determining the structure of the complex by employing the data of any one of FIGS. 2-3 or a portion thereof.

A method for determining the structure of a compound bound to EXPB1 protein, said method comprising: mixing EXPB1 protein with the compound; crystallizing an EXPB1 protein-compound complex; and determining the structure of the complex by employing the data of any one of Tables 1 or FIGS. 2-3 or a portion thereof.

A method for modifying the structure of a compound in order to alter its metabolism by an EXPB1, which method comprises: fitting a starting compound to one or more coordinates of at least one amino acid residue of the ligand-binding region of the EXPB1; modifying the starting compound structure so as to increase or decrease its interaction with the ligand-binding region.

A method for modifying the structure of a compound in order to alter its metabolism by an EXPB1, which method comprises: fitting a starting compound to one or more coordinates of at least one amino acid residue of the binding region of the EXPB1; modifying the starting compound structure so as to increase or decrease its interaction with the binding region.

A method for modifying the structure of a compound in order to alter its, or another compounds, metabolism by an EXPB1, which method comprises: fitting a starting compound to one or more coordinates of at least one amino acid residue of the peripheral binding region of the EXPB1; modifying the starting compound structure so as to increase or decrease its interaction with the peripheral binding region; wherein said peripheral binding region is defined as the EXPB1 residues numbered as: W26, Y27, G40, nd G44, Y160, and W194.

A method of obtaining a representation of the three dimensional structure of a crystal of EXPB1, which method comprises providing the data of any one of PDB accession #2HCZ or FIGS. 2-3 or selected coordinates thereof, and constructing a three-dimensional structure representing said coordinates.

A computer system, intended to generate structures and/or perform optimization of compounds which interact with EXPB1, EXPB1 homologues or analogues, complexes of EXPB1 with compounds, or complexes of EXPB1 homologues or analogues with compounds, the system containing computer-readable data comprising one or more of: (a) EXPB1 co-ordinate data of any one of PDB accession #2HCZ, of FIGS. 2-3, said data defining the three-dimensional structure of EXPB1 or at least selected coordinates thereof; (b) atomic coordinate data of a target EXPB1 protein generated by homology modeling of the target based on the coordinate data of any one of PDB accession #2HCZ, FIGS. 2-3 (c) atomic coordinate data of a target EXPB1 protein generated by interpreting X-ray crystallographic data or NMR data by reference to the co-ordinate data of any one of PDB accession #2HCZ, or FIGS. 2-3 (d) structure factor data derivable from the atomic coordinate data of (b) or (c). and (e) atomic coordinate data of any one of PDB accession #2HCZ, or FIGS. 2-3 or selected coordinates thereof.

A computer system according to paragraph comprising: (i) a computer-readable data storage medium comprising data storage material encoded with said computer-readable data; (ii) a working memory for storing instructions for processing said computer-readable data; and (iii) a central-processing unit coupled to said working memory and to said computer-readable data storage medium for processing said computer-readable data and thereby generating structures and/or performing rational compound design.

A computer system comprising a display coupled to said central-processing unit for displaying said structures.

A method of providing data for generating structures and/or performing optimization of compounds which interact with EXPB1, EXPB1 homologues or analogues; complexes of EXPB1 with compounds, or complexes of EXPB1 homologues or analogues with compounds, the method comprising: (i) establishing communication with a remote device containing (a) computer-readable data comprising atomic coordinate data of any one of Tables 1, or FIGS. 2-3 or selected coordinates thereof; (b) atomic coordinate data of a target EXPB1 homologue or analogue generated by homology modeling of the target based on the data (a); (c) atomic coordinate data of a protein generated by interpreting X-ray crystallographic data or NMR data by reference to the data of any one of PDB accession #2HCZ, or FIGS. 2-3 and (d) structure factor data derivable from the atomic coordinate data of (d) or (e); and (ii) receiving said computer-readable data from said remote device.

A computer-readable storage medium, comprising a data storage material encoded with computer readable data, wherein the data are defined by all or a portion of the structure coordinates of the EXPB1 protein of any one of PDB accession #2HCZ or FIGS. 2-3 or a homologue of EXPB1, wherein said homologue comprises backbone atoms that have a root mean square deviation from the backbone atoms of said any one of PDB accession #2HCZ, or table 1, or FIGS. 2-3 respectively of not more than 1.5 Å.

### A. Protein Crystals.

The present invention provides a crystal of EXPB1 having cell dimensions of about a=113.7 Å, b=45.2 Å and c=70.3 Å. With angles α=90.0°, β=124.6°, and γ=90.0. Unit cell variability of 5% may be observed in all dimensions.

Substrates include plant cell walls, or components thereof. Alternatively the ligand could be a compound whose interaction with EXPB1 is unknown.

Such crystals may be obtained using the methods described in the accompanying examples.

The EXPB1 may optionally comprise a tag, such as a C-terminal polyhistidine tag to allow for recovery and purification of the protein.

The methodology used to provide an EXPB1 crystal illustrated herein may be used generally to provide an EXPB1 crystal resolvable at a resolution of at least 3.0 Å and preferably at least 2.8 Å. The invention thus further provides an EXPB1 crystal having a resolution of at least 3.0 Å, preferably at least 2.8 Å. The proteins may be wild-type proteins or variants thereof, which are modified to promote crystal formation, for example by N-terminal truncations and/or deletion of loop regions, which prevent crystal formation.

In a further aspect, the invention provides a method for making an EXPB1 protein crystal, particularly of an EXPB1 protein comprising the core sequence of EXPB1 (as defined above) or a variant thereof, which method comprises growing a crystal by vapor diffusion using a reservoir buffer that contains 0.05-0.2 M HEPES pH 7.0-7.8, 2.5-10% IPA, 0-20% PEG 4000, 0-0.3 M sodium chloride, 0-10% PEG 400, 0-10% glycerol, preferably 0.1 M HEPES pH 7.2, 5% IPA, 10% PEG 4000. The crystal is grown by vapor diffusion and is performed by placing an aliquot of the solution on a cover slip as a hanging drop above a well containing the reservoir buffer. The concentration of the protein solution used was 0.3-0.7 mM.

Crystals of the invention also include crystals of EXPB1 mutants, chimeras, homologues in the expansin family (e.g. α-expansins, β-expansins, group 2/3 allergens, etc) and alleles.

### (i) Mutants

A mutant is an EXPB1 protein characterized by the replacement or deletion of at least one amino acid from the wild type EXPB1. Such a mutant may be prepared for example by site-specific mutagenesis, or incorporation of natural or unnatural amino acids.

The present invention contemplates “mutants” wherein a “mutant” refers to a polypeptide which is obtained by replacing at least one amino acid residue in a native or synthetic EXPB1 with a different amino acid residue and/or by adding and/or deleting amino acid residues within the native polypeptide or at the N- and/or C-terminus of a polypeptide corresponding to EXPB1, and which has substantially the same three-dimensional structure as EXPB1 from which it is derived. By having substantially the same three-dimensional structure is meant having a set of atomic structure co-ordinates that have a root mean square deviation (r.m.s.d.) of less than or equal to about 2.0 Å (preferably less than 1.55 or 1.5 Å, more preferably less than 1.0 Å, and most preferably less than 0.5 Å) when superimposed with the atomic structure co-ordinates of the EXPB1 from which the mutant is derived when at least about 50% to 100% of the C_{α} atoms of the EXPB1 are included in the superposition. A mutant may have, but need not have, enzymatic or catalytic activity.

To produce homologues or mutants, amino acids present in the said protein can be replaced by other amino acids having similar properties, for example hydrophobicity, hydrophobic moment, antigenicity, propensity to form or break α-helical or β-sheet structures, and so on. Substitutional variants of a protein are those in which at least one amino acid in the protein sequence has been removed and a different residue inserted in its place. Amino acid substitutions are typically of single residues but may be clustered depending on functional constraints e.g. at a crystal contact. Preferably amino acid substitutions will comprise conservative amino acid substitutions. Insertional amino acid variants are those in which one or more amino acids are introduced. This can be amino-terminal and/or carboxy-terminal fusion as well as intrasequence. Examples of amino-terminal and/or carboxy-terminal fusions are affinity tags, MBP tag, and epitope tags.

Amino acid substitutions, deletions and additions which do not significantly interfere with the three-dimensional structure of the EXPB1 will depend, in part, on the region of the EXPB1 where the substitution, addition or deletion occurs. In highly variable regions of the molecule, non-conservative substitutions as well as conservative substitutions may be tolerated without significantly disrupting the three-dimensional structure of the molecule. In highly conserved regions, or regions containing significant secondary structure, conservative amino acid substitutions are preferred.

Conservative amino acid substitutions are well-known in the art, and include substitutions made on the basis of similarity in polarity, charge, solubility, hydrophobicity, hydrophilicity and/or the amphipathic nature of the amino acid residues involved. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, alanine; asparagine, glutamine; serine, threonine; phenylalanine, tyrosine. Other conservative amino acid substitutions are well known in the art.

In some instances, it may be particularly advantageous or convenient to substitute, delete and/or add amino acid residues in order to provide convenient cloning sites in the cDNA encoding the polypeptide, to aid in purification of the polypeptide, etc. Such substitutions, deletions and/or additions which do not substantially alter the three dimensional structure of EXPB1 will be apparent to those having skills in the art.

It should be noted that the mutants contemplated herein need not exhibit enzymatic activity. Indeed, amino acid substitutions, additions or deletions that interfere with the catalytic activity of the EXPB1 but which do not significantly alter the three-dimensional structure of the catalytic region are specifically contemplated by the invention. Such crystalline polypeptides, or the atomic structure co-ordinates obtained there from, can be used to identify compounds that bind to the protein.

The residues for mutation could easily be identified by those skilled in the art and these mutations can be introduced by site-directed mutagenesis e.g. using a Stratagene QuikChange™ Site-Directed Mutagenesis Kit or cassette mutagenesis methods (see e.g. Ausubel et al., eds., Current Protocols in Molecular Biology, John Wiley & Sons, Inc., New York, and Sambrook et al., Molecular Cloning: a Laboratory Manual, 2nd ed., Cold Spring Harbor Laboratory Press, Cold Spring Harbor, N.Y., (1989)).

### (ii) Alleles

The present invention contemplates “alleles” wherein allele is used for two or more alternative forms of a gene resulting in different gene products and thus different phenotypes. An allele contains nucleotide changes that have been shown to affect transcription, splicing, translation, post-transcriptional or post-translational modifications or result in at least one amino acid change. These different alleles are particularly important in EXPB1s as some may confer different properties on cell wall expansion onto the phenotype. Alleles are often only different by one or two amino acids.

To the extent that the present invention relates to EXPB1-ligand complexes and mutant, homologue, analogue, allelic form, species variant proteins of EXPB1, crystals of such proteins may be formed. The skilled person would recognize that the conditions provided herein for crystallizing EXPB1 may be used to form such crystals. Alternatively, the skilled person would use the conditions as a basis for identifying modified conditions for forming the crystals.

Thus the aspects of the invention relating to crystals of EXPB1, may be extended to crystals of mutant and mutants of EXPB1 which result in homologue, allelic form, and species variant.

(iii) Crystallization of EXPB1

To produce crystals of EXPB1 protein the final protein is, conveniently, concentrated to 10-60, e.g. 20-40 mg/ml in 10-100 mM potassium phosphate with high salt (e.g. 500 mM NaCl or KCl), optionally also with about 1 mM EDTA and/or about 2 mM dithiothreitol, by using concentration devices which are commercially available. Crystallization of the protein is set up by the 0.5-2/1 hanging or sitting drop methods and the protein is crystallized by vapor diffusion at 5-25° C. against a range of vapor diffusion buffer compositions. It is customary to use a 1:1 ratio of protein solution and vapor diffusion buffer in the hanging drop, and this has been used herein unless stated to the contrary.

Typically the vapor diffusion buffer comprises 0-27.5%, preferably 2.5-27.5% PEG 1K-20 K, preferably 1-8K or PEG 2000MME-5000MME, preferably PEG 2000 MME, or 0-10% Jeffamine M-600 and/or 5-20%, e.g. 10-20% propanol or 15-20% ethanol or about 15%-30%, e.g. about 15% 2-methyl-2,4-pentanediol (MPD), optionally with 0.01 M-1.6 M salt or salts and/or 0-0.15, e.g. 0-0.1 M of a solution buffer and/or 0-35%, such as 0-15%, glycerol and/or 0-35% PEG300-400; but preferably: 10-25% PEG 1K-8K or PEG 2000MME or 0-10% Jeffamine M-600 and/or 5-15%, e.g. 10-15%, propanol or ethanol, optionally with 0.1 M-0.2 M salt or salts and/or 0-0.15, e.g. 0-0.1 M solution buffer and/or PEG400, but more preferably: 15-20% PEG 3350 or PEG 4000 or PEG 2000MME or 0-10% Jeffamine M-600 or 5-15%, e.g. 10-15% propanol or ethanol, optionally with 0.1 M-0.2 M salt or salts and/or 0-0.15 M solution buffer.

Alternatively the vapor diffusion buffer may be 0.1 M HEPES pH 7.5 0.2-0.3 M potassium chloride, 1-5% MPD, 7-14.0% PEG 3350 or PEG 4000, 25-50 mM calcium chloride more specifically 0.1 M HEPES pH 7.5, 0.20-0.30 M KCl, 10-14% PEG 4000, 5% MPD, 25 mM calcium chloride.

The salt may be an alkali metal (particularly lithium, sodium and potassium), alkaline earth metal (e.g. magnesium or calcium), ammonium, ferric, ferrous or transition metal salt (e.g. zinc) of a halide (e.g. bromide, chloride or fluoride), acetate, formate, nitrate, sulfate, tartrate, citrate or phosphate. This includes sodium fluoride, potassium fluoride, ammonium fluoride, ammonium acetate, lithium acetate, magnesium acetate, sodium acetate, potassium acetate, calcium acetate, zinc acetate, ammonium chloride, lithium chloride, magnesium chloride, potassium chloride, sodium chloride, potassium bromide, magnesium formate, sodium formate, potassium formate, ammonium formate, ammonium nitrate, lithium nitrate, potassium nitrate, sodium nitrate, ammonium sulfate, potassium sulfate, lithium sulfate, sodium sulfate, di-sodium tartrate, potassium sodium tartrate, di-ammonium tartrate, potassium dihydrogen phosphate, tri-sodium citrate, tri-potassium citrate, zinc acetate, ferric chloride, calcium chloride, magnesium nitrate, magnesium sulfate, sodium dihydrogen phosphate, di-sodium hydrogen phosphate, di-potassium hydrogen phosphate, ammonium dihydrogen phosphate, di-ammonium hydrogen phosphate, tri-lithium citrate, nickel chloride, ammonium iodide, di-ammonium hydrogen citrate.

Solution buffers if present include, for example, Hepes, Tris, imidazole, cacodylate, tri-sodium citrate/citric acid, tri-sodium citrate/HCl, acetic acid/sodium acetate, phosphate-citrate, sodium potassium phosphate, 2-(N-morpholino)-ethane sulphonic acid/NaOH (MES), CHES or bis-trispropane. The pH range is desirably maintained at pH 4.2-8.5, preferably 4.7-8.5. Solution buffers if present can also include, for example, bicine, bis-tris, CAPS, MOPS, ADA which allow the pH to be maintained in the range 5.8-11.

Crystals may be prepared using a Hampton Research Screening kits, Poly-ethylene glycol (PEG)/ion screens, PEG grid, Ammonium sulphate grid, PEG/ammonium sulphate grid or the like. Crystallization may also be performed in the presence of an inhibitor of EXPB1, e.g. fluvoxamine or 2-phenyl imidazole. EXPB1 crystallization may also be performed in the presence of one or more inhibitors e.g. ketoconazole, metyrapone, fluconazole or triadimefon and/or in the presence of one or more substrate(s) e.g. testosterone or progesterone.

Additives can be added to a crystallization condition identified to influence crystallization. Additive Screens are to be used during the optimization of preliminary crystallization conditions where the presence of additives may assist in the crystallization of the sample and the additives may improve the quality of the crystal e.g. Hampton Research additive screens which use glycerol, polyols and other protein stabilizing agents in protein crystallization (R. Sousa. Acta. Cryst. (1995) D51, 271-277) or divalent cations (Trakhanov, S. and Quiocho, F. A. Protein Science (1995) 4, 9, 1914-1919).

In addition, detergents may be added to a crystallization condition to improve the crystallization behavior e.g. the ionic, non-ionic and zwitterionic detergents found in the Hampton Research detergent screens (McPherson, A., et al., The effects of neutral detergents on the crystallization of soluble proteins, J. Crystal Growth (1986) 76, 547-553).

Alternatively, the vapor diffusion buffer typically comprises 0-27.5% PEG 1K-20 K, preferably 1-8K or PEG 2000MME-5000MME, preferably PEG 2000 MME, or 0-10% Jeffamine M-600 and/or 1-20%, e.g. 1-20% propanol or 15-20% ethanol or about 1%-30%, e.g. about 2-25% 2-methyl-2,4-pentanediol (MPD), optionally with 0.01 M-1.6 M salt or salts and/or 0-0.15 M, e.g. 0-0.1 M, of a solution buffer and/or 0-35%, such as 0-15%, glycerol and/or 0-35% PEG300-400; but preferably: 0-27.5%, preferably 2.5-27.5% PEG 1K-20 K, most preferably 5-20% PEG 4K or PEG 2000MME-5000MME, preferably PEG 2000 MME, and 1-20% alcohol, e.g. 1-20% propanol e.g. iso-propanol or 2-25% 2-methyl-2,4-pentanediol (MPD), optionally with 0.01 M-1.6 M salt or salts and/or 0-0.15 M, e.g. 0-0.1 M, of a solution buffer and/or 0-35%, such as 0-15%, glycerol and/or 0-35% PEG300-400.

### B. Crystal Coordinates.

In a further aspect, the invention also provides a crystal of EXPB1 having the three dimensional atomic coordinates of PDB accession #2HCZ, the description herein, table 1, and/or FIGS. 2-3.

Protein structure similarity is routinely expressed and measured by the root mean square deviation (r.m.s.d.), which measures the difference in positioning in space between two sets of atoms. The r.m.s.d. measures distance between equivalent atoms after their optimal superposition. The r.m.s.d. can be calculated over all atoms, over residue backbone atoms (i.e. the nitrogen-carbon-carbon backbone atoms of the protein amino acid residues), main chain atoms only (i.e. the nitrogen-carbon-oxygen-carbon backbone atoms of the protein amino acid residues), side chain atoms only or more usually over C-α atoms only. For the purposes of this invention, the r.m.s.d. can be calculated over any of these, using any of the methods outlined below.

Thus the coordinates disclosed herein provide a measure of atomic location in Angstroms, given to 3 decimal places. The coordinates are a relative set of positions that define a shape in three dimensions, but the skilled person would understand that an entirely different set of coordinates having a different origin and/or axes could define a similar or identical shape. Furthermore, the skilled person would understand that varying the relative atomic positions of the atoms of the structure so that the root mean square deviation of the residue backbone atoms (i.e. the nitrogen-carbon-carbon backbone atoms of the protein amino acid residues) is less than 2.0 Å, preferably less than 1.55 or 1.5 Å, more preferably less than 1.0 Å, more preferably less than 0.5 Å, more preferably less than 0.3 Å, such as less than 0.25 Å, or less than 0.2 Å, and most preferably less than 0.1 Å, when superimposed on the coordinates provided in PDB accession #2HCZ for the residue backbone atoms, will generally result in a structure which is substantially the same as the structures disclosed herein in terms of both its structural characteristics and usefulness for structure-based analysis of EXPB1-interactivity molecular structures.

A further rmsd value of less than 1.0 Å which is preferred is a value of less than 0.6 Å, and rmsd values of less than 0.5 Å which are preferred are values of less than 0.45 Å, preferably less than 0.35 Å.

Unless explicitly set out to the contrary, or otherwise clear from the context, reference throughout the present specification to the use of all or selected coordinates disclosed herein does not exclude the use of additional coordinates.

Methods of comparing protein structures are discussed in Methods of Enzymology, vol 115, pg 397-420. The necessary least-squares algebra to calculate r.m.s.d. has been given by Rossman and Argos (J. Biol. Chem., vol 250, pp 7525 (1975)) although faster methods have been described by Kabsch (Acta Crystallogr., Section A, A92, 922 (1976)); Acta Cryst. A34, 827-828 (1978)), Hendrickson (Acta Crystallogr., Section A, A35, 158 (1979)); McLachan (J. Mol. Biol., vol 128, pp 49 (1979)) and Kearsley (Acta Crystallogr., Section A, A45, 208 (1989)). Some algorithms use an iterative procedure in which the one molecule is moved relative to the other, such as that described by Ferro and Hermans (Ferro and Hermans, Acta Crystallographic, A33, 345-347 (1977)). Other methods e.g. Kabsch's algorithm locate the best fit directly.

Programs for determining rmsd include MNYFIT (part of a collection of programs called COMPOSER, Sutcliffe, M. J., Haneef, I., Carney, D. and Blundell, T. L. (1987) Protein Engineering, 1, 377-384), MAPS (Lu, G. An Approach for Multiple Alignment of Protein Structures (1998, in manuscript and on http://bioinfol.mbfys.lu.se/TOP/maps.html)).

It is usual to consider C-alpha atoms and the rmsd can then be calculated using programs such as LSQKAB (Collaborative Computational Project 4. The CCP4 Suite: Programs for Protein Crystallography, Acta Crystallographica, D50, (1994), 760-763), QUANTA (Jones et al., Acta Crystallography A47 (1991), 110-119 and commercially available from Accelerys, San Diego, Calif.), Insight (commercially available from Accelerys, San Diego, Calif.), Sybyl.®. (commercially available from Tripos, Inc., St Louis), O (Jones et al., Acta Crystallographica, A47, (1991), 110-119), and other coordinate fitting programs.

In, for example the programs LSQKAB and O, the user can define the residues in the two proteins that are to be paired for the purpose of the calculation. Alternatively, the pairing of residues can be determined by generating a sequence alignment of the two proteins, programs for sequence alignment are discussed in more detail in Section F. The atomic coordinates can then be superimposed according to this alignment and an r.m.s.d. value calculated. The program Sequoia (C. M. Bruns, I. Hubatsch, M. Ridderstrom, B. Mannervik, and J. A. Tainer (1999) Human Glutathione Transferase A4-4 Crystal Structures and Mutagenesis Reveal the Basis of High Catalytic Efficiency with Toxic Lipid Peroxidation Products, Journal of Molecular Biology 288(3): 427-439) performs the alignment of homologous protein sequences, and the superposition of homologous protein atomic coordinates. Alternatively, the program Astex-KFIT (published in WO2004/038015) can be used. Once aligned, the r.m.s.d. can be calculated using programs detailed above. For sequence identical, or highly identical, the structural alignment of proteins can be done manually or automatically as outlined above. Another approach would be to generate a superposition of protein atomic coordinates without considering the sequence.

It is more normal when comparing significantly different sets of coordinates to calculate the rmsd value over C-α atoms only. It is particularly useful when analyzing side chain movement to calculate the rmsd over all atoms and this can be done using LSQKAB and other programs.

Those of skill in the art will appreciate that in many applications of the invention, it is not necessary to utilize all the coordinates disclosed herein, but merely a portion of them. For example, as described below, in methods of modeling candidate compounds with EXPB1, selected coordinates of EXPB1 may be used.

By “selected coordinates” it is meant for example at least 5, preferably at least 10, more preferably at least 50 and even more preferably at least 100, for example at least 500 or at least 1000 atoms of the EXPB1 structure. Likewise, the other applications of the invention described herein, including homology modeling and structure solution, and data storage and computer assisted manipulation of the coordinates, may also utilize all or a portion of the coordinates (i.e. selected coordinates). The selected coordinates may include or may consist of atoms found in the EXPB1 binding pocket, as described herein below.

### C. Description of Structure.

EXPB1 contains two domains (residues 19-140 [D1] and 147-245 [D2]) connected by a short linker (residues 141-146) and aligned end to end so as to make a closely-packed irregular cylinder ˜66 Å long and 26 Å in diameter (FIG. 2A). At its N-terminus EXPB1 has a flexible sequence (residues 1-18) containing hydroxyproline (O9) and a glycan attached to N10, part of the glycosylation consensus sequence NXT. The end of the glycan comes close to the polysaccharide-binding groove (see below) of the symmetry-related protein in the crystalline lattice, with one of the mannose residues stacking against the planar surface formed by residues Gly39 and Gly40 and stabilized further by two hydrogen bonds with the side chain of D37. These interactions with the symmetry-related protein account in part for the unusual ordering of the glycan, as well as the ability to crystallize the glycosylated protein.

Based on its electron density, our model of this N-linked glycan consists of a (1→4)-linked backbone of GlcNac₁GlcNac₂Man₃ with two Man residues and a Xyl residue attached to Man₃ and a Fuc residue linked to GlcNac₁ (FIG. 5, which is published as supporting information on the PNAS web site). Such so-called paucimannosidic-type N-linked glycans are characteristically processed in the Golgi and in post-Golgi steps (31).

Residues 1-3 in the leader sequence were not modeled due to insufficient electron density, but N-terminal sequencing and mass spectrometry indicate their presence (24). The 24-aa signal peptide at the N-terminus, predicted from the EXPB1 cDNA, was absent and was presumably excised during ER processing prior to secretion. No other post-translational modifications, bound metals or ligands were evident from the crystal structure.

The two EXPB1 domains pack close to one another, making contact via H-bonds and salt bridges between basic residues (K65 and R137) in D1 and acidic residues (E217 and D171) in D2. These residues are highly conserved in the EXPB family (see annotated sequence logo in FIG. 3). Additional hydrogen bonding is found between S72 and D173, as well as between the peptide backbone for C42 and A196. The two domains also make contact via a hydrophobic patch consisting of 144, P51, Y52 and Y92 in D1 and L164, Y167 and the hydrocarbon chain of K166 in D2, residues that are mostly well conserved or have conservative substitutions in the EXPB family. Moreover, six highly conserved glycine residues (G43, 67, 69, 71, 172, 195) are found at the surfaces where the two domains make contact. The lack of side chains in the glycine residues permits close packing of the two domains.

Structure of Domain 1. Residues 19-140 form an irregular ovoid with rough dimensions of 35×30×24 Å. The protein fold is dominated by a six-stranded β-barrel flanked by short loops and α-helices (FIG. 2A). D1 has three disulfide bonds (FIG. 3), and the six participating cysteines are highly conserved in both EXPA and EXPB families.

Previous analysis (2, 3) indicated that D1 has distant sequence similarity to members of glycoside hydrolase family 45 (GH45), whose members have been characterized as inverting endo-β-(1→4)-D-glucanases (2, 3, 32, 33). Superposition of D1 with a GH45 protein (PDB #4ENG) using the secondary structure matching algorithm in CCP4 (34) gives good overlap of the two structures for 84 residues (60%) of the peptide backbone of D1 (FIG. 2B), with an root mean square deviation (rmsd) of 2.5 Å. Two of the three disulfide bonds in D1 superimpose exactly with 4ENG disulfides (the exception being C78-C84). Likewise, all of the β-strands in D1 superimpose on β-strands of 4ENG, although the β-strands in EXPB1 are generally shorter. Both structures have short α-helices, but these do not overlap in the two structures.

The GH45 enzyme is substantially larger than D1 (210 residues versus 121) and the “extra” structure in the GH45 enzyme is composed largely of loop regions and α-helices forming a large ridge and subtending structure lacking in D1 (FIG. 2B). In 4ENG this ridge makes a steep border on one side of the deep glucan-binding cleft. Because this ridge is missing in D1, the corresponding surface is more like an open groove than a deep cleft, with space to bind a large, branched polysaccharide (FIGS. 2F, G).

In addition to partial conservation of the protein fold, D1 has noteworthy, but incomplete, conservation of the catalytic site identified in GH45 enzymes (FIG. 2C). In 4ENG (residues designated with *) the catalytic site is centered on aromatic residue Y8* which binds a glucose residue and is flanked by two acidic residues, D10* and D121*, serving as catalytic base and proton donor, respectively, for hydrolysis of the glycosidic bond (33, 35). D121* is flanked on one side by the hydrophobic side chains of A74* and Y8* and on the other side is part of a hydrogen-bonded network with T6*, which in turn is hydrogen bonded to H119*. In D1, a nearly identical structure is found (FIG. 2C), where D107 corresponds to the proton donor D121*, with C58 and Y27 forming the hydrophobic pocket, while T25 and H105 overlap the corresponding residues in 4ENG. Thus D1 possesses much of the conserved catalytic machinery for glycan hydrolysis.

What is missing in EXPB1 is a residue corresponding to D10*, the catalytic base required for glucan hydrolysis by GH45 enzymes (35). As indicated in FIG. 2C D10* is located on a loop that is not aligned with any part of EXPB1. EXPB proteins do have a conserved acidic residue, D37, which is located in a loop (residues 29-38) in the general vicinity corresponding to D10* in 4ENG. This loop is well resolved in D1. However, D37 is located too far from D107 and Y27 to function as the required base. In 4ENG, the catalytic carboxylate groups are located 8.5 Å apart, which is sufficient distance to accommodate a water molecule needed for hydrolysis (35). In D1, the carboxylates for D107 and D37 are 15 Å apart, too distant for this catalytic mechanism. Moreover, simple lateral movement of the loop to bring D37 into a correct position seems unlikely as the loop residues following D37 are rigidly held in place by a several stabilizing interactions. Thus, a key part of the catalytic machinery required for hydrolytic activity of GH45 enzymes is lacking in EXPB1.

Inspection of the EXPB1 structure revealed another acidic residue, D95, which is close to D107 (the carboxylate groups are 8.5 Å away). D95 is highly conserved in group-1 allergens, as well as in β-expansins in general (FIG. 3), but not in α-expansins. However, D95 is not correctly positioned, relative to the D107/Y27 site and the presumed position of the glycan backbone to serve as the catalytic base for hydrolysis. D95 and D37 have an appropriate distance from each other to potentially serve in hydrolysis of a sugar residue, which might be bound to the planar hydrophobic surface made up of G39, G40 and A41 backbone atoms, but none of these residues are part of the site that is conserved with GH45 enzymes.

Enzymatic activity. Because of the structural similarity between D1 and GH45 and the configuration of D95/D37, we tested the ability of EXPB1 to hydrolyze the major polysaccharides of the cell wall. Even with 48-h incubations, we did not detect hydrolytic activity by EXPB1 (FIG. 4A).

Taking another tack, we tested two GH45 enzymes (32, 36) and a nonenzymatic GH45-related protein named “swollenin” (37) for their abilities to catalyze cell wall extension. For these experiments, heat-inactivated walls from cucumber hypocotyls and wheat coleoptiles were clamped in tension in an extensometer and changes in length were monitored upon addition of protein. We observed only small traces of wall extension activity for the GH45 enzymes and for swollenin. Thus, these related proteins lack significant expansin-type activity, at least with the cell walls tested here.

We conclude that, despite the structural similarity of D1 to GH45, EXPB1 does not induce wall extension via wall polysaccharide hydrolysis.

Structure of Domain 2 (D2). Residues 147-245 of EXPB1 make up a second domain (D2) composed of eight β strands assembled into two antiparallel β sheets (FIG. 2A). The two β sheets are at slight angles to each other and form a β-sandwich similar to the immunoglobulin fold. D2 has 36% sequence identity with Phl p 2, a group-2/3 grass pollen allergen (PDB #1WHO), and superposition of the two structures shows them to have identical folds (rmsd of 1.3 Å; FIG. 2D). In comparing the two structures, we find that D2 tends to have shorter β strands compared with Phl p 2 and the two proteins deviate slightly in the loop regions connecting the β-strands.

### Identification and Use of EXPB1 Binding Pocket Residues.

D1 and D2 form a long potential polysaccharide-binding site. The two EXPB1 domains align so as to form a long, shallow groove with highly conserved polar and aromatic residues suitably positioned to bind a twisted polysaccharide chain of 10 xylose residues (FIG. 2E-G). The groove extends from the conserved G129 at one end of D1, spans across a stretch of conserved residues in D1 and D2 (see numbered residues in FIG. 2E as well as annotated sequence logo in FIG. 3) and ends at N157, a distance of some 47 Å. Many of the conserved residues common to EXPA and EXPB make up this potential binding surface, including residues in the classic expansin motifs TWYG, GGACG, and HFD (see FIG. 3).

Residues that could bind a polysaccharide by van der Waals interactions with the sugar rings include W26, Y27, G40, and G44 from D1 as well as Y160 and W194 from D2. Conserved residues that might stabilize polysaccharide binding by H-bonding include T25, D37, D95 and D107 in D1 and N157, S193 and R199 in D2.

### D. Chimeras.

The use of chimeric proteins to achieve desired properties is now common in the scientific literature. Active site chimeras are also described: for example, Swairjo et al (Biochemistry (1998) 37:10928-10936) made loop chimeras of HIV-1 and HIV-2 protease to try to understand determinants of inhibitor-binding specificity.

Of particular relevance are cases where the active site is modified so as to provide a surrogate system to obtain structural information. Thus Ikuta et al (J Biol Chem (2001) 276:27548-27554) modified the active site of cdk2, for which they could obtain structural data, to resemble that of cdk4, for which no X-ray structure is currently available. In this way they were able to obtain protein/ligand structures from the chimeric protein which were useful in cdk4 inhibitor design. In a similar way, based on comparison of primary sequences of highly related isoforms the active site of the EXPB1 protein could be modified to resemble those isoforms. Protein structures or protein/ligand structures of the chimeric proteins could be used in structure-based alteration of the metabolism of compounds which are substrates of that related EXPB1 isoform.

### (i) Converting Other EXPB1 Proteins to EXPB1-Like Chimeras

Aspects of the present invention therefore relate to modification of EXPB1 proteins such that the active sites mimic those of related isoforms. For example, from a knowledge of the structure and residues of the active site of the maize EXPB1 structure contained herein, a person skilled in the art could modify an EXPB1 protein such that the active site mimicked that of maize EXPB1. This protein could then be used to obtain information on compound binding through the determination of protein/ligand complex structures using the chimeric EXPB1 protein.

For example, in one aspect the present invention provides a chimeric protein having a binding cavity which provides a substrate specificity substantially identical to that of EXPB1 protein, wherein the chimeric protein binding cavity is lined by a plurality of atoms which correspond to selected EXPB1 atoms lining the EXPB1 binding cavity, and the relative positions of the plurality of atoms corresponding to the relative positions, as defined herein.

### E. Homology Modeling.

The invention also provides a means for homology modeling of other proteins (referred to below as target EXPB1 proteins). By “homology modeling”, it is meant the prediction of related EXPB1 structures based either on X-ray crystallographic data or computer-assisted de novo prediction of structure, based upon manipulation of the coordinate data derivable herein or selected portions thereof.

“Homology modeling” extends to target EXPB1 proteins which are analogues or homologues of the EXPB1 protein whose structure has been determined in the accompanying examples. It also extends to EXPB1 protein mutants of EXPB1 protein itself.

The term “homologous regions” describes amino acid residues in two sequences that are identical or have similar (e.g. aliphatic, aromatic, polar, negatively charged, or positively charged) side-chain chemical groups. Identical and similar residues in homologous regions are sometimes described as being respectively “invariant” and “conserved” by those skilled in the art.

In general, the method involves comparing the amino acid sequences of the EXPB1 protein with a target EXPB1 protein by aligning the amino acid sequences. Amino acids in the sequences are then compared and groups of amino acids that are homologous (conveniently referred to as “corresponding regions”) are grouped together. This method detects conserved regions of the polypeptides and accounts for amino acid insertions or deletions as seen in FIG. 3.

Homology between amino acid sequences can be determined using commercially available algorithms. The programs BLAST, gapped BLAST, BLASTN, PSI-BLAST and BLAST2 (provided by the National Center for Biotechnology Information) are widely used in the art for this purpose, and can align homologous regions of two amino acid sequences. These may be used with default parameters to determine the degree of homology between the amino acid sequence of the protein and other target EXPB1 proteins which are to be modeled.

Analogues are defined as proteins with similar three-dimensional structures and/or functions with little evidence of a common ancestor at a sequence level.

Homologues are defined as proteins with evidence of a common ancestor, i.e. likely to be the result of evolutionary divergence and are divided into remote, medium and close sub-divisions based on the degree (usually expressed as a percentage) of sequence identity.

A homologue is defined here as a protein with at least 15% sequence identity or which has at least one functional domain, which is characteristic of EXPB1. This includes polymorphic forms of EXPB1.

There are two types of homologue: orthologues and paralogues. Orthologues are defined as homologous genes in different organisms, i.e. the genes share a common ancestor coincident with the speciation event that generated them. Paralogues are defined as homologous genes in the same organism derived from a gene/chromosome/genome duplication, i.e. the common ancestor of the genes occurred since the last speciation event.

The homologues could also be polymorphic forms of EXPB1 such as alleles or mutants as described in section (A).

Once the amino acid sequences of the polypeptides with known and unknown structures are aligned, the structures of the conserved amino acids in a computer representation of the polypeptide with known structure are transferred to the corresponding amino acids of the polypeptide whose structure is unknown. For example, a tyrosine in the amino acid sequence of known structure may be replaced by a phenylalanine, the corresponding homologous amino acid in the amino acid sequence of unknown structure.

The structures of amino acids located in non-conserved regions may be assigned manually by using standard peptide geometries or by molecular simulation techniques, such as molecular dynamics. The final step in the process is accomplished by refining the entire structure using molecular dynamics and/or energy minimization.

Homology modeling as such is a technique that is well known to those skilled in the art (see e.g. Greer, Science, Vol. 228:1055 (1985), and Blundell et al., Eur. J. Biochem, Vol. 172:513 (1988)). The techniques described in these references, as well as other homology modeling techniques, generally available in the art, may be used in performing the present invention.

Thus the invention provides a method of homology modeling comprising the steps of: (a) aligning a representation of an amino acid sequence of a target EXPB1 protein of unknown three-dimensional structure with the amino acid sequence of the EXPB1 herein to match homologous regions of the amino acid sequences; (b) modeling the structure of the matched homologous regions of said target EXPB1 of unknown structure on the corresponding regions of the EXPB1 structure as obtained as described above and/or that of any one of Tables 1-4 or selected coordinates thereof; and (c) determining a conformation (e.g. so that favorable interactions are formed within the target EXPB1 of unknown structure and/or so that a low energy conformation is formed) for said target EXPB1 of unknown structure which substantially preserves the structure of said matched homologous regions. Preferably one or all of steps (a) to (c) are performed by computer modeling.

The aspects of the invention described herein which utilize the EXPB1 structure in silico may be equally applied to homologue models of EXPB1 obtained by the above aspect of the invention, and this application forms a further aspect of the present invention. Thus having determined a conformation of an EXPB1 by the method described above, such a conformation may be used in a computer-based method of rational drug design as described herein.

### F. Structure Solution

The atomic coordinate data of EXPB1 can also be used to solve the crystal structure of other target EXPB1 proteins including other crystal forms of EXPB1, mutants, co-complexes of EXPB1, where X-ray diffraction data or NMR spectroscopic data of these target EXPB1 proteins has been generated and requires interpretation in order to provide a structure.

In the case of EXPB1, this protein may crystallize in more than one crystal form. The data, as provided by this invention, are particularly useful to solve the structure of those other crystal forms of EXPB1. It may also be used to solve the structure of EXPB1 mutants, EXPB1 co-complexes, or of the crystalline form of any other protein with significant amino acid sequence homology to any functional domain of EXPB1.

In the case of other target EXPB1 proteins, particularly the maize EXPB1 proteins referred to in Section E above, the present invention allows the structures of such targets to be obtained more readily where raw X-ray diffraction data is generated.

Thus, where X-ray crystallographic or NMR spectroscopic data is provided for a target EXPB1 of unknown three-dimensional structure, the atomic coordinate data derived herein, may be used to interpret that data to provide a likely structure for the other EXPB1 by techniques which are well known in the art, e.g. phasing in the case of X-ray crystallography and assisting peak assignments in NMR spectra.

One method that may be employed for these purposes is molecular replacement. In this method, the unknown crystal structure, whether it is another crystal form of EXPB1, an EXPB1 mutant, an EXPB1 chimera or an EXPB1 co-complex, or the crystal of a target EXPB1 protein with amino acid sequence homology to any functional domain of EXPB1, may be determined using the EXPB1 structure coordinates. This method will provide an accurate structural form for the unknown crystal more quickly and efficiently than attempting to determine such information ab initio.

Examples of computer programs known in the art for performing molecular replacement are CNX (Brunger A. T.; Adams P. D.; Rice L. M., Current Opinion in Structural Biology, Volume 8, Issue 5, October 1998, Pages 606-611 (also commercially available from Accelrys San Diego, Calif.), MOLREP (A. Vagin, A. Teplyakov, MOLREP: an automated program for molecular replacement, J. Appl. Cryst. (1997) 30, 1022-1025, part of the CCP4 suite) or AMoRe (Navaza, J. (1994). AMoRe: an automated package for molecular replacement. Acta Cryst. A50, 157-163).

### G. Computer Systems.

In another aspect, the present invention provides systems, particularly a computer system, the systems containing one of (a) EXPB1 co-ordinate data herein, said data defining the three-dimensional structure of EXPB1 or at least selected coordinates thereof; (b) atomic coordinate data of a target EXPB1 protein generated by homology modeling of the target based on the coordinate data herein, (c) atomic coordinate data of a target EXPB1 protein generated by interpreting X-ray crystallographic data or NMR data by reference to the co-ordinate data herein; or (d) structure factor data derivable from the atomic coordinate data of (b) or (c).

For example the computer system may comprise: (i) a computer-readable data storage medium comprising data storage material encoded with the computer-readable data; (ii) a working memory for storing instructions for processing said computer-readable data; and (iii) a central-processing unit coupled to said working memory and to said computer-readable data storage medium for processing said computer-readable data and thereby generating structures and/or performing rational compound design. The computer system may further comprise a display coupled to said central-processing unit for displaying said structures.

The invention also provides such systems containing atomic coordinate data of target EXPB1 proteins wherein such data has been generated according to the methods of the invention described herein based on the starting data provided the data herein or selected coordinates thereof.

Such data is useful for a number of purposes, including the generation of structures to analyze the mechanisms of action of EXPB1 proteins and/or to perform rational drug design of compounds, which interact with EXPB1.

In a further aspect, the present invention provides computer readable media with at least one of (a) EXPB1 co-ordinate data herein, said data defining the three-dimensional structure of EXPB1 or at least selected coordinates thereof; (b) atomic coordinate data of a target EXPB1 protein generated by homology modeling of the target based on the coordinate data herein, (c) atomic coordinate data of a target EXPB1 protein generated by interpreting X-ray crystallographic data or NMR data by reference to the co-ordinate data; or (d) structure factor data derivable from the atomic coordinate data of (b) or (c).

In another aspect, the invention provides a computer-readable storage medium, comprising a data storage material encoded with computer readable data, wherein the data are defined by all or a portion (e.g. selected coordinates as defined herein) of the structure coordinates of EXPB1 herein, or a homologue of said EXPB1, wherein said homologue comprises backbone atoms that have a root mean square deviation from the Cα or backbone atoms (nitrogen-carbon_{α}-carbon) of less than 2 Å, preferably less than 1.55 or 1.5 Å, more preferably less than 1.0 Å (e.g. less than 0.6 Å), and most preferably less than 0.5 Å (e.g. less than 0.45 Å such as less than 0.35 Å).

As used herein, “computer readable media” refers to any medium or media, which can be read and accessed directly by a computer. Such media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

By providing such computer readable media, the atomic coordinate data of the invention can be routinely accessed to model EXPB1s or selected coordinates thereof. For example, RASMOL (Sayle et al., TIBS, Vol. 20, (1995), 374) is a publicly available computer software package, which allows access and analysis of atomic coordinate data for structure determination and/or rational drug design.

As used herein, “a computer system” refers to the hardware means, software means and data storage means used to analyze the atomic coordinate data of the invention. The minimum hardware means of the computer-based systems of the present invention comprises a central processing unit (CPU), input means, output means and data storage means. Desirably a monitor is provided to visualize structure data. The data storage means may be RAM or means for accessing computer readable media of the invention. Examples of such systems are microcomputer workstations available from Silicon Graphics Incorporated and Sun Microsystems running Unix based, Windows NT or IBM OS/2 operating systems.

The invention also provides a computer-readable data storage medium comprising a data storage material encoded with a first set of computer-readable data comprising the EXPB1 coordinates herein or selected coordinates thereof; which, when combined with a second set of machine readable data comprising an X-ray diffraction pattern of a molecule or molecular complex of unknown structure, using a machine programmed with the instructions for using said first set of data and said second set of data, can determine at least a portion of the electron density corresponding to the second set of machine readable data.

### H. Uses of the Structures of the Invention.

The crystal structures obtained according to the present invention as well as the structures of target EXPB1 proteins obtained in accordance with the methods described herein), may be used in several ways for chemical compound design.

In the case where a molecule is bound by an EXPB1, information on the binding orientation by either co-crystallization, soaking or computationally docking the binding orientation of the compound in the binding pocket can be determined. This will guide specific modifications to the chemical structure designed to mediate or control the interaction of the compound with the protein. Such modifications can be designed with an aim to increase the enhancement of activity by EXPB1 or to increase the active life of the compound and so improve its enzymatic activity.

The crystal structure could also be useful to understand EXPB1-cellulose (substrate) interactions. The crystal structure of the present invention complexed to such a modulator or other compound (either in vitro or in silico) may also allow rational modifications either to modify the modulator such that it either increases or decreases activity, or to modify the EXPB1 such that it could bind better and so displace the modulator.

EXPB1s, as all expansins display significant polymorphic variations dependent on the plant species. This can manifest itself in adverse reactions from some uses. By using the crystal structures of the present invention to map the relevant mutation with respect to the binding mode of EXPB1, chemical modifications could also be made to the expansin to avoid interactions with the variable region of the protein. This could ensure more consistent polysaccharide binding and cell wall extension from EXPB1 for such segments of the population and avoid unwanted deleterious effects.

Some compounds may be converted by EXPB1s into active metabolites. In the case of such compounds, a greater understanding of how such compounds are converted by an EXPB1 will allow modification of the compound so that it can be converted at a different rate. For example, increasing the rate of conversion may allow a more rapid delivery of a desired wall loosening effect, whereas decreasing the rate of conversion may allow for higher sustained activity.

Thus, the determination of the three-dimensional structure of EXPB1 provides a basis for the design of new compounds, which interact with EXPB1 in novel ways. For example, knowing the three-dimensional structure of EXPB1, computer modeling programs may be used to design different molecules expected to interact with possible or confirmed active sites, such as binding sites or other structural or functional features of EXPB1.

### (i) Obtaining and Analyzing Crystal Complexes.

In one approach, the structure of a compound bound to an EXPB1 may be determined by experiment. This will provide a starting point in the analysis of the compound bound to EXPB1, thus providing those of skill in the art with a detailed insight as to how that particular compound interacts with EXPB1 and the mechanism by which it is metabolized.

Many of the techniques and approaches to structure-based compound design described above rely at some stage on X-ray analysis to identify the binding position of a ligand in a ligand-protein complex. A common way of doing this is to perform X-ray crystallography on the complex, produce a difference Fourier electron density map, and associate a particular pattern of electron density with the ligand. However, in order to produce the map (as explained e.g. by Blundell et al., in Protein Crystallography, Academic Press, New York, London and San Francisco, (1976)), it is necessary to know beforehand the protein 3D structure (or at least the protein structure factors). Therefore, determination of the EXPB1 structure also allows difference Fourier electron density maps of EXPB1-compound complexes to be produced, determination of the binding position of the drug and hence may greatly assist the process of rational drug design.

Accordingly, the invention provides a method for determining the structure of a compound bound to EXPB1, said method comprising: providing a crystal of EXPB1 according to the invention; soaking the crystal with said compounds; and determining the structure of said EXPB1 compound complex by employing the data described herein.

Alternatively, the EXPB1 and compound may be co-crystallized. Thus the invention provides a method for determining the structure of a compound bound to EXPB1, said method comprising; mixing the protein with the compound(s), crystallizing the protein-compound(s) complex; and determining the structure of said EXPB1-compound(s) complex by reference to the EXPB1 structural data herein.

The analysis of such structures may employ (i) X-ray crystallographic diffraction data from the complex and (ii) a three-dimensional structure of EXPB1, or at least selected coordinates thereof, to generate a difference Fourier electron density map of the complex, the three-dimensional structure being defined by atomic coordinate data provided herein. The difference Fourier electron density map may then be analyzed.

Therefore, such complexes can be crystallized and analyzed using X-ray diffraction methods, e.g. according to the approach described by Greer et al., J. of Medicinal Chemistry, Vol. 37, (1994), 1035-1054, and difference Fourier electron density maps can be calculated based on X-ray diffraction patterns of soaked or co-crystallized EXPB1 and the resolved structure of uncomplexed EXPB1. These maps can then be analyzed e.g. to determine whether and where a particular compound binds to EXPB1 and/or changes the conformation of EXPB1.

Electron density maps can be calculated using programs such as those from the CCP4 computing package (Collaborative Computational Project 4. The CCP4 Suite: Programs for Protein Crystallography, Acta Crystallographica, D50, (1994), 760-763.). For map visualization and model building programs such as “O” (Jones et al., Acta Crystallographica, A47, (1991), 110-119) can be used.

In addition, in accordance with this invention, EXPB1 mutants may be crystallized in co-complex with known EXPB1 substrates or inhibitors or novel compounds. The crystal structures of a series of such complexes may then be solved by molecular replacement and compared with that of the EXPB1 structure disclosed herein. Potential sites for modification within the various binding sites of the protein may thus be identified. This information provides an additional tool for determining the most efficient binding interactions, for example, increased hydrophobic interactions, between EXPB1 and a chemical entity or compound.

For example there are alleles of EXPB1, which differ from the native EXPB1 by only 1-2 amino acid substitutions, and yet individuals who express these allelic variants may exhibit different binding affinities or activities. The metabolism of enzymatic agents used in the hydrolysis of cellulose or plant cell wall extension applications can be investigated using the structure provided here and the agents then altered using the methods described herein.

This information may thus be used to optimize known classes of EXPB1 enhanced enzymes (e.g. cellulases), substrates or enhancers, and more importantly, to design and synthesize novel classes of compounds with modified or enhanced EXPB1 activity.

### (ii) In Silico Analysis and Design

Although the invention will facilitate the determination of actual crystal structures comprising an EXPB1 and a compound, which interacts with the EXPB1, current computational techniques provide a powerful alternative to the need to generate such crystals and generate and analyze diffraction date. Accordingly, a particularly preferred aspect of the invention relates to in silico methods directed to the analysis and development of compounds which interact with EXPB1 structures of the present invention.

Determination of the three-dimensional structure of EXPB1 provides important information about the binding sites of EXPB1, particularly when comparisons are made with similar expansins, and grass pollen allergens. This information may then be used for rational design and modification of EXPB1 substrates and inhibitors, e.g. by computational techniques which identify possible binding ligands for the binding sites, by enabling linked-fragment approaches to drug design, and by enabling the identification and location of bound ligands (e.g. including those ligands mentioned herein above) using X-ray crystallographic analysis. These techniques are discussed in more detail below.

Thus as a result of the determination of the EXPB1 three-dimensional structure, more purely computational techniques for chemical compound design may also be used to design structures whose interaction with EXPB1 is better understood (for an overview of these techniques see e.g. Walters et al (Drug Discovery Today, Vol. 3, No. 4, (1998), 160-178; Abagyan, R.; Totrov, M. Curr. Opin. Chem. Biol. 2001, 5:375-382). For example, automated ligand-receptor docking programs (discussed e.g. by Jones et al. in Current Opinion in Biotechnology, Vol. 6, (1995), 652-656 and Halperin, I.; Ma, B.; Wolfson, H.; Nussinov, R. Proteins 2002, 47:409-443), which require accurate information on the atomic coordinates of target receptors may be used.

The aspects of the invention described herein which utilize the EXPB1 structure in silico may be equally applied to both the EXPB1 structure of disclosed herein and the models of target EXPB1 proteins obtained by other aspects of the invention. Thus having determined a conformation of an EXPB1 by the method described above, such a conformation may be used in a computer-based method of rational drug design as described herein. In addition the availability of the structure of the EXPB1 will allow the generation of highly predictive models for virtual library screening or compound design.

Accordingly, the invention provides a computer-based method for the analysis of the interaction of a molecular structure with an EXPB1 structure of the invention, which comprises: providing the structure of an EXPB1 of the invention; providing a molecular structure to be fitted to said EXPB1 structure; and fitting the molecular structure to the EXPB1 structure.

In an alternative aspect, the method of the invention may utilize the coordinates of atoms of interest of the EXPB1 binding region, which are in the vicinity of a putative molecular structure, for example within 10-25 Å of the catalytic regions or within 5-10 Å of a compound bound, in order to model the pocket in which the structure binds. These coordinates may be used to define a space, which is then analyzed “in silico”. Thus the invention provides a computer-based method for the analysis of molecular structures which comprises; providing the coordinates of at least two atoms of an EXPB1 structure of the invention (“selected coordinates”); providing the structure of a molecular structure to be fitted to said coordinates; and fitting the structure to the selected coordinates of the EXPB1.

In practice, it will be desirable to model a sufficient number of atoms of the EXPB1 as defined herein, which represent a binding groove, e.g. the atoms of the residues identified in residues G1229-N157 which also preferably maintains the binding motifs TWYG, GGACG, HFD. Thus, in this embodiment of the invention, there will preferably be provided the coordinates of at least 5, preferably at least 10, more preferably at least 50 and even more preferably at least 100, e.g. at least 500 such as at least 1000, selected atoms of the EXPB1 structure.

Although every different compound metabolized by EXPB1 may interact with different parts of the binding pocket of the protein, the structure of this EXPB1 allows the identification of a number of particular sites which are likely to be involved in many of the interactions of EXPB1 with a candidate compound. The residues are set out in FIGS. 2 and 3. Thus in this aspect of the invention, the selected coordinates may comprise coordinates of some or all of these residues.

In order to provide a three-dimensional structure of compounds to be fitted to an EXPB1 structure of the invention, the compound structure may be modeled in three dimensions using commercially available software for this purpose or, if its crystal structure is available, the coordinates of the structure may be used to provide a representation of the compound for fitting to an EXPB1 structure of the invention.

The binding pockets of cytochrome EXPB1 molecules are of a size which can accommodate more than one ligand. Indeed, some interactions may occur as a result of interaction of the compounds within the binding pocket of the same EXPB1. In any event, the findings of the present invention may be used to examine or predict the interaction of two or more separate molecular structures within the EXPB1 binding pocket of the invention.

Thus the invention provides a computer-based method for the analysis of the interaction of two molecular structures within an EXPB1 binding pocket structure, which comprises: providing the EXPB1 structure; providing a first molecular structure; fitting the first molecular structure to said EXPB1 structure; providing a second molecular structure; and fitting the second molecular structure to a different part said EXPB1 structure.

Optionally the method of analysis further comprises providing a third molecular structure and also fitting that structure to the EXPB1 structure. Indeed, further molecular structures may be provided and fitted in the same way.

In one aspect, one or more of the molecular structures may be fitted to one or more of the polysaccharide binding area, residues G129 through N157 of the EXPB1 binding groove mentioned above, and one or more of the other molecular structures may be fitted to coordinates of amino acids from another part of the EXPB1 binding pocket, such as another part of the ligand-binding region.

Following the fitting of the molecular structures, a person of skill in the art may seek to use molecular modeling to determine to what extent the structures interact with each other (e.g. by hydrogen bonding, other non-covalent interactions, or by reaction to provide a covalent bond between parts of the structures) or the interaction of one structure with EXPB1 is altered by the presence of another structure.

The person of skill in the art may use in silico modeling methods to alter one or more of the structures in order to design new structures which interact in different ways with EXPB1, so as to speed up or slow down their metabolism, as the case may be.

Newly designed structures may be synthesized and their interaction with EXPB1 may be determined or predicted as to how the newly designed structure is metabolized by said EXPB1 structure. This process may be iterated so as to further alter the interaction between it and the EXPB1.

By “fitting”, it is meant determining by automatic, or semi-automatic means, interactions between at least one atom of a molecular structure and at least one atom of an EXPB1 structure of the invention, and calculating the extent to which such an interaction is stable. Interactions include attraction and repulsion, brought about by charge, steric considerations and the like. Various computer-based methods for fitting are described further herein.

More specifically, the interaction of a compound or compounds with EXPB1 can be examined through the use of computer modeling using a docking program such as GOLD (Jones et al., J. Mol. Biol., 245, 43-53 (1995), Jones et al., J. Mol. Biol., 267:727-748 (1997)), GRAMM (Vakser, I. A., Proteins, Suppl., 1:226-230 (1997)), DOCK (Kuntz et al, J. Mol. Biol. 1982, 161:269-288, Makino et al, J. Comput. Chem. 1997, 18:1812-1825), AUTODOCK (Goodsell et al, Proteins 1990, 8:195-202, Morris et al, J. Comput. Chem. 1998, 19:1639-1662.), FlexX, (Rarey et al, J. Mol. Biol. 1996, 261:470-489) or ICM (Abagyan et al, J. Comput. Chem. 1994, 15:488-506). This procedure can include computer fitting of compounds to EXPB1 to ascertain how well the shape and the chemical structure of the compound will bind to the EXPB1.

Also computer-assisted, manual examination of the active site structure of EXPB1 may be performed. The use of programs such as GRID (Goodford, J. Med. Chem., 28, (1985), 849-857)—a program that determines probable interaction sites between molecules with various functional groups and an the polysaccharide binding surface—may also be used to analyze the active site to predict, for example, the types of modifications which will alter the rate of conformational change, or cell wall extension a compound or plant cell type.

Computer programs can be employed to estimate the attraction, repulsion, and steric hindrance of the two binding partners (i.e. the EXPB1 and a compound).

If more than one EXPB1 active site is characterized and a plurality of respective smaller compounds are designed or selected, a compound may be formed by linking the respective small compounds into a larger compound, which maintains the relative positions and orientations of the respective compounds at the active sites. The larger compound may be formed as a real molecule or by computer modeling.

Detailed structural information can then be obtained about the binding of the compound to EXPB1, and in the light of this information adjustments can be made to the structure or functionality of the compound, e.g. to alter its interaction with EXPB1. The above steps may be repeated and re-repeated as necessary.

As indicated above, molecular structures, which may be fitted to the EXPB1 structure of the invention, include compounds under development as potential enzymatic agents. The agents may be fitted in order to determine how the action of EXPB1 modifies the agent and to provide a basis for modeling candidate agents, which are metabolized at a different rate by an EXPB1.

Molecular structures, which may be used in the present invention, will usually be compounds under development for pharmaceutical use. Generally such compounds will be organic molecules, which are typically from about 100 to 2000 Da, more preferably from about 100 to 1000 Da in molecular weight. Such compounds include peptides and derivatives thereof, and the like. In principle, any compound under development in the field of enzymology can be used in the present invention in order to facilitate its development or to allow further design to improve its properties.

(iii) Analysis of Compounds in Binding Pocket Regions

Our finding of a long grooved binding region allows the analysis and design methods described in the preceding subsections to be focused on compounds which interact with one or more of the residues which make up this area.

Thus in one embodiment, the present invention provides a method for modifying the structure of a compound (polysaccharide) in order to alter its binding to EXPB1 or hydrolysis when bound to EXPB1, which method comprises: fitting a starting compound to one or more coordinates of at least one amino acid residue of the ligand-binding region of the EXPB1; modifying the starting compound structure so as to increase or decrease its interaction with the ligand-binding region.

In another embodiment, the present invention provides a method for modifying the structure of a compound in order to alter its metabolism by an EXPB1, which method comprises: fitting a starting compound to one or more coordinates of at least one amino acid residue of the ligand-binding region of the EXPB1; modifying the starting compound structure so as to increase or decrease its interaction with the ligand-binding region; wherein said ligand-binding region is defined as including at least one, such as at least two, for example such as at least five, preferably at least ten of the EXPB1 residues in the binding groove.

In another embodiment, the invention provides a method for modifying the structure of a compound in order to alter its binding properties to EXPB1 or cell wall extension when bound, which method comprises: fitting a starting compound to one or more coordinates of at least one amino acid residue of the binding region of the EXPB1; modifying the starting compound structure so as to increase or decrease its interaction with the binding region.

Desirably, in the above aspects of the invention, coordinates from at least two, preferably at least five, and more preferably at least ten amino acid residues of the EXPB1 will be used.

For the avoidance of doubt, the term “modifying” is used as defined in the preceding subsection, and once such a compound has been developed it may be synthesized and tested also as described above.
(viii) Compounds of the Invention.

Where a potential modified compound has been developed by fitting a starting compound to the EXPB1 structure of the invention and predicting from this a modified compound with an altered rate of metabolism (including a slower, faster or zero rate), the invention further includes the step of synthesizing the modified compound and testing it in an in vivo or in vitro biological system in order to determine its activity and/or the rate at which it is metabolized.

The method comprises: (a) providing EXPB1 under conditions where, in the absence of modulator, the EXPB1 is able to metabolize known substrates; (b) providing the compound; and (c) determining the extent to which the compound is metabolized in the presence of EXPB1 or (d) determining the extent to which the compound inhibits metabolism of a known substrate of EXPB1.

More preferably, in the latter steps the compound is contacted with EXPB1 under conditions to determine its function.

For example, in the contacting step above the compound is contacted with EXPB1 in the presence of the compound, and typically a buffer and substrate, to determine the ability of said compound to inhibit EXPB1 or to be metabolized by EXPB1. So, for example, an assay mixture for EXPB1 may be produced which comprises the compound, substrate and buffer.

In another aspect, the invention includes a compound, which is identified by the methods of the invention described above.

Following identification of such a compound, it may be manufactured and/or used in the preparation, i.e. manufacture or formulation, of a composition such as an enzymatic composition used in ethanol production, paper recycling or other plant cell extension industrial applications.

Thus, the present invention extends in various aspects not only to a compound as provided by the invention, but also to formulations including acceptable excipients, vehicles or carriers, and optionally other ingredients.

The above-described processes of the invention may be iterated in that the modified compound may itself be the basis for further compound design.

By “optimizing the structure” we mean e.g. adding molecular scaffolding, adding or varying functional groups, or connecting the molecule with other molecules (e.g. using a fragment linking approach) such that the chemical structure of the modulator molecule is changed while its original modulating functionality is maintained or enhanced. Such optimization is regularly undertaken during chemical compound development programs to e.g. enhance potency, promote pharmacological acceptability, increase chemical stability etc. of lead compounds.

Modification will be those conventional in the art known to the skilled medicinal chemist, and will include, for example, substitutions or removal of groups containing residues which interact with the amino acid side chain groups of an EXPB1 structure of the invention. For example, the replacements may include the addition or removal of groups in order to decrease or increase the charge of a group in a test compound, the replacement of a charge group with a group of the opposite charge, or the replacement of a hydrophobic group with a hydrophilic group or vice versa. It will be understood that these are only examples of the type of substitutions considered by medicinal chemists in the development of new pharmaceutical compounds and other modifications may be made, depending upon the nature of the starting compound and its activity.

### EXAMPLE 1

### Crystal Structure and Activities of EXPB1 (Zea m 1), a Beta-Expansin and Group-1 Pollen Allergen from Maize

Expansins are small extracellular proteins that promote turgor-driven extension of plant cell walls. EXPB1 (also called Zea m 1) is a member of the β-expansin subfamily known in the allergen literature as group-1 grass pollen allergens. EXPB1 induces extension and stress relaxation of grass cell walls. To help elucidate expansin's mechanism of wall loosening, we determined the structure of EXPB1 by X-ray crystallography to 2.75 Å resolution. EXPB1 consists of two domains closely packed and aligned so as to form a long, shallow groove with potential to bind a glycan backbone of ˜10 sugar residues.

The structure of EXPB1 domain 1 resembles that of family-45 glucoside hydrolase (GH45), with conservation of most of the residues in the catalytic site. However, EXPB1 lacks a second aspartate that serves as the catalytic base required for hydrolytic activity in GH45 enzymes. Domain 2 of EXPB1 is an immunoglobulin-like β-sandwich with aromatic and polar residues that form a potential surface for polysaccharide binding in line with the glycan binding cleft of domain 1. EXPB1 binds to maize cell walls, most strongly to xylans, causing swelling of the cell wall. Tests for hydrolytic activity by EXPB1 with various wall polysaccharides proved negative. Moreover, GH45 enzymes and a GH45-related protein called “swollenin”, lacked wall extension activity comparable to that of expansins. We propose a model of expansin action in which EXPB1 facilitates the local movement and stress relaxation of arabinoxylan-cellulose networks within the wall by noncovalent rearrangement of its target.

Prior to maturation plant cells typically experience a period of prolonged cell enlargement, often resulting in a >10³ fold increase in volume. The impressive height of trees, some exceeding 100 m, depends on such enlargement, which entails massive vacuolar expansion and irreversible yielding of the cellulosic cell wall. In physical terms, the rate-limiting process for cell enlargement resides within the cell wall, which must be loosened so as to allow wall stress relaxation and consequent water uptake for vacuole enlargement and stretching of the wall (1, 2). Currently, the only plant proteins shown to cause cell wall relaxation are expansins (3, 4), although xyloglucan endotransglucosylase, pectate lyase, cellulase and other enzymes participate in cell wall restructuring during cell growth (5-8).

Expansins were originally discovered in a “fishing expedition” for catalysts of cell wall extension (9, 10). When walls are clamped in tension and incubated in acidic buffer, these proteins rapid induce wall extension and enhance wall stress relaxation. Their biological role in promoting cell enlargement is amply supported by in-vitro and in-vivo experiments, as well as by studies of gene expression, gene silencing, and ectopic expression (3, 11-13). In addition to cell enlargement, expansins are also implicated in other developmental processes where wall loosening occurs, such as in fruit softening, organ abscission, seed germination, and pollen tube invasion of the grass stigma (14-17).

Two expansin families with wall-loosening activity have been identified, named α-expansins (EXPA) and β-expansins (EXPB); both are found in all groups of land plants, from mosses to flowering plants (3, 18). Although they have only ˜20% amino acid identity, EXPA and EXPB proteins are of similar size (˜27 kD), their sequences align well with one another and they contain a number of conserved residues and characteristic motifs distributed throughout the length of the protein. EXPA and EXPB appear to act on different cell wall components, but their native targets have not yet been well defined.

A subset of β-expansins is known in the immunological literature as group-1 grass pollen allergens (19-21). These β-expansins are abundantly and specifically expressed in grass pollen, causing hay fever and seasonal asthma in an estimated 200-400 million humans (22, 23). The extraordinary abundance of group-1 allergens—comprising up to 4% of the protein extracted from grass pollen (24)—is unique (as far as we know) in the world of expansins, which are typically found in very low abundance and tightly bound to the cell wall. The abundance of group-1 allergens in grass pollen bespeaks a unique biological role, namely to loosen the cell walls of the grass stigma and style, thereby aiding pollen tube penetration and assisting delivery of its two sperm cells to the ovule, where a double fertilization occurs, forming the diploid zygote and the triploid endosperm. Seed development follows, and because cereal grasses provide the largest food source for humanity (e.g. rice, maize, wheat, and barley, to name but a few), the importance of these events for human welfare is hard to overestimate.

Other genes in the β-expansin family are expressed in a variety of other tissues in the plant body and in general lack the specific allergenic epitopes characteristic of group-1 allergens (24, 25). These so-called “vegetative β-expansins” are thought to have cell wall loosening activity and substrate specificity similar to the group-1 allergens, but these inferences have yet to be demonstrated experimentally.

The mechanism by which expansins loosen cell walls has not yet been worked out in molecular detail. Plant cell walls consist of a scaffold of long cellulose microfibrils ˜4 nm in diameter, embedded in a matrix of cellulose-binding glycans, such as xyloglucan and arabinoxylan, and gel-forming pectic polysaccharides (FIG. 1). The cellulose-binding glycans form a stable network with the cellulose microfibrils by binding to their surface via hydrogen bonds between hydroxyl groups and via van der Waals forces between the sugar rings; the network is further stabilized by calcium ions and borate diesters that link pectic polysaccharides together. Cell walls also contain small amounts of structural proteins with a reinforcing role (26, 27). Wall expansion entails rearrangement or modification of the matrix to allow turgor-driven movement or slippage of cellulose microfibrils within the matrix (1).

Most of the biochemical work on expansins to date has focused on α-expansins, which do not hydrolyze the major structural polysaccharides of the wall and indeed are devoid of every enzyme activity assayed to date (28). Our current model proposes that α-expansins disrupt the polysaccharide complexes that link cellulose microfibrils together. The pollen β-expansins (group-1 allergens) have a marked loosening action on cell walls from grasses, but not from dicots, whereas the reverse is true for α-expansins; therefore it seems that the two forms of expansin target different components of the cell wall (21, 24). Grass cell walls are notable for containing relatively small amounts of xyloglucan and pectin, which are replaced with β-(1→3),(1→4)-D-glucan and glucuronoarabinoxylan (29)—two potential targets of β-expansins in their wall-loosening activity.

Sequence analysis suggests that expansins consist of two domains (2, 3). The putative N-terminal domain (D1) has distant sequence similarity (˜20% identity) to the catalytic domain of family-45 glycoside hydrolases (GH45; http://afmb.cnrs-mrs.fr/CAZY/). Despite this resemblance, α-expansins do not hydrolyze wall polysaccharides and so the sequence similarity is enigmatic. The C-terminal domain (D2) has sequence similarity (from 35% to <10% identity) to another class of allergens, the group-2/3 grass pollen allergens, whose biological function is unknown (30).

In this study we present the crystal structure of a native β-expansin purified from maize pollen. In the allergen field it is designated Zea m 1 isoform d, whereas by expansin nomenclature it is called EXPB1 (GenBank accession AAO45608). The allergen name “Zea m 1” encompasses a group of at least four pollen proteins (EXPB1, EXPB9, EXPB10, EXPB11) in two rather divergent sequence classes (24). EXPB1 is the most abundant of the maize group-1 allergens. We also test EXPB1 for binding and activity on cell walls. At the end we discuss a molecular model of expansin action that is consistent with its structure and known biophysical and biochemical activities.

### Results

EXPB1 has two closely-packed domains. Native EXPB1 was purified from maize pollen and crystallized in 15% (w/v) polyethylene glycol 4000 with 0.1 or 0.2 M ammonium sulfate. Two crystals were analyzed, yielding X-ray diffraction patterns consistent with the monoclinic C₂ space group. EXPB1 structure was solved and refined to 2.75 Å resolution (see Methods) with a crystallographic R-factor of 0.233 and an R-free of 0.291 (Table 1).

EXPB1 contains two domains (residues 19-140 [D1] and 147-245 [D2]) connected by a short linker (residues 141-146) and aligned end to end so as to make a closely-packed irregular cylinder ˜66 Å long and 26 Å in diameter (FIG. 2A). At its N-terminus EXPB1 has a flexible sequence (residues 1-18) containing hydroxyproline (O9) and a glycan attached to N10, part of the glycosylation consensus sequence NXT. The end of the glycan comes close to the polysaccharide-binding groove (see figures) of the symmetry-related protein in the crystalline lattice, with one of the mannose residues stacking against the planar surface formed by residues Gly39 and Gly40 and stabilized further by two hydrogen bonds with the side chain of D37. These interactions with the symmetry-related protein account in part for the unusual ordering of the glycan, as well as the ability to crystallize the glycosylated protein.

Based on its electron density, our model of this N-linked glycan consists of a β-(1→4)-linked backbone of GlcNac₁GlcNac₂Man₃ with two Man residues and a Xyl residue attached to Man₃ and a Fuc residue linked to GlcNac₁. Such so-called paucimannosidic-type N-linked glycans are characteristically processed in the Golgi and in post-Golgi steps (31).

Residues 1-3 in the leader sequence were not modeled due to insufficient electron density, but N-terminal sequencing and mass spectrometry indicate their presence (24). The 24-aa signal peptide at the N-terminus, predicted from the EXPB1 cDNA, was absent and was presumably excised during ER processing prior to secretion. No other post-translational modifications, bound metals or ligands were evident from the crystal structure.

The two EXPB1 domains pack close to one another, making contact via H-bonds and salt bridges between basic residues (K65 and R137) in D1 and acidic residues (E217 and D171) in D2. These residues are highly conserved in the EXPB family (see annotated sequence logo in FIG. 3). Additional hydrogen bonding is found between S72 and D173, as well as between the peptide backbone for C42 and A196. The two domains also make contact via a hydrophobic patch consisting of I44, P51, Y52 and Y92 in D1 and L164, Y167 and the hydrocarbon chain of K166 in D2, residues that are mostly well conserved or have conservative substitutions in the EXPB family. Moreover, six highly conserved glycine residues (G43, 67, 69, 71, 172, 195) are found at the surfaces where the two domains make contact. The lack of side chains in the glycine residues permits close packing of the two domains.

Structure of Domain 1. Residues 19-140 form an irregular ovoid with rough dimensions of 35×30×24 Å. The protein fold is dominated by a six-stranded β-barrel flanked by short loops and α-helices (FIG. 2A). D1 has three disulfide bonds (FIG. 3), and the six participating cysteines are highly conserved in both EXPA and EXPB families.

Previous analysis (2, 3) indicated that D1 has distant sequence similarity to members of glycoside hydrolase family 45 (GH45), whose members have been characterized as inverting endo-β-(1→4)-D-glucanases (2, 3, 32, 33). Superposition of D1 with a GH45 enzyme (PDB #4ENG) using the secondary structure matching algorithm in CCP4 (34) gives good overlap of the two structures for 84 residues (60%) of the peptide backbone of D1 (FIG. 2B), with an root mean square deviation (rmsd) of 2.5 Å. Two of the three disulfide bonds in D1 superimpose exactly with 4ENG disulfides (the exception being C78-C84). Likewise, all of the β-strands in D1 superimpose on β-strands of 4ENG, although the β-strands in EXPB1 are generally shorter. Both structures have short α-helices, but these do not overlap in the two structures.

The GH45 enzyme is substantially larger than D1 (210 residues versus 121) and the “extra” structure in the GH45 enzyme is composed largely of loop regions and α-helices forming a large ridge and subtending structure lacking in D1 (FIG. 2B). In 4ENG this ridge makes a steep border on one side of the deep glucan-binding cleft. Because this ridge is missing in D1, the corresponding surface is more like an open groove than a deep cleft, with space to bind a large, branched polysaccharide (FIGS. 2F, G).

In addition to partial conservation of the protein fold, D1 has noteworthy, but incomplete, conservation of the catalytic site identified in GH45 enzymes (FIG. 2C). In 4ENG (residues designated with *) the catalytic site is centered on aromatic residue Y8* which binds a glucose residue and is flanked by two acidic residues, D10* and D121*, serving as catalytic base and proton donor, respectively, for hydrolysis of the glycosidic bond (33, 35). D121* is flanked on one side by the hydrophobic side chains of A74* and Y8* and on the other side is part of a hydrogen-bonded network with T6*, which in turn is hydrogen bonded to H119*. In D1, a nearly identical structure is found (FIG. 2C), where D107 corresponds to the proton donor D121*, with C58 and Y27 forming the hydrophobic pocket, while T25 and H105 overlap the corresponding residues in 4ENG. Thus D1 possesses much of the conserved catalytic machinery for glycan hydrolysis.

What is missing in EXPB1 is a residue corresponding to D10*, the catalytic base required for glucan hydrolysis by GH45 enzymes (35). As indicated in FIG. 2C, D10* is located on a loop that is not aligned with any part of EXPB1. EXPB proteins do have a conserved acidic residue, D37, which is located in a loop (residues 29-38) in the general vicinity corresponding to D10* in 4ENG. This loop is well resolved in D1. However, D37 is located too far from D107 and Y27 to function as the required base. In 4ENG, the catalytic carboxylate groups are located 8.5 Å apart, which is sufficient distance to accommodate a water molecule needed for hydrolysis (35). In D1, the carboxylates for D107 and D37 are 15 Å apart, too distant for this catalytic mechanism. Moreover, simple lateral movement of the loop to bring D37 into a correct position seems unlikely as the loop residues following D37 are rigidly held in place by a several stabilizing interactions. Thus, a key part of the catalytic machinery required for hydrolytic activity of GH45 enzymes is lacking in EXPB1.

Inspection of the EXPB1 structure revealed another acidic residue, D95, which is close to D107 (the carboxylate groups are 8.5 Å away). D95 is highly conserved in group-1 allergens, as well as in β-expansins in general (FIG. 3), but not in α-expansins. However, D95 is not correctly positioned, relative to the D107/Y27 site and the presumed position of the glycan backbone to serve as the catalytic base for hydrolysis. D95 and D37 have an appropriate distance from each other to potentially serve in hydrolysis of a sugar residue, which might be bound to the planar hydrophobic surface made up of G39, G40 and A41 backbone atoms, but none of these residues are part of the site that is conserved with GH45 enzymes.

Enzymatic activity. Because of the structural similarity between D1 and GH45 and the configuration of D95/D37, we tested the ability of EXPB1 to hydrolyze the major polysaccharides of the cell wall. Even with 48-h incubations, we did not detect hydrolytic activity by EXPB1 (FIG. 4A).

Taking another tack, we tested two GH45 enzymes (32, 36) and a nonenzymatic GH45-related protein named “swollenin” (37) for their abilities to catalyze cell wall extension. For these experiments, heat-inactivated walls from cucumber hypocotyls and wheat coleoptiles were clamped in tension in an extensometer and changes in length were monitored upon addition of protein. We observed only small traces of wall extension activity for the GH45 enzymes and for swollenin. Thus, these related proteins lack significant expansin-type activity, at least with the cell walls tested here.

We conclude that, despite the structural similarity of D1 to GH45, EXPB1 does not induce wall extension via wall polysaccharide hydrolysis.

Structure of Domain 2 (D2). Residues 147-245 of EXPB1 make up a second domain (D2) composed of eight β strands assembled into two antiparallel β sheets (FIG. 2A. The two β sheets are at slight angles to each other and form a β-sandwich similar to the immunoglobulin fold. D2 has 36% sequence identity with Phl p 2, a group-2/3 grass pollen allergen (PDB #1WHO), and superposition of the two structures shows them to have identical folds (rmsd of 1.3 Å; FIG. 2D). In comparing the two structures, we find that D2 tends to have shorter β strands compared with Phl p 2 and the two proteins deviate slightly in the loop regions connecting the β-strands.
D1 and D2 form a long potential polysaccharide-binding site. The two EXPB1 domains align so as to form a long, shallow groove with highly conserved polar and aromatic residues suitably positioned to bind a twisted polysaccharide chain of 10 xylose residues (FIGS. 2E-G). The groove extends from the conserved G129 at one end of D1, spans across a stretch of conserved residues in D1 and D2 (see numbered residues in FIG. 2E as well as annotated sequence logo in FIG. 3) and ends at N157, a distance of some 47 Å. Many of the conserved residues common to EXPA and EXPB make up this potential binding surface, including residues in the classic expansin motifs TWYG, GGACG, and HFD (see FIG. 3).

Residues that could bind a polysaccharide by van der Waals interactions with the sugar rings include W26, Y27, G40, and G44 from D1 as well as Y160 and W194 from D2. Conserved residues that might stabilize polysaccharide binding by H-bonding include T25, D37, D95 and D107 in D1 and N157, S193 and R199 in D2.

The openness of the long groove may enable EXPB1 to bind polysaccharides that are part of a bulky cell wall complex, such as on the surface of cellulose; that openness may also be important for binding branched glycans such as arabinoxylan which itself binds to the surface of cellulose microfibrils. Because EXPB1 binds preferentially to xylans (see below), we have modeled an arabinoxylan, characteristic of grass cell walls, bound to the long groove of EXPB1 (FIG. 2G). From this model it is clear that the open groove of EXPB1 can accommodate the side chains found on such polysaccharides.

A second conserved surface in D2 is far removed from D1 (arrows in FIG. 2G). There is a shallow cup formed by the conserved W232 and F210. Adjacent to this pocket is a hydrophobic surface patch formed by the conserved residues P209, P229, V227 and Y238. The pocket and adjacent region could provide a second glucan binding surface for ˜3 residues.

Binding. EXPB1 bound to isolated maize cell wall (FIG. 4B). We observed that cell walls incubated with EXPB1 swelled significantly when compared with control cell walls (FIG. 4D). When purified polysaccharide fractions were immobilized onto nitrocellulose membranes, EXPB1 bound preferentially to xylans, with negligible binding to β-(1→3),(1→4) D-glucan and glucomannan (FIG. 4C). Intermediate binding to xyloglucan was observed. Specific binding to cellulose and to nitrocellulose was also seen, although with less avidity than to xylan (A. Tabuchi and D. J. Cosgrove, manuscript in preparation).

### Discussion

With the molecular structure of EXPB1 in hand, we can examine previous inferences about expansin structure and its mechanism of cell wall loosening, but first the use of the group-1 pollen allergen for this study merits comment. Unlike other forms of expansin, which are found in very low abundance and have low solubility, the group-1 allergens are produced in copious amounts by grass pollen, from which they are readily extracted, purified, and concentrated to high levels without precipitation. Moreover, grasses produce abundant pollen, with maize being an especially liberal donor. In contrast to recombinant forms, use of the native protein insures correct processing and post-translational modifications. We note that expression of active expansins in various recombinant systems has proved problematic, due to improper folding, aggregation and hyperglycosylation (M. Shieh and D. J. Cosgrove, unpublished data). Other forms of \tilde{□}expansin (e.g. the vegetative homologs) require harsh conditions to extract them from plant tissues (38), resulting in denatured protein; in soybean cultures an EXPB accumulates in the medium, but in a degraded and inactive form (39). EXPA proteins have been purified from various plant tissues, but in our experience they are difficult to concentrate to levels suitable for crystallization.

The high solubility and abundance of the group-1 allergens thus commends them for crystallization studies, but it should be noted that some of their biochemical properties may be specialized for their unique biological role in grass pollination. A case in point is their atypical pH dependence (maximum activity at pH 5.5; (24)), which is shifted to less acidic values than that found for other expansins. Likewise, their high solubility seems to be exceptional. Nevertheless, the general features of EXPB1 structure should prove to be common to the whole expansin family.

EXPB1 is composed of two domains. Although D1 structurally resembles GH45 and indeed has conserved much of the GH45 catalytic site, it lacks the second Asp residue—the catalytic base—required for hydrolytic activity in GH45 enzymes (33, 35). Thus, expansin's lack of wall polysaccharide hydrolytic activity, documented here for EXPB1 and in previous work for EXPA (28, 40), can be understood in structural terms as due to the lack of the required catalytic base. Furthermore, our finding that bona fide GH45 enzymes lack expansin's wall extension activity lends additional support to the conclusion that expansin does not loosen the cell wall by polysaccharide hydrolysis.

D2 as binding module? We previously speculated that D2 may be a carbohydrate-binding module (CBM) (2, 4). This notion gains indirect support from the structure of D2, in which two surface aromatic residues (W194, Y160) are in line with two aromatic residues (W26, Y27) in D1, forming part of an extended, open, and highly conserved surface in EXPB1. D2 has an immunoglobulin-like fold. Proteins with this fold form a large superfamily of β-sandwich proteins implicated in binding interactions, but lacking in enzymatic activity (41). At least 16 of the currently recognized CBM families in the Carbohydrate-Active Enzymes (CAZY) database (http://afmb.cnrs-mrs.fr/CAZY/) have a β-sandwich fold. However, the specific fold topology of D2 does not match any of these CBM folds and D2 lacks a bound metal atom, found in nearly all of the β-sandwich CBMs (42).

Nevertheless, from the structure of EXPB1 we expect that D2 aids glycan binding, particularly via the two surface aromatic residues W194 and Y160, aided by polar residues S193, R199, C156 and N157. These potential sugar-binding residues do not correspond to those inferred from a homology model of Lol p 1, a group-1 allergen from rye grass (43). In this model, which was based on the structure of Phl p 2, a group-2 allergen (30, 44), the authors identified two potential polysaccharide binding surfaces, one of which corresponds to the buried D2 face contacting D1.

It is notable that endoglucanases are most often found in nature as modular enzymes, coupled to a CBM via a long, highly glycosylated linker. Crystallization of intact GH45 enzymes with their CBMs has not yet been achieved, probably because the two domains do not maintain a fixed spatial relationship to each other. This difficulty of crystallization is a common experience with many CBM-coupled enzymes, and so successful crystallization of the two-domain EXPB1 is notable in this regard. In EXPB1 the linker is very short and the multiple contacts between D1 and D2 enable close coupling of the two domains, which may function as a single unit in binding the cell wall.

Expansins as cysteine proteases? A controversial hypothesis has been proposed that group-1 allergens are papain-related cysteine proteinases, with conservation of papain's active site residues C25, H159 and N175 (the “catalytic triad”) (45, 46). According to this hypothesis, C73 in EXPB1 should correspond to papain's C25. However, from the structure of EXPB1 we see that C73 participates in a disulfide bond conserved with GH45 enzymes, is relatively inaccessible, and is nowhere near the conserved surface. Moreover, the residues claimed to correspond to papain's H159 and N175 are dispersed in D2, remote from C73 and are not conserved in expansins. We conclude that the resemblance to papain suggested by Grobe et al. (45, 46) is not supported by our crystallographic model of EXPB1.

The conserved surface of EXPB1 does contain two Cys residues (C58, C156), but their environment does not resemble that of papain's active site. C58, which is conserved in about half of the EXPB family, is relatively inaccessible, being mostly buried underneath Y27 at the bottom of the extended groove. C156 not conserved in the EXPB family, but is usually replaced by serine. Experimental assays failed to detect proteinase activity in native EXPB1 (47). Moreover, the group-1 allergens are noted for their remarkable stability, which is also the case for EXPB1. We deem it likely that recombinant expression of EXPB in Pichia induced a host protease that accounted for the protein instability observed by Grobe et al. (45, 46). In fact, such host proteinase induction has been reported upon recombinant expression of a group-1 allergen (48).

Comparison with vegetative β-expansins and with α-expansins. EXPB1 is a member of the group-1 grass pollen allergens, which comprise a subset of the larger EXPB family. The EXPB family is notably larger in grasses than in other groups of land plants, and part of this expansion involved the unique evolution and radiation of the pollen allergen class of EXPBs, which are encoded by multiple genes (49). For instance, we classified 5 of the 19 EXPB genes in the rice genome as group-1 allergens (49). Multiple EXPB genes of the pollen allergen class may account in part for the numerous group-1 “isoallergens” found in grass pollen (19, 20, 50, 51).

There are minor conserved differences between the allergen class and the remaining “vegetative” EXPBs. These are so slight that we expect the structural features of EXPB1 are characteristic of the vegetative EXPBs, with one exception: the N-terminal extension in EXPB1 contains a motif (VPPGPNITT) that is consistently found, with only minor variation, in group-1 grass pollen allergens, but not in other EXPBs. This motif contains one or more hydroxyprolines and a glycosylated asparagine, features common to the pollen allergen class of EXPB (52). The function of this N-terminal extension is unknown, but it may play a role in protein recognition, transport, packaging and processing by the pollen secretory apparatus. Additionally, the glycosylated extension may contribute to the exceptional solubility of the group-1 allergens (other expansins characterized to date have very low solubility) or may interact with other components of the cell wall. While this motif is a unique hallmark of the group-1 allergens, many EXPB proteins lack an N-terminal extension altogether, and so it is not an essential part of expansin function. However, an N-terminal extension with similar post-translational modifications was found as part of an EXPB expressed in soybean cell cultures (39).

The good sequence alignment and conservation of motifs between the EXPB and EXPA families make it likely that EXPA proteins will have the same three-dimensional structure as reported here for EXPB1. There are two notable regions where EXPA and EXPB differ. EXPA has an additional stretch of ˜12 amino acids in the region corresponding to E99/P100 in EXPB1. E99 and P100 are part of a loop between β strands IV and V in D1; these residues form part of the upraised flank to the left of the long groove identified in FIG. 2. The additional residues in EXPA may form a larger shoulder flanking this groove, stabilized by a disulfide bond between a pair of cysteines in this loop that are conserved in the EXPA family but are lacking in many EXPBs, mostly notably absent in the pollen allergens. This idea gains support from the structure of another GH45 enzyme (PDB #1WC2 (53)) which contains just such a loop (residues 102-114) stabilized by a disulfide bond. The loop creates a shoulder abutting the catalytic cleft. EXPAs therefore may have a steeper binding cleft than that does EXPB1.

A second difference is that EXPAs lack a segment corresponding to G120-H127 in EXPB1. This segment, which contains few conserved residues, forms α-helix c and constitutes part of the surface of the pointed end of D1. This surface is remote from the conserved regions we have identified, and so is unlikely to affect activity.

Allergenic epitopes. Allergies to grass pollen are widespread, afflicting an estimated 200-400 million people, and numerous studies have concluded that the group-1 allergens are the most important allergenic components of grass pollen (23, 23, 54, 55). Maize EXPB1 and its orthologs in turf grasses share common epitopes, as judged by antibody cross reactivity, with the predominant epitopes found in the protein portion of the molecule and the glycosyl residues being of secondary antigenic significance (52, 56, 57). The dominant group-1 allergenic epitopes, which have been identified by epitope mapping studies, can be readily located on the surface of EXPB1. For instance, the 15-residue c98 epitope identified by Ball et al. (58) includes D107 in the conserved catalytic site of EXPB1, but also includes residues that are exposed on the opposite side of the protein. “Site D” identified by Hiller et al. (59) overlaps part of the extended conserved groove of D1 containing the motif TWYG28 (FIG. 2E), whereas “site A” identified by Esch and Klapper (60) includes the small conserved pocket containing W232 and Y238, found on the far side of D2, as indicated in FIGS. 2E, G. This pocket is also part of “peptide 5” (22), a synthetic peptide derived from B cell epitopes of Phl p 1, the group-1 allergen of timothy grass pollen. Antibodies against peptide 5 showed great potency in reducing binding of IgEs from patients with strong grass pollen allergens, and so this peptide was considered a potentially useful component of an epitope-based vaccine for treating patients with severe allergies to grass pollen (22). With the structure of EXPB1 in hand, one may consider designing synthetic peptides that more closely resemble the natural epitopes occurring on the conserved surface of group-1 allergens. These may be of use for immunotherapy as well as mechanistic studies concerning the molecular and cellular bases for the potency of these proteins as allergens.

In view of the sequence conservation within the EXPB family, as well as within the entire expansin superfamily, it is surprising that the dominant antigenic epitopes of the group-1 allergens are not shared by vegetative EXPBs or by EXPA members. Nevertheless, this seems to be the case because antibodies raised against the group-1 allergens do not recognize other forms of expansin. This is indeed fortunate, for otherwise persons with strong allergies to grass pollen would also be allergic to fresh fruits, vegetables, grains and other plant tissues that express members of this large gene family that is ubiquitous in plants.

A molecular model of wall loosening by expansins. Expansin action may be summarized as follows: the protein binds one or more wall polysaccharides and within seconds induces wall stress relaxation followed by wall extension, without hydrolysis of the wall polymers. There is no requirement for ATP or other source of chemical energy, and the wall continues to extend so long as the wall bears sufficient tension and expansin is present (that is, expansin acts catalytically, not stoichiometrically).

In the case of EXPB1, we imagine that stress relaxation begins when it binds a taut arabinoxylan tethered to a cellulose microfibril, causing local release of the arabinoxylan from the cellulose surface. Movement of the β-expansin along the arabinoxylan-cellulose junction would enable it to unzip the hydrogen bonds between the polysaccharides, relaxing the taut tether and allowing turgor-driven displacement of cellulose and arabinoxylan, which may then reassociate in a relaxed state to restore wall strength. During this movement, the two expansin domains might shift in a hinge-like manner, binding and letting go of the arabinoxylan independently of each other, leading to an inchworm-like movement along the polysaccharide. We estimate that as little as 10° shift in angle between domains could cause a one-residue dislocation of the polysaccharide along the binding surface.

To assess the feasibility of such inter-domain movement, we estimated the buried surface area between the two domains, using CCP4. The value is 589 Å, which is indicative of a weak inter-domain interaction (61), is consistent with domain movements as imagined above. A potential source of energy for these movements is the mechanical strain energy stored by the taut polysaccharide in a turgor-stretched cell wall. In this model, expansin acts as molecular device that uses the strain energy stored in a taut cellulose-binding glycan to help dissociate the glycan from the surface of cellulose.

### Materials and Methods

Protein Purification, Crystallization and Data Collection. Native Zea m 1 was extracted from pollen of field-grown maize plants at 4° C. in 0.125 M sodium carbonate and then purified to electrophoretic homogeneity in the presence of 5 mM dithiothreitol using two chromatographic steps as described (24). With this method four Zea m 1 isoforms were readily distinguished and we used the most abundant isoform, Zea m 1d (=EXPB1), for crystallization and activity assays. For the binding experiments, EXPB1 was further purified by HPLC on a reverse phase column (Discovery C8, 15 cm×4.6 mm i.d., 5 μm, Supelco) pre-equilibrated with 10% acetonitrile containing 0.1% trifluoroacetic acid. Bound protein was eluted at 1 mL min⁻ with a linear gradient of 22 to 90% acetonitrile in the same solution for 20 min at a flow rate of 1 mL min⁻, at 25° C. We confirmed wall extension activity of EXPB1 purified in this way.

Crystals were grown at 21° C. for 9 days using EXPB1 at 10.5 mg/mL in 100 mM Na acetate, pH 4.6, in 5-μL hanging drops, with addition of 5-μL precipitant (15% (w/v) polyethylene glycol 4000 with 0.1 or 0.2 M ammonium sulfate) and with 1-mL reservoir volume. Two crystals were analyzed, yielding diffraction patterns consistent with the monoclinic C₂ space group. Crystal 1 had unit cell dimensions of a=113.7 Å, b=45.2 Å, and c=70.3 Å, with angles α=90.0°, β=124.6°, and γ=90.0°; crystal 2 had unit cell dimensions of a=112.6 Å, b=44.4 Å, and c=69.6 Å, with angles α=90.0°, β=124.4°, and γ=90.0°.

Data were collected using a RIGAKU RU200 rotating anode X-ray generator with CuK□ radiation, operating at 5 KW of power (50 kV, 100 mA) (Molecular Structure Corporation, The Woodlands, Tex.). Three-degree oscillation frames, each exposed for 120 minutes were collected on an R-AXIS IV detector. The two crystals were used to get a 93% complete dataset. DENZO and SCALEPACK software suite (62) were used for data processing.

Structure Solution and Refinement. Our final model of EXPB1 structure was based on the native crystal data set and was solved by molecular replacement calculations using the program AmoRe (63) with the structure of Phl p 1 (PDB entry code 1N10) which has 58% amino acid identity with EXPB1 over 240 residues. EXPB1 has four more residues at its C-terminus. The best molecular replacement solution in AMoRE was obtained by deleting the first 13 residues of the N-terminus (attempts that included this stretch did not yield a solution) and by including all the side-chains for the rest of the protein (attempts with just the backbone atoms did not yield a good solution as well) and including all the available data to 2.75 Å. The correlation co-efficient and the R-factor for the best solution was 55.1 and 51.0 respectively. The next best solution had an inferior correlation co-efficient and R-factor of 49.3 and 53.9, enabling us to proceed with further refinement and model building with confidence. For further refinement details and comparison with the 1N10 structure, see supplemental text, published on the PNAS web site. Coordinates and structure factors of the structure have been deposited in the protein data bank (PDB code 2HCZ; (64)). A summary of the refinement results is given in Table 1 (on PNAS web site).
Polysaccharide Hydrolysis. Two mg of dye-coupled insoluble polysaccharides (AZCL-polysaccharides, Megazyme, Wicklow, Ireland) were suspended in 100/L buffer (50 mM sodium acetate, pH 4.5, with 1 mM NaN₃ and 10 mM dithiothreitol) and incubated with shaking at 30° C. for 48 h+/−30 μg of EXPB1. At the end of the incubation, 300 μL of 2.5% Trizma base was added to each tube to stop reaction, the suspension was centrifuged, and the absorbance (590 nm) of the supernatant was measured.
Binding. Cell walls were collected from maize silks, cleaned by phenol/acetic acid/water washes (65) and lyophilized. EXPB1 was purified on a CM-Sepharose Fast Flow (Amersham Biosciences) column in a LP system (Bio-Rad) (24). EXPB1 (10 μg) was incubated with 1 mg cell wall in 400 μL of 50 mM sodium acetate, pH 5.5, for 1 h at 25° C. with agitation. After incubation, protein remaining in the supernatant was analyzed by SDS-PAGE (12% poly acrylamide), stained with SYPRO Ruby protein gel stain (Bio-Rad).

Commercial polysaccharides dissolved in 20 mM sodium acetate, pH 4.5 (200 μg, oat spelts xylan (Sigma), birch wood xylan (Fluka), barley β-glucan (Sigma, G-6513), konjac glucomanna (Megazyme) and tamarind xyloglucan (Megazyme) were applied to nitrocellulose membranes disks (ca. 7 mm diameter, Protran, BA83, pore size; 0.2 μm, Whatman). The disks were dried at 80° C. overnight. The coated disks were incubated with blocking reagent (Roche) dissolved in 0.1 M maleic acid buffer for 1 h at room temperature to reduce nonspecific binding of EXPB1. After the blocking, the disks were washed with 20 mM Na acetate 5 times for 3 min each, then incubated with EXPB1 (20 μg per tube; purified by reverse-phase chromatography; see above) in 400 μL of 20 mM sodium acetate, pH 5.5 at 25° C. for 1. After the incubation, the supernatant (unbound protein) was analyzed by reverse phase chromatography (above). The amount of EXPB1 bound to the coated nitrocellulose membrane disks was calculated from the reduction in the amount of unbound protein, assessed by reverse-phase HPLC of the supernatant.

Acknowledgments. This work was supported by DOE Grant FG02-84ER13179 and NIH Grant 5R01GM60397 to DJC. We thank: Dr. Greg Farber for instimable advice and assistance with growing the EXPB1 crystals; Dr. Javier Sampedro for useful discussions; Daniel M. Durachko, Edward Wagner and Dr. Hemant Yennawar for expert technical assistance; Dr. Colin Mitchison for gift of the swollenin sample; Dr. Inez Munoz for gift of the TrCel45 sample; Dr. Jan-Christer Janson for gift of the MeCel45 sample.
1. Cosgrove, D. J. (2005) Nat. Rev. Mol. Cell. Biol. 6, 850-861.
2. Cosgrove, D. J. (1997) Plant Cell 9, 1031-1041.
3. Sampedro, J. & Cosgrove, D. J. (2005) Genome Biol. 6, 242.1-242.11.
4. Cosgrove, D. J. (2000) Nature 407, 321-326.
5. Rose, J. K., Braam, J., Fry, S. C., & Nishitani, K. (2002) Plant Cell Physiol. 43, 1421-1435.
6. Domingo, C., Roberts, K., Stacey, N. J., Connerton, I., Ruiz-Teran, F., & McCann, M. C. (1998) Plant J. 13, 17-28.
7. Carpita, N. C. & Gibeaut, D. M. (1993) Plant J. 3, 1-30.
8. Park, Y. W., Tominaga, R., Sugiyama, J., Furuta, Y., Tanimoto, E., Samejima, M., Sakai, F., & Hayashi, T. (2003) Plant J. 33, 1099-1106.
9. McQueen-Mason, S., Durachko, D. M., & Cosgrove, D. J. (1992) Plant Cell 4, 1425-1433.
10. Li, Z.-C., Durachko, D. M., & Cosgrove, D. J. (1993) Planta 191, 349-356.
11. Darley, C. P., Forrester, A. M., & McQueen-Mason, S. J. (2001) Plant Mol. Biol. 47, 179-195.
12. Choi, D. S., Lee, Y., Cho, H. T., & Kende, H. (2003) Plant Cell 15, 1386-1398.
13. Pien, S., Wyrzykowska, J., McQueen-Mason, S., Smart, C., & Fleming, A. (2001) Proc. Natl. Acad. Sci. USA 98, 11812-11817.
14. Belfield, E. J., Ruperti, B., Roberts, J. A., & McQueen-Mason, S. J. (2005) J. Exp. Bot. 56, 817-823.
15. Cho, H. T. & Cosgrove, D. J. (2000) Proc. Natl. Acad. Sci. USA 97, 9783-9788.
16. Rose, J. K., Lee, H. H., & Bennett, A. B. (1997) Proc. Natl. Acad. Sci. USA 94, 5955-5960.
17. Chen, F., Dahal, P., & Bradford, K. J. (2001) Plant Physiol. 127, 928-936.
18. Li, Y., Darley, C. P., Ongaro, V., Fleming, A., Schipper, O., Baldauf, S. L., & McQueen-Mason, S. J. (2002) Plant Physiol. 128, 854-864.
19. Andersson, K. & Lidholm, J. (2003) Int. Arch. Allergy Immunol. 130, 87-107.
20. Ansari, A. A., Kihara, T. K., & Marsh, D. G. (1987) J. Immunology 12, 4034-4041.
21. Cosgrove, D. J., Bedinger, P., & Durachko, D. M. (1997) Proc. Natl. Acad. Sci. USA 94, 6559-6564.
22. Focke, M., Mahler, V., Ball, T., Sperr, W. R., Majlesi, Y., Valent, P., Kraft, D., & Valenta, R. (2001) FASEB J. 15, 2042-2044.
23. Ball, T., Edstrom, W., Mauch, L., Schmitt, J., Leistler, B., Fiebig, H., Sperr, W. R., Hauswirth, A. W., Valent, P., Kraft, D. et al. (2005) FEBS J. 272, 217-227.
24. Li, L. C., Bedinger, P. A., Volk, C., Jones, A. D., & Cosgrove, D. J. (2003) Plant Physiol. 132, 2073-2085.
25. Wu, Y., Meeley, R. B., & Cosgrove, D. J. (2001) Plant Physiol. 126, 222-232.
26. Hall, Q. & Cannon, M. C. (2002) Plant Cell 14, 1161-1172.
27. Ribeiro, J. M., Pereira, C. S., Soares, N. C., Vieira, A. M., Feijo, J. A., & Jackson, P. A. (2006) J. Exp. Bot.
28. McQueen-Mason, S. J. & Cosgrove, D. J. (1995) Plant Physiol. 107, 87-100.
29. Carpita, N. C. (1996) Annu. Rev. Plant Physiol. Plant Mol. Biol. 47, 445-476.
30. De Marino, S., Morelli, M. A., Fraternali, F., Tamborini, E., Musco, G., Vrtala, S., Dolecek, C., Arosio, P., Valenta, R., & Pastore, A. (1999) Structure Fold Des 7, 943-952.
31. Lerouge, P., Cabanes-Macheteau, M., Rayon, C., Fischette-Laine, A. C., Gomord, V., & Faye, L. (1998) Plant Mol. Biol. 38, 31-48.
32. Xu, B., Hellman, U., Ersson, B., & Janson, J. C. (2000) Eur. J. Biochem. 267, 4970-4977.
33. Davies, G. J., Tolley, S. P., Henrissat, B., Hjort, C., & Schulein, M. (1995) Biochemistry 34, 16210-16220.
34. COLLABORATIVE COMPUTATIONAL PROJECT, N. 4.1. (1994) Acta Cryst. D50, 760-763.
35. Davies, G. J., Dodson, G. G., Hubbard, R. E., Tolley, S. P., Dauter, Z., Wilson, K. S., Hjort, C., Mikkelsen, J. M., Rasmussen, G., & Schulein, M. (1993) Nature 365, 362-364.
36. Saloheimo, A., Henrissat, B., Hoffren, A. M., Teleman, O., & Penttila, M. (1994) Mol. Microbiol. 13, 219-228.
37. Saloheimo, M., Paloheimo, M., Hakola, S., Pere, J., Swanson, B., Nyyssonen, E., Bhatia, A., Ward, M., & Penttila, M. (2002) Eur. J. Biochem. 269, 4202-4211.
38. Lee, Y. & Choi, D. (2005) Mol. Cells. 20, 119-126.
39. Downes, B. P., Steinbaker, C. R., & Crowell, D. N. (2001) Plant Physiol. 126, 244-252.
40. McQueen-Mason, S. & Cosgrove, D. J. (1994) Proc. Natl. Acad. Sci. USA 91, 6574-6578.
41. Bork, P., Holm, L., & Sander, C. (1994) J. Mol. Biol. 242, 309-320.
42. Boraston, A. B., Bolam, D. N., Gilbert, H. J., & Davies, G. J. (2004) Biochem. J. 382, 769-781.
43. Barre, A. & Rouge, P. (2002) Biochem. Biophys. Res. Commun. 296, 1346.
44. Fedorov, A. A., Ball, T., Valenta, R., & Almo, S. C. (1997) Int. Arch. Allergy Immunol. 113, 109-113.
45. Grobe, K., Becker, W. M., & Petersen, A. (1999) Eur. J. Biochem. 263, 33-40.
46. Grobe, K., Poppelmann, M., Becker, W. M., & Petersen, A. (2002) Eur. J. Biochem. 269, 2083-2092.
47. Li, L. C. & Cosgrove, D. J. (2001) Eur. J. Biochem. 268, 4217-4226.
48. Poppelmann, M., Becker, W. M., & Petersen, A. (2002) Electrophoresis 23, 993-997.
49. Sampedro, J., Lee, Y., Carey, R. E., dePamphilis, C., & Cosgrove, D. J. (2005) Plant J. 44, 409-419.
50. Petersen, A., Becker, W. M., & Schlaak, M. (1993) J. Allergy Clin. Immunol. 92, 789-796.
51. Suphioglu, C., Singh, M. B., & Knox, R. B. (1993) Int. Arch. Allergy Appl. Immunol. 102, 144-151.
52. Petersen, A., Becker, W. M., Moll, H., Blumke, M., & Schlaak, M. (1995) Electrophoresis 16, 869-875.
53. Xu, B., Janson, J. C., & Sellos, D. (2001) Eur. J. Biochem. 268, 3718-3727.
54. Marsh, D. G., Milner, F. H., & Johnson, P. (1966) Int. Arch. Allergy Appl. Immunol. 29, 521-535.
55. Ball, T., Fuchs, T., Sperr, W. R., Valent, P., Vangelista, L., Kraft, D., & Valenta, R. (1999) FASEB J. 13, 1277-1290.
56. Howlett, B. J. & Clarke, A. E. (1981) Biochem. J. 197, 707-714.
57. Petersen, A., Schramm, G., Schlaak, M., & Becker, W. M. (1998) Clin. Exp. Allergy 28, 315-321.
58. Ball, T., Vrtala, S., Sperr, W. R., Valent, P., Susani, M., Kraft, D., & Valenta, R. (1994) J. Biol. Chem. 269, 28323-28328.
59. Hiller, K. M., Esch, R. E., & Klapper, D. G. (1997) J. Allergy & Clinical. Immunol. 100, 335-340.
60. Esch, R. E. & Klapper, D. G. (1989) Mol. Immunol. 26, 557-561.
61. Nooren, I. M. & Thornton, J. M. (2003) J. Mol. Biol. 325, 991-1018.
62. Otwinowski, Z. & Minor, W. (1997) Meth. Enzymol. 276, 307-326.
63. Navaza, J. (1994) Acta Crystallographica Section A 50, 157-163.
64. Berman, H. M., Westbrook, J., Feng, Z., Gilliland, G., Bhat, T. N., Weissig, H., Shindyalov, I. N., & Bourne, P. E. (2000) Nucleic Acids Res. 28, 235-242.
65. Fry, S. C. (1988) The Growing Plant Cell Wall: Chemical and Metabolic Analysis (Longman Scientific and Technical, London).
66. Jones, T. A., Zou, J. Y., Cowan, S. W., & Kjeldgaard, M. (1991) Acta Crystallographica Section A 47, 110-119.
67. Brunger, A. T., Adams, P. D., Clore, G. M., DeLano, W. L., Gros, P., Grosse-Kunstleve, R. W., Jiang, J. S., Kuszewski, J., Nilges, M., Pannu, N. S. et al. (1998) Acta Crystallogr. D. Biol. Crystallogr. 54, 905-921.
68. Glaser, F., Pupko, T., Paz, I., Bell, R. E., Bechor-Shental, D., Martz, E., & Ben-Tal, N. (2003) Bioinformatics. 19, 163-164.
69. Wu, S. S., Suen, D. F., Chang, H. C., & Huang, A. H. (2002) J. Biol. Chem. 277, 49055-49064.

TABLE 1

| | |
|---|---|
| HEADER | ALLERGEN 19-JUN-06 2HCZ |

| | |
|---|---|
| TITLE | CRYSTAL STRUCTURE OF EXPB1 (ZEA M 1), A BETA-EXPANSIN AND |
| TITLE | 2 GROUP-1 POLLEN ALLERGEN FROM MAIZE |

| | |
|---|---|
| COMPND | MOL_ID: 1; |
| COMPND | 2 MOLECULE: BETA-EXPANSIN 1A; |
| COMPND | 3 CHAIN: X; |
| COMPND | 4 SYNONYM: POLLEN ALLERGEN ZEA M 1, ZEA M I |
| SOURCE | MOL_ID: 1; |
| SOURCE | 2 ORGANISM_SCIENTIFIC: ZEA MAYS; |
| SOURCE | 3 ORGANISM_COMMON: MAIZE; |
| SOURCE | 4 TISSUE: POLLEN |
| KEYWDS | DOMAIN 1 IS A BETA BARREL AND DOMAIN 2 IS A |

IMMUNOGLOBULIN

| KEYWDS | 2 LIKE BETA-SANDWICH | | |
|---|---|---|---|
| EXPDTA | X-RAY DIFFRACTION | | |
| AUTHOR | N. H. YENNAWAR, D. J. COSGROVE | | |
| REVDAT | 2 24-OCT-06 2HCZ 1 HEADER | | |
| REVDAT | 1 22-AUG-06 2HCZ 0 | | |
| JRNL | AUTH N. H. YENNAWAR, L. C. LI, D. M. DUDZINSKI, A. TABUCHI, | | |
| JRNL | AUTH 2 D. J. COSGROVE | | |
| JRNL | TITL CRYSTAL STRUCTURE AND ACTIVITIES OF EXPB1 (ZEA M | | |
| JRNL | TITL 2 1), A BETA-EXPANSIN AND GROUP-1 POLLEN ALLERGEN | | |
| JRNL | TITL 3 FROM MAIZE. | | |
| JRNL | REF PROC.NATL.ACAD.SCI.USA V. 103 14664 2006 | | |
| JRNL | REFN ASTM PNASA6 US ISSN 0027-8424 | | |
| REMARK | 1 | | |
| REMARK | 2 | | |
| REMARK | 2 | RESOLUTION. 2.75 ANGSTROMS. | |
| REMARK | 3 | | |
| REMARK | 3 | REFINEMENT. | |
| REMARK | 3 | PROGRAM: CNS | |
| REMARK | 3 | AUTHORS: BRUNGER, ADAMS, CLORE, DELANO, GROS, GROSSE- | |
| REMARK | 3 | : KUNSTLEVE, JIANG, KUSZEWSKI, NILGES, PANNU, | |
| REMARK | 3 | : READ, RICE, SIMONSON, WARREN | |
| REMARK | 3 | | |
| REMARK | 3 | REFINEMENT TARGET: ENGH & HUBER | |
| REMARK | 3 | | |
| REMARK | 3 | DATA USED IN REFINEMENT. | |
| REMARK | 3 | RESOLUTION RANGE HIGH (ANGSTROMS): | 2.75 |
| REMARK | 3 | RESOLUTION RANGE LOW (ANGSTROMS): | 29.63 |

| REMARK | 3 | DATA CUTOFF (SIGMA(F)): | 0.000 |
|---|---|---|---|
| REMARK | 3 | DATA CUTOFF HIGH (ABS(F)): | NULL |
| REMARK | 3 | DATA CUTOFF LOW (ABS(F)): | NULL |
| REMARK | 3 | COMPLETENESS (WORKING + TEST) (%): 92.1 | |
| REMARK | 3 | NUMBER OF REFLECTIONS: | 7007 |

| REMARK | 3 | | |
|---|---|---|---|
| REMARK | 3 | FIT TO DATA USED IN REFINEMENT. | |
| REMARK | 3 | CROSS-VALIDATION METHOD: NULL | |
| REMARK | 3 | FREE R VALUE TEST SET SELECTION: RANDOM | |
| REMARK | 3 | R VALUE (WORKING SET): 0.233 | |
| REMARK | 3 | FREE R VALUE: 0.290 | |
| REMARK | 3 | FREE R VALUE TEST SET SIZE (%): 4.800 | |
| REMARK | 3 | FREE R VALUE TEST SET COUNT: 367 | |
| REMARK | 3 | ESTIMATED ERROR OF FREE R VALUE: NULL | |
| REMARK | 3 | | |
| REMARK | 3 | FIT N THE HIGHEST RESOLUTION BIN. | |
| REMARK | 3 | TOTAL NUMBER OF BINS USED: | 8 |
| REMARK | 3 | BIN RESOLUTION RANGE HIGH (A): | 2.75 |
| REMARK | 3 | BIN RESOLUTION RANGE LOW (A): | 2.87 |

| REMARK | 3 | BIN COMPLETENESS (WORKING + TEST) (%): NULL | |
|---|---|---|---|
| REMARK | 3 | REFLECTIONS IN BIN (WORKING SET): 780 | |
| REMARK | 3 | BIN R VALUE (WORKING SET): 0.4200 | |
| REMARK | 3 | BIN FREE R VALUE: 0.5450 | |
| REMARK | 3 | BIN FREE R VALUE TEST SET SIZE (%): NULL | |
| REMARK | 3 | BIN FREE R VALUE TEST SET COUNT: 41 | |
| REMARK | 3 | ESTIMATED ERROR OF BIN FREE R VALUE: NULL | |
| REMARK | 3 | | |
| REMARK | 3 | NUMBER OF NON-HYDROGEN ATOMS USED IN REFINEMENT. | |
| REMARK | 3 | PROTEIN ATOMS: | 1872 |
| REMARK | 3 | NUCLEIC ACID ATOMS: | 0 |
| REMARK | 3 | HETEROGEN ATOMS: | 80 |
| REMARK | 3 | SOLVENT ATOMS: | 17 |

| | | |
|---|---|---|
| REMARK | 3 | |
| REMARK | 3 | B VALUES. |
| REMARK | 3 | FROM WILSON PLOT (A**2): NULL |
| REMARK | 3 | MEAN B VALUE (OVERALL, A**2): 56.87 |
| REMARK | 3 | OVERALL ANISOTROPIC B VALUE. |
| REMARK | 3 | B11 (A**2): −1.64000 |
| REMARK | 3 | B22 (A**2): −4.79800 |
| REMARK | 3 | B33 (A**2): 6.43900 |
| REMARK | 3 | B12 (A**2): 0.00000 |
| REMARK | 3 | B13 (A**2): −1.44900 |
| REMARK | 3 | B23 (A**2): 0.00000 |
| REMARK | 3 | |
| REMARK | 3 | ESTIMATED COORDINATE ERROR. |
| REMARK | 3 | ESD FROM LUZZATI PLOT (A): NULL |
| REMARK | 3 | ESD FROM SIGMAA (A): NULL |
| REMARK | 3 | LOW RESOLUTION CUTOFF (A): NULL |
| REMARK | 3 | |
| REMARK | 3 | CROSS-VALIDATED ESTIMATED COORDINATE ERROR. |
| REMARK | 3 | ESD FROM C-V LUZZATI PLOT (A): NULL |
| REMARK | 3 | ESD FROM C-V SIGMAA (A): NULL |
| REMARK | 3 | |
| REMARK | 3 | RMS DEVIATIONS FROM IDEAL VALUES. |
| REMARK | 3 | BOND LENGTHS (A): 0.008 |
| REMARK | 3 | BOND ANGLES (DEGREES): 1.65 |
| REMARK | 3 | DIHEDRAL ANGLES (DEGREES): NULL |
| REMARK | 3 | IMPROPER ANGLES (DEGREES): NULL |
| REMARK | 3 | |
| REMARK | 3 | ISOTROPIC THERMAL MODEL: NULL |
| REMARK | 3 | |
| REMARK | 3 | ISOTROPIC THERMAL FACTOR RESTRAINTS. RMS SIGMA |
| REMARK | 3 | MAIN-CHAIN BOND (A**2): NULL; NULL |
| REMARK | 3 | MAIN-CHAIN ANGLE (A**2): NULL; NULL |
| REMARK | 3 | SIDE-CHAIN BOND (A**2): NULL; NULL |
| REMARK | 3 | SIDE-CHAIN ANGLE (A**2): NULL; NULL |
| REMARK | 3 | |
| REMARK | 3 | BULK SOLVENT MODELING. |
| REMARK | 3 | METHOD USED: NULL |
| REMARK | 3 | KSOL: NULL |
| REMARK | 3 | BSOL: 46.18 |
| REMARK | 3 | |
| REMARK | 3 | NCS MODEL: NULL |
| REMARK | 3 | |
| REMARK | 3 | NCS RESTRAINTS. RMS SIGMA/WEIGHT |
| REMARK | 3 | GROUP 1 POSITIONAL (A): NULL; NULL |
| REMARK | 3 | GROUP 1 B-FACTOR (A**2): NULL; NULL |
| REMARK | 3 | |
| REMARK | 3 | PARAMETER FILE 1: PROTEIN_REP.PARAM |
| REMARK | 3 | PARAMETER FILE 2: CARBOHYDRATE.PARAM |
| REMARK | 3 | PARAMETER FILE 3: CNS_TOPPAR:WATER_REP.PARAM |
| REMARK | 3 | PARAMETER FILE 4: NULL |
| REMARK | 3 | TOPOLOGY FILE 1: NULL |
| REMARK | 3 | TOPOLOGY FILE 2: NULL |
| REMARK | 3 | TOPOLOGY FILE 3: NULL |
| REMARK | 3 | TOPOLOGY FILE 4: NULL |
| REMARK | 3 | |
| REMARK | 3 | OTHER REFINEMENT REMARKS: NULL |
| REMARK | 4 | |
| REMARK | 4 | 2HCZ COMPLIES WITH FORMAT V. 3.0, 1-DEC-2006 |
| REMARK | 4 | |
| REMARK | 4 | THIS IS THE REMEDIATED VERSION OF THIS PDB ENTRY. |
| REMARK | 4 | REMEDIATED DATA FILE REVISION 3.101 (2007-05-29) |

| REMARK | 100 | | |
|---|---|---|---|
| REMARK | 100 | THIS ENTRY HAS BEEN PROCESSED BY RCSB. | |
| REMARK | 100 | THE RCSB ID CODE IS RCSB038210. | |
| REMARK | 200 | | |
| REMARK | 200 | EXPERIMENTAL DETAILS | |
| REMARK | 200 | EXPERIMENT TYPE: X-RAY DIFFRACTION | |
| REMARK | 200 | DATE OF DATA COLLECTION: 01-OCT-1998 | |
| REMARK | 200 | TEMPERATURE (KELVIN): 298.0 | |
| REMARK | 200 | PH: 4.50 | |
| REMARK | 200 | NUMBER OF CRYSTALS USED: 2 | |
| REMARK | 200 | | |
| REMARK | 200 | SYNCHROTRON (Y/N) | : N |
| REMARK | 200 | RADIATION SOURCE: | ROTATING ANODE |

| | | |
|---|---|---|
| REMARK | 200 | BEAMLINE: NULL |
| REMARK | 200 | X-RAY GENERATOR MODEL: RIGAKU RU200 |
| REMARK | 200 | MONOCHROMATIC OR LAUE (M/L): M |
| REMARK | 200 | WAVELENGTH OR RANGE (A): 1.5418 |
| REMARK | 200 | MONOCHROMATOR: GRAPHITE |
| REMARK | 200 | OPTICS: GRAPHITE |
| REMARK | 200 | |
| REMARK | 200 | DETECTOR TYPE: IMAGE PLATE |
| REMARK | 200 | DETECTOR MANUFACTURER: RIGAKU RAXIS IV |
| REMARK | 200 | INTENSITY-INTEGRATION SOFTWARE: CRYSTALCLEAR |

(MSC/RIGAKU)

| | | |
|---|---|---|
| REMARK | 200 | DATA SCALING SOFTWARE: SCALEPACK |
| REMARK | 200 | |
| REMARK | 200 | NUMBER OF UNIQUE REFLECTIONS: 7013 |
| REMARK | 200 | RESOLUTION RANGE HIGH (A): 2.750 |
| REMARK | 200 | RESOLUTION RANGE LOW (A): 100.000 |
| REMARK | 200 | REJECTION CRITERIA (SIGMA(I)): 0.000 |
| REMARK | 200 | |
| REMARK | 200 | OVERALL. |
| REMARK | 200 | COMPLETENESS FOR RANGE (%): 93.1 |
| REMARK | 200 | DATA REDUNDANCY: NULL |
| REMARK | 200 | R MERGE (I): 0.07900 |
| REMARK | 200 | R SYM (I): NULL |
| REMARK | 200 | <I/SIGMA(I)> FOR THE DATA SET: 7.3000 |
| REMARK | 200 | |
| REMARK | 200 | IN THE HIGHEST RESOLUTION SHELL. |
| REMARK | 200 | HIGHEST RESOLUTION SHELL, RANGE HIGH (A): 2.75 |
| REMARK | 200 | HIGHEST RESOLUTION SHELL, RANGE LOW (A): 2.85 |
| REMARK | 200 | COMPLETENESS FOR SHELL (%): 96.6 |
| REMARK | 200 | DATA REDUNDANCY IN SHELL: NULL |
| REMARK | 200 | R MERGE FOR SHELL (I): 0.40200 |
| REMARK | 200 | R SYM FOR SHELL (I): NULL |
| REMARK | 200 | <I/SIGMA(I)> FOR SHELL: NULL |
| REMARK | 200 | |
| REMARK | 200 | DIFFRACTION PROTOCOL: SINGLE WAVELENGTH |
| REMARK | 200 | METHOD USED TO DETERMINE THE STRUCTURE: MOLECULAR |

REPLACEMENT

| | | |
|---|---|---|
| REMARK | 200 | SOFTWARE USED: AMORE |
| REMARK | 200 | STARTING MODEL: 1N10 |
| REMARK | 200 | |
| REMARK | 200 | REMARK: NULL |
| REMARK | 280 | |
| REMARK | 280 | CRYSTAL |
| REMARK | 280 | SOLVENT CONTENT, VS (%): 54.03 |
| REMARK | 280 | MATTHEWS COEFFICIENT, VM (ANGSTROMS**3/DA): 2.68 |
| REMARK | 280 | |
| REMARK | 280 | CRYSTALLIZATION CONDITIONS: 15% PEG 4000, 50 MM SODIUM |

ACETATE

| | | |
|---|---|---|
| REMARK | 280 | AND 0.1M AMMONIUM SULPHATE, PH 4.5, VAPOR DIFFUSION, |

HANGING

| REMARK | 280 | DROP, TEMPERATURE 294 K | |
|---|---|---|---|
| REMARK | 290 | | |
| REMARK | 290 | CRYSTALLOGRAPHIC SYMMETRY | |
| REMARK | 290 | SYMMETRY OPERATORS FOR SPACE GROUP: C 1 2 1 | |
| REMARK | 290 | | |
| REMARK | 290 | SYMOP SYMMETRY | |
| REMARK | 290 | NNNMMM OPERATOR | |
| REMARK | 290 | 1555 | X, Y, Z |
| REMARK | 290 | 2555 | −X, Y, −Z |
| REMARK | 290 | 3555 | ½ + X, ½ + Y, Z |
| REMARK | 290 | 4555 | ½ − X, ½ + Y, −Z |

| | | |
|---|---|---|
| REMARK | 290 | |
| REMARK | 290 | WHERE NNN -> OPERATOR NUMBER |
| REMARK | 290 | MMM -> TRANSLATION VECTOR |
| REMARK | 290 | |
| REMARK | 290 | CRYSTALLOGRAPHIC SYMMETRY TRANSFORMATIONS |
| REMARK | 290 | THE FOLLOWING TRANSFORMATIONS OPERATE ON THE |

ATOM/HETATM

| | | |
|---|---|---|
| REMARK | 290 | RECORDS IN THIS ENTRY TO PRODUCE |

CRYSTALLOGRAPHICALLY

| REMARK | 290 | RELATED MOLECULES. | | | | | |
|---|---|---|---|---|---|---|---|
| REMARK | 290 | SMTRY1 | 1 | 1.000000 | 0.000000 | 0.000000 | 0.00000 |
| REMARK | 290 | SMTRY2 | 1 | 0.000000 | 1.000000 | 0.000000 | 0.00000 |
| REMARK | 290 | SMTRY3 | 1 | 0.000000 | 0.000000 | 1.000000 | 0.00000 |
| REMARK | 290 | SMTRY1 | 2 | −1.000000 | 0.000000 | 0.000000 | 0.00000 |
| REMARK | 290 | SMTRY2 | 2 | 0.000000 | 1.000000 | 0.000000 | 0.00000 |
| REMARK | 290 | SMTRY3 | 2 | 0.000000 | 0.000000 | −1.000000 | 0.00000 |
| REMARK | 290 | SMTRY1 | 3 | 1.000000 | 0.000000 | 0.000000 | 56.70400 |
| REMARK | 290 | SMTRY2 | 3 | 0.000000 | 1.000000 | 0.000000 | 22.25600 |
| REMARK | 290 | SMTRY3 | 3 | 0.000000 | 0.000000 | 1.000000 | 0.00000 |
| REMARK | 290 | SMTRY1 | 4 | −1.000000 | 0.000000 | 0.000000 | 56.70400 |
| REMARK | 290 | SMTRY2 | 4 | 0.000000 | 1.000000 | 0.000000 | 22.25600 |
| REMARK | 290 | SMTRY3 | 4 | 0.000000 | 0.000000 | −1.000000 | 0.00000 |

| | | |
|---|---|---|
| REMARK | 290 | |
| REMARK | 290 | REMARK: NULL |
| REMARK | 300 | |
| REMARK | 300 | BIOMOLECULE: 1 |
| REMARK | 300 | THIS ENTRY CONTAINS THE CRYSTALLOGRAPHIC |

ASYMMETRIC UNIT

| | | |
|---|---|---|
| REMARK | 300 | WHICH CONSISTS OF 1 CHAIN(S). SEE REMARK 350 FOR |
| REMARK | 300 | INFORMATION ON GENERATING THE BIOLOGICAL |

MOLECULE(S).

| | | |
|---|---|---|
| REMARK | 350 | |
| REMARK | 350 | GENERATING THE BIOMOLECULE |
| REMARK | 350 | COORDINATES FOR A COMPLETE MULTIMER REPRESENTING |

THE KNOWN

| | | |
|---|---|---|
| REMARK | 350 | BIOLOGICALLY SIGNIFICANT OLIGOMERIZATION STATE OF |
| THE | | |
| REMARK | 350 | MOLECULE CAN BE GENERATED BY APPLYING BIOMT |

TRANSFORMATIONS

| REMARK | 350 | GIVEN BELOW. BOTH NON-CRYSTALLOGRAPHIC AND | | | | | |
|---|---|---|---|---|---|---|---|
| REMARK | 350 | CRYSTALLOGRAPHIC OPERATIONS ARE GIVEN. | | | | | |
| REMARK | 350 | | | | | | |
| REMARK | 350 | BIOMOLECULE: 1 | | | | | |
| REMARK | 350 | APPLY THE FOLLOWING TO CHAINS: X, A | | | | | |
| REMARK | 350 | BIOMT1 | 1 | 1.000000 | 0.000000 | 0.000000 | 0.00000 |
| REMARK | 350 | BIOMT2 | 1 | 0.000000 | 1.000000 | 0.000000 | 0.00000 |
| REMARK | 350 | BIOMT3 | 1 | 0.000000 | 0.000000 | 1.000000 | 0.00000 |

| | | |
|---|---|---|
| REMARK | 465 | |
| REMARK | 465 | MISSING RESIDUES |
| REMARK | 465 | THE FOLLOWING RESIDUES WERE NOT LOCATED IN THE |
| REMARK | 465 | EXPERIMENT. (M = MODEL NUMBER; RES = RESIDUE NAME; |

C = CHAIN

| | | |
|---|---|---|
| REMARK | 465 | IDENTIFIER; SSSEQ = SEQUENCE NUMBER; I = INSERTION CODE.) |
| REMARK | 465 | |
| REMARK | 465 | M RES C SSSEQI |
| REMARK | 465 | GLY X 1 |
| REMARK | 465 | PRO X 2 |
| REMARK | 465 | PRO X 3 |
| REMARK | 500 | |
| REMARK | 500 | GEOMETRY AND STEREOCHEMISTRY |
| REMARK | 500 | SUBTOPIC: CLOSE CONTACTS IN SAME ASYMMETRIC UNIT |
| REMARK | 500 | |
| REMARK | 500 | THE FOLLOWING ATOMS ARE IN CLOSE CONTACT. |
| REMARK | 500 | |
| REMARK | 500 | ATM1 RES C SSEQI ATM2 RES C SSEQI |
| REMARK | 500 | C3 NAG A 1 C1 FCA A 7 2.10 |
| REMARK | 500 | O6 MAN A 3 C2 MAN A 5 2.11 |
| REMARK | 500 | O ASN X 16 N LYS X 18 2.14 |
| REMARK | 500 | |
| REMARK | 500 | GEOMETRY AND STEREOCHEMISTRY |
| REMARK | 500 | SUBTOPIC: COVALENT BOND LENGTHS |
| REMARK | 500 | |
| REMARK | 500 | THE STEREOCHEMICAL PARAMETERS OF THE FOLLOWING |

RESIDUES

| | | |
|---|---|---|
| REMARK | 500 | HAVE VALUES WHICH DEVIATE FROM EXPECTED VALUES BY |
| MORE | | |
| REMARK | 500 | THAN 6*RMSD (M = MODEL NUMBER; RES = RESIDUE NAME; |

C = CHAIN

| | | |
|---|---|---|
| REMARK | 500 | IDENTIFIER; SSEQ = SEQUENCE NUMBER; I = INSERTION CODE). |
| REMARK | 500 | |
| REMARK | 500 | STANDARD TABLE: |
| REMARK | 500 | FORMAT: (10X, I3, 1X, 2(A3, 1X, A1, I4, A1, 1X, A4, 3X), F6.3) |
| REMARK | 500 | |
| REMARK | 500 | EXPECTED VALUES: ENGH AND HUBER, 1991 |
| REMARK | 500 | |
| REMARK | 500 | M RES CSSEQI ATM1 RES CSSEQI ATM2 DEVIATION |
| REMARK | 500 | ILE X 44 CA ILE X 44 CB 0.052 |
| REMARK | 500 | |
| REMARK | 500 | GEOMETRY AND STEREOCHEMISTRY |
| REMARK | 500 | SUBTOPIC: COVALENT BOND ANGLES |
| REMARK | 500 | |
| REMARK | 500 | THE STEREOCHEMICAL PARAMETERS OF THE FOLLOWING |

RESIDUES

| | | |
|---|---|---|
| REMARK | 500 | HAVE VALUES WHICH DEVIATE FROM EXPECTED VALUES BY |
| MORE | | |
| REMARK | 500 | THAN 6*RMSD (M = MODEL NUMBER; RES = RESIDUE NAME; |
| | | C = CHAIN |
| REMARK | 500 | IDENTIFIER; SSEQ = SEQUENCE NUMBER; I = INSERTION CODE). |
| REMARK | 500 | |
| REMARK | 500 | STANDARD TABLE: |
| REMARK | 500 | FORMAT: (10X, I3, 1X, A3, 1X, A1, I4, A1, 3(1X, A4, 2X), 12X, F5.1) |
| REMARK | 500 | |
| REMARK | 500 | EXPECTED VALUES: ENGH AND HUBER, 1991 |
| REMARK | 500 | |
| REMARK | 500 | M RES CSSEQI ATM1 ATM2 ATM3 |
| REMARK | 500 | ASN X 10 N-CA-C ANGL. DEV. = −11.5 DEGREES |
| REMARK | 500 | HYP X 9 CA-C-N ANGL. DEV. = 10.9 DEGREES |
| REMARK | 500 | HYP X 9 O-C-N ANGL. DEV. = −12.0 DEGREES |
| REMARK | 500 | GLY X 17 N-CA-C ANGL. DEV. = 12.3 DEGREES |
| REMARK | 500 | TRP X 19 N-CA-C ANGL. DEV. = 10.7 DEGREES |
| REMARK | 500 | ILE X 44 N-CA-C ANGL. DEV. = −10.3 DEGREES |
| REMARK | 500 | CYS X 73 CA-CB-SG ANGL. DEV. = −9.8 DEGREES |
| REMARK | 500 | CYS X 128 N-CA-C ANGL. DEV. = −11.2 DEGREES |
| REMARK | 500 | |
| REMARK | 500 | GEOMETRY AND STEREOCHEMISTRY |
| REMARK | 500 | SUBTOPIC: TORSION ANGLES |
| REMARK | 500 | |
| REMARK | 500 | TORSION ANGLES OUTSIDE THE EXPECTED RAMACHANDRAN |

REGIONS:

| | | |
|---|---|---|
| REMARK | 500 | (M = MODEL NUMBER; RES = RESIDUE NAME; C = CHAIN |

IDENTIFIER;

| | | |
|---|---|---|
| REMARK | 500 | SSEQ = SEQUENCE NUMBER; I = INSERTION CODE). |
| REMARK | 500 | |
| REMARK | 500 | STANDARD TABLE: |
| REMARK | 500 | FORMAT: (10X, I3, 1X, A3, 1X, A1, I4, A1, 4X, F7.2, 3X, F7.2) |
| REMARK | 500 | |
| REMARK | 500 | M RES CSSEQI PSI PHI |
| REMARK | 500 | ASN X 10 113.05 138.93 |
| REMARK | 500 | CYS X 156 −117.96 −158.76 |
| REMARK | 500 | ALA X 162 111.35 121.14 |
| REMARK | 500 | ASP X 170 −97.85 81.46 |
| REMARK | 500 | LEU X 183 127.07 92.22 |
| REMARK | 500 | |
| REMARK | 500 | GEOMETRY AND STEREOCHEMISTRY |
| REMARK | 500 | SUBTOPIC: NON-CIS, NON-TRANS |
| REMARK | 500 | |
| REMARK | 500 | THE FOLLOWING PEPTIDE BONDS DEVIATE SIGNIFICANTLY |

FROM BOTH

| | | |
|---|---|---|
| REMARK | 500 | CIS AND TRANS CONFORMATION. CIS BONDS, IF ANY, ARE |
| LISTED | | |
| REMARK | 500 | ON CISPEP RECORDS. TRANS IS DEFINED AS 180 +/− 30 AND |
| REMARK | 500 | CIS IS DEFINED AS 0 +/− 30 DEGREES. |
| REMARK | 500 | MODEL OMEGA |
| REMARK | 500 | GLY X 8 HYP X 9 60.63 |
| REMARK | 500 | HYP X 9 ASN X 10 −64.54 |
| REMARK | 525 | |
| REMARK | 525 | SOLVENT |
| REMARK | 525 | THE FOLLOWING SOLVENT MOLECULES LIE FARTHER THAN |

EXPECTED

| | | |
|---|---|---|
| REMARK | 525 | FROM THE PROTEIN OR NUCLEIC ACID MOLECULE AND MAY |
| BE | | |
| REMARK | 525 | ASSOCIATED WITH A SYMMETRY RELATED MOLECULE |

(M = MODEL

| | | |
|---|---|---|
| REMARK | 525 | NUMBER; RES = RESIDUE NAME; C = CHAIN IDENTIFIER; |

SSEQ = SEQUENCE

| | | |
|---|---|---|
| REMARK | 525 | NUMBER; I = INSERTION CODE): |
| REMARK | 525 | |
| REMARK | 525 | M RES CSSEQI |
| REMARK | 525 | HOH 7 DISTANCE = 5.93 ANGSTROMS |
| REMARK | 525 | HOH 17 DISTANCE = 5.21 ANGSTROMS |
| REMARK | 900 | |
| REMARK | 900 | RELATED ENTRIES |
| REMARK | 900 | RELATED ID: 1N10 RELATED DB: PDB |
| REMARK | 900 | CRYSTAL STRUCTURE OF PHL P 1, A MAJOR TIMOTHY GRASS |
| POLLEN | | |
| REMARK | 900 | ALLERGEN |

DBREF 2HCZ X 1 245 UNP P58738 EXB1A_MAIZE 25 269
SEQADV 2HCZ HYP X 9 UNP P58738 PRO 33 MODIFIED RESIDUE

| | | | |
|---|---|---|---|
| SEQRES | 1 X | 245 | GLY PRO PRO LYS VAL PRO PRO GLY HYP ASN ILE THR |
| THR | | | |
| SEQRES | 2 X | 245 | ASN TYR ASN GLY LYS TRP LEU THR ALA ARG ALA THR |
| TRP | | | |
| SEQRES | 3 X | 245 | TYR GLY GLN PRO ASN GLY ALA GLY ALA PRO ASP ASN |
| GLY | | | |
| SEQRES | 4 X | 245 | GLY ALA CYS GLY ILE LYS ASN VAL ASN LEU PRO PRO |
| TYR | | | |
| SEQRES | 5 X | 245 | SER GLY MET THR ALA CYS GLY ASN VAL PRO ILE PHE |
| LYS | | | |
| SEQRES | 6 X | 245 | ASP GLY LYS GLY CYS GLY SER CYS TYR GLU VAL ARG |
| CYS | | | |
| SEQRES | 7 X | 245 | LYS GLU LYS PRO GLU CYS SER GLY ASN PRO VAL THR |
| VAL | | | |
| SEQRES | 8 X | 245 | TYR ILE THR ASP MET ASN TYR GLU PRO ILE ALA PRO TYR |
| SEQRES | 9 X | 245 | HIS PHE ASP LEU SER GLY LYS ALA PHE GLY SER LEU ALA |
| SEQRES | 10 X | 245 | LYS PRO GLY LEU ASN ASP LYS ILE ARG HIS CYS GLY ILE |
| SEQRES | 11 X | 245 | MET ASP VAL GLU PHE ARG ARG VAL ARG CYS LYS TYR |
| PRO | | | |
| SEQRES | 12 X | 245 | ALA GLY GLN LYS ILE VAL PHE HIS ILE GLU LYS GLY CYS |
| SEQRES | 13 X | 245 | ASN PRO ASN TYR LEU ALA VAL LEU VAL LYS TYR VAL |
| ALA | | | |
| SEQRES | 14 X | 245 | ASP ASP GLY ASP ILE VAL LEU MET GLU ILE GLN ASP LYS |
| SEQRES | 15 X | 245 | LEU SER ALA GLU TRP LYS PRO MET LYS LEU SER TRP |
| GLY | | | |
| SEQRES | 16 X | 245 | ALA ILE TRP ARG MET ASP THR ALA LYS ALA LEU LYS |
| GLY | | | |
| SEQRES | 17 X | 245 | PRO PHE SER ILE ARG LEU THR SER GLU SER GLY LYS LYS |
| SEQRES | 18 X | 245 | VAL ILE ALA LYS ASP VAL ILE PRO ALA ASN TRP ARG PRO |
| SEQRES | 19 X | 245 | ASP ALA VAL TYR THR SER ASN VAL GLN PHE TYR |

MODRES 2HCZ HYP X 9 PRO 4-HYDROXYPROLINE
MODRES 2HCZ ASN X 10 ASN GLYCOSYLATION SITE

| | | | | |
|---|---|---|---|---|
| HET | HYP | X | 9 | 8 |
| HET | NAG | A | 1 | 14 |
| HET | NAG | A | 2 | 14 |
| HET | MAN | A | 3 | 11 |
| HET | MAN | A | 4 | 11 |
| HET | XYS | A | 6 | 9 |
| HET | MAN | A | 5 | 11 |
| HET | FCA | A | 7 | 10 |

| | |
|---|---|
| HETNAM | HYP 4-HYDROXYPROLINE |
| HETNAM | NAG N-ACETYL-D-GLUCOSAMINE |
| HETNAM | MAN ALPHA-D-MANNOSE |
| HETNAM | XYS XYLOPYRANOSE |
| HETNAM | FCA ALPHA-D-FUCOSE |
| HETSYN | HYP HYDROXYPROLTNE |
| HETSYN | NAG NAG |
| FORMUL | 1 HYP C5 H9 N O3 |
| FORMUL | 2 NAG 2(C8 H15 N O6) |
| FORMUL | 2 MAN 3(C6 H12 O6) |
| FORMUL | 2 XYS C5 H10 O5 |
| FORMUL | 4 FCA C6 H12 O5 |
| FORMUL | 5 HOH *17(H2 O) |

| | | | |
|---|---|---|---|
| HELIX | 1 | 1 ASN X 60 LYS X 65 1 | 6 |
| HELIX | 2 | 2 ASP X 66 LYS X 68 5 | 3 |
| HELIX | 3 | 3 SER X 109 LEU X 116 1 | 8 |
| HELIX | 4 | 4 LEU X 121 ARG X 126 1 | 6 |

| | | |
|---|---|---|
| SHEET | 1 | A 7 LEU X 20 TRP X 26 0 |
| SHEET | 2 | A 7 HIS X 105 LEU X 108 1 O LEU X 108 N THR X 25 |
| SHEET | 3 | A 7 THR X 56 GLY X 59 −1 N CYS X 58 O ASP X 107 |
| SHEET | 4 | A 7 VAL X 89 MET X 96 1 O ASP X 95 N ALA X 57 |
| SHEET | 5 | A 7 CYS X 73 ARG X 77 −1 N VAL X 76 O VAL X 89 |
| SHEET | 6 | A 7 MET X 131 VAL X 138 −1 O GLU X 134 N ARG X 77 |
| SHEET | 7 | A 7 LEU X 20 TRP X 26 −1 N ALA X 24 O MET X 131 |
| SHEET | 1 | B 5 LYS X 191 TRP X 194 0 |
| SHEET | 2 | B 5 ILE X 197 ARG X 199 −1 O ARG X 199 N LYS X 191 |
| SHEET | 3 | B 5 VAL X 163 LYS X 166 −1 N VAL X 163 O TRP X 198 |
| SHEET | 4 | B 5 VAL X 149 ILE X 152 −1 N HIS X 151 O LEU X 164 |
| SHEET | 5 | B 5 VAL X 237 THR X 239 −1 O TYR X 238 N PHE X 150 |
| SHEET | 1 | C 3 ILE X 174 GLU X 178 0 |
| SHEET | 2 | C 3 ILE X 212 SER X 216 −1 O ARG X 213 N GLU X 178 |
| SHEET | 3 | C 3 LYS X 221 ALA X 224 −1 O ALA X 224 N ILE X 212 |

| | | |
|---|---|---|
| SSBOND | 1 | CYS X 42 CYS X 70 |
| SSBOND | 2 | CYS X 73 CYS X 140 |
| SSB0ND | 3 | CYS X 78 CYS X 84 |

| | | |
|---|---|---|
| LINK | ND2 ASN X 10 | C1 NAG A 1 |
| LINK | O4 NAG A 1 | C1 NAG A 2 |
| LINK | O4 NAG A 2 | C1 MAN A 3 |
| LINK | O2 MAN A 3 | C1 XYS A 6 |
| LINK | O6 MAN A 3 | C1 MAN A 5 |
| LINK | O3 NAG A 1 | C1 FCA A 7 |
| LINK | O3 MAN A 3 | C1 MAN A 4 |

CISPEP 1 PRO X 50 PRO X 51 0 −0.15
CRYST1 113.408 44.512 69.467 90.00 124.64 90.00 C 1 2 1 4

| | | | | |
|---|---|---|---|---|
| ORIGX1 | 1.000000 | 0.000000 | 0.000000 | 0.00000 |
| ORIGX2 | 0.000000 | 1.000000 | 0.000000 | 0.00000 |
| ORIGX3 | 0.000000 | 0.000000 | 1.000000 | 0.00000 |
| SCALE1 | 0.008818 | 0.000000 | 0.006092 | 0.00000 |
| SCALE2 | 0.000000 | 0.022466 | 0.000000 | 0.00000 |
| SCALE3 | 0.000000 | 0.000000 | 0.017497 | 0.00000 |

| | | | | | | | | | | | |
|---|---|---|---|---|---|---|---|---|---|---|---|
| ATOM | 1 | N | LYS | X | 4 | −11.044 | −7.638 | 34.841 | 1.00 | 92.39 | N |
| ATOM | 2 | CA | LYS | X | 4 | −9.973 | −7.749 | 33.810 | 1.00 | 92.39 | C |
| ATOM | 3 | C | LYS | X | 4 | −10.533 | −7.543 | 32.405 | 1.00 | 92.39 | C |
| ATOM | 4 | O | LYS | X | 4 | −11.535 | −8.149 | 32.032 | 1.00 | 92.39 | O |
| ATOM | 5 | CB | LYS | X | 4 | −9.313 | −9.126 | 33.897 | 1.00 | 90.09 | C |
| ATOM | 6 | CG | LYS | X | 4 | −8.209 | −9.354 | 32.881 | 1.00 | 90.09 | C |
| ATOM | 7 | CD | LYS | X | 4 | −7.712 | −10.784 | 32.940 | 1.00 | 90.09 | C |
| ATOM | 8 | CE | LYS | X | 4 | −6.665 | −11.045 | 31.875 | 1.00 | 90.09 | C |
| ATOM | 9 | NZ | LYS | X | 4 | −6.247 | −12.474 | 31.854 | 1.00 | 90.09 | N |
| ATOM | 10 | N | VAL | X | 5 | −9.882 | −6.684 | 31.629 | 1.00 | 61.66 | N |
| ATOM | 11 | CA | VAL | X | 5 | −10.323 | −6.418 | 30.267 | 1.00 | 61.66 | C |
| ATOM | 12 | C | VAL | X | 5 | −9.453 | −7.136 | 29.242 | 1.00 | 61.66 | C |
| ATOM | 13 | O | VAL | X | 5 | −8.236 | −6.963 | 29.217 | 1.00 | 61.66 | O |
| ATOM | 14 | CB | VAL | X | 5 | −10.316 | −4.906 | 29.959 | 1.00 | 43.90 | C |
| ATOM | 15 | CG1 | VAL | X | 5 | −10.042 | −4.672 | 28.489 | 1.00 | 43.90 | C |
| ATOM | 16 | CG2 | VAL | X | 5 | −11.659 | −4.301 | 30.332 | 1.00 | 43.90 | C |
| ATOM | 17 | N | PRO | X | 6 | −10.079 | −7.962 | 28.388 | 1.00 | 62.91 | N |
| ATOM | 18 | CA | PRO | X | 6 | −9.422 | −8.739 | 27.329 | 1.00 | 62.91 | C |
| ATOM | 19 | C | PRO | X | 6 | −9.096 | −7.921 | 26.067 | 1.00 | 62.91 | C |
| ATOM | 20 | O | PRO | X | 6 | −9.963 | −7.256 | 25.501 | 1.00 | 62.91 | O |
| ATOM | 21 | CB | PRO | X | 6 | −10.430 | −9.854 | 27.056 | 1.00 | 60.98 | C |
| ATOM | 22 | CG | PRO | X | 6 | −11.740 | −9.160 | 27.261 | 1.00 | 60.98 | C |
| ATOM | 23 | CD | PRO | X | 6 | −11.493 | −8.361 | 28.528 | 1.00 | 60.98 | C |
| ATOM | 24 | N | PRO | X | 7 | −7.837 | −7.987 | 25.605 | 1.00 | 44.83 | N |
| ATOM | 25 | CA | PRO | X | 7 | −7.310 | −7.287 | 24.426 | 1.00 | 44.83 | C |
| ATOM | 26 | C | PRO | X | 7 | −8.084 | −7.529 | 23.140 | 1.00 | 44.83 | C |
| ATOM | 27 | O | PRO | X | 7 | −8.671 | −8.583 | 22.968 | 1.00 | 44.83 | O |
| ATOM | 28 | CB | PRO | X | 7 | −5.872 | −7.794 | 24.336 | 1.00 | 45.78 | C |
| ATOM | 29 | CG | PRO | X | 7 | −5.955 | −9.164 | 24.956 | 1.00 | 45.78 | C |
| ATOM | 30 | CD | PRO | X | 7 | −6.831 | −8.916 | 26.148 | 1.00 | 45.78 | C |
| ATOM | 31 | N | GLY | X | 8 | −8.054 | −6.535 | 22.254 | 1.00 | 94.04 | N |
| ATOM | 32 | CA | GLY | X | 8 | −8.745 | −6.563 | 20.971 | 1.00 | 94.04 | C |
| ATOM | 33 | C | GLY | X | 8 | −9.377 | −7.843 | 20.445 | 1.00 | 94.04 | C |
| ATOM | 34 | O | GLY | X | 8 | −10.249 | −8.411 | 21.101 | 1.00 | 94.04 | O |
| HETATM | 35 | N | HYP | X | 9 | −9.033 | −8.354 | 19.261 | 1.00 | 57.41 | N |
| HETATM | 36 | CA | HYP | X | 9 | −7.613 | −8.797 | 18.932 | 1.00 | 57.41 | C |
| HETATM | 37 | C | HYP | X | 9 | −6.643 | −7.615 | 19.045 | 1.00 | 57.41 | C |
| HETATM | 38 | O | HYP | X | 9 | −6.058 | −7.717 | 20.111 | 1.00 | 57.41 | O |
| HETATM | 39 | CB | HYP | X | 9 | −7.650 | −9.469 | 17.547 | 1.00 | 57.41 | C |
| HETATM | 40 | CG | HYP | X | 9 | −8.931 | −9.087 | 16.923 | 1.00 | 57.41 | C |
| HETATM | 41 | CD | HYP | X | 9 | −9.620 | −8.151 | 17.908 | 1.00 | 57.41 | C |
| HETATM | 42 | OD1 | HYP | X | 9 | −9.742 | −10.250 | 16.748 | 1.00 | 57.41 | O |
| ATOM | 43 | N | ASN | X | 10 | −5.825 | −7.147 | 18.092 | 1.00 | 65.18 | N |
| ATOM | 44 | CA | ASN | X | 10 | −4.661 | −7.776 | 17.413 | 1.00 | 65.18 | C |
| ATOM | 45 | C | ASN | X | 10 | −4.911 | −7.316 | 16.006 | 1.00 | 65.18 | C |
| ATOM | 46 | O | ASN | X | 10 | −5.894 | −7.692 | 15.386 | 1.00 | 65.18 | O |
| ATOM | 47 | CB | ASN | X | 10 | −4.637 | −9.290 | 17.513 | 1.00 | 42.31 | C |
| ATOM | 48 | CG | ASN | X | 10 | −3.220 | −9.895 | 17.664 | 1.00 | 42.31 | C |
| ATOM | 49 | OD1 | ASN | X | 10 | −2.478 | −10.011 | 16.693 | 1.00 | 42.31 | O |
| ATOM | 50 | ND2 | ASN | X | 10 | −2.872 | −10.219 | 18.917 | 1.00 | 42.31 | N |
| ATOM | 51 | N | ILE | X | 11 | −4.019 | −6.463 | 15.524 | 1.00 | 32.17 | N |
| ATOM | 52 | CA | ILE | X | 11 | −4.162 | −5.836 | 14.228 | 1.00 | 32.17 | C |
| ATOM | 53 | C | ILE | X | 11 | −3.131 | −6.269 | 13.218 | 1.00 | 32.17 | C |
| ATOM | 54 | O | ILE | X | 11 | −1.929 | −6.249 | 13.471 | 1.00 | 32.17 | O |
| ATOM | 55 | CB | ILE | X | 11 | −4.196 | −4.291 | 14.444 | 1.00 | 23.05 | C |
| ATOM | 56 | CG1 | ILE | X | 11 | −5.539 | −3.944 | 15.077 | 1.00 | 23.05 | C |
| ATOM | 57 | CG2 | ILE | X | 11 | −4.015 | −3.525 | 13.158 | 1.00 | 23.05 | C |
| ATOM | 58 | CD1 | ILE | X | 11 | −5.648 | −2.564 | 15.569 | 1.00 | 23.05 | C |
| ATOM | 59 | N | THR | X | 12 | −3.636 | −6.670 | 12.059 | 1.00 | 42.32 | N |
| ATOM | 60 | CA | THR | X | 12 | −2.805 | −7.160 | 10.974 | 1.00 | 42.32 | C |
| ATOM | 61 | C | THR | X | 12 | −2.568 | −6.124 | 9.907 | 1.00 | 42.32 | C |
| ATOM | 62 | O | THR | X | 12 | −3.041 | −4.994 | 10.004 | 1.00 | 42.32 | O |
| ATOM | 63 | CB | THR | X | 12 | −3.449 | −8.365 | 10.314 | 1.00 | 51.84 | C |
| ATOM | 64 | OG1 | THR | X | 12 | −4.786 | −8.024 | 9.932 | 1.00 | 51.84 | O |
| ATOM | 65 | CG2 | THR | X | 12 | −3.484 | −9.540 | 11.277 | 1.00 | 51.84 | C |
| ATOM | 66 | N | THR | X | 13 | −1.826 | −6.521 | 8.882 | 1.00 | 31.52 | N |
| ATOM | 67 | CA | THR | X | 13 | −1.532 | −5.620 | 7.793 | 1.00 | 31.52 | C |
| ATOM | 68 | C | THR | X | 13 | −2.739 | −5.437 | 6.868 | 1.00 | 31.52 | C |
| ATOM | 69 | O | THR | X | 13 | −2.620 | −4.935 | 5.754 | 1.00 | 31.52 | O |
| ATOM | 70 | CB | THR | X | 13 | −0.353 | −6.120 | 7.003 | 1.00 | 31.48 | C |
| ATOM | 71 | OG1 | THR | X | 13 | −0.615 | −7.453 | 6.563 | 1.00 | 31.48 | O |
| ATOM | 72 | CG2 | THR | X | 13 | 0.889 | −6.096 | 7.862 | 1.00 | 31.48 | C |
| ATOM | 73 | N | ASN | X | 14 | −3.909 | −5.827 | 7.346 | 1.00 | 39.41 | N |
| ATOM | 74 | CA | ASN | X | 14 | −5.118 | −5.663 | 6.567 | 1.00 | 39.41 | C |
| ATOM | 75 | C | ASN | X | 14 | −5.551 | −4.194 | 6.652 | 1.00 | 39.41 | C |
| ATOM | 76 | O | ASN | X | 14 | −6.186 | −3.785 | 7.621 | 1.00 | 39.41 | O |
| ATOM | 77 | CB | ASN | X | 14 | −6.210 | −6.583 | 7.122 | 1.00 | 75.84 | C |
| ATOM | 78 | CG | ASN | X | 14 | −7.544 | −6.392 | 6.433 | 1.00 | 75.84 | C |
| ATOM | 79 | OD1 | ASN | X | 14 | −7.617 | −6.311 | 5.205 | 1.00 | 75.84 | O |
| ATOM | 80 | ND2 | ASN | X | 14 | −8.613 | −6.329 | 7.222 | 1.00 | 75.84 | N |
| ATOM | 81 | N | TYR | X | 15 | −5.205 | −3.398 | 5.645 | 1.00 | 34.29 | N |
| ATOM | 82 | CA | TYR | X | 15 | −5.574 | −1.986 | 5.648 | 1.00 | 34.29 | C |
| ATOM | 83 | C | TYR | X | 15 | −7.024 | −1.708 | 5.222 | 1.00 | 34.29 | C |
| ATOM | 84 | O | TYR | X | 15 | −7.256 | −1.197 | 4.135 | 1.00 | 34.29 | O |
| ATOM | 85 | CB | TYR | X | 15 | −4.605 | −1.202 | 4.750 | 1.00 | 28.79 | C |
| ATOM | 86 | CG | TYR | X | 15 | −3.152 | −1.309 | 5.199 | 1.00 | 28.79 | C |
| ATOM | 87 | CD1 | TYR | X | 15 | −2.227 | −2.065 | 4.487 | 1.00 | 28.79 | C |
| ATOM | 88 | CD2 | TYR | X | 15 | −2.724 | −0.689 | 6.381 | 1.00 | 28.79 | C |
| ATOM | 89 | CE1 | TYR | X | 15 | −0.927 | −2.206 | 4.941 | 1.00 | 28.79 | C |
| ATOM | 90 | CE2 | TYR | X | 15 | −1.426 | −0.822 | 6.842 | 1.00 | 28.79 | C |
| ATOM | 91 | CZ | TYR | X | 15 | −0.533 | −1.578 | 6.127 | 1.00 | 28.79 | C |
| ATOM | 92 | OH | TYR | X | 15 | 0.752 | −1.708 | 6.609 | 1.00 | 28.79 | O |
| ATOM | 93 | N | ASN | X | 16 | −7.998 | −2.021 | 6.081 | 1.00 | 45.44 | N |
| ATOM | 94 | CA | ASN | X | 16 | −9.414 | −1.793 | 5.758 | 1.00 | 45.44 | C |
| ATOM | 95 | C | ASN | X | 16 | −9.935 | −0.433 | 6.215 | 1.00 | 45.44 | C |
| ATOM | 96 | O | ASN | X | 16 | −10.921 | −0.343 | 6.942 | 1.00 | 45.44 | O |
| ATOM | 97 | CB | ASN | X | 16 | −10.301 | −2.887 | 6.363 | 1.00 | 49.59 | C |
| ATOM | 98 | CG | ASN | X | 16 | −10.458 | −2.758 | 7.868 | 1.00 | 49.59 | C |
| ATOM | 99 | OD1 | ASN | X | 16 | −9.623 | −3.225 | 8.626 | 1.00 | 49.59 | O |
| ATOM | 100 | ND2 | ASN | X | 16 | −11.534 | −2.116 | 8.301 | 1.00 | 49.59 | N |
| ATOM | 101 | N | GLY | X | 17 | −9.257 | 0.614 | 5.763 | 1.00 | 44.10 | N |
| ATOM | 102 | CA | GLY | X | 17 | −9.595 | 1.992 | 6.084 | 1.00 | 44.10 | C |
| ATOM | 103 | C | GLY | X | 17 | −10.736 | 2.415 | 6.993 | 1.00 | 44.10 | C |
| ATOM | 104 | O | GLY | X | 17 | −11.173 | 3.562 | 6.900 | 1.00 | 44.10 | O |
| ATOM | 105 | N | LYS | X | 18 | −11.240 | 1.558 | 7.871 | 1.00 | 46.12 | N |
| ATOM | 106 | CA | LYS | X | 18 | −12.306 | 2.036 | 8.730 | 1.00 | 46.12 | C |
| ATOM | 107 | C | LYS | X | 18 | −12.032 | 1.996 | 10.222 | 1.00 | 46.12 | C |
| ATOM | 108 | O | LYS | X | 18 | −11.373 | 1.103 | 10.737 | 1.00 | 46.12 | O |
| ATOM | 109 | CB | LYS | X | 18 | −13.639 | 1.380 | 8.374 | 1.00 | 99.00 | C |
| ATOM | 110 | CG | LYS | X | 18 | −14.290 | 2.045 | 7.150 | 1.00 | 99.00 | C |
| ATOM | 111 | CD | LYS | X | 18 | −14.283 | 3.571 | 7.291 | 1.00 | 99.00 | C |
| ATOM | 112 | CE | LYS | X | 18 | −14.408 | 4.267 | 5.946 | 1.00 | 99.00 | C |
| ATOM | 113 | NZ | LYS | X | 18 | −14.104 | 5.729 | 6.041 | 1.00 | 99.00 | N |
| ATOM | 114 | N | TRP | X | 19 | −12.568 | 3.005 | 10.894 | 1.00 | 43.83 | N |
| ATOM | 115 | CA | TRP | X | 19 | −12.366 | 3.248 | 12.308 | 1.00 | 43.83 | C |
| ATOM | 116 | C | TRP | X | 19 | −12.793 | 2.257 | 13.354 | 1.00 | 43.83 | C |
| ATOM | 117 | O | TRP | X | 19 | −13.855 | 1.659 | 13.283 | 1.00 | 43.83 | O |
| ATOM | 118 | CB | TRP | X | 19 | −12.938 | 4.620 | 12.638 | 1.00 | 49.74 | C |
| ATOM | 119 | CG | TRP | X | 19 | −12.492 | 5.613 | 11.625 | 1.00 | 49.74 | C |
| ATOM | 120 | CD1 | TRP | X | 19 | −13.056 | 5.850 | 10.398 | 1.00 | 49.74 | C |
| ATOM | 121 | CD2 | TRP | X | 19 | −11.315 | 6.422 | 11.688 | 1.00 | 49.74 | C |
| ATOM | 122 | NE1 | TRP | X | 19 | −12.295 | 6.754 | 9.694 | 1.00 | 49.74 | N |
| ATOM | 123 | CE2 | TRP | X | 19 | −11.222 | 7.122 | 10.462 | 1.00 | 49.74 | C |
| ATOM | 124 | CE3 | TRP | X | 19 | −10.328 | 6.624 | 12.660 | 1.00 | 49.74 | C |
| ATOM | 125 | CZ2 | TRP | X | 19 | −10.179 | 8.009 | 10.186 | 1.00 | 49.74 | C |
| ATOM | 126 | CZ3 | TRP | X | 19 | −9.291 | 7.505 | 12.386 | 1.00 | 49.74 | C |
| ATOM | 127 | CH2 | TRP | X | 19 | −9.225 | 8.188 | 11.158 | 1.00 | 49.74 | C |
| ATOM | 128 | N | LEU | X | 20 | −11.924 | 2.115 | 14.345 | 1.00 | 31.46 | N |
| ATOM | 129 | CA | LEU | X | 20 | −12.138 | 1.231 | 15.470 | 1.00 | 31.46 | C |
| ATOM | 130 | C | LEU | X | 20 | −12.099 | 2.090 | 16.730 | 1.00 | 31.46 | C |
| ATOM | 131 | O | LEU | X | 20 | −11.491 | 3.161 | 16.740 | 1.00 | 31.46 | O |
| ATOM | 132 | CB | LEU | X | 20 | −11.020 | 0.202 | 15.530 | 1.00 | 26.96 | C |
| ATOM | 133 | CG | LEU | X | 20 | −10.943 | −0.813 | 14.404 | 1.00 | 26.96 | C |
| ATOM | 134 | CD1 | LEU | X | 20 | −9.602 | −1.516 | 14.413 | 1.00 | 26.96 | C |
| ATOM | 135 | CD2 | LEU | X | 20 | −12.078 | −1.792 | 14.584 | 1.00 | 26.96 | C |
| ATOM | 136 | N | THR | X | 21 | −12.742 | 1.617 | 17.790 | 1.00 | 52.90 | N |
| ATOM | 137 | CA | THR | X | 21 | −12.760 | 2.347 | 19.046 | 1.00 | 52.90 | C |
| ATOM | 138 | C | THR | X | 21 | −11.534 | 1.975 | 19.873 | 1.00 | 52.90 | C |
| ATOM | 139 | O | THR | X | 21 | −10.907 | 0.939 | 19.650 | 1.00 | 52.90 | O |
| ATOM | 140 | CB | THR | X | 21 | −14.023 | 2.032 | 19.853 | 1.00 | 51.86 | C |
| ATOM | 141 | OG1 | THR | X | 21 | −15.171 | 2.252 | 19.033 | 1.00 | 51.86 | O |
| ATOM | 142 | CG2 | THR | X | 21 | −14.128 | 2.940 | 21.062 | 1.00 | 51.86 | C |
| ATOM | 143 | N | ALA | X | 22 | −11.194 | 2.834 | 20.824 | 1.00 | 55.16 | N |
| ATOM | 144 | CA | ALA | X | 22 | −10.050 | 2.612 | 21.686 | 1.00 | 55.16 | C |
| ATOM | 145 | C | ALA | X | 22 | −9.981 | 3.743 | 22.696 | 1.00 | 55.16 | C |
| ATOM | 146 | O | ALA | X | 22 | −10.505 | 4.825 | 22.450 | 1.00 | 55.16 | O |
| ATOM | 147 | CB | ALA | X | 22 | −8.781 | 2.579 | 20.858 | 1.00 | 9.51 | C |
| ATOM | 148 | N | ARG | X | 23 | −9.350 | 3.488 | 23.837 | 1.00 | 59.92 | N |
| ATOM | 149 | CA | ARG | X | 23 | −9.205 | 4.517 | 24.853 | 1.00 | 59.92 | C |
| ATOM | 150 | C | ARG | X | 23 | −7.787 | 5.051 | 24.811 | 1.00 | 59.92 | C |
| ATOM | 151 | O | ARG | X | 23 | −6.820 | 4.289 | 24.771 | 1.00 | 59.92 | O |
| ATOM | 152 | CB | ARG | X | 23 | −9.533 | 3.973 | 26.246 | 1.00 | 77.51 | C |
| ATOM | 153 | CG | ARG | X | 23 | −11.019 | 4.054 | 26.577 | 1.00 | 77.51 | C |
| ATOM | 154 | CD | ARG | X | 23 | −11.285 | 4.021 | 28.083 | 1.00 | 77.51 | C |
| ATOM | 155 | NE | ARG | X | 23 | −12.625 | 4.511 | 28.420 | 1.00 | 77.51 | N |
| ATOM | 156 | CZ | ARG | X | 23 | −13.048 | 5.757 | 28.215 | 1.00 | 77.51 | C |
| ATOM | 157 | NH1 | ARG | X | 23 | −12.243 | 6.661 | 27.672 | 1.00 | 77.51 | N |
| ATOM | 158 | NH2 | ARG | X | 23 | −14.283 | 6.102 | 28.552 | 1.00 | 77.51 | N |
| ATOM | 159 | N | ALA | X | 24 | −7.659 | 6.369 | 24.805 | 1.00 | 44.47 | N |
| ATOM | 160 | CA | ALA | X | 24 | −6.341 | 6.966 | 24.748 | 1.00 | 44.47 | C |
| ATOM | 161 | C | ALA | X | 24 | −6.021 | 7.717 | 26.014 | 1.00 | 44.47 | C |
| ATOM | 162 | O | ALA | X | 24 | −6.913 | 8.109 | 26.760 | 1.00 | 44.47 | O |
| ATOM | 163 | CB | ALA | X | 24 | −6.253 | 7.906 | 23.564 | 1.00 | 47.78 | C |
| ATOM | 164 | N | THR | X | 25 | −4.732 | 7.894 | 26.255 | 1.00 | 50.03 | N |
| ATOM | 165 | CA | THR | X | 25 | −4.239 | 8.644 | 27.402 | 1.00 | 50.03 | C |
| ATOM | 166 | C | THR | X | 25 | −2.843 | 9.018 | 26.977 | 1.00 | 50.03 | C |
| ATOM | 167 | O | THR | X | 25 | −2.365 | 8.568 | 25.931 | 1.00 | 50.03 | O |
| ATOM | 168 | CB | THR | X | 25 | −4.123 | 7.808 | 28.701 | 1.00 | 22.06 | C |
| ATOM | 169 | OG1 | THR | X | 25 | −3.065 | 6.849 | 28.560 | 1.00 | 22.06 | O |
| ATOM | 170 | CG2 | THR | X | 25 | −5.435 | 7.106 | 29.015 | 1.00 | 22.06 | C |
| ATOM | 171 | N | TRP | X | 26 | −2.181 | 9.833 | 27.778 | 1.00 | 39.67 | N |
| ATOM | 172 | CA | TRP | X | 26 | −0.832 | 10.237 | 27.435 | 1.00 | 39.67 | C |
| ATOM | 173 | C | TRP | X | 26 | 0.069 | 10.120 | 28.645 | 1.00 | 39.67 | C |
| ATOM | 174 | O | TRP | X | 26 | −0.404 | 10.061 | 29.777 | 1.00 | 39.67 | O |
| ATOM | 175 | CB | TRP | X | 26 | −0.825 | 11.673 | 26.900 | 1.00 | 46.69 | C |
| ATOM | 176 | CG | TRP | X | 26 | −1.363 | 12.699 | 27.859 | 1.00 | 46.69 | C |
| ATOM | 177 | CD1 | TRP | X | 26 | −2.668 | 12.895 | 28.210 | 1.00 | 46.69 | C |
| ATOM | 178 | CD2 | TRP | X | 26 | −0.605 | 13.684 | 28.562 | 1.00 | 46.69 | C |
| ATOM | 179 | NE1 | TRP | X | 26 | −2.767 | 13.948 | 29.086 | 1.00 | 46.69 | N |
| ATOM | 180 | CE2 | TRP | X | 26 | −1.515 | 14.451 | 29.318 | 1.00 | 46.69 | C |
| ATOM | 181 | CE3 | TRP | X | 26 | 0.760 | 13.995 | 28.624 | 1.00 | 46.69 | C |
| ATOM | 182 | CZ2 | TRP | X | 26 | −1.111 | 15.511 | 30.122 | 1.00 | 46.69 | C |
| ATOM | 183 | CZ3 | TRP | X | 26 | 1.165 | 15.045 | 29.421 | 1.00 | 46.69 | C |
| ATOM | 184 | CH2 | TRP | X | 26 | 0.230 | 15.795 | 30.162 | 1.00 | 46.69 | C |
| ATOM | 185 | N | TYR | X | 27 | 1.368 | 10.083 | 28.402 | 1.00 | 45.95 | N |
| ATOM | 186 | CA | TYR | X | 27 | 2.314 | 9.957 | 29.486 | 1.00 | 45.95 | C |
| ATOM | 187 | C | TYR | X | 27 | 3.535 | 10.801 | 29.163 | 1.00 | 45.95 | C |
| ATOM | 188 | O | TYR | X | 27 | 3.703 | 11.238 | 28.018 | 1.00 | 45.95 | O |
| ATOM | 189 | CB | TYR | X | 27 | 2.719 | 8.497 | 29.644 | 1.00 | 30.14 | C |
| ATOM | 190 | CG | TYR | X | 27 | 3.577 | 7.999 | 28.511 | 1.00 | 30.14 | C |
| ATOM | 191 | CD1 | TYR | X | 27 | 3.085 | 7.956 | 27.206 | 1.00 | 30.14 | C |
| ATOM | 192 | CD2 | TYR | X | 27 | 4.895 | 7.583 | 28.737 | 1.00 | 30.14 | C |
| ATOM | 193 | CE1 | TYR | X | 27 | 3.891 | 7.509 | 26.138 | 1.00 | 30.14 | C |
| ATOM | 194 | CE2 | TYR | X | 27 | 5.710 | 7.129 | 27.677 | 1.00 | 30.14 | C |
| ATOM | 195 | CZ | TYR | X | 27 | 5.197 | 7.098 | 26.378 | 1.00 | 30.14 | C |
| ATOM | 196 | OH | TYR | X | 27 | 5.981 | 6.670 | 25.328 | 1.00 | 30.14 | O |
| ATOM | 197 | N | GLY | X | 28 | 4.383 | 11.020 | 30.170 | 1.00 | 44.81 | N |
| ATOM | 198 | CA | GLY | X | 28 | 5.584 | 11.820 | 29.980 | 1.00 | 44.81 | C |
| ATOM | 199 | C | GLY | X | 28 | 5.305 | 13.312 | 30.079 | 1.00 | 44.81 | C |
| ATOM | 200 | O | GLY | X | 28 | 4.337 | 13.730 | 30.722 | 1.00 | 44.81 | O |
| ATOM | 201 | N | GLN | X | 29 | 6.148 | 14.122 | 29.443 | 1.00 | 65.60 | N |
| ATOM | 202 | CA | GLN | X | 29 | 5.943 | 15.565 | 29.470 | 1.00 | 65.60 | C |
| ATOM | 203 | C | GLN | X | 29 | 4.793 | 15.924 | 28.532 | 1.00 | 65.60 | C |
| ATOM | 204 | O | GLN | X | 29 | 4.520 | 15.209 | 27.570 | 1.00 | 65.60 | O |
| ATOM | 205 | CB | GLN | X | 29 | 7.221 | 16.299 | 29.066 | 1.00 | 87.69 | C |
| ATOM | 206 | CG | GLN | X | 29 | 7.660 | 17.361 | 30.078 | 1.00 | 87.69 | C |
| ATOM | 207 | CD | GLN | X | 29 | 7.957 | 16.790 | 31.467 | 1.00 | 87.69 | C |
| ATOM | 208 | OE1 | GLN | X | 29 | 7.073 | 16.255 | 32.141 | 1.00 | 87.69 | O |
| ATOM | 209 | NE2 | GLN | X | 29 | 9.209 | 16.905 | 31.897 | 1.00 | 87.69 | N |
| ATOM | 210 | N | PRO | X | 30 | 4.108 | 17.045 | 28.797 | 1.00 | 50.97 | N |
| ATOM | 211 | CA | PRO | X | 30 | 2.973 | 17.495 | 27.984 | 1.00 | 50.97 | C |
| ATOM | 212 | C | PRO | X | 30 | 3.313 | 17.904 | 26.555 | 1.00 | 50.97 | C |
| ATOM | 213 | O | PRO | X | 30 | 2.447 | 17.913 | 25.690 | 1.00 | 50.97 | O |
| ATOM | 214 | CB | PRO | X | 30 | 2.403 | 18.661 | 28.797 | 1.00 | 69.17 | C |
| ATOM | 215 | CG | PRO | X | 30 | 2.977 | 18.465 | 30.189 | 1.00 | 69.17 | C |
| ATOM | 216 | CD | PRO | X | 30 | 4.360 | 17.994 | 29.890 | 1.00 | 69.17 | C |
| ATOM | 217 | N | ASN | X | 31 | 4.568 | 18.260 | 26.317 | 1.00 | 33.36 | N |
| ATOM | 218 | CA | ASN | X | 31 | 5.017 | 18.658 | 24.991 | 1.00 | 33.36 | C |
| ATOM | 219 | C | ASN | X | 31 | 6.219 | 17.812 | 24.633 | 1.00 | 33.36 | C |
| ATOM | 220 | O | ASN | X | 31 | 7.083 | 18.221 | 23.854 | 1.00 | 33.36 | O |
| ATOM | 221 | CB | ASN | X | 31 | 5.394 | 20.142 | 24.963 | 1.00 | 78.89 | C |
| ATOM | 222 | CG | ASN | X | 31 | 4.264 | 21.022 | 24.463 | 1.00 | 78.89 | C |
| ATOM | 223 | OD1 | ASN | X | 31 | 3.174 | 21.040 | 25.036 | 1.00 | 78.89 | O |
| ATOM | 224 | ND2 | ASN | X | 31 | 4.519 | 21.756 | 23.386 | 1.00 | 78.89 | N |
| ATOM | 225 | N | GLY | X | 32 | 6.268 | 16.623 | 25.221 | 1.00 | 72.27 | N |
| ATOM | 226 | CA | GLY | X | 32 | 7.360 | 15.711 | 24.954 | 1.00 | 72.27 | C |
| ATOM | 227 | C | GLY | X | 32 | 6.895 | 14.567 | 24.077 | 1.00 | 72.27 | C |
| ATOM | 228 | O | GLY | X | 32 | 5.734 | 14.515 | 23.665 | 1.00 | 72.27 | O |
| ATOM | 229 | N | ALA | X | 33 | 7.808 | 13.647 | 23.795 | 1.00 | 50.75 | N |
| ATOM | 230 | CA | ALA | X | 33 | 7.501 | 12.495 | 22.966 | 1.00 | 50.75 | C |
| ATOM | 231 | C | ALA | X | 33 | 7.306 | 11.232 | 23.814 | 1.00 | 50.75 | C |
| ATOM | 232 | O | ALA | X | 33 | 7.839 | 10.174 | 23.493 | 1.00 | 50.75 | O |
| ATOM | 233 | CB | ALA | X | 33 | 8.622 | 12.287 | 21.950 | 1.00 | 41.40 | C |
| ATOM | 234 | N | GLY | X | 34 | 6.550 | 11.348 | 24.901 | 1.00 | 51.63 | N |
| ATOM | 235 | CA | GLY | X | 34 | 6.312 | 10.198 | 25.758 | 1.00 | 51.63 | C |
| ATOM | 236 | C | GLY | X | 34 | 7.402 | 9.870 | 26.774 | 1.00 | 51.63 | C |
| ATOM | 237 | O | GLY | X | 34 | 7.419 | 10.402 | 27.894 | 1.00 | 51.63 | O |
| ATOM | 238 | N | ALA | X | 35 | 8.313 | 8.980 | 26.388 | 1.00 | 81.75 | N |
| ATOM | 239 | CA | ALA | X | 35 | 9.407 | 8.570 | 27.263 | 1.00 | 81.75 | C |
| ATOM | 240 | C | ALA | X | 35 | 10.295 | 9.745 | 27.653 | 1.00 | 81.75 | C |
| ATOM | 241 | O | ALA | X | 35 | 10.804 | 10.460 | 26.792 | 1.00 | 81.75 | O |
| ATOM | 242 | CB | ALA | X | 35 | 10.241 | 7.502 | 26.581 | 1.00 | 76.43 | C |
| ATOM | 243 | N | PRO | X | 36 | 10.487 | 9.963 | 28.964 | 1.00 | 39.52 | N |
| ATOM | 244 | CA | PRO | X | 36 | 11.322 | 11.059 | 29.458 | 1.00 | 39.52 | C |
| ATOM | 245 | C | PRO | X | 36 | 12.663 | 11.134 | 28.730 | 1.00 | 39.52 | C |
| ATOM | 246 | O | PRO | X | 36 | 13.208 | 12.218 | 28.512 | 1.00 | 39.52 | O |
| ATOM | 247 | CB | PRO | X | 36 | 11.467 | 10.720 | 30.930 | 1.00 | 53.57 | C |
| ATOM | 248 | CG | PRO | X | 36 | 10.105 | 10.173 | 31.256 | 1.00 | 53.57 | C |
| ATOM | 249 | CD | PRO | X | 36 | 9.841 | 9.250 | 30.085 | 1.00 | 53.57 | C |
| ATOM | 250 | N | ASP | X | 37 | 13.172 | 9.970 | 28.338 | 1.00 | 47.65 | N |
| ATOM | 251 | CA | ASP | X | 37 | 14.447 | 9.865 | 27.624 | 1.00 | 47.65 | C |
| ATOM | 252 | C | ASP | X | 37 | 14.282 | 9.995 | 26.111 | 1.00 | 47.65 | C |
| ATOM | 253 | O | ASP | X | 37 | 15.265 | 10.016 | 25.369 | 1.00 | 47.65 | O |
| ATOM | 254 | CB | ASP | X | 37 | 15.106 | 8.519 | 27.930 | 1.00 | 65.15 | C |
| ATOM | 255 | CG | ASP | X | 37 | 14.152 | 7.350 | 27.754 | 1.00 | 65.15 | C |
| ATOM | 256 | OD1 | ASP | X | 37 | 14.633 | 6.206 | 27.628 | 1.00 | 65.15 | O |
| ATOM | 257 | OD2 | ASP | X | 37 | 12.919 | 7.572 | 27.752 | 1.00 | 65.15 | O |
| ATOM | 258 | N | ASN | X | 38 | 13.033 | 10.078 | 25.664 | 1.00 | 73.67 | N |
| ATOM | 259 | CA | ASN | X | 38 | 12.723 | 10.181 | 24.246 | 1.00 | 73.67 | C |
| ATOM | 260 | C | ASN | X | 38 | 13.202 | 8.932 | 23.514 | 1.00 | 73.67 | C |
| ATOM | 261 | O | ASN | X | 38 | 13.939 | 8.999 | 22.526 | 1.00 | 73.67 | O |
| ATOM | 262 | CB | ASN | X | 38 | 13.361 | 11.428 | 23.642 | 1.00 | 36.05 | C |
| ATOM | 263 | CG | ASN | X | 38 | 12.766 | 12.705 | 24.190 | 1.00 | 36.05 | C |
| ATOM | 264 | OD1 | ASN | X | 38 | 11.569 | 12.785 | 24.481 | 1.00 | 36.05 | O |
| ATOM | 265 | ND2 | ASN | X | 38 | 13.599 | 13.722 | 24.318 | 1.00 | 36.05 | N |
| ATOM | 266 | N | GLY | X | 39 | 12.768 | 7.790 | 24.031 | 1.00 | 36.67 | N |
| ATOM | 267 | CA | GLY | X | 39 | 13.106 | 6.500 | 23.465 | 1.00 | 36.67 | C |
| ATOM | 268 | C | GLY | X | 39 | 11.937 | 5.627 | 23.860 | 1.00 | 36.67 | C |
| ATOM | 269 | O | GLY | X | 39 | 10.855 | 6.154 | 24.139 | 1.00 | 36.67 | O |
| ATOM | 270 | N | GLY | X | 40 | 12.129 | 4.313 | 23.915 | 1.00 | 17.90 | N |
| ATOM | 271 | CA | GLY | X | 40 | 11.015 | 3.462 | 24.293 | 1.00 | 17.90 | C |
| ATOM | 272 | C | GLY | X | 40 | 11.279 | 1.982 | 24.121 | 1.00 | 17.90 | C |
| ATOM | 273 | O | GLY | X | 40 | 12.359 | 1.547 | 23.681 | 1.00 | 17.90 | O |
| ATOM | 274 | N | ALA | X | 41 | 10.260 | 1.205 | 24.465 | 1.00 | 32.57 | N |
| ATOM | 275 | CA | ALA | X | 41 | 10.332 | −0.233 | 24.390 | 1.00 | 32.57 | C |
| ATOM | 276 | C | ALA | X | 41 | 10.851 | −0.749 | 23.067 | 1.00 | 32.57 | C |
| ATOM | 277 | O | ALA | X | 41 | 11.387 | −1.848 | 23.023 | 1.00 | 32.57 | O |
| ATOM | 278 | CB | ALA | X | 41 | 8.985 | −0.817 | 24.675 | 1.00 | 38.27 | C |
| ATOM | 279 | N | CYS | X | 42 | 10.706 | 0.025 | 21.991 | 1.00 | 38.80 | N |
| ATOM | 280 | CA | CYS | X | 42 | 11.182 | −0.422 | 20.678 | 1.00 | 38.80 | C |
| ATOM | 281 | C | CYS | X | 42 | 12.672 | −0.248 | 20.500 | 1.00 | 38.80 | C |
| ATOM | 282 | O | CYS | X | 42 | 13.289 | −0.915 | 19.673 | 1.00 | 38.80 | O |
| ATOM | 283 | CB | CYS | X | 42 | 10.464 | 0.313 | 19.539 | 1.00 | 32.69 | C |
| ATOM | 284 | SG | CYS | X | 42 | 8.700 | −0.098 | 19.380 | 1.00 | 32.69 | S |
| ATOM | 285 | N | GLY | X | 43 | 13.257 | 0.665 | 21.259 | 1.00 | 43.16 | N |
| ATOM | 286 | CA | GLY | X | 43 | 14.683 | 0.869 | 21.133 | 1.00 | 43.16 | C |
| ATOM | 287 | C | GLY | X | 43 | 15.080 | 1.917 | 20.118 | 1.00 | 43.16 | C |
| ATOM | 288 | O | GLY | X | 43 | 16.264 | 2.083 | 19.830 | 1.00 | 43.16 | O |
| ATOM | 289 | N | ILE | X | 44 | 14.110 | 2.619 | 19.547 | 1.00 | 38.75 | N |
| ATOM | 290 | CA | ILE | X | 44 | 14.464 | 3.657 | 18.594 | 1.00 | 38.75 | C |
| ATOM | 291 | C | ILE | X | 44 | 14.605 | 4.852 | 19.513 | 1.00 | 38.75 | C |
| ATOM | 292 | O | ILE | X | 44 | 13.842 | 4.997 | 20.461 | 1.00 | 38.75 | O |
| ATOM | 293 | CB | ILE | X | 44 | 13.354 | 3.887 | 17.476 | 1.00 | 45.89 | C |
| ATOM | 294 | CG1 | ILE | X | 44 | 12.519 | 5.140 | 17.759 | 1.00 | 45.89 | C |
| ATOM | 295 | CG2 | ILE | X | 44 | 12.418 | 2.689 | 17.397 | 1.00 | 45.89 | C |
| ATOM | 296 | CD1 | ILE | X | 44 | 13.108 | 6.423 | 17.216 | 1.00 | 45.89 | C |
| ATOM | 297 | N | LYS | X | 45 | 15.595 | 5.690 | 19.247 | 1.00 | 63.81 | N |
| ATOM | 298 | CA | LYS | X | 45 | 15.833 | 6.860 | 20.074 | 1.00 | 63.81 | C |
| ATOM | 299 | C | LYS | X | 45 | 15.649 | 8.171 | 19.313 | 1.00 | 63.81 | C |
| ATOM | 300 | O | LYS | X | 45 | 15.740 | 8.207 | 18.090 | 1.00 | 63.81 | O |
| ATOM | 301 | CB | LYS | X | 45 | 17.248 | 6.785 | 20.656 | 1.00 | 81.98 | C |
| ATOM | 302 | CG | LYS | X | 45 | 18.299 | 6.215 | 19.698 | 1.00 | 81.98 | C |
| ATOM | 303 | CD | LYS | X | 45 | 18.194 | 6.820 | 18.292 | 1.00 | 81.98 | C |
| ATOM | 304 | CE | LYS | X | 45 | 19.449 | 6.584 | 17.460 | 1.00 | 81.98 | C |
| ATOM | 305 | NZ | LYS | X | 45 | 20.621 | 7.346 | 18.000 | 1.00 | 81.98 | N |
| ATOM | 306 | N | ASN | X | 46 | 15.384 | 9.239 | 20.055 | 1.00 | 47.32 | N |
| ATOM | 307 | CA | ASN | X | 46 | 15.201 | 10.576 | 19.493 | 1.00 | 47.32 | C |
| ATOM | 308 | C | ASN | X | 46 | 13.819 | 10.790 | 18.915 | 1.00 | 47.32 | C |
| ATOM | 309 | O | ASN | X | 46 | 13.603 | 11.725 | 18.152 | 1.00 | 47.32 | O |
| ATOM | 310 | CB | ASN | X | 46 | 16.245 | 10.848 | 18.412 | 1.00 | 45.80 | C |
| ATOM | 311 | CG | ASN | X | 46 | 17.661 | 10.672 | 18.921 | 1.00 | 45.80 | C |
| ATOM | 312 | OD1 | ASN | X | 46 | 18.061 | 11.308 | 19.894 | 1.00 | 45.80 | O |
| ATOM | 313 | ND2 | ASN | X | 46 | 18.428 | 9.807 | 18.262 | 1.00 | 45.80 | N |
| ATOM | 314 | N | VAL | X | 47 | 12.880 | 9.938 | 19.312 | 1.00 | 46.74 | N |
| ATOM | 315 | CA | VAL | X | 47 | 11.516 | 10.007 | 18.814 | 1.00 | 46.74 | C |
| ATOM | 316 | C | VAL | X | 47 | 10.898 | 11.396 | 18.903 | 1.00 | 46.74 | C |
| ATOM | 317 | O | VAL | X | 47 | 9.776 | 11.614 | 18.460 | 1.00 | 46.74 | O |
| ATOM | 318 | CB | VAL | X | 47 | 10.605 | 8.978 | 19.540 | 1.00 | 30.63 | C |
| ATOM | 319 | CG1 | VAL | X | 47 | 11.392 | 7.727 | 19.836 | 1.00 | 30.63 | C |
| ATOM | 320 | CG2 | VAL | X | 47 | 10.022 | 9.563 | 20.793 | 1.00 | 30.63 | C |
| ATOM | 321 | N | ASN | X | 48 | 11.627 | 12.344 | 19.466 | 1.00 | 52.85 | N |
| ATOM | 322 | CA | ASN | X | 48 | 11.104 | 13.694 | 19.568 | 1.00 | 52.85 | C |
| ATOM | 323 | C | ASN | X | 48 | 11.487 | 14.486 | 18.317 | 1.00 | 52.85 | C |
| ATOM | 324 | O | ASN | X | 48 | 10.726 | 15.340 | 17.852 | 1.00 | 52.85 | O |
| ATOM | 325 | CB | ASN | X | 48 | 11.641 | 14.364 | 20.837 | 1.00 | 79.29 | C |
| ATOM | 326 | CG | ASN | X | 48 | 13.153 | 14.347 | 20.911 | 1.00 | 79.29 | C |
| ATOM | 327 | OD1 | ASN | X | 48 | 13.824 | 15.149 | 20.271 | 1.00 | 79.29 | O |
| ATOM | 328 | ND2 | ASN | X | 48 | 13.698 | 13.419 | 21.684 | 1.00 | 79.29 | N |
| ATOM | 329 | N | LEU | X | 49 | 12.661 | 14.179 | 17.768 | 1.00 | 63.14 | N |
| ATOM | 330 | CA | LEU | X | 49 | 13.165 | 14.843 | 16.566 | 1.00 | 63.14 | C |
| ATOM | 331 | C | LEU | X | 49 | 12.485 | 14.274 | 15.317 | 1.00 | 63.14 | C |
| ATOM | 332 | O | LEU | X | 49 | 11.821 | 13.239 | 15.384 | 1.00 | 63.14 | O |
| ATOM | 333 | CB | LEU | X | 49 | 14.682 | 14.647 | 16.455 | 1.00 | 46.75 | C |
| ATOM | 334 | CG | LEU | X | 49 | 15.558 | 15.152 | 17.614 | 1.00 | 46.75 | C |
| ATOM | 335 | CD1 | LEU | X | 49 | 17.014 | 14.803 | 17.361 | 1.00 | 46.75 | C |
| ATOM | 336 | CD2 | LEU | X | 49 | 15.392 | 16.653 | 17.769 | 1.00 | 46.75 | C |
| ATOM | 337 | N | PRO | X | 50 | 12.636 | 14.944 | 14.159 | 1.00 | 51.87 | N |
| ATOM | 338 | CA | PRO | X | 50 | 12.004 | 14.431 | 12.945 | 1.00 | 51.87 | C |
| ATOM | 339 | C | PRO | X | 50 | 12.700 | 13.143 | 12.577 | 1.00 | 51.87 | C |
| ATOM | 340 | O | PRO | X | 50 | 13.838 | 12.916 | 12.995 | 1.00 | 51.87 | O |
| ATOM | 341 | CB | PRO | X | 50 | 12.272 | 15.529 | 11.935 | 1.00 | 58.58 | C |
| ATOM | 342 | CG | PRO | X | 50 | 13.632 | 15.969 | 12.322 | 1.00 | 58.58 | C |
| ATOM | 343 | CD | PRO | X | 50 | 13.504 | 16.087 | 13.833 | 1.00 | 58.58 | C |
| ATOM | 344 | N | PRO | X | 51 | 12.031 | 12.280 | 11.797 | 1.00 | 45.83 | N |
| ATOM | 345 | CA | PRO | X | 51 | 10.679 | 12.455 | 11.260 | 1.00 | 45.83 | C |
| ATOM | 346 | C | PRO | X | 51 | 9.569 | 12.239 | 12.278 | 1.00 | 45.83 | C |
| ATOM | 347 | O | PRO | X | 51 | 8.518 | 12.880 | 12.203 | 1.00 | 45.83 | O |
| ATOM | 348 | CB | PRO | X | 51 | 10.626 | 11.422 | 10.161 | 1.00 | 38.67 | C |
| ATOM | 349 | CG | PRO | X | 51 | 11.410 | 10.307 | 10.773 | 1.00 | 38.67 | C |
| ATOM | 350 | CD | PRO | X | 51 | 12.615 | 11.021 | 11.305 | 1.00 | 38.67 | C |
| ATOM | 351 | N | TYR | X | 52 | 9.787 | 11.322 | 13.215 | 1.00 | 34.77 | N |
| ATOM | 352 | CA | TYR | X | 52 | 8.778 | 11.038 | 14.237 | 1.00 | 34.77 | C |
| ATOM | 353 | C | TYR | X | 52 | 8.240 | 12.344 | 14.782 | 1.00 | 34.77 | C |
| ATOM | 354 | O | TYR | X | 52 | 7.027 | 12.546 | 14.834 | 1.00 | 34.77 | O |
| ATOM | 355 | CB | TYR | X | 52 | 9.380 | 10.208 | 15.367 | 1.00 | 30.14 | C |
| ATOM | 356 | CG | TYR | X | 52 | 10.006 | 8.933 | 14.872 | 1.00 | 30.14 | C |
| ATOM | 357 | CD1 | TYR | X | 52 | 11.372 | 8.841 | 14.666 | 1.00 | 30.14 | C |
| ATOM | 358 | CD2 | TYR | X | 52 | 9.218 | 7.830 | 14.564 | 1.00 | 30.14 | C |
| ATOM | 359 | CE1 | TYR | X | 52 | 11.943 | 7.680 | 14.163 | 1.00 | 30.14 | C |
| ATOM | 360 | CE2 | TYR | X | 52 | 9.773 | 6.669 | 14.059 | 1.00 | 30.14 | C |
| ATOM | 361 | CZ | TYR | X | 52 | 11.137 | 6.594 | 13.857 | 1.00 | 30.14 | C |
| ATOM | 362 | OH | TYR | X | 52 | 11.682 | 5.434 | 13.332 | 1.00 | 30.14 | O |
| ATOM | 363 | N | SER | X | 53 | 9.161 | 13.221 | 15.186 | 1.00 | 38.91 | N |
| ATOM | 364 | CA | SER | X | 53 | 8.838 | 14.545 | 15.713 | 1.00 | 38.91 | C |
| ATOM | 365 | C | SER | X | 53 | 7.830 | 14.542 | 16.846 | 1.00 | 38.91 | C |
| ATOM | 366 | O | SER | X | 53 | 6.898 | 15.340 | 16.848 | 1.00 | 38.91 | O |
| ATOM | 367 | CB | SER | X | 53 | 8.308 | 15.439 | 14.589 | 1.00 | 35.32 | C |
| ATOM | 368 | OG | SER | X | 53 | 9.279 | 15.627 | 13.573 | 1.00 | 35.32 | O |
| ATOM | 369 | N | GLY | X | 54 | 8.003 | 13.645 | 17.808 | 1.00 | 27.35 | N |
| ATOM | 370 | CA | GLY | X | 54 | 7.078 | 13.600 | 18.927 | 1.00 | 27.35 | C |
| ATOM | 371 | C | GLY | X | 54 | 5.672 | 13.104 | 18.646 | 1.00 | 27.35 | C |
| ATOM | 372 | O | GLY | X | 54 | 4.788 | 13.235 | 19.486 | 1.00 | 27.35 | O |
| ATOM | 373 | N | MET | X | 55 | 5.429 | 12.537 | 17.476 | 1.00 | 62.97 | N |
| ATOM | 374 | CA | MET | X | 55 | 4.094 | 12.028 | 17.216 | 1.00 | 62.97 | C |
| ATOM | 375 | C | MET | X | 55 | 4.096 | 10.533 | 17.522 | 1.00 | 62.97 | C |
| ATOM | 376 | O | MET | X | 55 | 3.645 | 9.716 | 16.716 | 1.00 | 62.97 | O |
| ATOM | 377 | CB | MET | X | 55 | 3.698 | 12.291 | 15.768 | 1.00 | 39.34 | C |
| ATOM | 378 | CG | MET | X | 55 | 3.443 | 13.765 | 15.437 | 1.00 | 39.34 | C |
| ATOM | 379 | SD | MET | X | 55 | 2.351 | 14.625 | 16.593 | 1.00 | 39.34 | S |
| ATOM | 380 | CE | MET | X | 55 | 0.775 | 13.923 | 16.287 | 1.00 | 39.34 | C |
| ATOM | 381 | N | THR | X | 56 | 4.602 | 10.209 | 18.718 | 1.00 | 19.36 | N |
| ATOM | 382 | CA | THR | X | 56 | 4.753 | 8.854 | 19.240 | 1.00 | 19.36 | C |
| ATOM | 383 | C | THR | X | 56 | 3.590 | 8.307 | 20.057 | 1.00 | 19.36 | C |
| ATOM | 384 | O | THR | X | 56 | 2.721 | 9.046 | 20.500 | 1.00 | 19.36 | O |
| ATOM | 385 | CB | THR | X | 56 | 5.980 | 8.809 | 20.114 | 1.00 | 52.97 | C |
| ATOM | 386 | OG1 | THR | X | 56 | 7.124 | 9.135 | 19.324 | 1.00 | 52.97 | O |
| ATOM | 387 | CG2 | THR | X | 56 | 6.149 | 7.449 | 20.725 | 1.00 | 52.97 | C |
| ATOM | 388 | N | ALA | X | 57 | 3.564 | 6.995 | 20.243 | 1.00 | 21.43 | N |
| ATOM | 389 | CA | ALA | X | 57 | 2.519 | 6.403 | 21.053 | 1.00 | 21.43 | C |
| ATOM | 390 | C | ALA | X | 57 | 2.769 | 4.972 | 21.507 | 1.00 | 21.43 | C |
| ATOM | 391 | O | ALA | X | 57 | 3.665 | 4.288 | 21.017 | 1.00 | 21.43 | O |
| ATOM | 392 | CB | ALA | X | 57 | 1.205 | 6.501 | 20.354 | 1.00 | 1.67 | C |
| ATOM | 393 | N | CYS | X | 58 | 1.954 | 4.551 | 22.468 | 1.00 | 41.38 | N |
| ATOM | 394 | CA | CYS | X | 58 | 2.023 | 3.228 | 23.067 | 1.00 | 41.38 | C |
| ATOM | 395 | C | CYS | X | 58 | 0.718 | 2.501 | 22.859 | 1.00 | 41.38 | C |
| ATOM | 396 | O | CYS | X | 58 | −0.361 | 3.097 | 22.887 | 1.00 | 41.38 | O |
| ATOM | 397 | CB | CYS | X | 58 | 2.255 | 3.318 | 24.585 | 1.00 | 51.16 | C |
| ATOM | 398 | SG | CYS | X | 58 | 3.890 | 3.849 | 25.146 | 1.00 | 51.16 | S |
| ATOM | 399 | N | GLY | X | 59 | 0.821 | 1.195 | 22.679 | 1.00 | 35.87 | N |
| ATOM | 400 | CA | GLY | X | 59 | −0.367 | 0.397 | 22.503 | 1.00 | 35.87 | C |
| ATOM | 401 | C | GLY | X | 59 | −0.130 | −0.927 | 23.172 | 1.00 | 35.87 | C |
| ATOM | 402 | O | GLY | X | 59 | 1.021 | −1.332 | 23.339 | 1.00 | 35.87 | O |
| ATOM | 403 | N | ASN | X | 60 | −1.220 | −1.587 | 23.554 | 1.00 | 34.54 | N |
| ATOM | 404 | CA | ASN | X | 60 | −1.160 | −2.882 | 24.199 | 1.00 | 34.54 | C |
| ATOM | 405 | C | ASN | X | 60 | −0.713 | −4.004 | 23.255 | 1.00 | 34.54 | C |
| ATOM | 406 | O | ASN | X | 60 | −0.140 | −3.750 | 22.194 | 1.00 | 34.54 | O |
| ATOM | 407 | CB | ASN | X | 60 | −2.515 | −3.225 | 24.839 | 1.00 | 40.64 | C |
| ATOM | 408 | CG | ASN | X | 60 | −3.697 | −2.642 | 24.083 | 1.00 | 40.64 | C |
| ATOM | 409 | OD1 | ASN | X | 60 | −3.534 | −1.992 | 23.055 | 1.00 | 40.64 | O |
| ATOM | 410 | ND2 | ASN | X | 60 | −4.901 | −2.872 | 24.598 | 1.00 | 40.64 | N |
| ATOM | 411 | N | VAL | X | 61 | −0.963 | −5.243 | 23.666 | 1.00 | 40.02 | N |
| ATOM | 412 | CA | VAL | X | 61 | −0.580 | −6.436 | 22.908 | 1.00 | 40.02 | C |
| ATOM | 413 | C | VAL | X | 61 | −0.985 | −6.397 | 21.435 | 1.00 | 40.02 | C |
| ATOM | 414 | O | VAL | X | 61 | −0.185 | −6.705 | 20.550 | 1.00 | 40.02 | O |
| ATOM | 415 | CB | VAL | X | 61 | −1.204 | −7.718 | 23.531 | 1.00 | 36.95 | C |
| ATOM | 416 | CG1 | VAL | X | 61 | −0.313 | −8.915 | 23.256 | 1.00 | 36.95 | C |
| ATOM | 417 | CG2 | VAL | X | 61 | −1.438 | −7.521 | 25.021 | 1.00 | 36.95 | C |
| ATOM | 418 | N | PRO | X | 62 | −2.242 | −6.025 | 21.161 | 1.00 | 33.00 | N |
| ATOM | 419 | CA | PRO | X | 62 | −2.803 | −5.935 | 19.814 | 1.00 | 33.00 | C |
| ATOM | 420 | C | PRO | X | 62 | −1.966 | −5.020 | 18.935 | 1.00 | 33.00 | C |
| ATOM | 421 | O | PRO | X | 62 | −1.440 | −5.418 | 17.894 | 1.00 | 33.00 | O |
| ATOM | 422 | CB | PRO | X | 62 | −4.178 | −5.352 | 20.066 | 1.00 | 21.95 | C |
| ATOM | 423 | CG | PRO | X | 62 | −4.502 | −5.810 | 21.448 | 1.00 | 21.95 | C |
| ATOM | 424 | CD | PRO | X | 62 | −3.225 | −5.573 | 22.155 | 1.00 | 21.95 | C |
| ATOM | 425 | N | ILE | X | 63 | −1.843 | −3.779 | 19.380 | 1.00 | 23.78 | N |
| ATOM | 426 | CA | ILE | X | 63 | −1.092 | −2.786 | 18.653 | 1.00 | 23.78 | C |
| ATOM | 427 | C | ILE | X | 63 | 0.388 | −3.074 | 18.680 | 1.00 | 23.78 | C |
| ATOM | 428 | O | ILE | X | 63 | 0.994 | −3.274 | 17.631 | 1.00 | 23.78 | O |
| ATOM | 429 | CB | ILE | X | 63 | −1.355 | −1.416 | 19.242 | 1.00 | 30.12 | C |
| ATOM | 430 | CG1 | ILE | X | 63 | −2.850 | −1.134 | 19.175 | 1.00 | 30.12 | C |
| ATOM | 431 | CG2 | ILE | X | 63 | −0.570 | −0.371 | 18.503 | 1.00 | 30.12 | C |
| ATOM | 432 | CD1 | ILE | X | 63 | −3.257 | 0.102 | 19.901 | 1.00 | 30.12 | C |
| ATOM | 433 | N | PHE | X | 64 | 0.953 | −3.121 | 19.888 | 1.00 | 27.84 | N |
| ATOM | 434 | CA | PHE | X | 64 | 2.382 | −3.352 | 20.101 | 1.00 | 27.84 | C |
| ATOM | 435 | C | PHE | X | 64 | 2.930 | −4.704 | 19.654 | 1.00 | 27.84 | C |
| ATOM | 436 | O | PHE | X | 64 | 3.998 | −4.764 | 19.053 | 1.00 | 27.84 | O |
| ATOM | 437 | CB | PHE | X | 64 | 2.738 | −3.120 | 21.573 | 1.00 | 27.48 | C |
| ATOM | 438 | CG | PHE | X | 64 | 4.215 | −3.101 | 21.831 | 1.00 | 27.48 | C |
| ATOM | 439 | CD1 | PHE | X | 64 | 5.012 | −2.124 | 21.266 | 1.00 | 27.48 | C |
| ATOM | 440 | CD2 | PHE | X | 64 | 4.825 | −4.122 | 22.549 | 1.00 | 27.48 | C |
| ATOM | 441 | CE1 | PHE | X | 64 | 6.391 | −2.164 | 21.397 | 1.00 | 27.48 | C |
| ATOM | 442 | CE2 | PHE | X | 64 | 6.211 | −4.169 | 22.684 | 1.00 | 27.48 | C |
| ATOM | 443 | CZ | PHE | X | 64 | 6.990 | −3.190 | 22.104 | 1.00 | 27.48 | C |
| ATOM | 444 | N | LYS | X | 65 | 2.210 | −5.784 | 19.947 | 1.00 | 32.57 | N |
| ATOM | 445 | CA | LYS | X | 65 | 2.639 | −7.130 | 19.555 | 1.00 | 32.57 | C |
| ATOM | 446 | C | LYS | X | 65 | 4.087 | −7.447 | 19.926 | 1.00 | 32.57 | C |
| ATOM | 447 | O | LYS | X | 65 | 4.945 | −7.570 | 19.054 | 1.00 | 32.57 | O |
| ATOM | 448 | CB | LYS | X | 65 | 2.439 | −7.330 | 18.048 | 1.00 | 29.02 | C |
| ATOM | 449 | CG | LYS | X | 65 | 0.975 | −7.456 | 17.637 | 1.00 | 29.02 | C |
| ATOM | 450 | CD | LYS | X | 65 | 0.784 | −7.344 | 16.120 | 1.00 | 29.02 | C |
| ATOM | 451 | CE | LYS | X | 65 | 0.914 | −5.904 | 15.625 | 1.00 | 29.02 | C |
| ATOM | 452 | NZ | LYS | X | 65 | 1.197 | −5.823 | 14.158 | 1.00 | 29.02 | N |
| ATOM | 453 | N | ASP | X | 66 | 4.335 | −7.586 | 21.226 | 1.00 | 29.13 | N |
| ATOM | 454 | CA | ASP | X | 66 | 5.656 | −7.896 | 21.777 | 1.00 | 29.13 | C |
| ATOM | 455 | C | ASP | X | 66 | 6.847 | −7.411 | 20.965 | 1.00 | 29.13 | C |
| ATOM | 456 | O | ASP | X | 66 | 7.877 | −8.097 | 20.894 | 1.00 | 29.13 | O |
| ATOM | 457 | CB | ASP | X | 66 | 5.816 | −9.401 | 22.020 | 1.00 | 31.30 | C |
| ATOM | 458 | CG | ASP | X | 66 | 4.797 | −9.953 | 23.008 | 1.00 | 31.30 | C |
| ATOM | 459 | OD1 | ASP | X | 66 | 4.157 | −9.159 | 23.740 | 1.00 | 31.30 | O |
| ATOM | 460 | OD2 | ASP | X | 66 | 4.646 | −11.195 | 23.049 | 1.00 | 31.30 | O |
| ATOM | 461 | N | GLY | X | 67 | 6.698 | −6.239 | 20.351 | 1.00 | 28.31 | N |
| ATOM | 462 | CA | GLY | X | 67 | 7.780 | −5.652 | 19.583 | 1.00 | 28.31 | C |
| ATOM | 463 | C | GLY | X | 67 | 7.749 | −5.839 | 18.088 | 1.00 | 28.31 | C |
| ATOM | 464 | O | GLY | X | 67 | 8.655 | −5.394 | 17.382 | 1.00 | 28.31 | O |
| ATOM | 465 | N | LYS | X | 68 | 6.717 | −6.496 | 17.587 | 1.00 | 49.24 | N |
| ATOM | 466 | CA | LYS | X | 68 | 6.623 | −6.727 | 16.155 | 1.00 | 49.24 | C |
| ATOM | 467 | C | LYS | X | 68 | 5.762 | −5.654 | 15.506 | 1.00 | 49.24 | C |
| ATOM | 468 | O | LYS | X | 68 | 5.610 | −5.613 | 14.290 | 1.00 | 49.24 | O |
| ATOM | 469 | CB | LYS | X | 68 | 6.076 | −8.135 | 15.902 | 1.00 | 87.29 | C |
| ATOM | 470 | CG | LYS | X | 68 | 7.063 | −9.218 | 16.328 | 1.00 | 87.29 | C |
| ATOM | 471 | CD | LYS | X | 68 | 6.391 | −10.408 | 16.990 | 1.00 | 87.29 | C |
| ATOM | 472 | CE | LYS | X | 68 | 5.658 | −11.287 | 15.992 | 1.00 | 87.29 | C |
| ATOM | 473 | NZ | LYS | X | 68 | 5.011 | −12.461 | 16.659 | 1.00 | 87.29 | N |
| ATOM | 474 | N | GLY | X | 69 | 5.214 | −4.780 | 16.344 | 1.00 | 37.58 | N |
| ATOM | 475 | CA | GLY | X | 69 | 4.394 | −3.685 | 15.866 | 1.00 | 37.58 | C |
| ATOM | 476 | C | GLY | X | 69 | 5.222 | −2.410 | 15.814 | 1.00 | 37.58 | C |
| ATOM | 477 | O | GLY | X | 69 | 4.752 | −1.364 | 15.384 | 1.00 | 37.58 | O |
| ATOM | 478 | N | CYS | X | 70 | 6.461 | −2.498 | 16.275 | 1.00 | 19.54 | N |
| ATOM | 479 | CA | CYS | X | 70 | 7.349 | −1.359 | 16.269 | 1.00 | 19.54 | C |
| ATOM | 480 | C | CYS | X | 70 | 7.524 | −0.878 | 14.859 | 1.00 | 19.54 | C |
| ATOM | 481 | O | CYS | X | 70 | 8.007 | −1.632 | 14.023 | 1.00 | 19.54 | O |
| ATOM | 482 | CB | CYS | X | 70 | 8.712 | −1.737 | 16.827 | 1.00 | 42.07 | C |
| ATOM | 483 | SG | CYS | X | 70 | 8.705 | −1.978 | 18.617 | 1.00 | 42.07 | S |
| ATOM | 484 | N | GLY | X | 71 | 7.161 | 0.379 | 14.612 | 1.00 | 32.49 | N |
| ATOM | 485 | CA | GLY | X | 71 | 7.274 | 0.952 | 13.284 | 1.00 | 32.49 | C |
| ATOM | 486 | C | GLY | X | 71 | 5.906 | 1.277 | 12.703 | 1.00 | 32.49 | C |
| ATOM | 487 | O | GLY | X | 71 | 5.750 | 2.235 | 11.960 | 1.00 | 32.49 | O |
| ATOM | 488 | N | SER | X | 72 | 4.907 | 0.471 | 13.038 | 1.00 | 24.93 | N |
| ATOM | 489 | CA | SER | X | 72 | 3.556 | 0.691 | 12.553 | 1.00 | 24.93 | C |
| ATOM | 490 | C | SER | X | 72 | 3.149 | 2.138 | 12.680 | 1.00 | 24.93 | C |
| ATOM | 491 | O | SER | X | 72 | 3.662 | 2.873 | 13.511 | 1.00 | 24.93 | O |
| ATOM | 492 | CB | SER | X | 72 | 2.532 | −0.115 | 13.360 | 1.00 | 24.64 | C |
| ATOM | 493 | OG | SER | X | 72 | 2.663 | −1.496 | 13.172 | 1.00 | 24.64 | O |
| ATOM | 494 | N | CYS | X | 73 | 2.175 | 2.510 | 11.866 | 1.00 | 26.85 | N |
| ATOM | 495 | CA | CYS | X | 73 | 1.600 | 3.831 | 11.871 | 1.00 | 26.85 | C |
| ATOM | 496 | C | CYS | X | 73 | 0.120 | 3.632 | 12.014 | 1.00 | 26.85 | C |
| ATOM | 497 | O | CYS | X | 73 | −0.454 | 2.716 | 11.449 | 1.00 | 26.85 | O |
| ATOM | 498 | CB | CYS | X | 73 | 1.909 | 4.530 | 10.567 | 1.00 | 30.66 | C |
| ATOM | 499 | SG | CYS | X | 73 | 3.687 | 4.560 | 10.483 | 1.00 | 30.66 | S |
| ATOM | 500 | N | TYR | X | 74 | −0.498 | 4.477 | 12.806 | 1.00 | 24.09 | N |
| ATOM | 501 | CA | TYR | X | 74 | −1.919 | 4.402 | 12.991 | 1.00 | 24.09 | C |
| ATOM | 502 | C | TYR | X | 74 | −2.373 | 5.830 | 12.839 | 1.00 | 24.09 | C |
| ATOM | 503 | O | TYR | X | 74 | −1.559 | 6.747 | 12.801 | 1.00 | 24.09 | O |
| ATOM | 504 | CB | TYR | X | 74 | −2.266 | 3.856 | 14.374 | 1.00 | 30.54 | C |
| ATOM | 505 | CG | TYR | X | 74 | −2.137 | 2.350 | 14.480 | 1.00 | 30.54 | C |
| ATOM | 506 | CD1 | TYR | X | 74 | −0.901 | 1.732 | 14.351 | 1.00 | 30.54 | C |
| ATOM | 507 | CD2 | TYR | X | 74 | −3.252 | 1.551 | 14.716 | 1.00 | 30.54 | C |
| ATOM | 508 | CE1 | TYR | X | 74 | −0.768 | 0.367 | 14.452 | 1.00 | 30.54 | C |
| ATOM | 509 | CE2 | TYR | X | 74 | −3.134 | 0.180 | 14.823 | 1.00 | 30.54 | C |
| ATOM | 510 | CZ | TYR | X | 74 | −1.882 | −0.406 | 14.690 | 1.00 | 30.54 | C |
| ATOM | 511 | OH | TYR | X | 74 | −1.733 | −1.768 | 14.818 | 1.00 | 30.54 | O |
| ATOM | 512 | N | GLU | X | 75 | −3.674 | 6.011 | 12.735 | 1.00 | 34.37 | N |
| ATOM | 513 | CA | GLU | X | 75 | −4.238 | 7.319 | 12.548 | 1.00 | 34.37 | C |
| ATOM | 514 | C | GLU | X | 75 | −5.313 | 7.346 | 13.599 | 1.00 | 34.37 | C |
| ATOM | 515 | O | GLU | X | 75 | −6.179 | 6.473 | 13.629 | 1.00 | 34.37 | O |
| ATOM | 516 | CB | GLU | X | 75 | −4.840 | 7.400 | 11.135 | 1.00 | 39.78 | C |
| ATOM | 517 | CG | GLU | X | 75 | −5.083 | 8.786 | 10.561 | 1.00 | 39.78 | C |
| ATOM | 518 | CD | GLU | X | 75 | −5.525 | 8.737 | 9.099 | 1.00 | 39.78 | C |
| ATOM | 519 | OE1 | GLU | X | 75 | −6.625 | 8.219 | 8.806 | 1.00 | 39.78 | O |
| ATOM | 520 | OE2 | GLU | X | 75 | −4.763 | 9.214 | 8.238 | 1.00 | 39.78 | O |
| ATOM | 521 | N | VAL | X | 76 | −5.250 | 8.323 | 14.488 | 1.00 | 31.76 | N |
| ATOM | 522 | CA | VAL | X | 76 | −6.253 | 8.409 | 15.523 | 1.00 | 31.76 | C |
| ATOM | 523 | C | VAL | X | 76 | −6.889 | 9.788 | 15.458 | 1.00 | 31.76 | C |
| ATOM | 524 | O | VAL | X | 76 | −6.260 | 10.757 | 15.034 | 1.00 | 31.76 | O |
| ATOM | 525 | CB | VAL | X | 76 | −5.627 | 8.134 | 16.907 | 1.00 | 39.03 | C |
| ATOM | 526 | CG1 | VAL | X | 76 | −4.505 | 9.104 | 17.168 | 1.00 | 39.03 | C |
| ATOM | 527 | CG2 | VAL | X | 76 | −6.696 | 8.206 | 17.985 | 1.00 | 39.03 | C |
| ATOM | 528 | N | ARG | X | 77 | −8.150 | 9.865 | 15.857 | 1.00 | 45.60 | N |
| ATOM | 529 | CA | ARG | X | 77 | −8.876 | 11.118 | 15.814 | 1.00 | 45.60 | C |
| ATOM | 530 | C | ARG | X | 77 | −9.867 | 11.163 | 16.958 | 1.00 | 45.60 | C |
| ATOM | 531 | O | ARG | X | 77 | −10.331 | 10.130 | 17.428 | 1.00 | 45.60 | O |
| ATOM | 532 | CB | ARG | X | 77 | −9.633 | 11.236 | 14.490 | 1.00 | 74.88 | C |
| ATOM | 533 | CG | ARG | X | 77 | −10.919 | 10.410 | 14.437 | 1.00 | 74.88 | C |
| ATOM | 534 | CD | ARG | X | 77 | −11.539 | 10.418 | 13.051 | 1.00 | 74.88 | C |
| ATOM | 535 | NE | ARG | X | 77 | −12.867 | 9.810 | 13.026 | 1.00 | 74.88 | N |
| ATOM | 536 | CZ | ARG | X | 77 | −13.539 | 9.531 | 11.912 | 1.00 | 74.88 | C |
| ATOM | 537 | NH1 | ARG | X | 77 | −13.006 | 9.800 | 10.726 | 1.00 | 74.88 | N |
| ATOM | 538 | NH2 | ARG | X | 77 | −14.751 | 8.994 | 11.981 | 1.00 | 74.88 | N |
| ATOM | 539 | N | CYS | X | 78 | −10.195 | 12.366 | 17.403 | 1.00 | 50.61 | N |
| ATOM | 540 | CA | CYS | X | 78 | −11.142 | 12.510 | 18.486 | 1.00 | 50.61 | C |
| ATOM | 541 | C | CYS | X | 78 | −12.384 | 13.247 | 18.037 | 1.00 | 50.61 | C |
| ATOM | 542 | O | CYS | X | 78 | −12.304 | 14.219 | 17.294 | 1.00 | 50.61 | O |
| ATOM | 543 | CB | CYS | X | 78 | −10.529 | 13.272 | 19.655 | 1.00 | 84.27 | C |
| ATOM | 544 | SG | CYS | X | 78 | −11.853 | 13.899 | 20.726 | 1.00 | 84.27 | S |
| ATOM | 545 | N | LYS | X | 79 | −13.536 | 12.788 | 18.501 | 1.00 | 55.20 | N |
| ATOM | 546 | CA | LYS | X | 79 | −14.784 | 13.435 | 18.159 | 1.00 | 55.20 | C |
| ATOM | 547 | C | LYS | X | 79 | −15.804 | 13.199 | 19.262 | 1.00 | 55.20 | C |
| ATOM | 548 | O | LYS | X | 79 | −17.007 | 13.194 | 19.018 | 1.00 | 55.20 | O |
| ATOM | 549 | CB | LYS | X | 79 | −15.310 | 12.906 | 16.822 | 1.00 | 64.46 | C |
| ATOM | 550 | CG | LYS | X | 79 | −16.551 | 13.644 | 16.321 | 1.00 | 64.46 | C |
| ATOM | 551 | CD | LYS | X | 79 | −16.319 | 15.161 | 16.245 | 1.00 | 64.46 | C |
| ATOM | 552 | CE | LYS | X | 79 | −17.621 | 15.940 | 16.100 | 1.00 | 64.46 | C |
| ATOM | 553 | NZ | LYS | X | 79 | −18.501 | 15.773 | 17.291 | 1.00 | 64.46 | N |
| ATOM | 554 | N | GLU | X | 80 | −15.320 | 13.005 | 20.485 | 1.00 | 69.81 | N |
| ATOM | 555 | CA | GLU | X | 80 | −16.216 | 12.773 | 21.610 | 1.00 | 69.81 | C |
| ATOM | 556 | C | GLU | X | 80 | −16.364 | 14.053 | 22.413 | 1.00 | 69.81 | C |
| ATOM | 557 | O | GLU | X | 80 | −17.442 | 14.643 | 22.482 | 1.00 | 69.81 | O |
| ATOM | 558 | CB | GLU | X | 80 | −15.674 | 11.667 | 22.517 | 1.00 | 99.00 | C |
| ATOM | 559 | CG | GLU | X | 80 | −16.758 | 10.860 | 23.228 | 1.00 | 99.00 | C |
| ATOM | 560 | CD | GLU | X | 80 | −17.868 | 11.725 | 23.814 | 1.00 | 99.00 | C |
| ATOM | 561 | OE1 | GLU | X | 80 | −18.648 | 12.320 | 23.036 | 1.00 | 99.00 | O |
| ATOM | 562 | OE2 | GLU | X | 80 | −17.963 | 11.812 | 25.057 | 1.00 | 99.00 | O |
| ATOM | 563 | N | LYS | X | 81 | −15.269 | 14.483 | 23.024 | 1.00 | 99.00 | N |
| ATOM | 564 | CA | LYS | X | 81 | −15.290 | 15.699 | 23.814 | 1.00 | 99.00 | C |
| ATOM | 565 | C | LYS | X | 81 | −14.927 | 16.869 | 22.907 | 1.00 | 99.00 | C |
| ATOM | 566 | O | LYS | X | 81 | −14.218 | 16.693 | 21.914 | 1.00 | 99.00 | O |
| ATOM | 567 | CB | LYS | X | 81 | −14.308 | 15.575 | 24.981 | 1.00 | 79.10 | C |
| ATOM | 568 | CG | LYS | X | 81 | −14.499 | 14.279 | 25.763 | 1.00 | 79.10 | C |
| ATOM | 569 | CD | LYS | X | 81 | −13.766 | 14.282 | 27.089 | 1.00 | 79.10 | C |
| ATOM | 570 | CE | LYS | X | 81 | −14.398 | 15.257 | 28.083 | 1.00 | 79.10 | C |
| ATOM | 571 | NZ | LYS | X | 81 | −15.785 | 14.877 | 28.493 | 1.00 | 79.10 | N |
| ATOM | 572 | N | PRO | X | 82 | −15.426 | 18.076 | 23.228 | 1.00 | 70.36 | N |
| ATOM | 573 | CA | PRO | X | 82 | −15.166 | 19.289 | 22.448 | 1.00 | 70.36 | C |
| ATOM | 574 | C | PRO | X | 82 | −13.689 | 19.488 | 22.169 | 1.00 | 70.36 | C |
| ATOM | 575 | O | PRO | X | 82 | −12.902 | 18.552 | 22.263 | 1.00 | 70.36 | O |
| ATOM | 576 | CB | PRO | X | 82 | −15.736 | 20.391 | 23.331 | 1.00 | 84.04 | C |
| ATOM | 577 | CG | PRO | X | 82 | −16.875 | 19.705 | 24.012 | 1.00 | 84.04 | C |
| ATOM | 578 | CD | PRO | X | 82 | −16.256 | 18.388 | 24.405 | 1.00 | 84.04 | C |
| ATOM | 579 | N | GLU | X | 83 | −13.321 | 20.717 | 21.836 | 1.00 | 55.17 | N |
| ATOM | 580 | CA | GLU | X | 83 | −11.933 | 21.063 | 21.534 | 1.00 | 55.17 | C |
| ATOM | 581 | C | GLU | X | 83 | −11.203 | 19.975 | 20.756 | 1.00 | 55.17 | C |
| ATOM | 582 | O | GLU | X | 83 | −9.971 | 19.939 | 20.723 | 1.00 | 55.17 | O |
| ATOM | 583 | CB | GLU | X | 83 | −11.150 | 21.397 | 22.821 | 1.00 | 75.84 | C |
| ATOM | 584 | CG | GLU | X | 83 | −10.952 | 20.259 | 23.812 | 1.00 | 75.84 | C |
| ATOM | 585 | CD | GLU | X | 83 | −11.921 | 20.318 | 24.981 | 1.00 | 75.84 | C |
| ATOM | 586 | OE1 | GLU | X | 83 | −13.117 | 20.012 | 24.794 | 1.00 | 75.84 | O |
| ATOM | 587 | OE2 | GLU | X | 83 | −11.483 | 20.680 | 26.093 | 1.00 | 75.84 | O |
| ATOM | 588 | N | CYS | X | 84 | −11.975 | 19.097 | 20.122 | 1.00 | 57.58 | N |
| ATOM | 589 | CA | CYS | X | 84 | −11.407 | 18.014 | 19.345 | 1.00 | 57.58 | C |
| ATOM | 590 | C | CYS | X | 84 | −11.399 | 18.340 | 17.881 | 1.00 | 57.58 | C |
| ATOM | 591 | O | CYS | X | 84 | −12.441 | 18.397 | 17.244 | 1.00 | 57.58 | O |
| ATOM | 592 | CB | CYS | X | 84 | −12.165 | 16.718 | 19.610 | 1.00 | 84.27 | C |
| ATOM | 593 | SG | CYS | X | 84 | −11.401 | 15.849 | 21.010 | 1.00 | 84.27 | S |
| ATOM | 594 | N | SER | X | 85 | −10.197 | 18.570 | 17.368 | 1.00 | 50.63 | N |
| ATOM | 595 | CA | SER | X | 85 | −9.987 | 18.911 | 15.972 | 1.00 | 50.63 | C |
| ATOM | 596 | C | SER | X | 85 | −10.933 | 18.155 | 15.041 | 1.00 | 50.63 | C |
| ATOM | 597 | O | SER | X | 85 | −11.336 | 18.669 | 13.989 | 1.00 | 50.63 | O |
| ATOM | 598 | CB | SER | X | 85 | −8.532 | 18.620 | 15.577 | 1.00 | 76.77 | C |
| ATOM | 599 | OG | SER | X | 85 | −8.219 | 17.239 | 15.698 | 1.00 | 76.77 | O |
| ATOM | 600 | N | GLY | X | 86 | −11.300 | 16.942 | 15.441 | 1.00 | 48.12 | N |
| ATOM | 601 | CA | GLY | X | 86 | −12.168 | 16.132 | 14.614 | 1.00 | 48.12 | C |
| ATOM | 602 | C | GLY | X | 86 | −11.291 | 15.422 | 13.600 | 1.00 | 48.12 | C |
| ATOM | 603 | O | GLY | X | 86 | −11.585 | 14.310 | 13.168 | 1.00 | 48.12 | O |
| ATOM | 604 | N | ASN | X | 87 | −10.196 | 16.072 | 13.221 | 1.00 | 69.99 | N |
| ATOM | 605 | CA | ASN | X | 87 | −9.266 | 15.502 | 12.261 | 1.00 | 69.99 | C |
| ATOM | 606 | C | ASN | X | 87 | −8.284 | 14.559 | 12.941 | 1.00 | 69.99 | C |
| ATOM | 607 | O | ASN | X | 87 | −7.938 | 14.740 | 14.110 | 1.00 | 69.99 | O |
| ATOM | 608 | CB | ASN | X | 87 | −8.515 | 16.616 | 11.529 | 1.00 | 95.79 | C |
| ATOM | 609 | CG | ASN | X | 87 | −9.240 | 17.080 | 10.283 | 1.00 | 95.79 | C |
| ATOM | 610 | OD1 | ASN | X | 87 | −10.441 | 17.352 | 10.312 | 1.00 | 95.79 | O |
| ATOM | 611 | ND2 | ASN | X | 87 | −8.511 | 17.174 | 9.177 | 1.00 | 95.79 | N |
| ATOM | 612 | N | PRO | X | 88 | −7.818 | 13.537 | 12.205 | 1.00 | 50.49 | N |
| ATOM | 613 | CA | PRO | X | 88 | −6.878 | 12.544 | 12.711 | 1.00 | 50.49 | C |
| ATOM | 614 | C | PRO | X | 88 | −5.429 | 12.998 | 12.684 | 1.00 | 50.49 | C |
| ATOM | 615 | O | PRO | X | 88 | −5.057 | 13.908 | 11.949 | 1.00 | 50.49 | O |
| ATOM | 616 | CB | PRO | X | 88 | −7.116 | 11.366 | 11.780 | 1.00 | 54.49 | C |
| ATOM | 617 | CG | PRO | X | 88 | −7.288 | 12.059 | 10.460 | 1.00 | 54.49 | C |
| ATOM | 618 | CD | PRO | X | 88 | −8.223 | 13.202 | 10.824 | 1.00 | 54.49 | C |
| ATOM | 619 | N | VAL | X | 89 | −4.616 | 12.340 | 13.499 | 1.00 | 61.42 | N |
| ATOM | 620 | CA | VAL | X | 89 | −3.192 | 12.621 | 13.575 | 1.00 | 61.42 | C |
| ATOM | 621 | C | VAL | X | 89 | −2.520 | 11.295 | 13.260 | 1.00 | 61.42 | C |
| ATOM | 622 | O | VAL | X | 89 | −3.058 | 10.242 | 13.581 | 1.00 | 61.42 | O |
| ATOM | 623 | CB | VAL | X | 89 | −2.769 | 13.088 | 15.003 | 1.00 | 47.66 | C |
| ATOM | 624 | CG1 | VAL | X | 89 | −2.241 | 14.496 | 14.949 | 1.00 | 47.66 | C |
| ATOM | 625 | CG2 | VAL | X | 89 | −3.948 | 13.029 | 15.957 | 1.00 | 47.66 | C |
| ATOM | 626 | N | THR | X | 90 | −1.369 | 11.333 | 12.609 | 1.00 | 52.85 | N |
| ATOM | 627 | CA | THR | X | 90 | −0.672 | 10.096 | 12.297 | 1.00 | 52.85 | C |
| ATOM | 628 | C | THR | X | 90 | 0.392 | 9.825 | 13.353 | 1.00 | 52.85 | C |
| ATOM | 629 | O | THR | X | 90 | 1.376 | 10.557 | 13.469 | 1.00 | 52.85 | O |
| ATOM | 630 | CB | THR | X | 90 | −0.007 | 10.130 | 10.889 | 1.00 | 49.85 | C |
| ATOM | 631 | OG1 | THR | X | 90 | −1.016 | 10.024 | 9.878 | 1.00 | 49.85 | O |
| ATOM | 632 | CG2 | THR | X | 90 | 0.959 | 8.974 | 10.723 | 1.00 | 49.85 | C |
| ATOM | 633 | N | VAL | X | 91 | 0.178 | 8.756 | 14.112 | 1.00 | 51.89 | N |
| ATOM | 634 | CA | VAL | X | 91 | 1.076 | 8.341 | 15.179 | 1.00 | 51.89 | C |
| ATOM | 635 | C | VAL | X | 91 | 1.984 | 7.167 | 14.796 | 1.00 | 51.89 | C |
| ATOM | 636 | O | VAL | X | 91 | 1.651 | 6.373 | 13.917 | 1.00 | 51.89 | O |
| ATOM | 637 | CB | VAL | X | 91 | 0.258 | 7.922 | 16.408 | 1.00 | 36.09 | C |
| ATOM | 638 | CG1 | VAL | X | 91 | 1.098 | 7.103 | 17.321 | 1.00 | 36.09 | C |
| ATOM | 639 | CG2 | VAL | X | 91 | −0.265 | 9.136 | 17.126 | 1.00 | 36.09 | C |
| ATOM | 640 | N | TYR | X | 92 | 3.143 | 7.079 | 15.446 | 1.00 | 54.85 | N |
| ATOM | 641 | CA | TYR | X | 92 | 4.063 | 5.967 | 15.228 | 1.00 | 54.85 | C |
| ATOM | 642 | C | TYR | X | 92 | 4.060 | 5.165 | 16.518 | 1.00 | 54.85 | C |
| ATOM | 643 | O | TYR | X | 92 | 3.809 | 5.712 | 17.589 | 1.00 | 54.85 | O |
| ATOM | 644 | CB | TYR | X | 92 | 5.483 | 6.441 | 14.949 | 1.00 | 51.94 | C |
| ATOM | 645 | CG | TYR | X | 92 | 5.677 | 7.072 | 13.592 | 1.00 | 51.94 | C |
| ATOM | 646 | CD1 | TYR | X | 92 | 5.221 | 8.367 | 13.332 | 1.00 | 51.94 | C |
| ATOM | 647 | CD2 | TYR | X | 92 | 6.344 | 6.387 | 12.569 | 1.00 | 51.94 | C |
| ATOM | 648 | CE1 | TYR | X | 92 | 5.429 | 8.970 | 12.088 | 1.00 | 51.94 | C |
| ATOM | 649 | CE2 | TYR | X | 92 | 6.554 | 6.981 | 11.327 | 1.00 | 51.94 | C |
| ATOM | 650 | CZ | TYR | X | 92 | 6.094 | 8.274 | 11.100 | 1.00 | 51.94 | C |
| ATOM | 651 | OH | TYR | X | 92 | 6.315 | 8.879 | 9.892 | 1.00 | 51.94 | O |
| ATOM | 652 | N | ILE | X | 93 | 4.310 | 3.866 | 16.422 | 1.00 | 35.81 | N |
| ATOM | 653 | CA | ILE | X | 93 | 4.339 | 3.034 | 17.616 | 1.00 | 35.81 | C |
| ATOM | 654 | C | ILE | X | 93 | 5.795 | 2.737 | 17.945 | 1.00 | 35.81 | C |
| ATOM | 655 | O | ILE | X | 93 | 6.447 | 1.924 | 17.295 | 1.00 | 35.81 | O |
| ATOM | 656 | CB | ILE | X | 93 | 3.542 | 1.732 | 17.407 | 1.00 | 25.05 | C |
| ATOM | 657 | CG1 | ILE | X | 93 | 2.067 | 2.069 | 17.238 | 1.00 | 25.05 | C |
| ATOM | 658 | CG2 | ILE | X | 93 | 3.705 | 0.815 | 18.589 | 1.00 | 25.05 | C |
| ATOM | 659 | CD1 | ILE | X | 93 | 1.550 | 3.019 | 18.292 | 1.00 | 25.05 | C |
| ATOM | 660 | N | THR | X | 94 | 6.304 | 3.425 | 18.955 | 1.00 | 44.25 | N |
| ATOM | 661 | CA | THR | X | 94 | 7.689 | 3.260 | 19.337 | 1.00 | 44.25 | C |
| ATOM | 662 | C | THR | X | 94 | 7.818 | 2.816 | 20.773 | 1.00 | 44.25 | C |
| ATOM | 663 | O | THR | X | 94 | 8.930 | 2.680 | 21.290 | 1.00 | 44.25 | O |
| ATOM | 664 | CB | THR | X | 94 | 8.450 | 4.569 | 19.182 | 1.00 | 31.85 | C |
| ATOM | 665 | OG1 | THR | X | 94 | 7.878 | 5.545 | 20.055 | 1.00 | 31.85 | O |
| ATOM | 666 | CG2 | THR | X | 94 | 8.365 | 5.067 | 17.764 | 1.00 | 31.85 | C |
| ATOM | 667 | N | ASP | X | 95 | 6.687 | 2.594 | 21.423 | 1.00 | 41.33 | N |
| ATOM | 668 | CA | ASP | X | 95 | 6.734 | 2.168 | 22.804 | 1.00 | 41.33 | C |
| ATOM | 669 | C | ASP | X | 95 | 5.582 | 1.248 | 23.159 | 1.00 | 41.33 | C |
| ATOM | 670 | O | ASP | X | 95 | 4.811 | 0.860 | 22.287 | 1.00 | 41.33 | O |
| ATOM | 671 | CB | ASP | X | 95 | 6.749 | 3.385 | 23.719 | 1.00 | 46.56 | C |
| ATOM | 672 | CG | ASP | X | 95 | 7.241 | 3.056 | 25.107 | 1.00 | 46.56 | C |
| ATOM | 673 | OD1 | ASP | X | 95 | 8.263 | 2.344 | 25.219 | 1.00 | 46.56 | O |
| ATOM | 674 | OD2 | ASP | X | 95 | 6.609 | 3.520 | 26.074 | 1.00 | 46.56 | O |
| ATOM | 675 | N | MET | X | 96 | 5.459 | 0.901 | 24.438 | 1.00 | 38.95 | N |
| ATOM | 676 | CA | MET | X | 96 | 4.403 | −0.006 | 24.854 | 1.00 | 38.95 | C |
| ATOM | 677 | C | MET | X | 96 | 3.818 | 0.242 | 26.226 | 1.00 | 38.95 | C |
| ATOM | 678 | O | MET | X | 96 | 4.406 | 0.917 | 27.052 | 1.00 | 38.95 | O |
| ATOM | 679 | CB | MET | X | 96 | 4.912 | −1.445 | 24.803 | 1.00 | 48.57 | C |
| ATOM | 680 | CG | MET | X | 96 | 6.080 | −1.708 | 25.727 | 1.00 | 48.57 | C |
| ATOM | 681 | SD | MET | X | 96 | 5.962 | −3.305 | 26.529 | 1.00 | 48.57 | S |
| ATOM | 682 | CE | MET | X | 96 | 4.657 | −2.957 | 27.709 | 1.00 | 48.57 | C |
| ATOM | 683 | N | ASN | X | 97 | 2.644 | −0.331 | 26.443 | 1.00 | 29.42 | N |
| ATOM | 684 | CA | ASN | X | 97 | 1.928 | −0.253 | 27.704 | 1.00 | 29.42 | C |
| ATOM | 685 | C | ASN | X | 97 | 0.850 | −1.350 | 27.692 | 1.00 | 29.42 | C |
| ATOM | 686 | O | ASN | X | 97 | −0.141 | −1.265 | 26.963 | 1.00 | 29.42 | O |
| ATOM | 687 | CB | ASN | X | 97 | 1.295 | 1.119 | 27.886 | 1.00 | 38.27 | C |
| ATOM | 688 | CG | ASN | X | 97 | 0.453 | 1.204 | 29.152 | 1.00 | 38.27 | C |
| ATOM | 689 | OD1 | ASN | X | 97 | −0.171 | 2.229 | 29.431 | 1.00 | 38.27 | O |
| ATOM | 690 | ND2 | ASN | X | 97 | 0.429 | 0.122 | 29.923 | 1.00 | 38.27 | N |
| ATOM | 691 | N | TYR | X | 98 | 1.048 | −2.383 | 28.506 | 1.00 | 52.46 | N |
| ATOM | 692 | CA | TYR | X | 98 | 0.112 | −3.496 | 28.541 | 1.00 | 52.46 | C |
| ATOM | 693 | C | TYR | X | 98 | −0.962 | −3.480 | 29.620 | 1.00 | 52.46 | C |
| ATOM | 694 | O | TYR | X | 98 | −1.615 | −4.491 | 29.844 | 1.00 | 52.46 | O |
| ATOM | 695 | CB | TYR | X | 98 | 0.891 | −4.809 | 28.610 | 1.00 | 44.42 | C |
| ATOM | 696 | CG | TYR | X | 98 | 1.520 | −5.214 | 27.289 | 1.00 | 44.42 | C |
| ATOM | 697 | CD1 | TYR | X | 98 | 2.016 | −4.258 | 26.400 | 1.00 | 44.42 | C |
| ATOM | 698 | CD2 | TYR | X | 98 | 1.627 | −6.556 | 26.929 | 1.00 | 44.42 | C |
| ATOM | 699 | CE1 | TYR | X | 98 | 2.603 | −4.634 | 25.180 | 1.00 | 44.42 | C |
| ATOM | 700 | CE2 | TYR | X | 98 | 2.213 | −6.943 | 25.718 | 1.00 | 44.42 | C |
| ATOM | 701 | CZ | TYR | X | 98 | 2.698 | −5.978 | 24.850 | 1.00 | 44.42 | C |
| ATOM | 702 | OH | TYR | X | 98 | 3.286 | −6.362 | 23.667 | 1.00 | 44.42 | O |
| ATOM | 703 | N | GLU | X | 99 | −1.168 | −2.345 | 30.277 | 1.00 | 31.89 | N |
| ATOM | 704 | CA | GLU | X | 99 | −2.189 | −2.275 | 31.316 | 1.00 | 31.89 | C |
| ATOM | 705 | C | GLU | X | 99 | −3.540 | −2.672 | 30.748 | 1.00 | 31.89 | C |
| ATOM | 706 | O | GLU | X | 99 | −4.013 | −2.100 | 29.769 | 1.00 | 31.89 | O |
| ATOM | 707 | CB | GLU | X | 99 | −2.264 | −0.864 | 31.902 | 1.00 | 68.33 | C |
| ATOM | 708 | CG | GLU | X | 99 | −1.155 | −0.542 | 32.893 | 1.00 | 68.33 | C |
| ATOM | 709 | CD | GLU | X | 99 | −1.185 | 0.905 | 33.349 | 1.00 | 68.33 | C |
| ATOM | 710 | OE1 | GLU | X | 99 | −2.288 | 1.406 | 33.659 | 1.00 | 68.33 | O |
| ATOM | 711 | OE2 | GLU | X | 99 | −0.107 | 1.538 | 33.403 | 1.00 | 68.33 | O |
| ATOM | 712 | N | PRO | X | 100 | −4.189 | −3.666 | 31.357 | 1.00 | 65.90 | N |
| ATOM | 713 | CA | PRO | X | 100 | −5.495 | −4.080 | 30.836 | 1.00 | 65.90 | C |
| ATOM | 714 | C | PRO | X | 100 | −6.634 | −3.103 | 31.124 | 1.00 | 65.90 | C |
| ATOM | 715 | O | PRO | X | 100 | −7.674 | −3.519 | 31.621 | 1.00 | 65.90 | O |
| ATOM | 716 | CB | PRO | X | 100 | −5.725 | −5.432 | 31.517 | 1.00 | 77.94 | C |
| ATOM | 717 | CG | PRO | X | 100 | −4.319 | −5.918 | 31.829 | 1.00 | 77.94 | C |
| ATOM | 718 | CD | PRO | X | 100 | −3.660 | −4.659 | 32.307 | 1.00 | 77.94 | C |
| ATOM | 719 | N | ILE | X | 101 | −6.465 | −1.819 | 30.812 | 1.00 | 48.28 | N |
| ATOM | 720 | CA | ILE | X | 101 | −7.542 | −0.859 | 31.085 | 1.00 | 48.28 | C |
| ATOM | 721 | C | ILE | X | 101 | −8.640 | −0.869 | 30.036 | 1.00 | 48.28 | C |
| ATOM | 722 | O | ILE | X | 101 | −9.745 | −0.406 | 30.302 | 1.00 | 48.28 | O |
| ATOM | 723 | CB | ILE | X | 101 | −7.046 | 0.587 | 31.172 | 1.00 | 55.27 | C |
| ATOM | 724 | CG1 | ILE | X | 101 | −6.564 | 1.050 | 29.800 | 1.00 | 55.27 | C |
| ATOM | 725 | CG2 | ILE | X | 101 | −5.954 | 0.700 | 32.210 | 1.00 | 55.27 | C |
| ATOM | 726 | CD1 | ILE | X | 101 | −6.231 | 2.515 | 29.745 | 1.00 | 55.27 | C |
| ATOM | 727 | N | ALA | X | 102 | −8.334 | −1.372 | 28.840 | 1.00 | 49.68 | N |
| ATOM | 728 | CA | ALA | X | 102 | −9.327 | −1.442 | 27.771 | 1.00 | 49.68 | C |
| ATOM | 729 | C | ALA | X | 102 | −8.861 | −2.352 | 26.651 | 1.00 | 49.68 | C |
| ATOM | 730 | O | ALA | X | 102 | −7.669 | −2.602 | 26.509 | 1.00 | 49.68 | O |
| ATOM | 731 | CB | ALA | X | 102 | −9.604 | −0.058 | 27.224 | 1.00 | 50.56 | C |
| ATOM | 732 | N | PRO | X | 103 | −9.804 | −2.862 | 25.840 | 1.00 | 33.37 | N |
| ATOM | 733 | CA | PRO | X | 103 | −9.506 | −3.750 | 24.716 | 1.00 | 33.37 | C |
| ATOM | 734 | C | PRO | X | 103 | −8.323 | −3.215 | 23.928 | 1.00 | 33.37 | C |
| ATOM | 735 | O | PRO | X | 103 | −7.287 | −3.865 | 23.839 | 1.00 | 33.37 | O |
| ATOM | 736 | CB | PRO | X | 103 | −10.804 | −3.727 | 23.922 | 1.00 | 31.39 | C |
| ATOM | 737 | CG | PRO | X | 103 | −11.829 | −3.671 | 24.995 | 1.00 | 31.39 | C |
| ATOM | 738 | CD | PRO | X | 103 | −11.256 | −2.619 | 25.931 | 1.00 | 31.39 | C |
| ATOM | 739 | N | TYR | X | 104 | −8.482 | −2.029 | 23.345 | 1.00 | 49.21 | N |
| ATOM | 740 | CA | TYR | X | 104 | −7.392 | −1.393 | 22.599 | 1.00 | 49.21 | C |
| ATOM | 741 | C | TYR | X | 104 | −7.017 | −0.122 | 23.343 | 1.00 | 49.21 | C |
| ATOM | 742 | O | TYR | X | 104 | −7.884 | 0.685 | 23.675 | 1.00 | 49.21 | O |
| ATOM | 743 | CB | TYR | X | 104 | −7.806 | −1.035 | 21.168 | 1.00 | 45.54 | C |
| ATOM | 744 | CG | TYR | X | 104 | −8.016 | −2.213 | 20.242 | 1.00 | 45.54 | C |
| ATOM | 745 | CD1 | TYR | X | 104 | −9.302 | −2.643 | 19.911 | 1.00 | 45.54 | C |
| ATOM | 746 | CD2 | TYR | X | 104 | −6.934 | −2.864 | 19.659 | 1.00 | 45.54 | C |
| ATOM | 747 | CE1 | TYR | X | 104 | −9.500 | −3.684 | 19.018 | 1.00 | 45.54 | C |
| ATOM | 748 | CE2 | TYR | X | 104 | −7.120 | −3.903 | 18.770 | 1.00 | 45.54 | C |
| ATOM | 749 | CZ | TYR | X | 104 | −8.405 | −4.306 | 18.450 | 1.00 | 45.54 | C |
| ATOM | 750 | OH | TYR | X | 104 | −8.596 | −5.315 | 17.540 | 1.00 | 45.54 | O |
| ATOM | 751 | N | HIS | X | 105 | −5.726 | 0.059 | 23.591 | 1.00 | 29.90 | N |
| ATOM | 752 | CA | HIS | X | 105 | −5.264 | 1.220 | 24.327 | 1.00 | 29.90 | C |
| ATOM | 753 | C | HIS | X | 105 | −4.078 | 1.933 | 23.707 | 1.00 | 29.90 | C |
| ATOM | 754 | O | HIS | X | 105 | −3.072 | 1.310 | 23.381 | 1.00 | 29.90 | O |
| ATOM | 755 | CB | HIS | X | 105 | −4.915 | 0.794 | 25.748 | 1.00 | 64.97 | C |
| ATOM | 756 | CG | HIS | X | 105 | −4.261 | 1.865 | 26.558 | 1.00 | 64.97 | C |
| ATOM | 757 | ND1 | HIS | X | 105 | −3.024 | 1.701 | 27.140 | 1.00 | 64.97 | N |
| ATOM | 758 | CD2 | HIS | X | 105 | −4.678 | 3.106 | 26.899 | 1.00 | 64.97 | C |
| ATOM | 759 | CE1 | HIS | X | 105 | −2.707 | 2.796 | 27.808 | 1.00 | 64.97 | C |
| ATOM | 760 | NE2 | HIS | X | 105 | −3.694 | 3.664 | 27.678 | 1.00 | 64.97 | N |
| ATOM | 761 | N | PHE | X | 106 | −4.211 | 3.247 | 23.542 | 1.00 | 36.72 | N |
| ATOM | 762 | CA | PHE | X | 106 | −3.138 | 4.074 | 22.994 | 1.00 | 36.72 | C |
| ATOM | 763 | C | PHE | X | 106 | −2.725 | 4.999 | 24.112 | 1.00 | 36.72 | C |
| ATOM | 764 | O | PHE | X | 106 | −3.571 | 5.569 | 24.803 | 1.00 | 36.72 | O |
| ATOM | 765 | CB | PHE | X | 106 | −3.599 | 4.955 | 21.818 | 1.00 | 34.76 | C |
| ATOM | 766 | CG | PHE | X | 106 | −3.737 | 4.230 | 20.515 | 1.00 | 34.76 | C |
| ATOM | 767 | CD1 | PHE | X | 106 | −4.923 | 3.576 | 20.183 | 1.00 | 34.76 | C |
| ATOM | 768 | CD2 | PHE | X | 106 | −2.676 | 4.194 | 19.613 | 1.00 | 34.76 | C |
| ATOM | 769 | CE1 | PHE | X | 106 | −5.049 | 2.899 | 18.972 | 1.00 | 34.76 | C |
| ATOM | 770 | CE2 | PHE | X | 106 | −2.794 | 3.517 | 18.397 | 1.00 | 34.76 | C |
| ATOM | 771 | CZ | PHE | X | 106 | −3.981 | 2.872 | 18.080 | 1.00 | 34.76 | C |
| ATOM | 772 | N | ASP | X | 107 | −1.422 | 5.145 | 24.287 | 1.00 | 25.99 | N |
| ATOM | 773 | CA | ASP | X | 107 | −0.879 | 6.021 | 25.310 | 1.00 | 25.99 | C |
| ATOM | 774 | C | ASP | X | 107 | −0.043 | 6.976 | 24.495 | 1.00 | 25.99 | C |
| ATOM | 775 | O | ASP | X | 107 | 1.114 | 6.703 | 24.183 | 1.00 | 25.99 | O |
| ATOM | 776 | CB | ASP | X | 107 | 0.004 | 5.234 | 26.286 | 1.00 | 56.65 | C |
| ATOM | 777 | CG | ASP | X | 107 | −0.010 | 5.817 | 27.690 | 1.00 | 56.65 | C |
| ATOM | 778 | OD1 | ASP | X | 107 | 0.816 | 5.389 | 28.517 | 1.00 | 56.65 | O |
| ATOM | 779 | OD2 | ASP | X | 107 | −0.850 | 6.692 | 27.974 | 1.00 | 56.65 | O |
| ATOM | 780 | N | LEU | X | 108 | −0.643 | 8.096 | 24.134 | 1.00 | 46.68 | N |
| ATOM | 781 | CA | LEU | X | 108 | 0.037 | 9.062 | 23.300 | 1.00 | 46.68 | C |
| ATOM | 782 | C | LEU | X | 108 | 1.006 | 9.953 | 24.046 | 1.00 | 46.68 | C |
| ATOM | 783 | O | LEU | X | 108 | 1.015 | 10.012 | 25.270 | 1.00 | 46.68 | O |
| ATOM | 784 | CB | LEU | X | 108 | −1.003 | 9.919 | 22.578 | 1.00 | 36.48 | C |
| ATOM | 785 | CG | LEU | X | 108 | −2.051 | 9.128 | 21.783 | 1.00 | 36.48 | C |
| ATOM | 786 | CD1 | LEU | X | 108 | −3.179 | 10.040 | 21.345 | 1.00 | 36.48 | C |
| ATOM | 787 | CD2 | LEU | X | 108 | −1.392 | 8.462 | 20.592 | 1.00 | 36.48 | C |
| ATOM | 788 | N | SER | X | 109 | 1.844 | 10.630 | 23.283 | 1.00 | 37.04 | N |
| ATOM | 789 | CA | SER | X | 109 | 2.792 | 11.572 | 23.834 | 1.00 | 37.04 | C |
| ATOM | 790 | C | SER | X | 109 | 1.946 | 12.809 | 24.130 | 1.00 | 37.04 | C |
| ATOM | 791 | O | SER | X | 109 | 0.773 | 12.868 | 23.752 | 1.00 | 37.04 | O |
| ATOM | 792 | CB | SER | X | 109 | 3.839 | 11.918 | 22.781 | 1.00 | 35.78 | C |
| ATOM | 793 | OG | SER | X | 109 | 3.209 | 12.477 | 21.642 | 1.00 | 35.78 | O |
| ATOM | 794 | N | GLY | X | 110 | 2.528 | 13.797 | 24.801 | 1.00 | 45.00 | N |
| ATOM | 795 | CA | GLY | X | 110 | 1.775 | 15.006 | 25.086 | 1.00 | 45.00 | C |
| ATOM | 796 | C | GLY | X | 110 | 1.492 | 15.712 | 23.774 | 1.00 | 45.00 | C |
| ATOM | 797 | O | GLY | X | 110 | 0.375 | 16.162 | 23.515 | 1.00 | 45.00 | O |
| ATOM | 798 | N | LYS | X | 111 | 2.525 | 15.793 | 22.943 | 1.00 | 42.59 | N |
| ATOM | 799 | CA | LYS | X | 111 | 2.441 | 16.423 | 21.630 | 1.00 | 42.59 | C |
| ATOM | 800 | C | LYS | X | 111 | 1.277 | 15.820 | 20.850 | 1.00 | 42.59 | C |
| ATOM | 801 | O | LYS | X | 111 | 0.321 | 16.518 | 20.494 | 1.00 | 42.59 | O |
| ATOM | 802 | CB | LYS | X | 111 | 3.748 | 16.183 | 20.878 | 1.00 | 36.13 | C |
| ATOM | 803 | CG | LYS | X | 111 | 3.972 | 17.011 | 19.635 | 1.00 | 36.13 | C |
| ATOM | 804 | CD | LYS | X | 111 | 5.397 | 16.781 | 19.164 | 1.00 | 36.13 | C |
| ATOM | 805 | CE | LYS | X | 111 | 5.834 | 17.775 | 18.107 | 1.00 | 36.13 | C |
| ATOM | 806 | NZ | LYS | X | 111 | 5.108 | 17.577 | 16.820 | 1.00 | 36.13 | N |
| ATOM | 807 | N | ALA | X | 112 | 1.368 | 14.514 | 20.596 | 1.00 | 54.93 | N |
| ATOM | 808 | CA | ALA | X | 112 | 0.339 | 13.786 | 19.863 | 1.00 | 54.93 | C |
| ATOM | 809 | C | ALA | X | 112 | −1.027 | 13.978 | 20.498 | 1.00 | 54.93 | C |
| ATOM | 810 | O | ALA | X | 112 | −1.941 | 14.493 | 19.872 | 1.00 | 54.93 | O |
| ATOM | 811 | CB | ALA | X | 112 | 0.678 | 12.307 | 19.819 | 1.00 | 46.83 | C |
| ATOM | 812 | N | PHE | X | 113 | −1.167 | 13.571 | 21.748 | 1.00 | 52.54 | N |
| ATOM | 813 | CA | PHE | X | 113 | −2.448 | 13.701 | 22.422 | 1.00 | 52.54 | C |
| ATOM | 814 | C | PHE | X | 113 | −3.049 | 15.087 | 22.222 | 1.00 | 52.54 | C |
| ATOM | 815 | O | PHE | X | 113 | −4.263 | 15.229 | 22.051 | 1.00 | 52.54 | O |
| ATOM | 816 | CB | PHE | X | 113 | −2.294 | 13.433 | 23.915 | 1.00 | 34.42 | C |
| ATOM | 817 | CG | PHE | X | 113 | −3.591 | 13.167 | 24.606 | 1.00 | 34.42 | C |
| ATOM | 818 | CD1 | PHE | X | 113 | −3.941 | 11.876 | 24.969 | 1.00 | 34.42 | C |
| ATOM | 819 | CD2 | PHE | X | 113 | −4.486 | 14.196 | 24.841 | 1.00 | 34.42 | C |
| ATOM | 820 | CE1 | PHE | X | 113 | −5.167 | 11.607 | 25.552 | 1.00 | 34.42 | C |
| ATOM | 821 | CE2 | PHE | X | 113 | −5.723 | 13.939 | 25.427 | 1.00 | 34.42 | C |
| ATOM | 822 | CZ | PHE | X | 113 | −6.067 | 12.637 | 25.783 | 1.00 | 34.42 | C |
| ATOM | 823 | N | GLY | X | 114 | −2.194 | 16.107 | 22.254 | 1.00 | 62.96 | N |
| ATOM | 824 | CA | GLY | X | 114 | −2.659 | 17.472 | 22.078 | 1.00 | 62.96 | C |
| ATOM | 825 | C | GLY | X | 114 | −3.149 | 17.732 | 20.670 | 1.00 | 62.96 | C |
| ATOM | 826 | O | GLY | X | 114 | −4.180 | 18.375 | 20.475 | 1.00 | 62.96 | O |
| ATOM | 827 | N | SER | X | 115 | −2.404 | 17.220 | 19.693 | 1.00 | 41.75 | N |
| ATOM | 828 | CA | SER | X | 115 | −2.734 | 17.373 | 18.282 | 1.00 | 41.75 | C |
| ATOM | 829 | C | SER | X | 115 | −4.139 | 16.942 | 17.914 | 1.00 | 41.75 | C |
| ATOM | 830 | O | SER | X | 115 | −4.631 | 17.271 | 16.842 | 1.00 | 41.75 | O |
| ATOM | 831 | CB | SER | X | 115 | −1.743 | 16.599 | 17.427 | 1.00 | 31.27 | C |
| ATOM | 832 | OG | SER | X | 115 | −0.458 | 17.184 | 17.502 | 1.00 | 31.27 | O |
| ATOM | 833 | N | LEU | X | 116 | −4.797 | 16.200 | 18.785 | 1.00 | 36.02 | N |
| ATOM | 834 | CA | LEU | X | 116 | −6.149 | 15.774 | 18.473 | 1.00 | 36.02 | C |
| ATOM | 835 | C | LEU | X | 116 | −7.111 | 16.927 | 18.683 | 1.00 | 36.02 | C |
| ATOM | 836 | O | LEU | X | 116 | −8.313 | 16.787 | 18.456 | 1.00 | 36.02 | O |
| ATOM | 837 | CB | LEU | X | 116 | −6.563 | 14.606 | 19.369 | 1.00 | 67.90 | C |
| ATOM | 838 | CG | LEU | X | 116 | −5.945 | 13.238 | 19.085 | 1.00 | 67.90 | C |
| ATOM | 839 | CD1 | LEU | X | 116 | −6.298 | 12.271 | 20.203 | 1.00 | 67.90 | C |
| ATOM | 840 | CD2 | LEU | X | 116 | −6.460 | 12.723 | 17.756 | 1.00 | 67.90 | C |
| ATOM | 841 | N | ALA | X | 117 | −6.580 | 18.074 | 19.104 | 1.00 | 67.56 | N |
| ATOM | 842 | CA | ALA | X | 117 | −7.409 | 19.247 | 19.400 | 1.00 | 67.56 | C |
| ATOM | 843 | C | ALA | X | 117 | −7.461 | 20.372 | 18.376 | 1.00 | 67.56 | C |
| ATOM | 844 | O | ALA | X | 117 | −6.535 | 20.577 | 17.595 | 1.00 | 67.56 | O |
| ATOM | 845 | CB | ALA | X | 117 | −6.996 | 19.826 | 20.749 | 1.00 | 67.72 | C |
| ATOM | 846 | N | LYS | X | 118 | −8.566 | 21.107 | 18.405 | 1.00 | 62.31 | N |
| ATOM | 847 | CA | LYS | X | 118 | −8.756 | 22.246 | 17.520 | 1.00 | 62.31 | C |
| ATOM | 848 | C | LYS | X | 118 | −7.646 | 23.219 | 17.901 | 1.00 | 62.31 | C |
| ATOM | 849 | O | LYS | X | 118 | −7.308 | 23.349 | 19.076 | 1.00 | 62.31 | O |
| ATOM | 850 | CB | LYS | X | 118 | −10.121 | 22.887 | 17.774 | 1.00 | 70.06 | C |
| ATOM | 851 | CG | LYS | X | 118 | −11.287 | 21.909 | 17.729 | 1.00 | 70.06 | C |
| ATOM | 852 | CD | LYS | X | 118 | −12.588 | 22.547 | 18.219 | 1.00 | 70.06 | C |
| ATOM | 853 | CE | LYS | X | 118 | −13.162 | 23.554 | 17.225 | 1.00 | 70.06 | C |
| ATOM | 854 | NZ | LYS | X | 118 | −13.743 | 22.908 | 16.013 | 1.00 | 70.06 | N |
| ATOM | 855 | N | PRO | X | 119 | −7.070 | 23.920 | 16.916 | 1.00 | 70.89 | N |
| ATOM | 856 | CA | PRO | X | 119 | −5.989 | 24.880 | 17.166 | 1.00 | 70.89 | C |
| ATOM | 857 | C | PRO | X | 119 | −6.164 | 25.752 | 18.409 | 1.00 | 70.89 | C |
| ATOM | 858 | O | PRO | X | 119 | −7.208 | 26.385 | 18.598 | 1.00 | 70.89 | O |
| ATOM | 859 | CB | PRO | X | 119 | −5.958 | 25.697 | 15.881 | 1.00 | 73.89 | C |
| ATOM | 860 | CG | PRO | X | 119 | −6.308 | 24.670 | 14.851 | 1.00 | 73.89 | C |
| ATOM | 861 | CD | PRO | X | 119 | −7.472 | 23.948 | 15.498 | 1.00 | 73.89 | C |
| ATOM | 862 | N | GLY | X | 120 | −5.130 | 25.764 | 19.252 | 1.00 | 72.64 | N |
| ATOM | 863 | CA | GLY | X | 120 | −5.138 | 26.561 | 20.470 | 1.00 | 72.64 | C |
| ATOM | 864 | C | GLY | X | 120 | −5.803 | 25.937 | 21.682 | 1.00 | 72.64 | C |
| ATOM | 865 | O | GLY | X | 120 | −5.872 | 26.557 | 22.744 | 1.00 | 72.64 | O |
| ATOM | 866 | N | LEU | X | 121 | −6.278 | 24.704 | 21.534 | 1.00 | 98.68 | N |
| ATOM | 867 | CA | LEU | X | 121 | −6.955 | 24.012 | 22.625 | 1.00 | 98.68 | C |
| ATOM | 868 | C | LEU | X | 121 | −6.145 | 22.839 | 23.185 | 1.00 | 98.68 | C |
| ATOM | 869 | O | LEU | X | 121 | −6.619 | 22.100 | 24.047 | 1.00 | 98.68 | O |
| ATOM | 870 | CB | LEU | X | 121 | −8.324 | 23.533 | 22.134 | 1.00 | 57.37 | C |
| ATOM | 871 | CG | LEU | X | 121 | −9.223 | 24.670 | 21.636 | 1.00 | 57.37 | C |
| ATOM | 872 | CD1 | LEU | X | 121 | −10.441 | 24.133 | 20.912 | 1.00 | 57.37 | C |
| ATOM | 873 | CD2 | LEU | X | 121 | −9.643 | 25.510 | 22.814 | 1.00 | 57.37 | C |
| ATOM | 874 | N | ASN | X | 122 | −4.920 | 22.688 | 22.697 | 1.00 | 66.63 | N |
| ATOM | 875 | CA | ASN | X | 122 | −4.028 | 21.617 | 23.125 | 1.00 | 66.63 | C |
| ATOM | 876 | C | ASN | X | 122 | −4.042 | 21.372 | 24.630 | 1.00 | 66.63 | C |
| ATOM | 877 | O | ASN | X | 122 | −4.832 | 20.574 | 25.136 | 1.00 | 66.63 | O |
| ATOM | 878 | CB | ASN | X | 122 | −2.600 | 21.935 | 22.689 | 1.00 | 75.00 | C |
| ATOM | 879 | CG | ASN | X | 122 | −2.524 | 22.355 | 21.247 | 1.00 | 75.00 | C |
| ATOM | 880 | OD1 | ASN | X | 122 | −3.178 | 23.313 | 20.843 | 1.00 | 75.00 | O |
| ATOM | 881 | ND2 | ASN | X | 122 | −1.729 | 21.642 | 20.456 | 1.00 | 75.00 | N |
| ATOM | 882 | N | ASP | X | 123 | −3.140 | 22.058 | 25.329 | 1.00 | 76.12 | N |
| ATOM | 883 | CA | ASP | X | 123 | −2.991 | 21.952 | 26.780 | 1.00 | 76.12 | C |
| ATOM | 884 | C | ASP | X | 123 | −4.332 | 21.807 | 27.480 | 1.00 | 76.12 | C |
| ATOM | 885 | O | ASP | X | 123 | −4.436 | 21.173 | 28.527 | 1.00 | 76.12 | O |
| ATOM | 886 | CB | ASP | X | 123 | −2.230 | 23.171 | 27.320 | 1.00 | 85.08 | C |
| ATOM | 887 | CG | ASP | X | 123 | −1.873 | 24.177 | 26.226 | 1.00 | 85.08 | C |
| ATOM | 888 | OD1 | ASP | X | 123 | −0.770 | 24.768 | 26.285 | 1.00 | 85.08 | O |
| ATOM | 889 | OD2 | ASP | X | 123 | −2.701 | 24.389 | 25.313 | 1.00 | 85.08 | O |
| ATOM | 890 | N | LYS | X | 124 | −5.362 | 22.394 | 26.894 | 1.00 | 66.79 | N |
| ATOM | 891 | CA | LYS | X | 124 | −6.688 | 22.294 | 27.465 | 1.00 | 66.79 | C |
| ATOM | 892 | C | LYS | X | 124 | −7.091 | 20.828 | 27.457 | 1.00 | 66.79 | C |
| ATOM | 893 | O | LYS | X | 124 | −7.331 | 20.227 | 28.504 | 1.00 | 66.79 | O |
| ATOM | 894 | CB | LYS | X | 124 | −7.675 | 23.093 | 26.626 | 1.00 | 84.27 | C |
| ATOM | 895 | CG | LYS | X | 124 | −9.096 | 23.060 | 27.139 | 1.00 | 84.27 | C |
| ATOM | 896 | CD | LYS | X | 124 | −10.013 | 23.849 | 26.232 | 1.00 | 84.27 | C |
| ATOM | 897 | CE | LYS | X | 124 | −9.509 | 25.278 | 26.057 | 1.00 | 84.27 | C |
| ATOM | 898 | NZ | LYS | X | 124 | −8.222 | 25.348 | 25.282 | 1.00 | 84.27 | N |
| ATOM | 899 | N | ILE | X | 125 | −7.143 | 20.266 | 26.252 | 1.00 | 75.63 | N |
| ATOM | 900 | CA | ILE | X | 125 | −7.529 | 18.876 | 26.019 | 1.00 | 75.63 | C |
| ATOM | 901 | C | ILE | X | 125 | −6.622 | 17.872 | 26.746 | 1.00 | 75.63 | C |
| ATOM | 902 | O | ILE | X | 125 | −7.072 | 16.790 | 27.126 | 1.00 | 75.63 | O |
| ATOM | 903 | CB | ILE | X | 125 | −7.529 | 18.560 | 24.487 | 1.00 | 75.19 | C |
| ATOM | 904 | CG1 | ILE | X | 125 | −8.471 | 17.387 | 24.172 | 1.00 | 35.80 | C |
| ATOM | 905 | CG2 | ILE | X | 125 | −6.102 | 18.233 | 24.020 | 1.00 | 35.80 | C |
| ATOM | 906 | CD1 | ILE | X | 125 | −8.440 | 16.933 | 22.709 | 1.00 | 75.19 | C |
| ATOM | 907 | N | ARG | X | 126 | −5.352 | 18.218 | 26.934 | 1.00 | 64.76 | N |
| ATOM | 908 | CA | ARG | X | 126 | −4.440 | 17.315 | 27.626 | 1.00 | 64.76 | C |
| ATOM | 909 | C | ARG | X | 126 | −4.852 | 17.059 | 29.069 | 1.00 | 64.76 | C |
| ATOM | 910 | O | ARG | X | 126 | −4.330 | 16.144 | 29.719 | 1.00 | 64.76 | O |
| ATOM | 911 | CB | ARG | X | 126 | −3.013 | 17.857 | 27.625 | 1.00 | 44.07 | C |
| ATOM | 912 | CG | ARG | X | 126 | −2.272 | 17.627 | 26.344 | 1.00 | 44.07 | C |
| ATOM | 913 | CD | ARG | X | 126 | −2.649 | 18.658 | 25.291 | 1.00 | 44.07 | C |
| ATOM | 914 | NE | ARG | X | 126 | −1.742 | 19.806 | 25.268 | 1.00 | 44.07 | N |
| ATOM | 915 | CZ | ARG | X | 126 | −0.417 | 19.716 | 25.322 | 1.00 | 44.07 | C |
| ATOM | 916 | NH1 | ARG | X | 126 | 0.172 | 18.535 | 25.419 | 1.00 | 44.07 | N |
| ATOM | 917 | NH2 | ARG | X | 126 | 0.324 | 20.805 | 25.243 | 1.00 | 44.07 | N |
| ATOM | 918 | N | HIS | X | 127 | −5.768 | 17.873 | 29.586 | 1.00 | 61.54 | N |
| ATOM | 919 | CA | HIS | X | 127 | −6.202 | 17.674 | 30.954 | 1.00 | 61.54 | C |
| ATOM | 920 | C | HIS | X | 127 | −7.577 | 17.054 | 31.054 | 1.00 | 61.54 | C |
| ATOM | 921 | O | HIS | X | 127 | −8.510 | 17.614 | 31.633 | 1.00 | 61.54 | O |
| ATOM | 922 | CB | HIS | X | 127 | −6.154 | 18.967 | 31.759 | 1.00 | 99.00 | C |
| ATOM | 923 | CG | HIS | X | 127 | −5.643 | 18.768 | 33.151 | 1.00 | 99.00 | C |
| ATOM | 924 | ND1 | HIS | X | 127 | −4.333 | 18.429 | 33.416 | 1.00 | 99.00 | N |
| ATOM | 925 | CD2 | HIS | X | 127 | −6.277 | 18.778 | 34.347 | 1.00 | 99.00 | C |
| ATOM | 926 | CE1 | HIS | X | 127 | −4.184 | 18.235 | 34.714 | 1.00 | 99.00 | C |
| ATOM | 927 | NE2 | HIS | X | 127 | −5.349 | 18.440 | 35.301 | 1.00 | 99.00 | N |
| ATOM | 928 | N | CYS | X | 128 | −7.675 | 15.881 | 30.451 | 1.00 | 66.83 | N |
| ATOM | 929 | CA | CYS | X | 128 | −8.870 | 15.068 | 30.466 | 1.00 | 66.83 | C |
| ATOM | 930 | C | CYS | X | 128 | −8.256 | 13.704 | 30.661 | 1.00 | 66.83 | C |
| ATOM | 931 | O | CYS | X | 128 | −8.936 | 12.728 | 30.954 | 1.00 | 66.83 | O |
| ATOM | 932 | CB | CYS | X | 128 | −9.621 | 15.162 | 29.141 | 1.00 | 99.00 | C |
| ATOM | 933 | SG | CYS | X | 128 | −10.751 | 16.576 | 29.066 | 1.00 | 99.00 | S |
| ATOM | 934 | N | GLY | X | 129 | −6.936 | 13.675 | 30.503 | 1.00 | 72.04 | N |
| ATOM | 935 | CA | GLY | X | 129 | −6.165 | 12.462 | 30.684 | 1.00 | 72.04 | C |
| ATOM | 936 | C | GLY | X | 129 | −6.580 | 11.275 | 29.849 | 1.00 | 72.04 | C |
| ATOM | 937 | O | GLY | X | 129 | −5.813 | 10.814 | 29.010 | 1.00 | 72.04 | O |
| ATOM | 938 | N | ILE | X | 130 | −7.786 | 10.773 | 30.089 | 1.00 | 37.73 | N |
| ATOM | 939 | CA | ILE | X | 130 | −8.290 | 9.630 | 29.360 | 1.00 | 37.73 | C |
| ATOM | 940 | C | ILE | X | 130 | −9.574 | 9.940 | 28.604 | 1.00 | 37.73 | C |
| ATOM | 941 | O | ILE | X | 130 | −10.493 | 10.543 | 29.148 | 1.00 | 37.73 | O |
| ATOM | 942 | CB | ILE | X | 130 | −8.562 | 8.450 | 30.307 | 1.00 | 63.78 | C |
| ATOM | 943 | CG1 | ILE | X | 130 | −8.890 | 7.200 | 29.489 | 1.00 | 63.78 | C |
| ATOM | 944 | CG2 | ILE | X | 130 | −9.723 | 8.781 | 31.233 | 1.00 | 63.78 | C |
| ATOM | 945 | CD1 | ILE | X | 130 | −9.015 | 5.949 | 30.317 | 1.00 | 63.78 | C |
| ATOM | 946 | N | MET | X | 131 | −9.639 | 9.501 | 27.351 | 1.00 | 69.22 | N |
| ATOM | 947 | CA | MET | X | 131 | −10.819 | 9.721 | 26.519 | 1.00 | 69.22 | C |
| ATOM | 948 | C | MET | X | 131 | −10.923 | 8.658 | 25.440 | 1.00 | 69.22 | C |
| ATOM | 949 | O | MET | X | 131 | −9.938 | 8.001 | 25.102 | 1.00 | 69.22 | O |
| ATOM | 950 | CB | MET | X | 131 | −10.747 | 11.083 | 25.839 | 1.00 | 74.80 | C |
| ATOM | 951 | CG | MET | X | 131 | −9.551 | 11.220 | 24.922 | 1.00 | 74.80 | C |
| ATOM | 952 | SD | MET | X | 131 | −9.720 | 12.587 | 23.787 | 1.00 | 74.80 | S |
| ATOM | 953 | CE | MET | X | 131 | −8.990 | 13.896 | 24.741 | 1.00 | 74.80 | C |
| ATOM | 954 | N | ASP | X | 132 | −12.125 | 8.501 | 24.899 | 1.00 | 63.49 | N |
| ATOM | 955 | CA | ASP | X | 132 | −12.368 | 7.541 | 23.830 | 1.00 | 63.49 | C |
| ATOM | 956 | C | ASP | X | 132 | −11.990 | 8.165 | 22.486 | 1.00 | 63.49 | C |
| ATOM | 957 | O | ASP | X | 132 | −12.501 | 9.223 | 22.112 | 1.00 | 63.49 | O |
| ATOM | 958 | CB | ASP | X | 132 | −13.849 | 7.129 | 23.783 | 1.00 | 56.30 | C |
| ATOM | 959 | CG | ASP | X | 132 | −14.245 | 6.214 | 24.923 | 1.00 | 56.30 | C |
| ATOM | 960 | OD1 | ASP | X | 132 | −13.497 | 5.257 | 25.198 | 1.00 | 56.30 | O |
| ATOM | 961 | OD2 | ASP | X | 132 | −15.310 | 6.438 | 25.536 | 1.00 | 56.30 | O |
| ATOM | 962 | N | VAL | X | 133 | −11.090 | 7.513 | 21.764 | 1.00 | 49.41 | N |
| ATOM | 963 | CA | VAL | X | 133 | −10.678 | 7.994 | 20.454 | 1.00 | 49.41 | C |
| ATOM | 964 | C | VAL | X | 133 | −11.121 | 7.015 | 19.361 | 1.00 | 49.41 | C |
| ATOM | 965 | O | VAL | X | 133 | −11.863 | 6.071 | 19.626 | 1.00 | 49.41 | O |
| ATOM | 966 | CB | VAL | X | 133 | −9.151 | 8.168 | 20.385 | 1.00 | 47.86 | C |
| ATOM | 967 | CG1 | VAL | X | 133 | −8.751 | 9.469 | 21.049 | 1.00 | 47.86 | C |
| ATOM | 968 | CG2 | VAL | X | 133 | −8.468 | 7.000 | 21.066 | 1.00 | 47.86 | C |
| ATOM | 969 | N | GLU | X | 134 | −10.679 | 7.257 | 18.133 | 1.00 | 37.50 | N |
| ATOM | 970 | CA | GLU | X | 134 | −11.005 | 6.382 | 17.013 | 1.00 | 37.50 | C |
| ATOM | 971 | C | GLU | X | 134 | −9.755 | 6.207 | 16.186 | 1.00 | 37.50 | C |
| ATOM | 972 | O | GLU | X | 134 | −8.998 | 7.159 | 16.016 | 1.00 | 37.50 | O |
| ATOM | 973 | CB | GLU | X | 134 | −12.116 | 6.982 | 16.166 | 1.00 | 39.35 | C |
| ATOM | 974 | CG | GLU | X | 134 | −13.483 | 6.443 | 16.519 | 1.00 | 39.35 | C |
| ATOM | 975 | CD | GLU | X | 134 | −14.587 | 7.108 | 15.735 | 1.00 | 39.35 | C |
| ATOM | 976 | OE1 | GLU | X | 134 | −14.347 | 7.434 | 14.553 | 1.00 | 39.35 | O |
| ATOM | 977 | OE2 | GLU | X | 134 | −15.691 | 7.295 | 16.298 | 1.00 | 39.35 | O |
| ATOM | 978 | N | PHE | X | 135 | −9.521 | 5.004 | 15.674 | 1.00 | 41.16 | N |
| ATOM | 979 | CA | PHE | X | 135 | −8.309 | 4.788 | 14.893 | 1.00 | 41.16 | C |
| ATOM | 980 | C | PHE | X | 135 | −8.401 | 3.698 | 13.832 | 1.00 | 41.16 | C |
| ATOM | 981 | O | PHE | X | 135 | −9.391 | 2.972 | 13.732 | 1.00 | 41.16 | O |
| ATOM | 982 | CB | PHE | X | 135 | −7.165 | 4.406 | 15.817 | 1.00 | 16.80 | C |
| ATOM | 983 | CG | PHE | X | 135 | −7.208 | 2.972 | 16.236 | 1.00 | 16.80 | C |
| ATOM | 984 | CD1 | PHE | X | 135 | −8.232 | 2.509 | 17.050 | 1.00 | 16.80 | C |
| ATOM | 985 | CD2 | PHE | X | 135 | −6.276 | 2.064 | 15.737 | 1.00 | 16.80 | C |
| ATOM | 986 | CE1 | PHE | X | 135 | −8.332 | 1.163 | 17.359 | 1.00 | 16.80 | C |
| ATOM | 987 | CE2 | PHE | X | 135 | −6.365 | 0.715 | 16.037 | 1.00 | 16.80 | C |
| ATOM | 988 | CZ | PHE | X | 135 | −7.393 | 0.258 | 16.848 | 1.00 | 16.80 | C |
| ATOM | 989 | N | ARG | X | 136 | −7.328 | 3.591 | 13.059 | 1.00 | 11.01 | N |
| ATOM | 990 | CA | ARG | X | 136 | −7.210 | 2.586 | 12.039 | 1.00 | 11.01 | C |
| ATOM | 991 | C | ARG | X | 136 | −5.763 | 2.595 | 11.607 | 1.00 | 11.01 | C |
| ATOM | 992 | O | ARG | X | 136 | −5.156 | 3.644 | 11.524 | 1.00 | 11.01 | O |
| ATOM | 993 | CB | ARG | X | 136 | −8.135 | 2.889 | 10.858 | 1.00 | 49.76 | C |
| ATOM | 994 | CG | ARG | X | 136 | −7.888 | 4.187 | 10.107 | 1.00 | 49.76 | C |
| ATOM | 995 | CD | ARG | X | 136 | −8.927 | 4.292 | 9.003 | 1.00 | 49.76 | C |
| ATOM | 996 | NE | ARG | X | 136 | −9.281 | 5.662 | 8.636 | 1.00 | 49.76 | N |
| ATOM | 997 | CZ | ARG | X | 136 | −8.576 | 6.427 | 7.814 | 1.00 | 49.76 | C |
| ATOM | 998 | NH1 | ARG | X | 136 | −7.465 | 5.971 | 7.255 | 1.00 | 49.76 | N |
| ATOM | 999 | NH2 | ARG | X | 136 | −8.988 | 7.651 | 7.547 | 1.00 | 49.76 | N |
| ATOM | 1000 | N | ARG | X | 137 | −5.178 | 1.435 | 11.366 | 1.00 | 24.96 | N |
| ATOM | 1001 | CA | ARG | X | 137 | −3.788 | 1.417 | 10.944 | 1.00 | 24.96 | C |
| ATOM | 1002 | C | ARG | X | 137 | −3.711 | 2.019 | 9.538 | 1.00 | 24.96 | C |
| ATOM | 1003 | O | ARG | X | 137 | −4.736 | 2.177 | 8.872 | 1.00 | 24.96 | O |
| ATOM | 1004 | CB | ARG | X | 137 | −3.256 | −0.019 | 10.952 | 1.00 | 17.89 | C |
| ATOM | 1005 | CG | ARG | X | 137 | −1.799 | −0.133 | 10.557 | 1.00 | 17.89 | C |
| ATOM | 1006 | CD | ARG | X | 137 | −1.233 | −1.521 | 10.847 | 1.00 | 17.89 | C |
| ATOM | 1007 | NE | ARG | X | 137 | 0.169 | −1.625 | 10.438 | 1.00 | 17.89 | N |
| ATOM | 1008 | CZ | ARG | X | 137 | 0.861 | −2.757 | 10.472 | 1.00 | 17.89 | C |
| ATOM | 1009 | NH1 | ARG | X | 137 | 0.278 | −3.869 | 10.897 | 1.00 | 17.89 | N |
| ATOM | 1010 | NH2 | ARG | X | 137 | 2.125 | −2.779 | 10.073 | 1.00 | 17.89 | N |
| ATOM | 1011 | N | VAL | X | 138 | −2.505 | 2.362 | 9.093 | 1.00 | 25.83 | N |
| ATOM | 1012 | CA | VAL | X | 138 | −2.310 | 2.951 | 7.780 | 1.00 | 25.83 | C |
| ATOM | 1013 | C | VAL | X | 138 | −0.899 | 2.646 | 7.314 | 1.00 | 25.83 | C |
| ATOM | 1014 | O | VAL | X | 138 | −0.034 | 2.325 | 8.113 | 1.00 | 25.83 | O |
| ATOM | 1015 | CB | VAL | X | 138 | −2.465 | 4.491 | 7.815 | 1.00 | 15.38 | C |
| ATOM | 1016 | CG1 | VAL | X | 138 | −3.714 | 4.875 | 8.569 | 1.00 | 15.38 | C |
| ATOM | 1017 | CG2 | VAL | X | 138 | −1.243 | 5.137 | 8.463 | 1.00 | 15.38 | C |
| ATOM | 1018 | N | ARG | X | 139 | −0.650 | 2.725 | 6.022 | 1.00 | 43.70 | N |
| ATOM | 1019 | CA | ARG | X | 139 | 0.697 | 2.481 | 5.569 | 1.00 | 43.70 | C |
| ATOM | 1020 | C | ARG | X | 139 | 1.483 | 3.669 | 6.126 | 1.00 | 43.70 | C |
| ATOM | 1021 | O | ARG | X | 139 | 0.890 | 4.660 | 6.537 | 1.00 | 43.70 | O |
| ATOM | 1022 | CB | ARG | X | 139 | 0.733 | 2.436 | 4.043 | 1.00 | 50.01 | C |
| ATOM | 1023 | CG | ARG | X | 139 | −0.096 | 1.301 | 3.464 | 1.00 | 50.01 | C |
| ATOM | 1024 | CD | ARG | X | 139 | 0.066 | 1.214 | 1.961 | 1.00 | 50.01 | C |
| ATOM | 1025 | NE | ARG | X | 139 | 0.028 | −0.165 | 1.470 | 1.00 | 50.01 | N |
| ATOM | 1026 | CZ | ARG | X | 139 | −1.074 | −0.896 | 1.362 | 1.00 | 50.01 | C |
| ATOM | 1027 | NH1 | ARG | X | 139 | −2.246 | −0.385 | 1.705 | 1.00 | 50.01 | N |
| ATOM | 1028 | NH2 | ARG | X | 139 | −0.998 | −2.140 | 0.916 | 1.00 | 50.01 | N |
| ATOM | 1029 | N | CYS | X | 140 | 2.804 | 3.578 | 6.172 | 1.00 | 28.61 | N |
| ATOM | 1030 | CA | CYS | X | 140 | 3.580 | 4.684 | 6.702 | 1.00 | 28.61 | C |
| ATOM | 1031 | C | CYS | X | 140 | 4.266 | 5.422 | 5.587 | 1.00 | 28.61 | C |
| ATOM | 1032 | O | CYS | X | 140 | 4.840 | 4.807 | 4.698 | 1.00 | 28.61 | O |
| ATOM | 1033 | CB | CYS | X | 140 | 4.671 | 4.202 | 7.650 | 1.00 | 34.90 | C |
| ATOM | 1034 | SG | CYS | X | 140 | 4.213 | 3.206 | 9.085 | 1.00 | 34.90 | S |
| ATOM | 1035 | N | LYS | X | 141 | 4.259 | 6.744 | 5.672 | 1.00 | 40.28 | N |
| ATOM | 1036 | CA | LYS | X | 141 | 4.885 | 7.581 | 4.668 | 1.00 | 40.28 | C |
| ATOM | 1037 | C | LYS | X | 141 | 5.914 | 8.457 | 5.346 | 1.00 | 40.28 | C |
| ATOM | 1038 | O | LYS | X | 141 | 5.638 | 9.067 | 6.376 | 1.00 | 40.28 | O |
| ATOM | 1039 | CB | LYS | X | 141 | 3.803 | 8.423 | 3.986 | 1.00 | 85.00 | C |
| ATOM | 1040 | CG | LYS | X | 141 | 4.259 | 9.683 | 3.267 | 1.00 | 85.00 | C |
| ATOM | 1041 | CD | LYS | X | 141 | 3.512 | 10.896 | 3.845 | 1.00 | 85.00 | C |
| ATOM | 1042 | CE | LYS | X | 141 | 3.441 | 12.087 | 2.887 | 1.00 | 85.00 | C |
| ATOM | 1043 | NZ | LYS | X | 141 | 2.483 | 11.883 | 1.758 | 1.00 | 85.00 | N |
| ATOM | 1044 | N | TYR | X | 142 | 7.111 | 8.497 | 4.782 | 1.00 | 44.40 | N |
| ATOM | 1045 | CA | TYR | X | 142 | 8.200 | 9.320 | 5.310 | 1.00 | 44.40 | C |
| ATOM | 1046 | C | TYR | X | 142 | 8.589 | 10.333 | 4.231 | 1.00 | 44.40 | C |
| ATOM | 1047 | O | TYR | X | 142 | 8.116 | 10.258 | 3.098 | 1.00 | 44.40 | O |
| ATOM | 1048 | CB | TYR | X | 142 | 9.429 | 8.451 | 5.641 | 1.00 | 63.47 | C |
| ATOM | 1049 | CG | TYR | X | 142 | 9.331 | 7.661 | 6.927 | 1.00 | 63.47 | C |
| ATOM | 1050 | CD1 | TYR | X | 142 | 9.514 | 8.276 | 8.161 | 1.00 | 63.47 | C |
| ATOM | 1051 | CD2 | TYR | X | 142 | 9.015 | 6.308 | 6.912 | 1.00 | 63.47 | C |
| ATOM | 1052 | CE1 | TYR | X | 142 | 9.378 | 7.564 | 9.348 | 1.00 | 63.47 | C |
| ATOM | 1053 | CE2 | TYR | X | 142 | 8.876 | 5.588 | 8.090 | 1.00 | 63.47 | C |
| ATOM | 1054 | CZ | TYR | X | 142 | 9.056 | 6.221 | 9.307 | 1.00 | 63.47 | C |
| ATOM | 1055 | OH | TYR | X | 142 | 8.892 | 5.515 | 10.481 | 1.00 | 63.47 | O |
| ATOM | 1056 | N | PRO | X | 143 | 9.438 | 11.309 | 4.575 | 1.00 | 58.88 | N |
| ATOM | 1057 | CA | PRO | X | 143 | 9.821 | 12.267 | 3.542 | 1.00 | 58.88 | C |
| ATOM | 1058 | C | PRO | X | 143 | 10.342 | 11.480 | 2.342 | 1.00 | 58.88 | C |
| ATOM | 1059 | O | PRO | X | 143 | 10.807 | 10.350 | 2.483 | 1.00 | 58.88 | O |
| ATOM | 1060 | CB | PRO | X | 143 | 10.901 | 13.082 | 4.234 | 1.00 | 81.89 | C |
| ATOM | 1061 | CG | PRO | X | 143 | 10.376 | 13.156 | 5.632 | 1.00 | 81.89 | C |
| ATOM | 1062 | CD | PRO | X | 143 | 9.957 | 11.724 | 5.889 | 1.00 | 81.89 | C |
| ATOM | 1063 | N | ALA | X | 144 | 10.265 | 12.071 | 1.160 | 1.00 | 30.74 | N |
| ATOM | 1064 | CA | ALA | X | 144 | 10.704 | 11.382 | −0.041 | 1.00 | 30.74 | C |
| ATOM | 1065 | C | ALA | X | 144 | 12.199 | 11.199 | −0.068 | 1.00 | 30.74 | C |
| ATOM | 1066 | O | ALA | X | 144 | 12.951 | 12.033 | 0.421 | 1.00 | 30.74 | O |
| ATOM | 1067 | CB | ALA | X | 144 | 10.238 | 12.141 | −1.283 | 1.00 | 49.37 | C |
| ATOM | 1068 | N | GLY | X | 145 | 12.630 | 10.093 | −0.651 | 1.00 | 48.20 | N |
| ATOM | 1069 | CA | GLY | X | 145 | 14.047 | 9.822 | −0.738 | 1.00 | 48.20 | C |
| ATOM | 1070 | C | GLY | X | 145 | 14.568 | 9.167 | 0.516 | 1.00 | 48.20 | C |
| ATOM | 1071 | O | GLY | X | 145 | 15.619 | 8.543 | 0.479 | 1.00 | 48.20 | O |
| ATOM | 1072 | N | GLN | X | 146 | 13.832 | 9.308 | 1.618 | 1.00 | 42.12 | N |
| ATOM | 1073 | CA | GLN | X | 146 | 14.215 | 8.739 | 2.913 | 1.00 | 42.12 | C |
| ATOM | 1074 | C | GLN | X | 146 | 14.158 | 7.220 | 2.904 | 1.00 | 42.12 | C |
| ATOM | 1075 | O | GLN | X | 146 | 13.075 | 6.641 | 2.993 | 1.00 | 42.12 | O |
| ATOM | 1076 | CB | GLN | X | 146 | 13.285 | 9.265 | 4.009 | 1.00 | 58.85 | C |
| ATOM | 1077 | CG | GLN | X | 146 | 13.442 | 8.563 | 5.347 | 1.00 | 58.85 | C |
| ATOM | 1078 | CD | GLN | X | 146 | 14.754 | 8.888 | 6.018 | 1.00 | 58.85 | C |
| ATOM | 1079 | OE1 | GLN | X | 146 | 14.943 | 9.989 | 6.537 | 1.00 | 58.85 | O |
| ATOM | 1080 | NE2 | GLN | X | 146 | 15.675 | 7.936 | 6.003 | 1.00 | 58.85 | N |
| ATOM | 1081 | N | LYS | X | 147 | 15.319 | 6.577 | 2.818 | 1.00 | 48.15 | N |
| ATOM | 1082 | CA | LYS | X | 147 | 15.373 | 5.119 | 2.784 | 1.00 | 48.15 | C |
| ATOM | 1083 | C | LYS | X | 147 | 15.513 | 4.518 | 4.184 | 1.00 | 48.15 | C |
| ATOM | 1084 | O | LYS | X | 147 | 15.515 | 5.243 | 5.178 | 1.00 | 48.15 | O |
| ATOM | 1085 | CB | LYS | X | 147 | 16.522 | 4.659 | 1.885 | 1.00 | 50.56 | C |
| ATOM | 1086 | CG | LYS | X | 147 | 16.720 | 5.537 | 0.650 | 1.00 | 50.56 | C |
| ATOM | 1087 | CD | LYS | X | 147 | 15.428 | 5.738 | −0.166 | 1.00 | 50.56 | C |
| ATOM | 1088 | CE | LYS | X | 147 | 15.290 | 4.724 | −1.284 | 1.00 | 50.56 | C |
| ATOM | 1089 | NZ | LYS | X | 147 | 16.461 | 4.788 | −2.198 | 1.00 | 50.56 | N |
| ATOM | 1090 | N | ILE | X | 148 | 15.619 | 3.193 | 4.257 | 1.00 | 55.60 | N |
| ATOM | 1091 | CA | ILE | X | 148 | 15.732 | 2.504 | 5.539 | 1.00 | 55.60 | C |
| ATOM | 1092 | C | ILE | X | 148 | 17.118 | 2.575 | 6.160 | 1.00 | 55.60 | C |
| ATOM | 1093 | O | ILE | X | 148 | 18.124 | 2.319 | 5.497 | 1.00 | 55.60 | O |
| ATOM | 1094 | CB | ILE | X | 148 | 15.333 | 1.040 | 5.408 | 1.00 | 49.44 | C |
| ATOM | 1095 | CG1 | ILE | X | 148 | 13.836 | 0.943 | 5.139 | 1.00 | 49.44 | C |
| ATOM | 1096 | CG2 | ILE | X | 148 | 15.678 | 0.296 | 6.673 | 1.00 | 49.44 | C |
| ATOM | 1097 | CD1 | ILE | X | 148 | 13.346 | −0.468 | 4.945 | 1.00 | 49.44 | C |
| ATOM | 1098 | N | VAL | X | 149 | 17.154 | 2.902 | 7.450 | 1.00 | 57.64 | N |
| ATOM | 1099 | CA | VAL | X | 149 | 18.412 | 3.031 | 8.168 | 1.00 | 57.64 | C |
| ATOM | 1100 | C | VAL | X | 149 | 18.690 | 1.992 | 9.254 | 1.00 | 57.64 | C |
| ATOM | 1101 | O | VAL | X | 149 | 17.831 | 1.691 | 10.087 | 1.00 | 57.64 | O |
| ATOM | 1102 | CB | VAL | X | 149 | 18.515 | 4.414 | 8.797 | 1.00 | 56.80 | C |
| ATOM | 1103 | CG1 | VAL | X | 149 | 19.850 | 4.565 | 9.494 | 1.00 | 56.80 | C |
| ATOM | 1104 | CG2 | VAL | X | 149 | 18.351 | 5.466 | 7.722 | 1.00 | 56.80 | C |
| ATOM | 1105 | N | PHE | X | 150 | 19.910 | 1.459 | 9.223 | 1.00 | 43.64 | N |
| ATOM | 1106 | CA | PHE | X | 150 | 20.376 | 0.470 | 10.193 | 1.00 | 43.64 | C |
| ATOM | 1107 | C | PHE | X | 150 | 21.378 | 1.098 | 11.164 | 1.00 | 43.64 | C |
| ATOM | 1108 | O | PHE | X | 150 | 22.375 | 1.699 | 10.751 | 1.00 | 43.64 | O |
| ATOM | 1109 | CB | PHE | X | 150 | 21.056 | −0.697 | 9.488 | 1.00 | 68.61 | C |
| ATOM | 1110 | CG | PHE | X | 150 | 20.250 | −1.282 | 8.375 | 1.00 | 68.61 | C |
| ATOM | 1111 | CD1 | PHE | X | 150 | 20.217 | −0.666 | 7.125 | 1.00 | 68.61 | C |
| ATOM | 1112 | CD2 | PHE | X | 150 | 19.525 | −2.457 | 8.568 | 1.00 | 68.61 | C |
| ATOM | 1113 | CE1 | PHE | X | 150 | 19.478 | −1.212 | 6.084 | 1.00 | 68.61 | C |
| ATOM | 1114 | CE2 | PHE | X | 150 | 18.781 | −3.012 | 7.533 | 1.00 | 68.61 | C |
| ATOM | 1115 | CZ | PHE | X | 150 | 18.758 | −2.387 | 6.288 | 1.00 | 68.61 | C |
| ATOM | 1116 | N | HIS | X | 151 | 21.109 | 0.944 | 12.455 | 1.00 | 51.27 | N |
| ATOM | 1117 | CA | HIS | X | 151 | 21.975 | 1.489 | 13.497 | 1.00 | 51.27 | C |
| ATOM | 1118 | C | HIS | X | 151 | 22.589 | 0.356 | 14.313 | 1.00 | 51.27 | C |
| ATOM | 1119 | O | HIS | X | 151 | 21.877 | −0.536 | 14.777 | 1.00 | 51.27 | O |
| ATOM | 1120 | CB | HIS | X | 151 | 21.166 | 2.398 | 14.421 | 1.00 | 61.52 | C |
| ATOM | 1121 | CG | HIS | X | 151 | 21.981 | 3.059 | 15.486 | 1.00 | 61.52 | C |
| ATOM | 1122 | ND1 | HIS | X | 151 | 22.887 | 2.375 | 16.266 | 1.00 | 61.52 | N |
| ATOM | 1123 | CD2 | HIS | X | 151 | 22.000 | 4.339 | 15.923 | 1.00 | 61.52 | C |
| ATOM | 1124 | CE1 | HIS | X | 151 | 23.429 | 3.205 | 17.138 | 1.00 | 61.52 | C |
| ATOM | 1125 | NE2 | HIS | X | 151 | 22.908 | 4.403 | 16.951 | 1.00 | 61.52 | N |
| ATOM | 1126 | N | ILE | X | 152 | 23.909 | 0.399 | 14.487 | 1.00 | 66.30 | N |
| ATOM | 1127 | CA | ILE | X | 152 | 24.622 | −0.620 | 15.256 | 1.00 | 66.30 | C |
| ATOM | 1128 | C | ILE | X | 152 | 24.543 | −0.279 | 16.741 | 1.00 | 66.30 | C |
| ATOM | 1129 | O | ILE | X | 152 | 24.780 | 0.862 | 17.125 | 1.00 | 66.30 | O |
| ATOM | 1130 | CB | ILE | X | 152 | 26.098 | −0.680 | 14.852 | 1.00 | 59.48 | C |
| ATOM | 1131 | CG1 | ILE | X | 152 | 26.218 | −0.727 | 13.328 | 1.00 | 59.48 | C |
| ATOM | 1132 | CG2 | ILE | X | 152 | 26.743 | −1.914 | 15.445 | 1.00 | 59.48 | C |
| ATOM | 1133 | CD1 | ILE | X | 152 | 25.636 | −1.978 | 12.698 | 1.00 | 59.48 | C |
| ATOM | 1134 | N | GLU | X | 153 | 24.217 | −1.262 | 17.577 | 1.00 | 70.76 | N |
| ATOM | 1135 | CA | GLU | X | 153 | 24.103 | −1.017 | 19.014 | 1.00 | 70.76 | C |
| ATOM | 1136 | C | GLU | X | 153 | 25.381 | −1.317 | 19.815 | 1.00 | 70.76 | C |
| ATOM | 1137 | O | GLU | X | 153 | 26.319 | −1.946 | 19.306 | 1.00 | 70.76 | O |
| ATOM | 1138 | CB | GLU | X | 153 | 22.929 | −1.815 | 19.587 | 1.00 | 42.71 | C |
| ATOM | 1139 | CG | GLU | X | 153 | 21.567 | −1.481 | 18.986 | 1.00 | 42.71 | C |
| ATOM | 1140 | CD | GLU | X | 153 | 21.257 | 0.010 | 18.998 | 1.00 | 42.71 | C |
| ATOM | 1141 | OE1 | GLU | X | 153 | 21.934 | 0.764 | 18.260 | 1.00 | 42.71 | O |
| ATOM | 1142 | OE2 | GLU | X | 153 | 20.343 | 0.435 | 19.742 | 1.00 | 42.71 | O |
| ATOM | 1143 | N | LYS | X | 154 | 25.406 | −0.859 | 21.069 | 1.00 | 71.01 | N |
| ATOM | 1144 | CA | LYS | X | 154 | 26.555 | −1.046 | 21.960 | 1.00 | 71.01 | C |
| ATOM | 1145 | C | LYS | X | 154 | 26.836 | −2.519 | 22.275 | 1.00 | 71.01 | C |
| ATOM | 1146 | O | LYS | X | 154 | 26.030 | −3.192 | 22.908 | 1.00 | 71.01 | O |
| ATOM | 1147 | CB | LYS | X | 154 | 26.345 | −0.253 | 23.256 | 1.00 | 69.14 | C |
| ATOM | 1148 | CG | LYS | X | 154 | 25.140 | −0.653 | 24.113 | 1.00 | 69.14 | C |
| ATOM | 1149 | CD | LYS | X | 154 | 23.783 | −0.316 | 23.477 | 1.00 | 69.14 | C |
| ATOM | 1150 | CE | LYS | X | 154 | 22.667 | −0.349 | 24.540 | 1.00 | 69.14 | C |
| ATOM | 1151 | NZ | LYS | X | 154 | 21.294 | −0.587 | 24.014 | 1.00 | 69.14 | N |
| ATOM | 1152 | N | GLY | X | 155 | 28.000 | −3.004 | 21.850 | 1.00 | 74.77 | N |
| ATOM | 1153 | CA | GLY | X | 155 | 28.342 | −4.405 | 22.049 | 1.00 | 74.77 | C |
| ATOM | 1154 | C | GLY | X | 155 | 28.940 | −4.886 | 23.359 | 1.00 | 74.77 | C |
| ATOM | 1155 | O | GLY | X | 155 | 28.896 | −4.195 | 24.379 | 1.00 | 74.77 | O |
| ATOM | 1156 | N | CYS | X | 156 | 29.488 | −6.103 | 23.311 | 1.00 | 60.33 | N |
| ATOM | 1157 | CA | CYS | X | 156 | 30.128 | −6.753 | 24.459 | 1.00 | 60.33 | C |
| ATOM | 1158 | C | CYS | X | 156 | 31.083 | −7.845 | 23.972 | 1.00 | 60.33 | C |
| ATOM | 1159 | O | CYS | X | 156 | 32.055 | −7.558 | 23.268 | 1.00 | 60.33 | O |
| ATOM | 1160 | CB | CYS | X | 156 | 29.077 | −7.375 | 25.382 | 1.00 | 97.61 | C |
| ATOM | 1161 | SG | CYS | X | 156 | 28.139 | −8.745 | 24.652 | 1.00 | 97.61 | S |
| ATOM | 1162 | N | ASN | X | 157 | 30.802 | −9.093 | 24.353 | 1.00 | 99.00 | N |
| ATOM | 1163 | CA | ASN | X | 157 | 31.626 | −10.228 | 23.945 | 1.00 | 99.00 | C |
| ATOM | 1164 | C | ASN | X | 157 | 31.942 | −10.077 | 22.462 | 1.00 | 99.00 | C |
| ATOM | 1165 | O | ASN | X | 157 | 31.062 | −9.773 | 21.656 | 1.00 | 99.00 | O |
| ATOM | 1166 | CB | ASN | X | 157 | 30.903 | −11.568 | 24.191 | 1.00 | 99.00 | C |
| ATOM | 1167 | CG | ASN | X | 157 | 30.843 | −11.955 | 25.677 | 1.00 | 99.00 | C |
| ATOM | 1168 | OD1 | ASN | X | 157 | 30.135 | −11.327 | 26.469 | 1.00 | 99.00 | O |
| ATOM | 1169 | ND2 | ASN | X | 157 | 31.588 | −12.996 | 26.052 | 1.00 | 99.00 | N |
| ATOM | 1170 | N | PRO | X | 158 | 33.211 | −10.287 | 22.088 | 1.00 | 99.00 | N |
| ATOM | 1171 | CA | PRO | X | 158 | 33.707 | −10.181 | 20.715 | 1.00 | 99.00 | C |
| ATOM | 1172 | C | PRO | X | 158 | 32.691 | −10.389 | 19.593 | 1.00 | 99.00 | C |
| ATOM | 1173 | O | PRO | X | 158 | 32.405 | −11.518 | 19.186 | 1.00 | 99.00 | O |
| ATOM | 1174 | CB | PRO | X | 158 | 34.835 | −11.200 | 20.693 | 1.00 | 99.00 | C |
| ATOM | 1175 | CG | PRO | X | 158 | 35.453 | −10.973 | 22.039 | 1.00 | 99.00 | C |
| ATOM | 1176 | CD | PRO | X | 158 | 34.241 | −10.883 | 22.960 | 1.00 | 99.00 | C |
| ATOM | 1177 | N | ASN | X | 159 | 32.151 | −9.276 | 19.109 | 1.00 | 98.30 | N |
| ATOM | 1178 | CA | ASN | X | 159 | 31.191 | −9.279 | 18.016 | 1.00 | 98.30 | C |
| ATOM | 1179 | C | ASN | X | 159 | 30.003 | −10.213 | 18.169 | 1.00 | 98.30 | C |
| ATOM | 1180 | O | ASN | X | 159 | 30.070 | −11.388 | 17.800 | 1.00 | 98.30 | O |
| ATOM | 1181 | CB | ASN | X | 159 | 31.900 | −9.610 | 16.703 | 1.00 | 99.00 | C |
| ATOM | 1182 | CG | ASN | X | 159 | 33.025 | −8.645 | 16.388 | 1.00 | 99.00 | C |
| ATOM | 1183 | OD1 | ASN | X | 159 | 32.799 | −7.453 | 16.177 | 1.00 | 99.00 | O |
| ATOM | 1184 | ND2 | ASN | X | 159 | 34.250 | −9.157 | 16.357 | 1.00 | 99.00 | N |
| ATOM | 1185 | N | TYR | X | 160 | 28.917 | −9.685 | 18.722 | 1.00 | 99.00 | N |
| ATOM | 1186 | CA | TYR | X | 160 | 27.686 | −10.449 | 18.864 | 1.00 | 99.00 | C |
| ATOM | 1187 | C | TYR | X | 160 | 26.638 | −9.595 | 18.140 | 1.00 | 99.00 | C |
| ATOM | 1188 | O | TYR | X | 160 | 25.451 | −9.930 | 18.082 | 1.00 | 99.00 | O |
| ATOM | 1189 | CB | TYR | X | 160 | 27.316 | −10.650 | 20.343 | 1.00 | 90.79 | C |
| ATOM | 1190 | CG | TYR | X | 160 | 26.677 | −9.457 | 21.018 | 1.00 | 90.79 | C |
| ATOM | 1191 | CD1 | TYR | X | 160 | 25.604 | −9.627 | 21.897 | 1.00 | 90.79 | C |
| ATOM | 1192 | CD2 | TYR | X | 160 | 27.131 | −8.159 | 20.775 | 1.00 | 90.79 | C |
| ATOM | 1193 | CE1 | TYR | X | 160 | 24.995 | −8.534 | 22.514 | 1.00 | 90.79 | C |
| ATOM | 1194 | CE2 | TYR | X | 160 | 26.529 | −7.059 | 21.386 | 1.00 | 90.79 | C |
| ATOM | 1195 | CZ | TYR | X | 160 | 25.461 | −7.252 | 22.252 | 1.00 | 90.79 | C |
| ATOM | 1196 | OH | TYR | X | 160 | 24.855 | −6.164 | 22.841 | 1.00 | 90.79 | O |
| ATOM | 1197 | N | LEU | X | 161 | 27.124 | −8.487 | 17.579 | 1.00 | 99.00 | N |
| ATOM | 1198 | CA | LEU | X | 161 | 26.328 | −7.509 | 16.835 | 1.00 | 99.00 | C |
| ATOM | 1199 | C | LEU | X | 161 | 25.051 | −7.014 | 17.498 | 1.00 | 99.00 | C |
| ATOM | 1200 | O | LEU | X | 161 | 25.016 | −6.793 | 18.708 | 1.00 | 99.00 | O |
| ATOM | 1201 | CB | LEU | X | 161 | 26.002 | −8.026 | 15.427 | 1.00 | 54.38 | C |
| ATOM | 1202 | CG | LEU | X | 161 | 26.665 | −7.134 | 14.367 | 1.00 | 54.38 | C |
| ATOM | 1203 | CD1 | LEU | X | 161 | 26.392 | −7.653 | 12.971 | 1.00 | 54.38 | C |
| ATOM | 1204 | CD2 | LEU | X | 161 | 26.152 | −5.708 | 14.525 | 1.00 | 54.38 | C |
| ATOM | 1205 | N | ALA | X | 162 | 24.022 | −6.827 | 16.674 | 1.00 | 66.37 | N |
| ATOM | 1206 | CA | ALA | X | 162 | 22.701 | −6.337 | 17.073 | 1.00 | 66.37 | C |
| ATOM | 1207 | C | ALA | X | 162 | 22.420 | −5.061 | 16.288 | 1.00 | 66.37 | C |
| ATOM | 1208 | O | ALA | X | 162 | 23.061 | −4.029 | 16.501 | 1.00 | 66.37 | O |
| ATOM | 1209 | CB | ALA | X | 162 | 22.630 | −6.054 | 18.575 | 1.00 | 75.27 | C |
| ATOM | 1210 | N | VAL | X | 163 | 21.459 | −5.140 | 15.375 | 1.00 | 52.28 | N |
| ATOM | 1211 | CA | VAL | X | 163 | 21.098 | −4.003 | 14.541 | 1.00 | 52.28 | C |
| ATOM | 1212 | C | VAL | X | 163 | 19.701 | −3.488 | 14.809 | 1.00 | 52.28 | C |
| ATOM | 1213 | O | VAL | X | 163 | 18.766 | −4.258 | 15.009 | 1.00 | 52.28 | O |
| ATOM | 1214 | CB | VAL | X | 163 | 21.143 | −4.362 | 13.047 | 1.00 | 52.25 | C |
| ATOM | 1215 | CG1 | VAL | X | 163 | 20.834 | −3.139 | 12.219 | 1.00 | 52.25 | C |
| ATOM | 1216 | CG2 | VAL | X | 163 | 22.495 | −4.924 | 12.681 | 1.00 | 52.25 | C |
| ATOM | 1217 | N | LEU | X | 164 | 19.569 | −2.173 | 14.816 | 1.00 | 57.49 | N |
| ATOM | 1218 | CA | LEU | X | 164 | 18.276 | −1.551 | 14.999 | 1.00 | 57.49 | C |
| ATOM | 1219 | C | LEU | X | 164 | 17.901 | −1.108 | 13.593 | 1.00 | 57.49 | C |
| ATOM | 1220 | O | LEU | X | 164 | 18.770 | −0.718 | 12.812 | 1.00 | 57.49 | O |
| ATOM | 1221 | CB | LEU | X | 164 | 18.383 | −0.330 | 15.905 | 1.00 | 33.33 | C |
| ATOM | 1222 | CG | LEU | X | 164 | 17.168 | 0.603 | 15.832 | 1.00 | 33.33 | C |
| ATOM | 1223 | CD1 | LEU | X | 164 | 16.044 | 0.058 | 16.695 | 1.00 | 33.33 | C |
| ATOM | 1224 | CD2 | LEU | X | 164 | 17.554 | 1.996 | 16.284 | 1.00 | 33.33 | C |
| ATOM | 1225 | N | VAL | X | 165 | 16.617 | −1.181 | 13.262 | 1.00 | 40.21 | N |
| ATOM | 1226 | CA | VAL | X | 165 | 16.138 | −0.768 | 11.946 | 1.00 | 40.21 | C |
| ATOM | 1227 | C | VAL | X | 165 | 15.061 | 0.286 | 12.109 | 1.00 | 40.21 | C |
| ATOM | 1228 | O | VAL | X | 165 | 14.048 | 0.046 | 12.756 | 1.00 | 40.21 | O |
| ATOM | 1229 | CB | VAL | X | 165 | 15.559 | −1.956 | 11.183 | 1.00 | 42.21 | C |
| ATOM | 1230 | CG1 | VAL | X | 165 | 16.648 | −2.623 | 10.380 | 1.00 | 42.21 | C |
| ATOM | 1231 | CG2 | VAL | X | 165 | 14.963 | −2.956 | 12.161 | 1.00 | 42.21 | C |
| ATOM | 1232 | N | LYS | X | 166 | 15.279 | 1.456 | 11.529 | 1.00 | 42.70 | N |
| ATOM | 1233 | CA | LYS | X | 166 | 14.301 | 2.527 | 11.652 | 1.00 | 42.70 | C |
| ATOM | 1234 | C | LYS | X | 166 | 13.844 | 3.022 | 10.303 | 1.00 | 42.70 | C |
| ATOM | 1235 | O | LYS | X | 166 | 14.486 | 2.748 | 9.295 | 1.00 | 42.70 | O |
| ATOM | 1236 | CB | LYS | X | 166 | 14.897 | 3.720 | 12.387 | 1.00 | 55.19 | C |
| ATOM | 1237 | CG | LYS | X | 166 | 15.686 | 3.387 | 13.613 | 1.00 | 55.19 | C |
| ATOM | 1238 | CD | LYS | X | 166 | 16.028 | 4.659 | 14.369 | 1.00 | 55.19 | C |
| ATOM | 1239 | CE | LYS | X | 166 | 16.810 | 5.634 | 13.518 | 1.00 | 55.19 | C |
| ATOM | 1240 | NZ | LYS | X | 166 | 16.940 | 6.917 | 14.245 | 1.00 | 55.19 | N |
| ATOM | 1241 | N | TYR | X | 167 | 12.747 | 3.776 | 10.302 | 1.00 | 47.87 | N |
| ATOM | 1242 | CA | TYR | X | 167 | 12.210 | 4.373 | 9.079 | 1.00 | 47.87 | C |
| ATOM | 1243 | C | TYR | X | 167 | 11.809 | 3.349 | 8.041 | 1.00 | 47.87 | C |
| ATOM | 1244 | O | TYR | X | 167 | 12.330 | 3.345 | 6.927 | 1.00 | 47.87 | O |
| ATOM | 1245 | CB | TYR | X | 167 | 13.241 | 5.316 | 8.455 | 1.00 | 63.86 | C |
| ATOM | 1246 | CG | TYR | X | 167 | 13.829 | 6.310 | 9.422 | 1.00 | 63.86 | C |
| ATOM | 1247 | CD1 | TYR | X | 167 | 14.972 | 7.029 | 9.093 | 1.00 | 63.86 | C |
| ATOM | 1248 | CD2 | TYR | X | 167 | 13.255 | 6.525 | 10.672 | 1.00 | 63.86 | C |
| ATOM | 1249 | CE1 | TYR | X | 167 | 15.532 | 7.932 | 9.984 | 1.00 | 63.86 | C |
| ATOM | 1250 | CE2 | TYR | X | 167 | 13.807 | 7.424 | 11.567 | 1.00 | 63.86 | C |
| ATOM | 1251 | CZ | TYR | X | 167 | 14.944 | 8.123 | 11.219 | 1.00 | 63.86 | C |
| ATOM | 1252 | OH | TYR | X | 167 | 15.495 | 9.014 | 12.108 | 1.00 | 63.86 | O |
| ATOM | 1253 | N | VAL | X | 168 | 10.891 | 2.472 | 8.415 | 1.00 | 42.72 | N |
| ATOM | 1254 | CA | VAL | X | 168 | 10.428 | 1.461 | 7.496 | 1.00 | 42.72 | C |
| ATOM | 1255 | C | VAL | X | 168 | 9.031 | 1.880 | 7.078 | 1.00 | 42.72 | C |
| ATOM | 1256 | O | VAL | X | 168 | 8.087 | 1.824 | 7.859 | 1.00 | 42.72 | O |
| ATOM | 1257 | CB | VAL | X | 168 | 10.393 | 0.085 | 8.152 | 1.00 | 30.21 | C |
| ATOM | 1258 | CG1 | VAL | X | 168 | 10.142 | −0.959 | 7.102 | 1.00 | 30.21 | C |
| ATOM | 1259 | CG2 | VAL | X | 168 | 11.713 | −0.194 | 8.842 | 1.00 | 30.21 | C |
| ATOM | 1260 | N | ALA | X | 169 | 8.918 | 2.325 | 5.835 | 1.00 | 38.54 | N |
| ATOM | 1261 | CA | ALA | X | 169 | 7.649 | 2.779 | 5.307 | 1.00 | 38.54 | C |
| ATOM | 1262 | C | ALA | X | 169 | 6.695 | 1.645 | 4.985 | 1.00 | 38.54 | C |
| ATOM | 1263 | O | ALA | X | 169 | 7.091 | 0.506 | 4.804 | 1.00 | 38.54 | O |
| ATOM | 1264 | CB | ALA | X | 169 | 7.885 | 3.624 | 4.066 | 1.00 | 21.47 | C |
| ATOM | 1265 | N | ASP | X | 170 | 5.422 | 1.992 | 4.933 | 1.00 | 28.44 | N |
| ATOM | 1266 | CA | ASP | X | 170 | 4.354 | 1.077 | 4.598 | 1.00 | 28.44 | C |
| ATOM | 1267 | C | ASP | X | 170 | 3.917 | 0.242 | 5.744 | 1.00 | 28.44 | C |
| ATOM | 1268 | O | ASP | X | 170 | 3.105 | 0.691 | 6.544 | 1.00 | 28.44 | O |
| ATOM | 1269 | CB | ASP | X | 170 | 4.737 | 0.182 | 3.415 | 1.00 | 24.17 | C |
| ATOM | 1270 | CG | ASP | X | 170 | 3.569 | −0.615 | 2.897 | 1.00 | 24.17 | C |
| ATOM | 1271 | OD1 | ASP | X | 170 | 2.423 | −0.165 | 3.077 | 1.00 | 24.17 | O |
| ATOM | 1272 | OD2 | ASP | X | 170 | 3.805 | −1.685 | 2.303 | 1.00 | 24.17 | O |
| ATOM | 1273 | N | ASP | X | 171 | 4.419 | −0.983 | 5.823 | 1.00 | 30.15 | N |
| ATOM | 1274 | CA | ASP | X | 171 | 4.022 | −1.846 | 6.922 | 1.00 | 30.15 | C |
| ATOM | 1275 | C | ASP | X | 171 | 4.545 | −1.247 | 8.201 | 1.00 | 30.15 | C |
| ATOM | 1276 | O | ASP | X | 171 | 3.897 | −1.330 | 9.230 | 1.00 | 30.15 | O |
| ATOM | 1277 | CB | ASP | X | 171 | 4.564 | −3.253 | 6.736 | 1.00 | 44.91 | C |
| ATOM | 1278 | CG | ASP | X | 171 | 3.683 | −4.090 | 5.848 | 1.00 | 44.91 | C |
| ATOM | 1279 | OD1 | ASP | X | 171 | 2.453 | −4.007 | 6.011 | 1.00 | 44.91 | O |
| ATOM | 1280 | OD2 | ASP | X | 171 | 4.214 | −4.830 | 5.000 | 1.00 | 44.91 | O |
| ATOM | 1281 | N | GLY | X | 172 | 5.712 | −0.620 | 8.109 | 1.00 | 50.12 | N |
| ATOM | 1282 | CA | GLY | X | 172 | 6.311 | 0.019 | 9.257 | 1.00 | 50.12 | C |
| ATOM | 1283 | C | GLY | X | 172 | 7.092 | −0.913 | 10.160 | 1.00 | 50.12 | C |
| ATOM | 1284 | O | GLY | X | 172 | 8.214 | −0.604 | 10.559 | 1.00 | 50.12 | O |
| ATOM | 1285 | N | ASP | X | 173 | 6.508 | −2.065 | 10.468 | 1.00 | 39.19 | N |
| ATOM | 1286 | CA | ASP | X | 173 | 7.127 | −3.025 | 11.361 | 1.00 | 39.19 | C |
| ATOM | 1287 | C | ASP | X | 173 | 7.811 | −4.206 | 10.703 | 1.00 | 39.19 | C |
| ATOM | 1288 | O | ASP | X | 173 | 7.370 | −4.707 | 9.669 | 1.00 | 39.19 | O |
| ATOM | 1289 | CB | ASP | X | 173 | 6.077 | −3.543 | 12.326 | 1.00 | 28.64 | C |
| ATOM | 1290 | CG | ASP | X | 173 | 4.857 | −4.059 | 11.612 | 1.00 | 28.64 | C |
| ATOM | 1291 | OD1 | ASP | X | 173 | 4.972 | −4.405 | 10.420 | 1.00 | 28.64 | O |
| ATOM | 1292 | OD2 | ASP | X | 173 | 3.786 | −4.137 | 12.243 | 1.00 | 28.64 | O |
| ATOM | 1293 | N | ILE | X | 174 | 8.888 | −4.658 | 11.340 | 1.00 | 59.27 | N |
| ATOM | 1294 | CA | ILE | X | 174 | 9.666 | −5.800 | 10.876 | 1.00 | 59.27 | C |
| ATOM | 1295 | C | ILE | X | 174 | 9.296 | −7.052 | 11.669 | 1.00 | 59.27 | C |
| ATOM | 1296 | O | ILE | X | 174 | 8.774 | −6.966 | 12.774 | 1.00 | 59.27 | O |
| ATOM | 1297 | CB | ILE | X | 174 | 11.148 | −5.531 | 11.049 | 1.00 | 32.86 | C |
| ATOM | 1298 | CG1 | ILE | X | 174 | 11.563 | −4.360 | 10.158 | 1.00 | 32.86 | C |
| ATOM | 1299 | CG2 | ILE | X | 174 | 11.945 | −6.767 | 10.719 | 1.00 | 32.86 | C |
| ATOM | 1300 | CD1 | ILE | X | 174 | 11.525 | −4.652 | 8.676 | 1.00 | 32.86 | C |
| ATOM | 1301 | N | VAL | X | 175 | 9.554 | −8.217 | 11.091 | 1.00 | 33.73 | N |
| ATOM | 1302 | CA | VAL | X | 175 | 9.238 | −9.478 | 11.743 | 1.00 | 33.73 | C |
| ATOM | 1303 | C | VAL | X | 175 | 10.327 | −10.516 | 11.496 | 1.00 | 33.73 | C |
| ATOM | 1304 | O | VAL | X | 175 | 10.178 | −11.685 | 11.849 | 1.00 | 33.73 | O |
| ATOM | 1305 | CB | VAL | X | 175 | 7.875 | −10.044 | 11.259 | 1.00 | 28.91 | C |
| ATOM | 1306 | CG1 | VAL | X | 175 | 6.745 | −9.134 | 11.707 | 1.00 | 28.91 | C |
| ATOM | 1307 | CG2 | VAL | X | 175 | 7.865 | −10.175 | 9.760 | 1.00 | 28.91 | C |
| ATOM | 1308 | N | LEU | X | 176 | 11.425 | −10.083 | 10.894 | 1.00 | 32.04 | N |
| ATOM | 1309 | CA | LEU | X | 176 | 12.534 | −10.974 | 10.631 | 1.00 | 32.04 | C |
| ATOM | 1310 | C | LEU | X | 176 | 13.648 | −10.275 | 9.895 | 1.00 | 32.04 | C |
| ATOM | 1311 | O | LEU | X | 176 | 13.410 | −9.588 | 8.909 | 1.00 | 32.04 | O |
| ATOM | 1312 | CB | LEU | X | 176 | 12.083 | −12.182 | 9.805 | 1.00 | 58.82 | C |
| ATOM | 1313 | CG | LEU | X | 176 | 13.235 | −13.042 | 9.270 | 1.00 | 58.82 | C |
| ATOM | 1314 | CD1 | LEU | X | 176 | 14.053 | −13.581 | 10.434 | 1.00 | 58.82 | C |
| ATOM | 1315 | CD2 | LEU | X | 176 | 12.694 | −14.179 | 8.427 | 1.00 | 58.82 | C |
| ATOM | 1316 | N | MET | X | 177 | 14.870 | −10.452 | 10.376 | 1.00 | 50.60 | N |
| ATOM | 1317 | CA | MET | X | 177 | 16.027 | −9.867 | 9.728 | 1.00 | 50.60 | C |
| ATOM | 1318 | C | MET | X | 177 | 16.967 | −11.047 | 9.526 | 1.00 | 50.60 | C |
| ATOM | 1319 | O | MET | X | 177 | 16.817 | −12.067 | 10.195 | 1.00 | 50.60 | O |
| ATOM | 1320 | CB | MET | X | 177 | 16.664 | −8.812 | 10.630 | 1.00 | 45.74 | C |
| ATOM | 1321 | CG | MET | X | 177 | 17.599 | −7.862 | 9.902 | 1.00 | 45.74 | C |
| ATOM | 1322 | SD | MET | X | 177 | 18.263 | −6.531 | 10.940 | 1.00 | 45.74 | S |
| ATOM | 1323 | CE | MET | X | 177 | 16.776 | −5.732 | 11.441 | 1.00 | 45.74 | C |
| ATOM | 1324 | N | GLU | X | 178 | 17.908 | −10.936 | 8.594 | 1.00 | 55.01 | N |
| ATOM | 1325 | CA | GLU | X | 178 | 18.851 | −12.023 | 8.347 | 1.00 | 55.01 | C |
| ATOM | 1326 | C | GLU | X | 178 | 20.025 | −11.540 | 7.532 | 1.00 | 55.01 | C |
| ATOM | 1327 | O | GLU | X | 178 | 19.942 | −10.498 | 6.887 | 1.00 | 55.01 | O |
| ATOM | 1328 | CB | GLU | X | 178 | 18.168 | −13.181 | 7.638 | 1.00 | 62.11 | C |
| ATOM | 1329 | CG | GLU | X | 178 | 17.396 | −12.775 | 6.435 | 1.00 | 62.11 | C |
| ATOM | 1330 | CD | GLU | X | 178 | 16.243 | −13.704 | 6.199 | 1.00 | 62.11 | C |
| ATOM | 1331 | OE1 | GLU | X | 178 | 15.512 | −13.996 | 7.170 | 1.00 | 62.11 | O |
| ATOM | 1332 | OE2 | GLU | X | 178 | 16.060 | −14.137 | 5.048 | 1.00 | 62.11 | O |
| ATOM | 1333 | N | ILE | X | 179 | 21.118 | −12.300 | 7.554 | 1.00 | 70.48 | N |
| ATOM | 1334 | CA | ILE | X | 179 | 22.323 | −11.885 | 6.849 | 1.00 | 70.48 | C |
| ATOM | 1335 | C | ILE | X | 179 | 22.946 | −12.938 | 5.944 | 1.00 | 70.48 | C |
| ATOM | 1336 | O | ILE | X | 179 | 22.351 | −13.970 | 5.659 | 1.00 | 70.48 | O |
| ATOM | 1337 | CB | ILE | X | 179 | 23.398 | −11.421 | 7.868 | 1.00 | 99.00 | C |
| ATOM | 1338 | CG1 | ILE | X | 179 | 22.735 | −10.609 | 8.985 | 1.00 | 99.00 | C |
| ATOM | 1339 | CG2 | ILE | X | 179 | 24.438 | −10.545 | 7.186 | 1.00 | 99.00 | C |
| ATOM | 1340 | CD1 | ILE | X | 179 | 23.707 | −10.056 | 10.002 | 1.00 | 99.00 | C |
| ATOM | 1341 | N | GLN | X | 180 | 24.158 | −12.634 | 5.491 | 1.00 | 99.00 | N |
| ATOM | 1342 | CA | GLN | X | 180 | 24.966 | −13.494 | 4.632 | 1.00 | 99.00 | C |
| ATOM | 1343 | C | GLN | X | 180 | 26.225 | −12.702 | 4.291 | 1.00 | 99.00 | C |
| ATOM | 1344 | O | GLN | X | 180 | 26.155 | −11.653 | 3.646 | 1.00 | 99.00 | O |
| ATOM | 1345 | CB | GLN | X | 180 | 24.216 | −13.858 | 3.344 | 1.00 | 99.00 | C |
| ATOM | 1346 | CG | GLN | X | 180 | 23.878 | −12.680 | 2.445 | 1.00 | 99.00 | C |
| ATOM | 1347 | CD | GLN | X | 180 | 23.917 | −13.042 | 0.971 | 1.00 | 99.00 | C |
| ATOM | 1348 | OE1 | GLN | X | 180 | 23.293 | −14.009 | 0.542 | 1.00 | 99.00 | O |
| ATOM | 1349 | NE2 | GLN | X | 180 | 24.655 | −12.261 | 0.188 | 1.00 | 99.00 | N |
| ATOM | 1350 | N | ASP | X | 181 | 27.378 | −13.181 | 4.741 | 1.00 | 72.36 | N |
| ATOM | 1351 | CA | ASP | X | 181 | 28.612 | −12.466 | 4.445 | 1.00 | 72.36 | C |
| ATOM | 1352 | C | ASP | X | 181 | 29.149 | −12.860 | 3.080 | 1.00 | 72.36 | C |
| ATOM | 1353 | O | ASP | X | 181 | 28.543 | −13.672 | 2.379 | 1.00 | 72.36 | O |
| ATOM | 1354 | CB | ASP | X | 181 | 29.683 | −12.699 | 5.531 | 1.00 | 62.59 | C |
| ATOM | 1355 | CG | ASP | X | 181 | 29.954 | −14.175 | 5.804 | 1.00 | 62.59 | C |
| ATOM | 1356 | OD1 | ASP | X | 181 | 29.128 | −14.825 | 6.484 | 1.00 | 62.59 | O |
| ATOM | 1357 | OD2 | ASP | X | 181 | 30.999 | −14.684 | 5.341 | 1.00 | 62.59 | O |
| ATOM | 1358 | N | LYS | X | 182 | 30.281 | −12.272 | 2.707 | 1.00 | 99.00 | N |
| ATOM | 1359 | CA | LYS | X | 182 | 30.904 | −12.552 | 1.421 | 1.00 | 99.00 | C |
| ATOM | 1360 | C | LYS | X | 182 | 31.287 | −14.024 | 1.320 | 1.00 | 99.00 | C |
| ATOM | 1361 | O | LYS | X | 182 | 32.335 | −14.440 | 1.821 | 1.00 | 99.00 | O |
| ATOM | 1362 | CB | LYS | X | 182 | 32.147 | −11.681 | 1.236 | 1.00 | 97.92 | C |
| ATOM | 1363 | CG | LYS | X | 182 | 31.853 | −10.191 | 1.240 | 1.00 | 97.92 | C |
| ATOM | 1364 | CD | LYS | X | 182 | 33.110 | −9.380 | 0.978 | 1.00 | 97.92 | C |
| ATOM | 1365 | CE | LYS | X | 182 | 33.681 | −9.663 | −0.402 | 1.00 | 97.92 | C |
| ATOM | 1366 | NZ | LYS | X | 182 | 34.881 | −8.833 | −0.681 | 1.00 | 97.92 | N |
| ATOM | 1367 | N | LEU | X | 183 | 30.427 | −14.801 | 0.668 | 1.00 | 99.00 | N |
| ATOM | 1368 | CA | LEU | X | 183 | 30.629 | −16.232 | 0.475 | 1.00 | 99.00 | C |
| ATOM | 1369 | C | LEU | X | 183 | 29.986 | −17.064 | 1.570 | 1.00 | 99.00 | C |
| ATOM | 1370 | O | LEU | X | 183 | 30.214 | −16.843 | 2.763 | 1.00 | 99.00 | O |
| ATOM | 1371 | CB | LEU | X | 183 | 32.119 | −16.565 | 0.353 | 1.00 | 99.00 | C |
| ATOM | 1372 | CG | LEU | X | 183 | 32.709 | −16.093 | −0.980 | 1.00 | 99.00 | C |
| ATOM | 1373 | CD1 | LEU | X | 183 | 34.174 | −16.474 | −1.067 | 1.00 | 99.00 | C |
| ATOM | 1374 | CD2 | LEU | X | 183 | 31.908 | −16.709 | −2.134 | 1.00 | 99.00 | C |
| ATOM | 1375 | N | SER | X | 184 | 29.174 | −18.021 | 1.122 | 1.00 | 99.00 | N |
| ATOM | 1376 | CA | SER | X | 184 | 28.427 | −18.943 | 1.971 | 1.00 | 99.00 | C |
| ATOM | 1377 | C | SER | X | 184 | 27.017 | −18.402 | 2.157 | 1.00 | 99.00 | C |
| ATOM | 1378 | O | SER | X | 184 | 26.432 | −18.520 | 3.232 | 1.00 | 99.00 | O |
| ATOM | 1379 | CB | SER | X | 184 | 29.107 | −19.117 | 3.333 | 1.00 | 91.50 | C |
| ATOM | 1380 | OG | SER | X | 184 | 30.426 | −19.613 | 3.183 | 1.00 | 91.50 | O |
| ATOM | 1381 | N | ALA | X | 185 | 26.479 | −17.813 | 1.091 | 1.00 | 66.67 | N |
| ATOM | 1382 | CA | ALA | X | 185 | 25.133 | −17.245 | 1.116 | 1.00 | 66.67 | C |
| ATOM | 1383 | C | ALA | X | 185 | 24.068 | −18.284 | 1.472 | 1.00 | 66.67 | C |
| ATOM | 1384 | O | ALA | X | 185 | 24.283 | −19.483 | 1.314 | 1.00 | 66.67 | O |
| ATOM | 1385 | CB | ALA | X | 185 | 24.814 | −16.617 | −0.231 | 1.00 | 66.28 | C |
| ATOM | 1386 | N | GLU | X | 186 | 22.924 | −17.813 | 1.960 | 1.00 | 87.47 | N |
| ATOM | 1387 | CA | GLU | X | 186 | 21.808 | −18.676 | 2.346 | 1.00 | 87.47 | C |
| ATOM | 1388 | C | GLU | X | 186 | 20.919 | −17.957 | 3.357 | 1.00 | 87.47 | C |
| ATOM | 1389 | O | GLU | X | 186 | 19.931 | −18.509 | 3.850 | 1.00 | 87.47 | O |
| ATOM | 1390 | CB | GLU | X | 186 | 22.311 | −19.998 | 2.936 | 1.00 | 83.62 | C |
| ATOM | 1391 | CG | GLU | X | 186 | 23.364 | −19.848 | 4.005 | 1.00 | 83.62 | C |
| ATOM | 1392 | CD | GLU | X | 186 | 24.134 | −21.126 | 4.232 | 1.00 | 83.62 | C |
| ATOM | 1393 | OE1 | GLU | X | 186 | 23.519 | −22.115 | 4.684 | 1.00 | 83.62 | O |
| ATOM | 1394 | OE2 | GLU | X | 186 | 25.354 | −21.145 | 3.950 | 1.00 | 83.62 | O |
| ATOM | 1395 | N | TRP | X | 187 | 21.283 | −16.716 | 3.659 | 1.00 | 71.32 | N |
| ATOM | 1396 | CA | TRP | X | 187 | 20.527 | −15.890 | 4.589 | 1.00 | 71.32 | C |
| ATOM | 1397 | C | TRP | X | 187 | 20.250 | −16.555 | 5.926 | 1.00 | 71.32 | C |
| ATOM | 1398 | O | TRP | X | 187 | 19.266 | −17.283 | 6.071 | 1.00 | 71.32 | O |
| ATOM | 1399 | CB | TRP | X | 187 | 19.188 | −15.473 | 3.972 | 1.00 | 72.32 | C |
| ATOM | 1400 | CG | TRP | X | 187 | 19.311 | −14.731 | 2.683 | 1.00 | 72.32 | C |
| ATOM | 1401 | CD1 | TRP | X | 187 | 18.936 | −15.169 | 1.450 | 1.00 | 72.32 | C |
| ATOM | 1402 | CD2 | TRP | X | 187 | 19.858 | −13.421 | 2.495 | 1.00 | 72.32 | C |
| ATOM | 1403 | NE1 | TRP | X | 187 | 19.215 | −14.215 | 0.499 | 1.00 | 72.32 | N |
| ATOM | 1404 | CE2 | TRP | X | 187 | 19.782 | −13.130 | 1.114 | 1.00 | 72.32 | C |
| ATOM | 1405 | CE3 | TRP | X | 187 | 20.405 | −12.462 | 3.359 | 1.00 | 72.32 | C |
| ATOM | 1406 | CZ2 | TRP | X | 187 | 20.235 | −11.923 | 0.574 | 1.00 | 72.32 | C |
| ATOM | 1407 | CZ3 | TRP | X | 187 | 20.855 | −11.260 | 2.822 | 1.00 | 72.32 | C |
| ATOM | 1408 | CH2 | TRP | X | 187 | 20.766 | −11.002 | 1.441 | 1.00 | 72.32 | C |
| ATOM | 1409 | N | LYS | X | 188 | 21.112 | −16.306 | 6.904 | 1.00 | 84.38 | N |
| ATOM | 1410 | CA | LYS | X | 188 | 20.903 | −16.859 | 8.233 | 1.00 | 84.38 | C |
| ATOM | 1411 | C | LYS | X | 188 | 20.061 | −15.849 | 8.989 | 1.00 | 84.38 | C |
| ATOM | 1412 | O | LYS | X | 188 | 20.342 | −14.652 | 8.957 | 1.00 | 84.38 | O |
| ATOM | 1413 | CB | LYS | X | 188 | 22.228 | −17.067 | 8.968 | 1.00 | 99.00 | C |
| ATOM | 1414 | CG | LYS | X | 188 | 22.052 | −17.280 | 10.474 | 1.00 | 99.00 | C |
| ATOM | 1415 | CD | LYS | X | 188 | 23.371 | −17.562 | 11.183 | 1.00 | 99.00 | C |
| ATOM | 1416 | CE | LYS | X | 188 | 23.195 | −17.576 | 12.701 | 1.00 | 99.00 | C |
| ATOM | 1417 | NZ | LYS | X | 188 | 22.144 | −18.530 | 13.146 | 1.00 | 99.00 | N |
| ATOM | 1418 | N | PRO | X | 189 | 19.012 | −16.319 | 9.674 | 1.00 | 61.36 | N |
| ATOM | 1419 | CA | PRO | X | 189 | 18.094 | −15.491 | 10.457 | 1.00 | 61.36 | C |
| ATOM | 1420 | C | PRO | X | 189 | 18.811 | −14.582 | 11.449 | 1.00 | 61.36 | C |
| ATOM | 1421 | O | PRO | X | 189 | 20.022 | −14.374 | 11.360 | 1.00 | 61.36 | O |
| ATOM | 1422 | CB | PRO | X | 189 | 17.220 | −16.522 | 11.163 | 1.00 | 38.13 | C |
| ATOM | 1423 | CG | PRO | X | 189 | 17.155 | −17.615 | 10.197 | 1.00 | 38.13 | C |
| ATOM | 1424 | CD | PRO | X | 189 | 18.595 | −17.726 | 9.720 | 1.00 | 38.13 | C |
| ATOM | 1425 | N | MET | X | 190 | 18.048 | −14.044 | 12.395 | 1.00 | 43.42 | N |
| ATOM | 1426 | CA | MET | X | 190 | 18.596 | −13.163 | 13.412 | 1.00 | 43.42 | C |
| ATOM | 1427 | C | MET | X | 190 | 17.694 | −13.209 | 14.632 | 1.00 | 43.42 | C |
| ATOM | 1428 | O | MET | X | 190 | 16.489 | −13.442 | 14.515 | 1.00 | 43.42 | O |
| ATOM | 1429 | CB | MET | X | 190 | 18.669 | −11.735 | 12.888 | 1.00 | 58.50 | C |
| ATOM | 1430 | CG | MET | X | 190 | 19.474 | −11.581 | 11.623 | 1.00 | 58.50 | C |
| ATOM | 1431 | SD | MET | X | 190 | 21.062 | −10.854 | 11.938 | 1.00 | 58.50 | S |
| ATOM | 1432 | CE | MET | X | 190 | 20.559 | −9.277 | 12.584 | 1.00 | 58.50 | C |
| ATOM | 1433 | N | LYS | X | 191 | 18.281 | −12.980 | 15.801 | 1.00 | 57.01 | N |
| ATOM | 1434 | CA | LYS | X | 191 | 17.521 | −13.010 | 17.038 | 1.00 | 57.01 | C |
| ATOM | 1435 | C | LYS | X | 191 | 16.869 | −11.679 | 17.282 | 1.00 | 57.01 | C |
| ATOM | 1436 | O | LYS | X | 191 | 17.504 | −10.641 | 17.127 | 1.00 | 57.01 | O |
| ATOM | 1437 | CB | LYS | X | 191 | 18.420 | −13.309 | 18.234 | 1.00 | 99.00 | C |
| ATOM | 1438 | CG | LYS | X | 191 | 19.173 | −14.618 | 18.195 | 1.00 | 99.00 | C |
| ATOM | 1439 | CD | LYS | X | 191 | 19.786 | −14.914 | 19.561 | 1.00 | 99.00 | C |
| ATOM | 1440 | CE | LYS | X | 191 | 20.654 | −13.758 | 20.086 | 1.00 | 99.00 | C |
| ATOM | 1441 | NZ | LYS | X | 191 | 19.887 | −12.549 | 20.511 | 1.00 | 99.00 | N |
| ATOM | 1442 | N | LEU | X | 192 | 15.602 | −11.711 | 17.670 | 1.00 | 54.42 | N |
| ATOM | 1443 | CA | LEU | X | 192 | 14.886 | −10.487 | 17.975 | 1.00 | 54.42 | C |
| ATOM | 1444 | C | LEU | X | 192 | 15.202 | −10.166 | 19.416 | 1.00 | 54.42 | C |
| ATOM | 1445 | O | LEU | X | 192 | 14.400 | −10.467 | 20.296 | 1.00 | 54.42 | O |
| ATOM | 1446 | CB | LEU | X | 192 | 13.376 | −10.675 | 17.838 | 1.00 | 82.95 | C |
| ATOM | 1447 | CG | LEU | X | 192 | 12.559 | −9.581 | 18.541 | 1.00 | 82.95 | C |
| ATOM | 1448 | CD1 | LEU | X | 192 | 12.809 | −8.234 | 17.888 | 1.00 | 82.95 | C |
| ATOM | 1449 | CD2 | LEU | X | 192 | 11.092 | −9.929 | 18.500 | 1.00 | 82.95 | C |
| ATOM | 1450 | N | SER | X | 193 | 16.368 | −9.575 | 19.669 | 1.00 | 53.14 | N |
| ATOM | 1451 | CA | SER | X | 193 | 16.741 | −9.235 | 21.039 | 1.00 | 53.14 | C |
| ATOM | 1452 | C | SER | X | 193 | 15.525 | −8.577 | 21.688 | 1.00 | 53.14 | C |
| ATOM | 1453 | O | SER | X | 193 | 14.972 | −9.097 | 22.651 | 1.00 | 53.14 | O |
| ATOM | 1454 | CB | SER | X | 193 | 17.931 | −8.281 | 21.056 | 1.00 | 67.21 | C |
| ATOM | 1455 | OG | SER | X | 193 | 17.492 | −6.939 | 21.063 | 1.00 | 67.21 | O |
| ATOM | 1456 | N | TRP | X | 194 | 15.090 | −7.448 | 21.145 | 1.00 | 38.60 | N |
| ATOM | 1457 | CA | TRP | X | 194 | 13.918 | −6.771 | 21.677 | 1.00 | 38.60 | C |
| ATOM | 1458 | C | TRP | X | 194 | 13.545 | −5.569 | 20.806 | 1.00 | 38.60 | C |
| ATOM | 1459 | O | TRP | X | 194 | 14.420 | −4.887 | 20.259 | 1.00 | 38.60 | O |
| ATOM | 1460 | CB | TRP | X | 194 | 14.169 | −6.351 | 23.134 | 1.00 | 50.51 | C |
| ATOM | 1461 | CG | TRP | X | 194 | 14.507 | −4.918 | 23.312 | 1.00 | 50.51 | C |
| ATOM | 1462 | CD1 | TRP | X | 194 | 13.629 | −3.872 | 23.411 | 1.00 | 50.51 | C |
| ATOM | 1463 | CD2 | TRP | X | 194 | 15.817 | −4.348 | 23.353 | 1.00 | 50.51 | C |
| ATOM | 1464 | NE1 | TRP | X | 194 | 14.316 | −2.682 | 23.508 | 1.00 | 50.51 | N |
| ATOM | 1465 | CE2 | TRP | X | 194 | 15.659 | −2.943 | 23.475 | 1.00 | 50.51 | C |
| ATOM | 1466 | CE3 | TRP | X | 194 | 17.109 | −4.884 | 23.298 | 1.00 | 50.51 | C |
| ATOM | 1467 | CZ2 | TRP | X | 194 | 16.742 | −2.070 | 23.539 | 1.00 | 50.51 | C |
| ATOM | 1468 | CZ3 | TRP | X | 194 | 18.190 | −4.015 | 23.361 | 1.00 | 50.51 | C |
| ATOM | 1469 | CH2 | TRP | X | 194 | 17.997 | −2.620 | 23.481 | 1.00 | 50.51 | C |
| ATOM | 1470 | N | GLY | X | 195 | 12.239 | −5.331 | 20.679 | 1.00 | 45.90 | N |
| ATOM | 1471 | CA | GLY | X | 195 | 11.733 | −4.228 | 19.878 | 1.00 | 45.90 | C |
| ATOM | 1472 | C | GLY | X | 195 | 12.161 | −4.328 | 18.426 | 1.00 | 45.90 | C |
| ATOM | 1473 | O | GLY | X | 195 | 11.788 | −5.260 | 17.715 | 1.00 | 45.90 | O |
| ATOM | 1474 | N | ALA | X | 196 | 12.953 | −3.359 | 17.985 | 1.00 | 29.15 | N |
| ATOM | 1475 | CA | ALA | X | 196 | 13.446 | −3.323 | 16.622 | 1.00 | 29.15 | C |
| ATOM | 1476 | C | ALA | X | 196 | 14.925 | −3.663 | 16.628 | 1.00 | 29.15 | C |
| ATOM | 1477 | O | ALA | X | 196 | 15.644 | −3.376 | 15.668 | 1.00 | 29.15 | O |
| ATOM | 1478 | CB | ALA | X | 196 | 13.225 | −1.934 | 16.024 | 1.00 | 5.86 | C |
| ATOM | 1479 | N | ILE | X | 197 | 15.377 | −4.261 | 17.730 | 1.00 | 53.88 | N |
| ATOM | 1480 | CA | ILE | X | 197 | 16.775 | −4.655 | 17.880 | 1.00 | 53.88 | C |
| ATOM | 1481 | C | ILE | X | 197 | 16.911 | −6.124 | 17.548 | 1.00 | 53.88 | C |
| ATOM | 1482 | O | ILE | X | 197 | 16.249 | −6.963 | 18.149 | 1.00 | 53.88 | O |
| ATOM | 1483 | CB | ILE | X | 197 | 17.277 | −4.468 | 19.312 | 1.00 | 40.28 | C |
| ATOM | 1484 | CG1 | ILE | X | 197 | 16.994 | −3.049 | 19.798 | 1.00 | 40.28 | C |
| ATOM | 1485 | CG2 | ILE | X | 197 | 18.769 | −4.746 | 19.358 | 1.00 | 40.28 | C |
| ATOM | 1486 | CD1 | ILE | X | 197 | 17.877 | −2.015 | 19.180 | 1.00 | 40.28 | C |
| ATOM | 1487 | N | TRP | X | 198 | 17.776 | −6.430 | 16.593 | 1.00 | 36.85 | N |
| ATOM | 1488 | CA | TRP | X | 198 | 17.987 | −7.807 | 16.170 | 1.00 | 36.85 | C |
| ATOM | 1489 | C | TRP | X | 198 | 19.419 | −8.229 | 16.458 | 1.00 | 36.85 | C |
| ATOM | 1490 | O | TRP | X | 198 | 20.360 | −7.479 | 16.213 | 1.00 | 36.85 | O |
| ATOM | 1491 | CB | TRP | X | 198 | 17.623 | −7.953 | 14.680 | 1.00 | 40.41 | C |
| ATOM | 1492 | CG | TRP | X | 198 | 16.134 | −7.773 | 14.466 | 1.00 | 40.41 | C |
| ATOM | 1493 | CD1 | TRP | X | 198 | 15.428 | −6.616 | 14.603 | 1.00 | 40.41 | C |
| ATOM | 1494 | CD2 | TRP | X | 198 | 15.160 | −8.808 | 14.248 | 1.00 | 40.41 | C |
| ATOM | 1495 | NE1 | TRP | X | 198 | 14.082 | −6.862 | 14.501 | 1.00 | 40.41 | N |
| ATOM | 1496 | CE2 | TRP | X | 198 | 13.888 | −8.199 | 14.284 | 1.00 | 40.41 | C |
| ATOM | 1497 | CE3 | TRP | X | 198 | 15.241 | −10.194 | 14.038 | 1.00 | 40.41 | C |
| ATOM | 1498 | CZ2 | TRP | X | 198 | 12.700 | −8.927 | 14.118 | 1.00 | 40.41 | C |
| ATOM | 1499 | CZ3 | TRP | X | 198 | 14.062 | −10.918 | 13.876 | 1.00 | 40.41 | C |
| ATOM | 1500 | CH2 | TRP | X | 198 | 12.809 | −10.282 | 13.919 | 1.00 | 40.41 | C |
| ATOM | 1501 | N | ARG | X | 199 | 19.578 | −9.438 | 16.982 | 1.00 | 72.25 | N |
| ATOM | 1502 | CA | ARG | X | 199 | 20.891 | −9.932 | 17.369 | 1.00 | 72.25 | C |
| ATOM | 1503 | C | ARG | X | 199 | 21.432 | −11.084 | 16.542 | 1.00 | 72.25 | C |
| ATOM | 1504 | O | ARG | X | 199 | 20.686 | −11.937 | 16.062 | 1.00 | 72.25 | O |
| ATOM | 1505 | CB | ARG | X | 199 | 20.848 | −10.362 | 18.835 | 1.00 | 99.00 | C |
| ATOM | 1506 | CG | ARG | X | 199 | 22.185 | −10.374 | 19.537 | 1.00 | 99.00 | C |
| ATOM | 1507 | CD | ARG | X | 199 | 22.349 | −9.125 | 20.397 | 1.00 | 99.00 | C |
| ATOM | 1508 | NE | ARG | X | 199 | 21.267 | −8.987 | 21.375 | 1.00 | 99.00 | N |
| ATOM | 1509 | CZ | ARG | X | 199 | 21.233 | −8.071 | 22.342 | 1.00 | 99.00 | C |
| ATOM | 1510 | NH1 | ARG | X | 199 | 22.223 | −7.200 | 22.476 | 1.00 | 99.00 | N |
| ATOM | 1511 | NH2 | ARG | X | 199 | 20.202 | −8.023 | 23.177 | 1.00 | 99.00 | N |
| ATOM | 1512 | N | MET | X | 200 | 22.752 | −11.097 | 16.407 | 1.00 | 88.97 | N |
| ATOM | 1513 | CA | MET | X | 200 | 23.469 | −12.129 | 15.677 | 1.00 | 88.97 | C |
| ATOM | 1514 | C | MET | X | 200 | 24.538 | −12.663 | 16.626 | 1.00 | 88.97 | C |
| ATOM | 1515 | O | MET | X | 200 | 25.643 | −12.128 | 16.677 | 1.00 | 88.97 | O |
| ATOM | 1516 | CB | MET | X | 200 | 24.124 | −11.527 | 14.434 | 1.00 | 92.06 | C |
| ATOM | 1517 | CG | MET | X | 200 | 25.079 | −12.460 | 13.714 | 1.00 | 92.06 | C |
| ATOM | 1518 | SD | MET | X | 200 | 24.329 | −14.048 | 13.334 | 1.00 | 92.06 | S |
| ATOM | 1519 | CE | MET | X | 200 | 23.717 | −13.762 | 11.661 | 1.00 | 92.06 | C |
| ATOM | 1520 | N | ASP | X | 201 | 24.200 | −13.709 | 17.379 | 1.00 | 69.71 | N |
| ATOM | 1521 | CA | ASP | X | 201 | 25.112 | −14.321 | 18.357 | 1.00 | 69.71 | C |
| ATOM | 1522 | C | ASP | X | 201 | 26.523 | −14.561 | 17.832 | 1.00 | 69.71 | C |
| ATOM | 1523 | O | ASP | X | 201 | 27.489 | −13.970 | 18.314 | 1.00 | 69.71 | O |
| ATOM | 1524 | CB | ASP | X | 201 | 24.538 | −15.650 | 18.836 | 1.00 | 99.00 | C |
| ATOM | 1525 | CG | ASP | X | 201 | 23.074 | −15.553 | 19.171 | 1.00 | 99.00 | C |
| ATOM | 1526 | OD1 | ASP | X | 201 | 22.300 | −15.117 | 18.293 | 1.00 | 99.00 | O |
| ATOM | 1527 | OD2 | ASP | X | 201 | 22.697 | −15.913 | 20.304 | 1.00 | 99.00 | O |
| ATOM | 1528 | N | THR | X | 202 | 26.627 | −15.450 | 16.853 | 1.00 | 93.73 | N |
| ATOM | 1529 | CA | THR | X | 202 | 27.903 | −15.787 | 16.242 | 1.00 | 93.73 | C |
| ATOM | 1530 | C | THR | X | 202 | 28.319 | −14.692 | 15.260 | 1.00 | 93.73 | C |
| ATOM | 1531 | O | THR | X | 202 | 27.472 | −14.086 | 14.604 | 1.00 | 93.73 | O |
| ATOM | 1532 | CB | THR | X | 202 | 27.800 | −17.137 | 15.502 | 1.00 | 87.54 | C |
| ATOM | 1533 | OG1 | THR | X | 202 | 29.031 | −17.406 | 14.826 | 1.00 | 87.54 | O |
| ATOM | 1534 | CG2 | THR | X | 202 | 26.656 | −17.112 | 14.492 | 1.00 | 87.54 | C |
| ATOM | 1535 | N | ALA | X | 203 | 29.618 | −14.432 | 15.159 | 1.00 | 96.00 | N |
| ATOM | 1536 | CA | ALA | X | 203 | 30.106 | −13.399 | 14.251 | 1.00 | 96.00 | C |
| ATOM | 1537 | C | ALA | X | 203 | 31.551 | −13.627 | 13.831 | 1.00 | 96.00 | C |
| ATOM | 1538 | O | ALA | X | 203 | 32.146 | −12.782 | 13.160 | 1.00 | 96.00 | O |
| ATOM | 1539 | CB | ALA | X | 203 | 29.968 | −12.030 | 14.900 | 1.00 | 53.35 | C |
| ATOM | 1540 | N | LYS | X | 204 | 32.112 | −14.767 | 14.226 | 1.00 | 99.00 | N |
| ATOM | 1541 | CA | LYS | X | 204 | 33.489 | −15.097 | 13.881 | 1.00 | 99.00 | C |
| ATOM | 1542 | C | LYS | X | 204 | 33.627 | −15.336 | 12.377 | 1.00 | 99.00 | C |
| ATOM | 1543 | O | LYS | X | 204 | 34.480 | −14.732 | 11.722 | 1.00 | 99.00 | O |
| ATOM | 1544 | CB | LYS | X | 204 | 33.950 | −16.343 | 14.645 | 1.00 | 98.90 | C |
| ATOM | 1545 | CG | LYS | X | 204 | 33.280 | −17.647 | 14.212 | 1.00 | 98.90 | C |
| ATOM | 1546 | CD | LYS | X | 204 | 31.800 | −17.666 | 14.564 | 1.00 | 98.90 | C |
| ATOM | 1547 | CE | LYS | X | 204 | 31.103 | −18.923 | 14.045 | 1.00 | 98.90 | C |
| ATOM | 1548 | NZ | LYS | X | 204 | 30.802 | −18.870 | 12.586 | 1.00 | 98.90 | N |
| ATOM | 1549 | N | ALA | X | 205 | 32.782 | −16.216 | 11.839 | 1.00 | 99.00 | N |
| ATOM | 1550 | CA | ALA | X | 205 | 32.798 | −16.549 | 10.414 | 1.00 | 99.00 | C |
| ATOM | 1551 | C | ALA | X | 205 | 32.252 | −15.391 | 9.589 | 1.00 | 99.00 | C |
| ATOM | 1552 | O | ALA | X | 205 | 32.922 | −14.887 | 8.684 | 1.00 | 99.00 | O |
| ATOM | 1553 | CB | ALA | X | 205 | 31.971 | −17.803 | 10.161 | 1.00 | 61.42 | C |
| ATOM | 1554 | N | LEU | X | 206 | 31.026 | −14.983 | 9.903 | 1.00 | 71.39 | N |
| ATOM | 1555 | CA | LEU | X | 206 | 30.388 | −13.873 | 9.217 | 1.00 | 71.39 | C |
| ATOM | 1556 | C | LEU | X | 206 | 31.364 | −12.711 | 9.110 | 1.00 | 71.39 | C |
| ATOM | 1557 | O | LEU | X | 206 | 31.505 | −11.940 | 10.057 | 1.00 | 71.39 | O |
| ATOM | 1558 | CB | LEU | X | 206 | 29.170 | −13.401 | 10.005 | 1.00 | 80.72 | C |
| ATOM | 1559 | CG | LEU | X | 206 | 28.090 | −14.427 | 10.319 | 1.00 | 80.72 | C |
| ATOM | 1560 | CD1 | LEU | X | 206 | 27.042 | −13.808 | 11.241 | 1.00 | 80.72 | C |
| ATOM | 1561 | CD2 | LEU | X | 206 | 27.465 | −14.907 | 9.019 | 1.00 | 80.72 | C |
| ATOM | 1562 | N | LYS | X | 207 | 32.041 | −12.586 | 7.974 | 1.00 | 81.49 | N |
| ATOM | 1563 | CA | LYS | X | 207 | 32.979 | −11.486 | 7.773 | 1.00 | 81.49 | C |
| ATOM | 1564 | C | LYS | X | 207 | 32.843 | −10.942 | 6.356 | 1.00 | 81.49 | C |
| ATOM | 1565 | O | LYS | X | 207 | 32.485 | −11.677 | 5.436 | 1.00 | 81.49 | O |
| ATOM | 1566 | CB | LYS | X | 207 | 34.419 | −11.944 | 8.020 | 1.00 | 89.15 | C |
| ATOM | 1567 | CG | LYS | X | 207 | 34.689 | −12.555 | 9.404 | 1.00 | 89.15 | C |
| ATOM | 1568 | CD | LYS | X | 207 | 34.048 | −11.773 | 10.554 | 1.00 | 89.15 | C |
| ATOM | 1569 | CE | LYS | X | 207 | 34.328 | −10.281 | 10.497 | 1.00 | 89.15 | C |
| ATOM | 1570 | NZ | LYS | X | 207 | 33.463 | −9.553 | 11.466 | 1.00 | 89.15 | N |
| ATOM | 1571 | N | GLY | X | 208 | 33.130 | −9.654 | 6.183 | 1.00 | 99.00 | N |
| ATOM | 1572 | CA | GLY | X | 208 | 33.010 | −9.038 | 4.870 | 1.00 | 99.00 | C |
| ATOM | 1573 | C | GLY | X | 208 | 31.688 | −8.300 | 4.756 | 1.00 | 99.00 | C |
| ATOM | 1574 | O | GLY | X | 208 | 30.742 | −8.656 | 5.454 | 1.00 | 99.00 | O |
| ATOM | 1575 | N | PRO | X | 209 | 31.578 | −7.275 | 3.892 | 1.00 | 80.63 | N |
| ATOM | 1576 | CA | PRO | X | 209 | 30.329 | −6.517 | 3.739 | 1.00 | 80.63 | C |
| ATOM | 1577 | C | PRO | X | 209 | 29.106 | −7.418 | 3.762 | 1.00 | 80.63 | C |
| ATOM | 1578 | O | PRO | X | 209 | 28.922 | −8.253 | 2.878 | 1.00 | 80.63 | O |
| ATOM | 1579 | CB | PRO | X | 209 | 30.521 | −5.816 | 2.404 | 1.00 | 87.69 | C |
| ATOM | 1580 | CG | PRO | X | 209 | 31.985 | −5.507 | 2.429 | 1.00 | 87.69 | C |
| ATOM | 1581 | CD | PRO | X | 209 | 32.585 | −6.814 | 2.920 | 1.00 | 87.69 | C |
| ATOM | 1582 | N | PHE | X | 210 | 28.271 | −7.237 | 4.780 | 1.00 | 77.58 | N |
| ATOM | 1583 | CA | PHE | X | 210 | 27.084 | −8.064 | 4.943 | 1.00 | 77.58 | C |
| ATOM | 1584 | C | PHE | X | 210 | 25.840 | −7.498 | 4.301 | 1.00 | 77.58 | C |
| ATOM | 1585 | O | PHE | X | 210 | 25.646 | −6.287 | 4.246 | 1.00 | 77.58 | O |
| ATOM | 1586 | CB | PHE | X | 210 | 26.787 | −8.296 | 6.423 | 1.00 | 99.00 | C |
| ATOM | 1587 | CG | PHE | X | 210 | 27.988 | −8.662 | 7.234 | 1.00 | 99.00 | C |
| ATOM | 1588 | CD1 | PHE | X | 210 | 28.916 | −7.693 | 7.601 | 1.00 | 99.00 | C |
| ATOM | 1589 | CD2 | PHE | X | 210 | 28.192 | −9.973 | 7.639 | 1.00 | 99.00 | C |
| ATOM | 1590 | CE1 | PHE | X | 210 | 30.028 | −8.021 | 8.361 | 1.00 | 99.00 | C |
| ATOM | 1591 | CE2 | PHE | X | 210 | 29.300 | −10.314 | 8.400 | 1.00 | 99.00 | C |
| ATOM | 1592 | CZ | PHE | X | 210 | 30.222 | −9.335 | 8.763 | 1.00 | 99.00 | C |
| ATOM | 1593 | N | SER | X | 211 | 24.991 | −8.409 | 3.839 | 1.00 | 83.22 | N |
| ATOM | 1594 | CA | SER | X | 211 | 23.728 | −8.066 | 3.210 | 1.00 | 83.22 | C |
| ATOM | 1595 | C | SER | X | 211 | 22.641 | −8.368 | 4.234 | 1.00 | 83.22 | C |
| ATOM | 1596 | O | SER | X | 211 | 22.730 | −9.341 | 4.978 | 1.00 | 83.22 | O |
| ATOM | 1597 | CB | SER | X | 211 | 23.533 | −8.914 | 1.960 | 1.00 | 78.36 | C |
| ATOM | 1598 | OG | SER | X | 211 | 24.712 | −8.906 | 1.175 | 1.00 | 78.36 | O |
| ATOM | 1599 | N | ILE | X | 212 | 21.615 | −7.534 | 4.277 | 1.00 | 61.70 | N |
| ATOM | 1600 | CA | ILE | X | 212 | 20.547 | −7.727 | 5.237 | 1.00 | 61.70 | C |
| ATOM | 1601 | C | ILE | X | 212 | 19.205 | −7.750 | 4.548 | 1.00 | 61.70 | C |
| ATOM | 1602 | O | ILE | X | 212 | 18.935 | −6.913 | 3.691 | 1.00 | 61.70 | O |
| ATOM | 1603 | CB | ILE | X | 212 | 20.531 | −6.598 | 6.257 | 1.00 | 67.55 | C |
| ATOM | 1604 | CG1 | ILE | X | 212 | 21.906 | −6.467 | 6.901 | 1.00 | 67.55 | C |
| ATOM | 1605 | CG2 | ILE | X | 212 | 19.471 | −6.863 | 7.300 | 1.00 | 67.55 | C |
| ATOM | 1606 | CD1 | ILE | X | 212 | 22.007 | −5.306 | 7.859 | 1.00 | 67.55 | C |
| ATOM | 1607 | N | ARG | X | 213 | 18.365 | −8.702 | 4.937 | 1.00 | 56.98 | N |
| ATOM | 1608 | CA | ARG | X | 213 | 17.044 | −8.832 | 4.356 | 1.00 | 56.98 | C |
| ATOM | 1609 | C | ARG | X | 213 | 15.976 | −8.671 | 5.425 | 1.00 | 56.98 | C |
| ATOM | 1610 | O | ARG | X | 213 | 15.969 | −9.402 | 6.410 | 1.00 | 56.98 | O |
| ATOM | 1611 | CB | ARG | X | 213 | 16.905 | −10.185 | 3.684 | 1.00 | 56.46 | C |
| ATOM | 1612 | CG | ARG | X | 213 | 15.598 | −10.344 | 2.977 | 1.00 | 56.46 | C |
| ATOM | 1613 | CD | ARG | X | 213 | 15.631 | −11.537 | 2.065 | 1.00 | 56.46 | C |
| ATOM | 1614 | NE | ARG | X | 213 | 15.543 | −12.800 | 2.784 | 1.00 | 56.46 | N |
| ATOM | 1615 | CZ | ARG | X | 213 | 15.651 | −13.982 | 2.190 | 1.00 | 56.46 | C |
| ATOM | 1616 | NH1 | ARG | X | 213 | 15.855 | −14.040 | 0.880 | 1.00 | 56.46 | N |
| ATOM | 1617 | NH2 | ARG | X | 213 | 15.544 | −15.101 | 2.891 | 1.00 | 56.46 | N |
| ATOM | 1618 | N | LEU | X | 214 | 15.073 | −7.712 | 5.215 | 1.00 | 44.58 | N |
| ATOM | 1619 | CA | LEU | X | 214 | 14.000 | −7.415 | 6.155 | 1.00 | 44.58 | C |
| ATOM | 1620 | C | LEU | X | 214 | 12.691 | −7.946 | 5.622 | 1.00 | 44.58 | C |
| ATOM | 1621 | O | LEU | X | 214 | 12.476 | −7.962 | 4.418 | 1.00 | 44.58 | O |
| ATOM | 1622 | CB | LEU | X | 214 | 13.883 | −5.907 | 6.329 | 1.00 | 52.06 | C |
| ATOM | 1623 | CG | LEU | X | 214 | 15.193 | −5.169 | 6.600 | 1.00 | 52.06 | C |
| ATOM | 1624 | CD1 | LEU | X | 214 | 14.992 | −3.673 | 6.417 | 1.00 | 52.06 | C |
| ATOM | 1625 | CD2 | LEU | X | 214 | 15.684 | −5.500 | 8.007 | 1.00 | 52.06 | C |
| ATOM | 1626 | N | THR | X | 215 | 11.808 | −8.371 | 6.515 | 1.00 | 35.43 | N |
| ATOM | 1627 | CA | THR | X | 215 | 10.515 | −8.893 | 6.090 | 1.00 | 35.43 | C |
| ATOM | 1628 | C | THR | X | 215 | 9.438 | −8.180 | 6.868 | 1.00 | 35.43 | C |
| ATOM | 1629 | O | THR | X | 215 | 9.335 | −8.359 | 8.080 | 1.00 | 35.43 | O |
| ATOM | 1630 | CB | THR | X | 215 | 10.361 | −10.394 | 6.391 | 1.00 | 36.84 | C |
| ATOM | 1631 | OG1 | THR | X | 215 | 11.300 | −11.151 | 5.625 | 1.00 | 36.84 | O |
| ATOM | 1632 | CG2 | THR | X | 215 | 8.973 | −10.845 | 6.044 | 1.00 | 36.84 | C |
| ATOM | 1633 | N | SER | X | 216 | 8.637 | −7.379 | 6.176 | 1.00 | 18.36 | N |
| ATOM | 1634 | CA | SER | X | 216 | 7.564 | −6.640 | 6.815 | 1.00 | 18.36 | C |
| ATOM | 1635 | C | SER | X | 216 | 6.573 | −7.610 | 7.416 | 1.00 | 18.36 | C |
| ATOM | 1636 | O | SER | X | 216 | 6.673 | −8.815 | 7.225 | 1.00 | 18.36 | O |
| ATOM | 1637 | CB | SER | X | 216 | 6.861 | −5.763 | 5.797 | 1.00 | 48.23 | C |
| ATOM | 1638 | OG | SER | X | 216 | 6.404 | −6.540 | 4.709 | 1.00 | 48.23 | O |
| ATOM | 1639 | N | GLU | X | 217 | 5.607 | −7.079 | 8.147 | 1.00 | 33.43 | N |
| ATOM | 1640 | CA | GLU | X | 217 | 4.603 | −7.909 | 8.782 | 1.00 | 33.43 | C |
| ATOM | 1641 | C | GLU | X | 217 | 3.864 | −8.652 | 7.692 | 1.00 | 33.43 | C |
| ATOM | 1642 | O | GLU | X | 217 | 3.401 | −9.755 | 7.910 | 1.00 | 33.43 | O |
| ATOM | 1643 | CB | GLU | X | 217 | 3.628 | −7.034 | 9.591 | 1.00 | 30.80 | C |
| ATOM | 1644 | CG | GLU | X | 217 | 2.786 | −7.756 | 10.654 | 1.00 | 30.80 | C |
| ATOM | 1645 | CD | GLU | X | 217 | 1.842 | −6.805 | 11.392 | 1.00 | 30.80 | C |
| ATOM | 1646 | OE1 | GLU | X | 217 | 2.216 | −5.625 | 11.565 | 1.00 | 30.80 | O |
| ATOM | 1647 | OE2 | GLU | X | 217 | 0.738 | −7.226 | 11.812 | 1.00 | 30.80 | O |
| ATOM | 1648 | N | SER | X | 218 | 3.781 | −8.041 | 6.514 | 1.00 | 50.46 | N |
| ATOM | 1649 | CA | SER | X | 218 | 3.075 | −8.604 | 5.358 | 1.00 | 50.46 | C |
| ATOM | 1650 | C | SER | X | 218 | 3.734 | −9.745 | 4.641 | 1.00 | 50.46 | C |
| ATOM | 1651 | O | SER | X | 218 | 3.058 | −10.570 | 4.044 | 1.00 | 50.46 | O |
| ATOM | 1652 | CB | SER | X | 218 | 2.808 | −7.523 | 4.326 | 1.00 | 27.90 | C |
| ATOM | 1653 | OG | SER | X | 218 | 1.700 | −6.764 | 4.734 | 1.00 | 27.90 | O |
| ATOM | 1654 | N | GLY | X | 219 | 5.053 | −9.778 | 4.674 | 1.00 | 34.08 | N |
| ATOM | 1655 | CA | GLY | X | 219 | 5.771 | −10.827 | 3.992 | 1.00 | 34.08 | C |
| ATOM | 1656 | C | GLY | X | 219 | 6.695 | −10.167 | 3.000 | 1.00 | 34.08 | C |
| ATOM | 1657 | O | GLY | X | 219 | 7.691 | −10.750 | 2.559 | 1.00 | 34.08 | O |
| ATOM | 1658 | N | LYS | X | 220 | 6.373 | −8.932 | 2.642 | 1.00 | 42.43 | N |
| ATOM | 1659 | CA | LYS | X | 220 | 7.207 | −8.222 | 1.697 | 1.00 | 42.43 | C |
| ATOM | 1660 | C | LYS | X | 220 | 8.645 | −8.261 | 2.221 | 1.00 | 42.43 | C |
| ATOM | 1661 | O | LYS | X | 220 | 8.872 | −8.348 | 3.419 | 1.00 | 42.43 | O |
| ATOM | 1662 | CB | LYS | X | 220 | 6.678 | −6.799 | 1.518 | 1.00 | 73.70 | C |
| ATOM | 1663 | CG | LYS | X | 220 | 5.259 | −6.783 | 0.943 | 1.00 | 73.70 | C |
| ATOM | 1664 | CD | LYS | X | 220 | 4.838 | −5.401 | 0.434 | 1.00 | 73.70 | C |
| ATOM | 1665 | CE | LYS | X | 220 | 3.562 | −5.457 | −0.439 | 1.00 | 73.70 | C |
| ATOM | 1666 | NZ | LYS | X | 220 | 2.275 | −5.735 | 0.293 | 1.00 | 73.70 | N |
| ATOM | 1667 | N | LYS | X | 221 | 9.615 | −8.240 | 1.321 | 1.00 | 58.48 | N |
| ATOM | 1668 | CA | LYS | X | 221 | 11.011 | −8.294 | 1.725 | 1.00 | 58.48 | C |
| ATOM | 1669 | C | LYS | X | 221 | 11.868 | −7.358 | 0.896 | 1.00 | 58.48 | C |
| ATOM | 1670 | O | LYS | X | 221 | 11.467 | −6.915 | −0.174 | 1.00 | 58.48 | O |
| ATOM | 1671 | CB | LYS | X | 221 | 11.544 | −9.715 | 1.571 | 1.00 | 62.60 | C |
| ATOM | 1672 | CG | LYS | X | 221 | 10.755 | −10.746 | 2.336 | 1.00 | 62.60 | C |
| ATOM | 1673 | CD | LYS | X | 221 | 11.248 | −12.142 | 2.017 | 1.00 | 62.60 | C |
| ATOM | 1674 | CE | LYS | X | 221 | 10.363 | −13.186 | 2.679 | 1.00 | 62.60 | C |
| ATOM | 1675 | NZ | LYS | X | 221 | 10.650 | −14.566 | 2.186 | 1.00 | 62.60 | N |
| ATOM | 1676 | N | VAL | X | 222 | 13.057 | −7.064 | 1.403 | 1.00 | 72.80 | N |
| ATOM | 1677 | CA | VAL | X | 222 | 13.992 | −6.195 | 0.711 | 1.00 | 72.80 | C |
| ATOM | 1678 | C | VAL | X | 222 | 15.401 | −6.640 | 1.027 | 1.00 | 72.80 | C |
| ATOM | 1679 | O | VAL | X | 222 | 15.625 | −7.394 | 1.973 | 1.00 | 72.80 | O |
| ATOM | 1680 | CB | VAL | X | 222 | 13.836 | −4.737 | 1.139 | 1.00 | 49.89 | C |
| ATOM | 1681 | CG1 | VAL | X | 222 | 12.594 | −4.154 | 0.507 | 1.00 | 49.89 | C |
| ATOM | 1682 | CG2 | VAL | X | 222 | 13.770 | −4.647 | 2.660 | 1.00 | 49.89 | C |
| ATOM | 1683 | N | ILE | X | 223 | 16.353 | −6.163 | 0.235 | 1.00 | 61.92 | N |
| ATOM | 1684 | CA | ILE | X | 223 | 17.736 | −6.535 | 0.430 | 1.00 | 61.92 | C |
| ATOM | 1685 | C | ILE | X | 223 | 18.686 | −5.360 | 0.275 | 1.00 | 61.92 | C |
| ATOM | 1686 | O | ILE | X | 223 | 18.678 | −4.659 | −0.743 | 1.00 | 61.92 | O |
| ATOM | 1687 | CB | ILE | X | 223 | 18.126 | −7.648 | −0.551 | 1.00 | 57.10 | C |
| ATOM | 1688 | CG1 | ILE | X | 223 | 17.293 | −8.895 | −0.252 | 1.00 | 57.10 | C |
| ATOM | 1689 | CG2 | ILE | X | 223 | 19.607 | −7.962 | −0.438 | 1.00 | 57.10 | C |
| ATOM | 1690 | CD1 | ILE | X | 223 | 17.449 | −10.002 | −1.266 | 1.00 | 57.10 | C |
| ATOM | 1691 | N | ALA | X | 224 | 19.492 | −5.152 | 1.314 | 1.00 | 67.06 | N |
| ATOM | 1692 | CA | ALA | X | 224 | 20.492 | −4.095 | 1.346 | 1.00 | 67.06 | C |
| ATOM | 1693 | C | ALA | X | 224 | 21.816 | −4.847 | 1.277 | 1.00 | 67.06 | C |
| ATOM | 1694 | O | ALA | X | 224 | 22.138 | −5.598 | 2.191 | 1.00 | 67.06 | O |
| ATOM | 1695 | CB | ALA | X | 224 | 20.379 | −3.338 | 2.637 | 1.00 | 24.94 | C |
| ATOM | 1696 | N | LYS | X | 225 | 22.582 | −4.658 | 0.206 | 1.00 | 60.46 | N |
| ATOM | 1697 | CA | LYS | X | 225 | 23.830 | −5.399 | 0.054 | 1.00 | 60.46 | C |
| ATOM | 1698 | C | LYS | X | 225 | 24.886 | −5.140 | 1.111 | 1.00 | 60.46 | C |
| ATOM | 1699 | O | LYS | X | 225 | 25.169 | −6.009 | 1.936 | 1.00 | 60.46 | O |
| ATOM | 1700 | CB | LYS | X | 225 | 24.439 | −5.163 | −1.331 | 1.00 | 74.44 | C |
| ATOM | 1701 | CG | LYS | X | 225 | 25.649 | −6.077 | −1.656 | 1.00 | 74.44 | C |
| ATOM | 1702 | CD | LYS | X | 225 | 26.948 | −5.655 | −0.947 | 1.00 | 74.44 | C |
| ATOM | 1703 | CE | LYS | X | 225 | 28.105 | −6.602 | −1.248 | 1.00 | 74.44 | C |
| ATOM | 1704 | NZ | LYS | X | 225 | 27.919 | −7.947 | −0.630 | 1.00 | 74.44 | N |
| ATOM | 1705 | N | ASP | X | 226 | 25.486 | −3.955 | 1.073 | 1.00 | 73.47 | N |
| ATOM | 1706 | CA | ASP | X | 226 | 26.532 | −3.608 | 2.024 | 1.00 | 73.47 | C |
| ATOM | 1707 | C | ASP | X | 226 | 25.971 | −3.093 | 3.346 | 1.00 | 73.47 | C |
| ATOM | 1708 | O | ASP | X | 226 | 25.731 | −3.871 | 4.268 | 1.00 | 73.47 | O |
| ATOM | 1709 | CB | ASP | X | 226 | 27.454 | −2.556 | 1.413 | 1.00 | 99.00 | C |
| ATOM | 1710 | CG | ASP | X | 226 | 26.739 | −1.248 | 1.143 | 1.00 | 99.00 | C |
| ATOM | 1711 | OD1 | ASP | X | 226 | 25.656 | −1.286 | 0.520 | 1.00 | 99.00 | O |
| ATOM | 1712 | OD2 | ASP | X | 226 | 27.254 | −0.186 | 1.559 | 1.00 | 99.00 | O |
| ATOM | 1713 | N | VAL | X | 227 | 25.756 | −1.782 | 3.426 | 1.00 | 69.61 | N |
| ATOM | 1714 | CA | VAL | X | 227 | 25.248 | −1.131 | 4.625 | 1.00 | 69.61 | C |
| ATOM | 1715 | C | VAL | X | 227 | 25.956 | −1.600 | 5.892 | 1.00 | 69.61 | C |
| ATOM | 1716 | O | VAL | X | 227 | 25.578 | −1.239 | 7.008 | 1.00 | 69.61 | O |
| ATOM | 1717 | CB | VAL | X | 227 | 23.724 | −1.323 | 4.777 | 1.00 | 47.31 | C |
| ATOM | 1718 | CG1 | VAL | X | 227 | 23.017 | −0.455 | 3.767 | 1.00 | 47.31 | C |
| ATOM | 1719 | CG2 | VAL | X | 227 | 23.333 | −2.789 | 4.607 | 1.00 | 47.31 | C |
| ATOM | 1720 | N | ILE | X | 228 | 26.996 | −2.402 | 5.696 | 1.00 | 59.48 | N |
| ATOM | 1721 | CA | ILE | X | 228 | 27.812 | −2.945 | 6.771 | 1.00 | 59.48 | C |
| ATOM | 1722 | C | ILE | X | 228 | 29.143 | −3.325 | 6.131 | 1.00 | 59.48 | C |
| ATOM | 1723 | O | ILE | X | 228 | 29.323 | −4.446 | 5.652 | 1.00 | 59.48 | O |
| ATOM | 1724 | CB | ILE | X | 228 | 27.186 | −4.202 | 7.391 | 1.00 | 91.02 | C |
| ATOM | 1725 | CG1 | ILE | X | 228 | 25.775 | −3.900 | 7.893 | 1.00 | 91.02 | C |
| ATOM | 1726 | CG2 | ILE | X | 228 | 28.047 | −4.678 | 8.547 | 1.00 | 91.02 | C |
| ATOM | 1727 | CD1 | ILE | X | 228 | 25.093 | −5.087 | 8.526 | 1.00 | 91.02 | C |
| ATOM | 1728 | N | PRO | X | 229 | 30.088 | −2.380 | 6.102 | 1.00 | 94.81 | N |
| ATOM | 1729 | CA | PRO | X | 229 | 31.418 | −2.574 | 5.523 | 1.00 | 94.81 | C |
| ATOM | 1730 | C | PRO | X | 229 | 32.168 | −3.765 | 6.109 | 1.00 | 94.81 | C |
| ATOM | 1731 | O | PRO | X | 229 | 32.286 | −4.815 | 5.479 | 1.00 | 94.81 | O |
| ATOM | 1732 | CB | PRO | X | 229 | 32.112 | −1.249 | 5.823 | 1.00 | 54.91 | C |
| ATOM | 1733 | CG | PRO | X | 229 | 30.978 | −0.268 | 5.818 | 1.00 | 54.91 | C |
| ATOM | 1734 | CD | PRO | X | 229 | 29.934 | −1.005 | 6.605 | 1.00 | 54.91 | C |
| ATOM | 1735 | N | ALA | X | 230 | 32.672 | −3.574 | 7.323 | 1.00 | 99.00 | N |
| ATOM | 1736 | CA | ALA | X | 230 | 33.433 | −4.576 | 8.067 | 1.00 | 99.00 | C |
| ATOM | 1737 | C | ALA | X | 230 | 34.068 | −3.764 | 9.179 | 1.00 | 99.00 | C |
| ATOM | 1738 | O | ALA | X | 230 | 34.157 | −4.199 | 10.329 | 1.00 | 99.00 | O |
| ATOM | 1739 | CB | ALA | X | 230 | 34.511 | −5.199 | 7.189 | 1.00 | 44.67 | C |
| ATOM | 1740 | N | ASN | X | 231 | 34.501 | −2.564 | 8.806 | 1.00 | 99.00 | N |
| ATOM | 1741 | CA | ASN | X | 231 | 35.111 | −1.626 | 9.735 | 1.00 | 99.00 | C |
| ATOM | 1742 | C | ASN | X | 231 | 33.962 | −0.974 | 10.493 | 1.00 | 99.00 | C |
| ATOM | 1743 | O | ASN | X | 231 | 34.052 | 0.173 | 10.931 | 1.00 | 99.00 | O |
| ATOM | 1744 | CB | ASN | X | 231 | 35.909 | −0.561 | 8.974 | 1.00 | 99.00 | C |
| ATOM | 1745 | CG | ASN | X | 231 | 35.043 | 0.248 | 8.020 | 1.00 | 99.00 | C |
| ATOM | 1746 | OD1 | ASN | X | 231 | 34.447 | −0.296 | 7.092 | 1.00 | 99.00 | O |
| ATOM | 1747 | ND2 | ASN | X | 231 | 34.969 | 1.554 | 8.249 | 1.00 | 99.00 | N |
| ATOM | 1748 | N | TRP | X | 232 | 32.871 | −1.717 | 10.628 | 1.00 | 65.25 | N |
| ATOM | 1749 | CA | TRP | X | 232 | 31.705 | −1.223 | 11.331 | 1.00 | 65.25 | C |
| ATOM | 1750 | C | TRP | X | 232 | 31.989 | −1.144 | 12.823 | 1.00 | 65.25 | C |
| ATOM | 1751 | O | TRP | X | 232 | 32.555 | −2.067 | 13.408 | 1.00 | 65.25 | O |
| ATOM | 1752 | CB | TRP | X | 232 | 30.533 | −2.156 | 11.101 | 1.00 | 61.76 | C |
| ATOM | 1753 | CG | TRP | X | 232 | 30.747 | −3.504 | 11.691 | 1.00 | 61.76 | C |
| ATOM | 1754 | CD1 | TRP | X | 232 | 31.242 | −4.607 | 11.063 | 1.00 | 61.76 | C |
| ATOM | 1755 | CD2 | TRP | X | 232 | 30.461 | −3.898 | 13.037 | 1.00 | 61.76 | C |
| ATOM | 1756 | NE1 | TRP | X | 232 | 31.275 | −5.671 | 11.934 | 1.00 | 61.76 | N |
| ATOM | 1757 | CE2 | TRP | X | 232 | 30.802 | −5.262 | 13.153 | 1.00 | 61.76 | C |
| ATOM | 1758 | CE3 | TRP | X | 232 | 29.948 | −3.229 | 14.155 | 1.00 | 61.76 | C |
| ATOM | 1759 | CZ2 | TRP | X | 232 | 30.645 | −5.975 | 14.346 | 1.00 | 61.76 | C |
| ATOM | 1760 | CZ3 | TRP | X | 232 | 29.792 | −3.934 | 15.338 | 1.00 | 61.76 | C |
| ATOM | 1761 | CH2 | TRP | X | 232 | 30.140 | −5.296 | 15.425 | 1.00 | 61.76 | C |
| ATOM | 1762 | N | ARG | X | 233 | 31.583 | −0.045 | 13.440 | 1.00 | 68.52 | N |
| ATOM | 1763 | CA | ARG | X | 233 | 31.802 | 0.148 | 14.864 | 1.00 | 68.52 | C |
| ATOM | 1764 | C | ARG | X | 233 | 30.484 | 0.471 | 15.551 | 1.00 | 68.52 | C |
| ATOM | 1765 | O | ARG | X | 233 | 29.679 | 1.242 | 15.032 | 1.00 | 68.52 | O |
| ATOM | 1766 | CB | ARG | X | 233 | 32.792 | 1.287 | 15.082 | 1.00 | 98.84 | C |
| ATOM | 1767 | CG | ARG | X | 233 | 32.353 | 2.602 | 14.465 | 1.00 | 98.84 | C |
| ATOM | 1768 | CD | ARG | X | 233 | 33.383 | 3.682 | 14.721 | 1.00 | 98.84 | C |
| ATOM | 1769 | NE | ARG | X | 233 | 32.923 | 4.997 | 14.288 | 1.00 | 98.84 | N |
| ATOM | 1770 | CZ | ARG | X | 233 | 33.654 | 6.103 | 14.368 | 1.00 | 98.84 | C |
| ATOM | 1771 | NH1 | ARG | X | 233 | 34.883 | 6.054 | 14.865 | 1.00 | 98.84 | N |
| ATOM | 1772 | NH2 | ARG | X | 233 | 33.157 | 7.259 | 13.952 | 1.00 | 98.84 | N |
| ATOM | 1773 | N | PRO | X | 234 | 30.247 | −0.110 | 16.738 | 1.00 | 70.12 | N |
| ATOM | 1774 | CA | PRO | X | 234 | 28.992 | 0.164 | 17.440 | 1.00 | 70.12 | C |
| ATOM | 1775 | C | PRO | X | 234 | 28.644 | 1.644 | 17.406 | 1.00 | 70.12 | C |
| ATOM | 1776 | O | PRO | X | 234 | 29.528 | 2.496 | 17.428 | 1.00 | 70.12 | O |
| ATOM | 1777 | CB | PRO | X | 234 | 29.270 | −0.348 | 18.847 | 1.00 | 75.00 | C |
| ATOM | 1778 | CG | PRO | X | 234 | 30.156 | −1.524 | 18.582 | 1.00 | 75.00 | C |
| ATOM | 1779 | CD | PRO | X | 234 | 31.126 | −0.966 | 17.554 | 1.00 | 75.00 | C |
| ATOM | 1780 | N | ASP | X | 235 | 27.350 | 1.929 | 17.334 | 1.00 | 99.00 | N |
| ATOM | 1781 | CA | ASP | X | 235 | 26.842 | 3.292 | 17.285 | 1.00 | 99.00 | C |
| ATOM | 1782 | C | ASP | X | 235 | 26.915 | 3.837 | 15.855 | 1.00 | 99.00 | C |
| ATOM | 1783 | O | ASP | X | 235 | 26.174 | 4.755 | 15.495 | 1.00 | 99.00 | O |
| ATOM | 1784 | CB | ASP | X | 235 | 27.632 | 4.191 | 18.245 | 1.00 | 99.00 | C |
| ATOM | 1785 | CG | ASP | X | 235 | 26.953 | 5.527 | 18.495 | 1.00 | 99.00 | C |
| ATOM | 1786 | OD1 | ASP | X | 235 | 26.740 | 6.286 | 17.524 | 1.00 | 99.00 | O |
| ATOM | 1787 | OD2 | ASP | X | 235 | 26.632 | 5.820 | 19.667 | 1.00 | 99.00 | O |
| ATOM | 1788 | N | ALA | X | 236 | 27.797 | 3.265 | 15.037 | 1.00 | 67.41 | N |
| ATOM | 1789 | CA | ALA | X | 236 | 27.935 | 3.711 | 13.655 | 1.00 | 67.41 | C |
| ATOM | 1790 | C | ALA | X | 236 | 26.593 | 3.570 | 12.943 | 1.00 | 67.41 | C |
| ATOM | 1791 | O | ALA | X | 236 | 25.871 | 2.594 | 13.158 | 1.00 | 67.41 | O |
| ATOM | 1792 | CB | ALA | X | 236 | 28.993 | 2.895 | 12.942 | 1.00 | 42.14 | C |
| ATOM | 1793 | N | VAL | X | 237 | 26.266 | 4.545 | 12.097 | 1.00 | 99.00 | N |
| ATOM | 1794 | CA | VAL | X | 237 | 24.999 | 4.535 | 11.369 | 1.00 | 99.00 | C |
| ATOM | 1795 | C | VAL | X | 237 | 25.158 | 4.366 | 9.862 | 1.00 | 99.00 | C |
| ATOM | 1796 | O | VAL | X | 237 | 25.962 | 5.048 | 9.229 | 1.00 | 99.00 | O |
| ATOM | 1797 | CB | VAL | X | 237 | 24.211 | 5.830 | 11.618 | 1.00 | 54.48 | C |
| ATOM | 1798 | CG1 | VAL | X | 237 | 22.843 | 5.733 | 10.981 | 1.00 | 54.48 | C |
| ATOM | 1799 | CG2 | VAL | X | 237 | 24.081 | 6.075 | 13.098 | 1.00 | 54.48 | C |
| ATOM | 1800 | N | TYR | X | 238 | 24.369 | 3.462 | 9.292 | 1.00 | 46.71 | N |
| ATOM | 1801 | CA | TYR | X | 238 | 24.417 | 3.194 | 7.860 | 1.00 | 46.71 | C |
| ATOM | 1802 | C | TYR | X | 238 | 23.058 | 3.423 | 7.224 | 1.00 | 46.71 | C |
| ATOM | 1803 | O | TYR | X | 238 | 22.033 | 3.006 | 7.749 | 1.00 | 46.71 | O |
| ATOM | 1804 | CB | TYR | X | 238 | 24.868 | 1.756 | 7.619 | 1.00 | 70.02 | C |
| ATOM | 1805 | CG | TYR | X | 238 | 26.169 | 1.445 | 8.313 | 1.00 | 70.02 | C |
| ATOM | 1806 | CD1 | TYR | X | 238 | 27.392 | 1.696 | 7.694 | 1.00 | 70.02 | C |
| ATOM | 1807 | CD2 | TYR | X | 238 | 26.179 | 0.952 | 9.616 | 1.00 | 70.02 | C |
| ATOM | 1808 | CE1 | TYR | X | 238 | 28.595 | 1.464 | 8.354 | 1.00 | 70.02 | C |
| ATOM | 1809 | CE2 | TYR | X | 238 | 27.371 | 0.718 | 10.289 | 1.00 | 70.02 | C |
| ATOM | 1810 | CZ | TYR | X | 238 | 28.577 | 0.975 | 9.653 | 1.00 | 70.02 | C |
| ATOM | 1811 | OH | TYR | X | 238 | 29.760 | 0.739 | 10.323 | 1.00 | 70.02 | O |
| ATOM | 1812 | N | THR | X | 239 | 23.061 | 4.109 | 6.092 | 1.00 | 60.41 | N |
| ATOM | 1813 | CA | THR | X | 239 | 21.833 | 4.383 | 5.370 | 1.00 | 60.41 | C |
| ATOM | 1814 | C | THR | X | 239 | 21.829 | 3.569 | 4.078 | 1.00 | 60.41 | C |
| ATOM | 1815 | O | THR | X | 239 | 22.831 | 3.508 | 3.369 | 1.00 | 60.41 | O |
| ATOM | 1816 | CB | THR | X | 239 | 21.717 | 5.879 | 5.060 | 1.00 | 82.69 | C |
| ATOM | 1817 | OG1 | THR | X | 239 | 20.762 | 6.083 | 4.012 | 1.00 | 82.69 | O |
| ATOM | 1818 | CG2 | THR | X | 239 | 23.067 | 6.437 | 4.658 | 1.00 | 82.69 | C |
| ATOM | 1819 | N | SER | X | 240 | 20.700 | 2.935 | 3.784 | 1.00 | 50.19 | N |
| ATOM | 1820 | CA | SER | X | 240 | 20.569 | 2.105 | 2.590 | 1.00 | 50.19 | C |
| ATOM | 1821 | C | SER | X | 240 | 19.832 | 2.801 | 1.460 | 1.00 | 50.19 | C |
| ATOM | 1822 | O | SER | X | 240 | 19.341 | 3.910 | 1.606 | 1.00 | 50.19 | O |
| ATOM | 1823 | CB | SER | X | 240 | 19.794 | 0.843 | 2.925 | 1.00 | 47.09 | C |
| ATOM | 1824 | OG | SER | X | 240 | 18.447 | 1.184 | 3.214 | 1.00 | 47.09 | O |
| ATOM | 1825 | N | ASN | X | 241 | 19.762 | 2.115 | 0.328 | 1.00 | 62.17 | N |
| ATOM | 1826 | CA | ASN | X | 241 | 19.053 | 2.591 | −0.846 | 1.00 | 62.17 | C |
| ATOM | 1827 | C | ASN | X | 241 | 18.068 | 1.464 | −1.088 | 1.00 | 62.17 | C |
| ATOM | 1828 | O | ASN | X | 241 | 18.227 | 0.663 | −2.001 | 1.00 | 62.17 | O |
| ATOM | 1829 | CB | ASN | X | 241 | 20.010 | 2.735 | −2.023 | 1.00 | 98.83 | C |
| ATOM | 1830 | CG | ASN | X | 241 | 20.975 | 1.575 | −2.123 | 1.00 | 98.83 | C |
| ATOM | 1831 | OD1 | ASN | X | 241 | 20.568 | 0.424 | −2.272 | 1.00 | 98.83 | O |
| ATOM | 1832 | ND2 | ASN | X | 241 | 22.267 | 1.872 | −2.034 | 1.00 | 98.83 | N |
| ATOM | 1833 | N | VAL | X | 242 | 17.059 | 1.404 | −0.229 | 1.00 | 51.38 | N |
| ATOM | 1834 | CA | VAL | X | 242 | 16.034 | 0.374 | −0.269 | 1.00 | 51.38 | C |
| ATOM | 1835 | C | VAL | X | 242 | 14.926 | 0.852 | 0.655 | 1.00 | 51.38 | C |
| ATOM | 1836 | O | VAL | X | 242 | 15.202 | 1.466 | 1.678 | 1.00 | 51.38 | O |
| ATOM | 1837 | CB | VAL | X | 242 | 16.602 | −0.968 | 0.255 | 1.00 | 61.13 | C |
| ATOM | 1838 | CG1 | VAL | X | 242 | 15.549 | −1.721 | 1.061 | 1.00 | 61.13 | C |
| ATOM | 1839 | CG2 | VAL | X | 242 | 17.090 | −1.815 | −0.914 | 1.00 | 61.13 | C |
| ATOM | 1840 | N | GLN | X | 243 | 13.677 | 0.575 | 0.313 | 1.00 | 34.48 | N |
| ATOM | 1841 | CA | GLN | X | 243 | 12.571 | 1.025 | 1.149 | 1.00 | 34.48 | C |
| ATOM | 1842 | C | GLN | X | 243 | 11.300 | 0.280 | 0.758 | 1.00 | 34.48 | C |
| ATOM | 1843 | O | GLN | X | 243 | 11.295 | −0.443 | −0.229 | 1.00 | 34.48 | O |
| ATOM | 1844 | CB | GLN | X | 243 | 12.394 | 2.539 | 0.984 | 1.00 | 31.17 | C |
| ATOM | 1845 | CG | GLN | X | 243 | 11.115 | 3.083 | 1.553 | 1.00 | 31.17 | C |
| ATOM | 1846 | CD | GLN | X | 243 | 11.088 | 3.108 | 3.067 | 1.00 | 31.17 | C |
| ATOM | 1847 | OE1 | GLN | X | 243 | 11.813 | 3.875 | 3.685 | 1.00 | 31.17 | O |
| ATOM | 1848 | NE2 | GLN | X | 243 | 10.246 | 2.283 | 3.668 | 1.00 | 31.17 | N |
| ATOM | 1849 | N | PHE | X | 244 | 10.236 | 0.427 | 1.537 | 1.00 | 42.15 | N |
| ATOM | 1850 | CA | PHE | X | 244 | 8.980 | −0.237 | 1.229 | 1.00 | 42.15 | C |
| ATOM | 1851 | C | PHE | X | 244 | 8.001 | 0.746 | 0.600 | 1.00 | 42.15 | C |
| ATOM | 1852 | O | PHE | X | 244 | 7.683 | 1.792 | 1.174 | 1.00 | 42.15 | O |
| ATOM | 1853 | CB | PHE | X | 244 | 8.344 | −0.833 | 2.485 | 1.00 | 37.58 | C |
| ATOM | 1854 | CG | PHE | X | 244 | 9.016 | −2.080 | 2.982 | 1.00 | 37.58 | C |
| ATOM | 1855 | CD1 | PHE | X | 244 | 10.165 | −2.010 | 3.760 | 1.00 | 37.58 | C |
| ATOM | 1856 | CD2 | PHE | X | 244 | 8.503 | −3.330 | 2.668 | 1.00 | 37.58 | C |
| ATOM | 1857 | CE1 | PHE | X | 244 | 10.790 | −3.169 | 4.216 | 1.00 | 37.58 | C |
| ATOM | 1858 | CE2 | PHE | X | 244 | 9.120 | −4.491 | 3.118 | 1.00 | 37.58 | C |
| ATOM | 1859 | CZ | PHE | X | 244 | 10.264 | −4.411 | 3.892 | 1.00 | 37.58 | C |
| ATOM | 1860 | N | TYR | X | 245 | 7.511 | 0.409 | −0.585 | 1.00 | 36.46 | N |
| ATOM | 1861 | CA | TYR | X | 245 | 6.570 | 1.281 | −1.260 | 1.00 | 36.46 | C |
| ATOM | 1862 | C | TYR | X | 245 | 5.168 | 0.740 | −1.163 | 1.00 | 36.46 | C |
| ATOM | 1863 | O | TYR | X | 245 | 4.224 | 1.551 | −1.177 | 1.00 | 36.46 | O |
| ATOM | 1864 | CB | TYR | X | 245 | 7.000 | 1.446 | −2.698 | 1.00 | 35.82 | C |
| ATOM | 1865 | CG | TYR | X | 245 | 8.261 | 2.249 | −2.803 | 1.00 | 35.82 | C |
| ATOM | 1866 | CD1 | TYR | X | 245 | 8.212 | 3.632 | −2.903 | 1.00 | 35.82 | C |
| ATOM | 1867 | CD2 | TYR | X | 245 | 9.496 | 1.634 | −2.778 | 1.00 | 35.82 | C |
| ATOM | 1868 | CE1 | TYR | X | 245 | 9.357 | 4.388 | −2.981 | 1.00 | 35.82 | C |
| ATOM | 1869 | CE2 | TYR | X | 245 | 10.659 | 2.379 | −2.854 | 1.00 | 35.82 | C |
| ATOM | 1870 | CZ | TYR | X | 245 | 10.584 | 3.763 | −2.958 | 1.00 | 35.82 | C |
| ATOM | 1871 | OH | TYR | X | 245 | 11.735 | 4.521 | −3.050 | 1.00 | 35.82 | O |
| ATOM | 1872 | OXT | TYR | X | 245 | 5.050 | −0.497 | −1.065 | 1.00 | 35.82 | O |
| TER | 1873 | | TYR | X | 245 | | | | | | |
| HETATM | 1874 | C1 | NAG | A | 1 | −1.681 | −10.997 | 19.292 | 1.00 | 56.00 | C |
| HETATM | 1875 | C2 | NAG | A | 1 | −1.683 | −12.434 | 18.767 | 1.00 | 56.00 | C |
| HETATM | 1876 | C3 | NAG | A | 1 | −0.703 | −13.347 | 19.525 | 1.00 | 56.00 | C |
| HETATM | 1877 | C4 | NAG | A | 1 | 0.560 | −12.674 | 20.069 | 1.00 | 56.00 | C |
| HETATM | 1878 | C5 | NAG | A | 1 | 0.445 | −11.170 | 20.325 | 1.00 | 56.00 | C |
| HETATM | 1879 | C6 | NAG | A | 1 | 1.802 | −10.486 | 20.363 | 1.00 | 56.00 | C |
| HETATM | 1880 | C7 | NAG | A | 1 | −3.344 | −14.077 | 18.220 | 1.00 | 56.00 | C |
| HETATM | 1881 | C8 | NAG | A | 1 | −4.366 | −14.981 | 18.878 | 1.00 | 56.00 | C |
| HETATM | 1882 | N2 | NAG | A | 1 | −3.022 | −12.975 | 18.880 | 1.00 | 56.00 | N |
| HETATM | 1883 | O3 | NAG | A | 1 | −0.278 | −14.394 | 18.666 | 1.00 | 56.00 | O |
| HETATM | 1884 | O4 | NAG | A | 1 | 0.902 | −13.309 | 21.313 | 1.00 | 56.00 | O |
| HETATM | 1885 | O5 | NAG | A | 1 | −0.334 | −10.527 | 19.298 | 1.00 | 56.00 | O |
| HETATM | 1886 | O6 | NAG | A | 1 | 2.829 | −11.363 | 19.856 | 1.00 | 56.00 | O |
| HETATM | 1887 | O7 | NAG | A | 1 | −2.847 | −14.380 | 17.132 | 1.00 | 56.00 | O |
| HETATM | 1888 | C1 | NAG | A | 2 | 2.191 | −13.761 | 21.447 | 1.00 | 55.26 | C |
| HETATM | 1889 | C2 | NAG | A | 2 | 2.393 | −14.216 | 22.902 | 1.00 | 55.26 | C |
| HETATM | 1890 | C3 | NAG | A | 2 | 3.683 | −15.060 | 23.081 | 1.00 | 55.26 | C |
| HETATM | 1891 | C4 | NAG | A | 2 | 3.738 | −16.138 | 21.974 | 1.00 | 55.26 | C |
| HETATM | 1892 | C5 | NAG | A | 2 | 3.626 | −15.476 | 20.608 | 1.00 | 55.26 | C |
| HETATM | 1893 | C6 | NAG | A | 2 | 3.683 | −16.491 | 19.482 | 1.00 | 55.26 | C |
| HETATM | 1894 | C7 | NAG | A | 2 | 1.524 | −12.759 | 24.641 | 1.00 | 55.26 | C |
| HETATM | 1895 | C8 | NAG | A | 2 | 0.071 | −13.061 | 24.309 | 1.00 | 55.26 | C |
| HETATM | 1896 | N2 | NAG | A | 2 | 2.461 | −13.026 | 23.732 | 1.00 | 55.26 | N |
| HETATM | 1897 | O3 | NAG | A | 2 | 3.667 | −15.679 | 24.368 | 1.00 | 55.26 | O |
| HETATM | 1898 | O4 | NAG | A | 2 | 4.927 | −16.957 | 22.011 | 1.00 | 55.26 | O |
| HETATM | 1899 | O5 | NAG | A | 2 | 2.351 | −14.831 | 20.512 | 1.00 | 55.26 | O |
| HETATM | 1900 | O6 | NAG | A | 2 | 4.926 | −16.440 | 18.810 | 1.00 | 55.26 | O |
| HETATM | 1901 | O7 | NAG | A | 2 | 1.797 | −12.257 | 25.727 | 1.00 | 55.26 | O |
| HETATM | 1902 | C1 | MAN | A | 3 | 5.898 | −16.746 | 22.959 | 1.00 | 61.20 | C |
| HETATM | 1903 | C2 | MAN | A | 3 | 7.261 | −17.190 | 22.393 | 1.00 | 61.20 | C |
| HETATM | 1904 | C3 | MAN | A | 3 | 7.457 | −18.720 | 22.360 | 1.00 | 61.20 | C |
| HETATM | 1905 | C4 | MAN | A | 3 | 6.971 | −19.365 | 23.647 | 1.00 | 61.20 | C |
| HETATM | 1906 | C5 | MAN | A | 3 | 5.541 | −18.915 | 23.923 | 1.00 | 61.20 | C |
| HETATM | 1907 | C6 | MAN | A | 3 | 5.004 | −19.556 | 25.180 | 1.00 | 61.20 | C |
| HETATM | 1908 | O2 | MAN | A | 3 | 8.331 | −16.550 | 23.106 | 1.00 | 61.20 | O |
| HETATM | 1909 | O3 | MAN | A | 3 | 8.855 | −19.047 | 22.160 | 1.00 | 61.20 | O |
| HETATM | 1910 | O4 | MAN | A | 3 | 7.033 | −20.776 | 23.535 | 1.00 | 61.20 | O |
| HETATM | 1911 | O5 | MAN | A | 3 | 5.530 | −17.484 | 24.130 | 1.00 | 61.20 | O |
| HETATM | 1912 | O6 | MAN | A | 3 | 6.066 | −19.823 | 26.077 | 1.00 | 61.20 | O |
| HETATM | 1913 | C1 | MAN | A | 4 | 9.104 | −20.270 | 21.508 | 1.00 | 77.31 | C |
| HETATM | 1914 | C2 | MAN | A | 4 | 10.609 | −20.481 | 21.314 | 1.00 | 77.31 | C |
| HETATM | 1915 | C3 | MAN | A | 4 | 11.163 | −19.499 | 20.305 | 1.00 | 77.31 | C |
| HETATM | 1916 | C4 | MAN | A | 4 | 10.406 | −19.704 | 18.986 | 1.00 | 77.31 | C |
| HETATM | 1917 | C5 | MAN | A | 4 | 8.929 | −19.441 | 19.251 | 1.00 | 77.31 | C |
| HETATM | 1918 | C6 | MAN | A | 4 | 8.043 | −19.588 | 18.040 | 1.00 | 77.31 | C |
| HETATM | 1919 | O2 | MAN | A | 4 | 10.817 | −21.782 | 20.798 | 1.00 | 77.31 | O |
| HETATM | 1920 | O3 | MAN | A | 4 | 12.550 | −19.756 | 20.127 | 1.00 | 77.31 | O |
| HETATM | 1921 | O4 | MAN | A | 4 | 10.893 | −18.814 | 17.992 | 1.00 | 77.31 | O |
| HETATM | 1922 | O5 | MAN | A | 4 | 8.444 | −20.365 | 20.248 | 1.00 | 77.31 | O |
| HETATM | 1923 | O6 | MAN | A | 4 | 6.676 | −19.417 | 18.401 | 1.00 | 77.31 | O |
| HETATM | 1924 | C1 | XYS | A | 6 | 8.653 | −15.288 | 22.495 | 1.00 | 55.63 | C |
| HETATM | 1925 | C2 | XYS | A | 6 | 9.694 | −15.434 | 21.383 | 1.00 | 55.63 | C |
| HETATM | 1926 | C3 | XYS | A | 6 | 11.060 | −14.827 | 21.679 | 1.00 | 55.63 | C |
| HETATM | 1927 | C4 | XYS | A | 6 | 10.882 | −13.425 | 22.289 | 1.00 | 55.63 | C |
| HETATM | 1928 | C5 | XYS | A | 6 | 10.206 | −13.683 | 23.667 | 1.00 | 55.63 | C |
| HETATM | 1929 | O2 | XYS | A | 6 | 10.016 | −16.823 | 21.218 | 1.00 | 55.63 | O |
| HETATM | 1930 | O3 | XYS | A | 6 | 11.722 | −14.753 | 20.373 | 1.00 | 55.63 | O |
| HETATM | 1931 | O4 | XYS | A | 6 | 12.180 | −12.880 | 22.548 | 1.00 | 55.63 | O |
| HETATM | 1932 | O5 | XYS | A | 6 | 8.852 | −14.240 | 23.500 | 1.00 | 55.63 | O |
| HETATM | 1933 | C1 | MAN | A | 5 | 5.864 | −20.604 | 27.410 | 1.00 | 75.97 | C |
| HETATM | 1934 | C2 | MAN | A | 5 | 5.121 | −19.336 | 27.902 | 1.00 | 75.97 | C |
| HETATM | 1935 | C3 | MAN | A | 5 | 6.010 | −18.068 | 27.970 | 1.00 | 75.97 | C |
| HETATM | 1936 | C4 | MAN | A | 5 | 7.422 | −18.366 | 28.475 | 1.00 | 75.97 | C |
| HETATM | 1937 | C5 | MAN | A | 5 | 7.992 | −19.582 | 27.747 | 1.00 | 75.97 | C |
| HETATM | 1938 | C6 | MAN | A | 5 | 9.400 | −19.942 | 28.166 | 1.00 | 75.97 | C |
| HETATM | 1939 | O2 | MAN | A | 5 | 4.525 | −19.580 | 29.163 | 1.00 | 75.97 | O |
| HETATM | 1940 | O3 | MAN | A | 5 | 5.406 | −17.116 | 28.838 | 1.00 | 75.97 | O |
| HETATM | 1941 | O4 | MAN | A | 5 | 8.248 | −17.234 | 28.248 | 1.00 | 75.97 | O |
| HETATM | 1942 | O5 | MAN | A | 5 | 7.162 | −20.729 | 28.007 | 1.00 | 75.97 | O |
| HETATM | 1943 | O6 | MAN | A | 5 | 10.361 | −19.272 | 27.362 | 1.00 | 75.97 | O |
| HETATM | 1944 | O | HOH | | 1 | 11.188 | 8.002 | −3.637 | 1.00 | 57.41 | O |
| HETATM | 1945 | O | HOH | | 2 | −0.416 | −10.450 | 15.145 | 1.00 | 57.41 | O |
| HETATM | 1946 | O | HOH | | 3 | −0.492 | −9.251 | 12.441 | 1.00 | 57.41 | O |
| HETATM | 1947 | O | HOH | | 4 | −7.200 | −4.855 | 27.320 | 1.00 | 57.41 | O |
| HETATM | 1948 | O | HOH | | 5 | 1.904 | −3.678 | 3.473 | 1.00 | 57.41 | O |
| HETATM | 1949 | O | HOH | | 6 | −0.543 | −16.030 | 16.418 | 1.00 | 57.41 | O |
| HETATM | 1950 | O | HOH | | 7 | −17.147 | 0.652 | 5.613 | 1.00 | 57.41 | O |
| HETATM | 1951 | O | HOH | | 8 | −16.533 | 10.658 | 9.845 | 1.00 | 57.41 | O |
| HETATM | 1952 | O | HOH | | 9 | 17.841 | −14.159 | −2.339 | 1.00 | 57.41 | O |
| HETATM | 1953 | O | HOH | | 10 | −13.522 | −1.089 | 9.451 | 1.00 | 63.85 | O |
| HETATM | 1954 | O | HOH | | 11 | −16.163 | 21.987 | 15.522 | 1.00 | 66.61 | O |
| HETATM | 1955 | O | HOH | | 12 | 25.884 | −21.759 | 9.015 | 1.00 | 42.35 | O |
| HETATM | 1956 | O | HOH | | 13 | 10.841 | 17.340 | 13.889 | 1.00 | 79.50 | O |
| HETATM | 1957 | O | HOH | | 14 | −0.184 | 13.888 | 5.045 | 1.00 | 44.33 | O |
| HETATM | 1958 | O | HOH | | 15 | 9.810 | −20.435 | 24.512 | 1.00 | 75.26 | O |
| HETATM | 1959 | O | HOH | | 16 | −2.242 | −0.783 | 28.049 | 1.00 | 45.90 | O |
| HETATM | 1960 | O | HOH | | 17 | −1.601 | −15.062 | 12.117 | 1.00 | 46.17 | O |
| HETATM | 1961 | C1 | FCA | A | 7 | −0.851 | −15.400 | 19.952 | 1.00 | 88.23 | C |
| HETATM | 1962 | C2 | FCA | A | 7 | −1.198 | −15.522 | 21.457 | 1.00 | 88.23 | C |
| HETATM | 1963 | C3 | FCA | A | 7 | −1.707 | −16.987 | 21.710 | 1.00 | 88.23 | C |
| HETATM | 1964 | C4 | FCA | A | 7 | −0.606 | −18.028 | 21.285 | 1.00 | 88.23 | C |
| HETATM | 1965 | C5 | FCA | A | 7 | −0.267 | −17.763 | 19.785 | 1.00 | 88.23 | C |
| HETATM | 1966 | C6 | FCA | A | 7 | 0.811 | −18.713 | 19.204 | 1.00 | 88.23 | C |
| HETATM | 1967 | O2 | FCA | A | 7 | −2.202 | −14.574 | 21.849 | 1.00 | 88.23 | O |
| HETATM | 1968 | O3 | FCA | A | 7 | −2.050 | −17.184 | 23.084 | 1.00 | 88.23 | O |
| HETATM | 1969 | O4 | FCA | A | 7 | 0.575 | −17.816 | 22.092 | 1.00 | 88.23 | O |
| HETATM | 1970 | O5 | FCA | A | 7 | 0.171 | −16.377 | 19.623 | 1.00 | 88.23 | O |

| | | | | |
|---|---|---|---|---|
| CONECT | 35 | 36 | 41 | |
| CONECT | 36 | 35 | 37 | 39 |
| CONECT | 37 | 36 | 38 | |
| CONECT | 38 | 37 | | |
| CONECT | 39 | 36 | 40 | |
| CONECT | 40 | 39 | 41 | 42 |
| CONECT | 41 | 35 | 40 | |
| CONECT | 42 | 40 | | |
| CONECT | 50 | 1874 | | |
| CONECT | 284 | 483 | | |
| CONECT | 483 | 284 | | |
| CONECT | 499 | 1034 | | |
| CONECT | 544 | 593 | | |
| CONECT | 593 | 544 | | |
| CONECT | 1034 | 499 | | |
| CONECT | 1874 | 50 | 1875 | 1885 |
| CONECT | 1875 | 1874 | 1876 | 1882 |
| CONECT | 1876 | 1875 | 1877 | 1883 |
| CONECT | 1877 | 1876 | 1878 | 1884 |
| CONECT | 1878 | 1877 | 1879 | 1885 |
| CONECT | 1879 | 1878 | 1886 | |
| CONECT | 1880 | 1881 | 1882 | 1887 |
| CONECT | 1881 | 1880 | | |
| CONECT | 1882 | 1875 | 1880 | |
| CONECT | 1883 | 1876 | 1961 | |
| CONECT | 1884 | 1877 | 1888 | |
| CONECT | 1885 | 1874 | 1878 | |
| CONECT | 1886 | 1879 | | |
| CONECT | 1887 | 1880 | | |
| CONECT | 1888 | 1884 | 1889 | 1899 |
| CONECT | 1889 | 1888 | 1890 | 1896 |
| CONECT | 1890 | 1889 | 1891 | 1897 |
| CONECT | 1891 | 1890 | 1892 | 1898 |
| CONECT | 1892 | 1891 | 1893 | 1899 |
| CONECT | 1893 | 1892 | 1900 | |
| CONECT | 1894 | 1895 | 1896 | 1901 |
| CONECT | 1895 | 1894 | | |
| CONECT | 1896 | 1889 | 1894 | |
| CONECT | 1897 | 1890 | | |
| CONECT | 1898 | 1891 | 1902 | |
| CONECT | 1899 | 1888 | 1892 | |
| CONECT | 1900 | 1893 | | |
| CONECT | 1901 | 1894 | | |
| CONECT | 1902 | 1898 | 1903 | 1911 |
| CONECT | 1903 | 1902 | 1904 | 1908 |
| CONECT | 1904 | 1903 | 1905 | 1909 |
| CONECT | 1905 | 1904 | 1906 | 1910 |
| CONECT | 1906 | 1905 | 1907 | 1911 |
| CONECT | 1907 | 1906 | 1912 | |
| CONECT | 1908 | 1903 | 1924 | |
| CONECT | 1909 | 1904 | 1913 | |
| CONECT | 1910 | 1905 | | |
| CONECT | 1911 | 1902 | 1906 | |
| CONECT | 1912 | 1907 | 1933 | |
| CONECT | 1913 | 1909 | 1914 | 1922 |
| CONECT | 1914 | 1913 | 1915 | 1919 |
| CONECT | 1915 | 1914 | 1916 | 1920 |
| CONECT | 1916 | 1915 | 1917 | 1921 |
| CONECT | 1917 | 1916 | 1918 | 1922 |
| CONECT | 1918 | 1917 | 1923 | |
| CONECT | 1919 | 1914 | | |
| CONECT | 1920 | 1915 | | |
| CONECT | 1921 | 1916 | | |
| CONECT | 1922 | 1913 | 1917 | |
| CONECT | 1923 | 1918 | | |
| CONECT | 1924 | 1908 | 1925 | 1932 |
| CONECT | 1925 | 1924 | 1926 | 1929 |
| CONECT | 1926 | 1925 | 1927 | 1930 |
| CONECT | 1927 | 1926 | 1928 | 1931 |
| CONECT | 1928 | 1927 | 1932 | |
| CONECT | 1929 | 1925 | | |
| CONECT | 1930 | 1926 | | |
| CONECT | 1931 | 1927 | | |
| CONECT | 1932 | 1924 | 1928 | |
| CONECT | 1933 | 1912 | 1934 | 1942 |
| CONECT | 1934 | 1933 | 1935 | 1939 |
| CONECT | 1935 | 1934 | 1936 | 1940 |
| CONECT | 1936 | 1935 | 1937 | 1941 |
| CONECT | 1937 | 1936 | 1938 | 1942 |
| CONECT | 1938 | 1937 | 1943 | |
| CONECT | 1939 | 1934 | | |
| CONECT | 1940 | 1935 | | |
| CONECT | 1941 | 1936 | | |
| CONECT | 1942 | 1933 | 1937 | |
| CONECT | 1943 | 1938 | | |
| CONECT | 1961 | 1883 | 1962 | 1970 |
| CONECT | 1962 | 1961 | 1963 | 1967 |
| CONECT | 1963 | 1962 | 1964 | 1968 |
| CONECT | 1964 | 1963 | 1965 | 1969 |
| CONECT | 1965 | 1964 | 1966 | 1970 |
| CONECT | 1966 | 1965 | | |
| CONECT | 1967 | 1962 | | |
| CONECT | 1968 | 1963 | | |
| CONECT | 1969 | 1964 | | |
| CONECT | 1970 | 1961 | 1965 | |

MASTER 320 0 8 4 15 0 0 6 1969 1 95 19
END

TABLE 2
Refinement statistics and final model statistics for EXPB1

| | |
|---|---|
| Resolution, Å | 2.75 |
| Cell | |
| a, Å | 113.408 |
| b, Å | 44.512 |
| c, Å | 69.467 |
| β, ° | 124.64 |
| Space group | C 2 |
| Final R_{free}, % | 29.07 |
| Final R, % | 23.32 |
| No. of reflections | 6,640 (working)/367(test) |
| No. of atoms | 1,969 |
| B(iso) 2 of protein atoms, Å | 55.5 |
| Main chain | 53.4 |
| Side chain | 57.6 |
| rmsd in bond lengths, Å | 0.008 |
| rmsd in bond angles, ° | 1.68 |
| Solvent content, % | 53.9 |
| Ramachandran plot, % | |
| Most favored region, % | 91.9 |
| Generously allowed, % | 6.6 |

### Supporting Text

Structure Solution and Refinement. After several cycles of rigid body refinement the maps still looked noisy. To improve this, density modification was performed by using the program CNS (1). Solvent density modification and density truncation features were used. The resulting maps gradually helped in modeling regions of the missing N-terminal residues and the loop between residues 29 and 38. The side chains that were different in Phl p 1 compared to EXPB1 could also be corrected, and the four extra residues at the C terminus could be located. The polysaccharide covalently linked to Asn-10 was modeled as shown in FIG. 2. Only group β-factor refinement was used, given that the resolution of the data was 2.75 Å. After several iterations of modeling using the program 0 (2) and density modification and refinement using the program CNS, the R-factors converged at 23.32% and 29.07%, respectively. At the very end of the refinement a total of nine water molecules could be located. As reported by PROCHECK (3), all residues lie in or close to the allowed regions of the Ramachandran plot. The first three residues at the N terminus are disordered and are not part of the model.
Comparison with Phl p 1 (PDB ID code 1N10). Compared to the 2.9-Å structure of 1N10, which has a dimer in the asymmetric unit, EXPB1, with a monomer in the asymmetric unit, is solved to a better resolution (2.75 Å). The loop consisting of residues 29-38 is not resolved in 1N10 but has good electron density in EXPB1. This is an important loop because it contains D37, a potential candidate for the catalytic base. The first 15 residues at the N-terminal extension are oriented entirely differently in the two structures (leading to successful molecular replacement when omitted). The N-terminal strand in 1N10 extends out away from the protein and interacts with a second monomer. Because the recombinantly produced Phl p 1 used to solve the 1N10 structure was not native protein, the processing of the N-terminal extension appears to be atypical (the hydroxylation of prolines is lacking, and glycosylation pattern at N10 is probably different and was resolved to only one GlcNac).

When the Cα carbon atoms of both D1 and D2 are superimposed for the two proteins, the rmsd is 1.84 Å. Superposition of the D1s alone (excluding the first 15 residues at the N terminus) reveals a good overlap (rmsd of 0.88 Å) whereas the D2s overlap poorly (rmsd of 1.82 Å). Comparison of the overlapping structures shows that the Cα of W194 (D2), which is a crucial part of the putative binding groove, is displaced by 4 Å in 1N10 and its side chain is rotated and displaced by almost 12 Å. However, the tryptophan ring continues to stay in the same plane as the other residues at the base of the groove, and hence a sugar could still bind in a fashion similar to that for EXPB1.
1. Brunger A T, Adams P D, Clore G M, DeLano W L, Gros P, Grosse-Kunstleve R W, Jiang J S, Kuszewski J, Nilges M, Pannu N S, et al. (1998) Acta Crystallogr D 54:905-921.
2. Jones T A, Zou J Y, Cowan S W, Kjeldgaard M (1991) Acta Crystallogr A 47:110-119.
3. Laskowski R A, Macarthur M W, Moss D S, Thornton J M (1993) J Appl Crystallogr 26:283-291.

Expansins have the many conserved domains as shown in FIG. 6. The signal peptide directs the nascent polypeptide into the ER/Golgi secretory pathway. This part of the protein (typically 22-25 amino acids) is removed as the protein enters the ER.

The mature protein is ˜25-27 kDa and consists of two domains, an amino-terminal domain of ˜120 amino acid residues (green in structure) with structural and sequence similarity to family-45 endoglucanases (EG45-like domain) and a carboxy-terminal domain of ˜98 amino acid residues (cyan in structure) that is hypothesized to function as a polysaccharide-binding domain (this is not experimentally established).

FIG. 4 shows is a crystal structure model (PDB code 2HCZ) of a beta-expansin in which the two domains are colored green and cyan. The two domains form a long, open planar surface with conserved polar residues that could hydrogen bond to a polysaccharide and four conserved aromatic residues (red below) that could bind the sugar rings by Van der Waals forces.

What is claimed is:

1. A modified expansin protein comprising:
a protein sequence with 50% or greater homology to SEQ ID NO:1, and which retains the three dimensional polysaccharide biding site created by residues T25, W26, Y27, G28, D37, G40, A41, G43, D95, N97, D107, G129, C156, N157, Y160, S193, W194, R199, and D201 of SEQ ID NO:1; and wherein the protein has expansin activity.

2. The protein of claim 1 further comprising: one or more regions: TWYG, GGACG, HFD within said binding site.

3. The protein of claim 1 further comprising one or more conserved residues selected from the group consisting of T25, D37, D95, D107, N157, S193, and R199.

3. The protein of claim 1 further comprising conserved residues T25, W26, Y27, G28, D37, G40, A41, G43, D95, N97, D107, G129, C156, N157, Y160, S193, W194, R199, and D200 of SEQ ID NO:1.

4. An isolated, purified protein comprising an EXPB1 crystal.

5. The EXPB1 according to claim 1 wherein the protein comprises amino acids T25, W26, Y27, G28, D37, G40, A41, G43, D95, N97, D107, G129, C156, N157, Y160, S193, W194, R199, and D200 of SEQ ID NO:1.

6. The EXPB1 protein according to claim 1 wherein the protein comprises amino acids T25, D37, D95, D107, N157, S193, and R199 of SEQ ID NO:1.

7. A crystal comprising an EXPB1 protein.

8. A crystal comprising an EXPB1 domain 1.

9. The crystal of claim 8 further comprising a amino acids 19-140 of SEQ ID NO:1.

10. A crystal comprising an EXPB1 domain 2.

11. The crystal of claim 10 further comprising amino acids 147-245 of SEQ ID NO:1.

12. A crystal comprising an EXPB1 protein/polysaccharide complex.

13. A crystal comprising an EXPB1 polysaccharide binding domain.

14. A crystallizable composition comprising an:
a) EXPB1 protein;
b) EXPB1 polysaccharide binding domain;
c) polysaccharide associated with said domain; or
d) a complex comprising any of a)-c).

15. A computer comprising:
a) a machine-readable data storage medium, comprising a data storage material encoded with machine-readable data, wherein said data defines an EXPB1 binding domain;
b) a working memory for storing instructions for processing the machine-readable data;
c) a central processing unit coupled to the working memory and to the machine-readable data storage medium for processing the machine-readable data and a means for generating three-dimensional structural information of the binding domain; and
d) output hardware coupled to the central processing unit for outputting three-dimensional structural information of said binding pocket or domain, or information produced using said three-dimensional structural information of the binding domain.

16. The computer according to claim 15, wherein said means for generating three-dimensional structural information is provided by means for generating a three-dimensional graphical representation of said binding domain.

17. The computer according to claim 16, wherein said output hardware is a display terminal, a printer, CD or DVD recorder, ZIP™ or JAZ™ drive, a disk drive, or other machine-readable data storage device.

18. A method of utilizing molecular replacement to obtain structural information about a molecule or a molecular complex of unknown structure, wherein the molecule is sufficiently homologous to EXPB1, comprising the steps of:
a) crystallizing said molecule or molecular complex;
b) generating an X-ray diffraction pattern from said crystallized molecule or molecular complex;
c) applying at least a portion of the structure coordinates set forth herein or a homology model thereof to the X-ray diffraction pattern to generate a three-dimensional electron density map of at least a portion of the molecule or molecular complex of unknown structure; and
d) generating a structural model of the molecule or molecular complex from the three-dimensional electron density map.

19. The method according to claim 18, wherein the molecule is selected from the group consisting of an EXPB1 and an EXPB1 domain.

20. The method according to claim 19, wherein the molecular complex is selected from the group consisting EXPB1 and an EXPB1 domain.

21. A method of identifying an EXPB1 binding compound, comprising the step of using a three-dimensional structural representation of EXPB1 or a fragment thereof comprising a polysaccharide binding site, to computationally screen a candidate compound for an ability to bind the EXPB1/polysaccharide binding site.

22. A composition comprising EXPB1 protein in a crystalline form, wherein said protein crystal is a monoclinic C₂ space group with a crystallographic R-factor of 0.233 and an R-free of 0.291 with crystal unit cell dimensions of a=113.7 Å, b=45.2 Å, and c=70.3 Å, with angles α=90.0°, β=124.6°, and γ=90.0°.

23. The composition according to claim 22 wherein said EXPB1 protein is characterized by a long shallow polysaccharide binding groove with polar and aromatic residues conserved from SEQ ID NO:1.

24. The composition according to claim 23 wherein said aromatic and polar residues are selected from the group consisting of: T25, D37, D95, D107, N157, S193, and R199.

25. The composition according to claim 22 wherein said protein comprises amino acids T25, W26, Y27, G28, D37, G40, A41, G43, D95, N97, D107, G129, C156, N157, Y160, S193, W194, R199, and D201 of SEQ ID NO:1.

26. A process of identifying an agonist or an antagonist of EXPB1 selected from the group consisting of: a peptide, a non-peptide and a small molecule; wherein said agonist or antagonist is capable of enhancing, eliciting or blocking the interaction between human EXPB1 and polysaccharides; wherein said process comprises:
a) crystallizing the composition of claim 22 and determining the three-dimensional structural coordinates defined in PDB accession #2HCZ;
b) introducing into a suitable computer program, said three-dimensional structural coordinates and having the program display said coordinates;
c) creating a three-dimensional model of a test compound in said computer program;
d) displaying and superimposing the model of said test compound onto the three-dimensional structural coordinates of the EXPB1 protein;
e) assessing whether said test compound model is capable of affecting the interaction between EXPB1 and its polysaccharide; and
f) incorporating said test compound in an EXPB1 activity assay and determining whether said test compound inhibits or enhances the biological activity of EXPB1 wherein said compounds are identified as agonists or antagonists.

27. A process of identifying an agonist or an antagonist capable of modifying the activity of the composition of claim 22, wherein said process comprises: carrying out an in vitro assay by introducing said compound into an expansin activity assay mixture; and determining whether said test compound inhibits or enhances the activity of EXPB1 mediated cell wall extension, wherein said compounds are identified as agonists or antagonists.
